SEQUENCE LISTING (505 sequences)

SEQ ID NO: 1 - DNA, 61 bp, Artificial Sequence
  Description of Artificial Sequence Synthetic oligonucleotide
cttctggcaattgaagaaaaaaaattgagcagctgtaactgcatgcacattatgcaaattt

SEQ ID NO: 2 - DNA, 61 bp, Artificial Sequence
  Description of Artificial Sequence Synthetic oligonucleotide
aaatttgcataatgtgcatgcagttacagctgctcaattttttttcttcaattgccagaag

SEQ ID NO: 3 - DNA, 61 bp, Artificial Sequence
  Description of Artificial Sequence Synthetic oligonucleotide
cttctggcaattgaagaaaaaaaattgagcggctgtaactgcatgcacattatgcaaattt

SEQ ID NO: 4 - DNA, 61 bp, Artificial Sequence
  Description of Artificial Sequence Synthetic oligonucleotide
aaatttgcataatgtgcatgcagttacagccgctcaattttttttcttcaattgccagaag

SEQ ID NO: 5 - DNA, 61 bp, Artificial Sequence
  Description of Artificial Sequence Synthetic oligonucleotide
tgcagcacaagggctggcacacagcaggccgccatattcatgtgctgttctgccagacgtt

SEQ ID NO: 6 - DNA, 61 bp, Artificial Sequence
  Description of Artificial Sequence Synthetic oligonucleotide
aacgtctggcagaacagcacatgaatatggcggcctgctgtgtgccagcccttgtgctgca

SEQ ID NO: 7 - DNA, 61 bp, Artificial Sequence
  Description of Artificial Sequence Synthetic oligonucleotide
tgcagcacaagggctggcacacagcaggcctccatattcatgtgctgttctgccagacgtt

SEQ ID NO: 8 - DNA, 61 bp, Artificial Sequence
  Description of Artificial Sequence Synthetic oligonucleotide
aacgtctggcagaacagcacatgaatatggaggcctgctgtgtgccagcccttgtgctgca

SEQ ID NO: 9 - DNA, 61 bp, Artificial Sequence
  Description of Artificial Sequence Synthetic oligonucleotide
ggaggcctcggtgaagggcatgctgggacgactcactagcacattgggtggctcagcttcc

SEQ ID NO: 10 - DNA, 61 bp, Artificial Sequence
  Description of Artificial Sequence Synthetic oligonucleotide
ggaagctgagccacccaatgtgctagtgagtcgtcccagcatgcccttcaccgaggcctcc

SEQ ID NO: 11 - DNA, 61 bp, Artificial Sequence
  Description of Artificial Sequence Synthetic oligonucleotide
ggaggcctcggtgaagggcatgctgggacggctcactagcacattgggtggctcagcttcc

SEQ ID NO: 12 - DNA, 61 bp, Artificial Sequence
  Description of Artificial Sequence Synthetic oligonucleotide
ggaagctgagccacccaatgtgctagtgagccgtcccagcatgcccttcaccgaggcctcc

SEQ ID NO: 13 - DNA, 56 bp, Artificial Sequence
  Description of Artificial Sequence Synthetic oligonucleotide
agtatagccccagcgtgtctacgagcttctggcaattgaagaaaaaaaattgagca

SEQ ID NO: 14 - DNA, 56 bp, Artificial Sequence
  Description of Artificial Sequence Synthetic oligonucleotide
agtatagccccagcgtgtctacgagcttctggcaattgaagaaaaaaaattgagcg

SEQ ID NO: 15 - DNA, 56 bp, Artificial Sequence
  Description of Artificial Sequence Synthetic oligonucleotide
aatcgctactgtcgcaaggggtcctcttctggcaattgaagaaaaaaaattgagca

SEQ ID NO: 16 - DNA, 56 bp, Artificial Sequence
  Description of Artificial Sequence Synthetic oligonucleotide
aatcgctactgtcgcaaggggtcctcttctggcaattgaagaaaaaaaattgagcg

SEQ ID NO: 17 - DNA, 56 bp, Artificial Sequence
  Description of Artificial Sequence Synthetic oligonucleotide
ggctgtagtcataccatagtgcatccttctggcaattgaagaaaaaaaattgagca

SEQ ID NO: 18 - DNA, 56 bp, Artificial Sequence
  Description of Artificial Sequence Synthetic oligonucleotide
ggctgtagtcataccatagtgcatccttctggcaattgaagaaaaaaaattgagcg

SEQ ID NO: 19 - DNA, 56 bp, Artificial Sequence
  Description of Artificial Sequence Synthetic oligonucleotide
agtatagccccagcgtgtctacgagtgcagcacaagggctggcacacagcaggccg

SEQ ID NO: 20 - DNA, 56 bp, Artificial Sequence
  Description of Artificial Sequence Synthetic oligonucleotide
agtatagccccagcgtgtctacgagtgcagcacaagggctggcacacagcaggcct

SEQ ID NO: 21 - DNA, 56 bp, Artificial Sequence
  Description of Artificial Sequence Synthetic oligonucleotide
aatcgctactgtcgcaaggggtccttgcagcacaagggctggcacacagcaggccg

SEQ ID NO: 22 - DNA, 56 bp, Artificial Sequence
  Description of Artificial Sequence Synthetic oligonucleotide
aatcgctactgtcgcaaggggtccttgcagcacaagggctggcacacagcaggcct

SEQ ID NO: 23 - DNA, 56 bp, Artificial Sequence
  Description of Artificial Sequence Synthetic oligonucleotide
ggctgtagtcataccatagtgcatctgcagcacaagggctggcacacagcaggccg

SEQ ID NO: 24 - DNA, 56 bp, Artificial Sequence
  Description of Artificial Sequence Synthetic oligonucleotide
ggctgtagtcataccatagtgcatctgcagcacaagggctggcacacagcaggcct

SEQ ID NO: 25 - DNA, 50 bp, Artificial Sequence
  Description of Artificial Sequence Synthetic oligonucleotide
agtatagccccagcgtgtctacgagctcggtgaagggcatgctgggacga

SEQ ID NO: 26 - DNA, 50 bp, Artificial Sequence
  Description of Artificial Sequence Synthetic oligonucleotide
agtatagccccagcgtgtctacgagctcggtgaagggcatgctgggacgg

SEQ ID NO: 27 - DNA, 50 bp, Artificial Sequence
  Description of Artificial Sequence Synthetic oligonucleotide
aatcgctactgtcgcaaggggtcctctcggtgaagggcatgctgggacga

SEQ ID NO: 28 - DNA, 50 bp, Artificial Sequence
  Description of Artificial Sequence Synthetic oligonucleotide
aatcgctactgtcgcaaggggtcctctcggtgaagggcatgctgggacgg

SEQ ID NO: 29 - DNA, 50 bp, Artificial Sequence
  Description of Artificial Sequence Synthetic oligonucleotide
ggctgtagtcataccatagtgcatcctcggtgaagggcatgctgggacga

SEQ ID NO: 30 - DNA, 50 bp, Artificial Sequence
  Description of Artificial Sequence Synthetic oligonucleotide
ggctgtagtcataccatagtgcatcctcggtgaagggcatgctgggacgg

SEQ ID NO: 31 - DNA, 55 bp, Artificial Sequence
  Description of Artificial Sequence Synthetic oligonucleotide
gctgtaactgcatgcacattatgcaaatttttccagctatcctgtaaggcaacgt

SEQ ID NO: 32 - DNA, 55 bp, Artificial Sequence
  Description of Artificial Sequence Synthetic oligonucleotide
ccatattcatgtgctgttctgccagacgttttccagctatcctgtaaggcaacgt

SEQ ID NO: 33 - DNA, 55 bp, Artificial Sequence
  Description of Artificial Sequence Synthetic oligonucleotide
ctcactagcacattgggtggctcagcttccttccagctatcctgtaaggcaacgt

SEQ ID NO: 34 - DNA, 25 bp, Artificial Sequence
  Description of Artificial Sequence Synthetic primer
agtatagccccagcgtgtctacgag

SEQ ID NO: 35 - DNA, 25 bp, Artificial Sequence
  Description of Artificial Sequence Synthetic primer
aatcgctactgtcgcaaggggtcct

SEQ ID NO: 36 - DNA, 25 bp, Artificial Sequence
  Description of Artificial Sequence Synthetic primer
ggctgtagtcataccatagtgcatc

SEQ ID NO: 37 - DNA, 25 bp, Artificial Sequence
  Description of Artificial Sequence Synthetic primer
acgttgccttacaggatagctggaa

SEQ ID NO: 38 - DNA, 25 bp, Artificial Sequence
  Description of Artificial Sequence Synthetic primer
actctgcgctctggaacttaccgga

SEQ ID NO: 39 - DNA, 25 bp, Artificial Sequence
  Description of Artificial Sequence Synthetic primer
gatcttggacgaagtcgtcctatga

SEQ ID NO: 40 - DNA, 25 bp, Artificial Sequence
  Description of Artificial Sequence Synthetic primer
gtttgcatgaagacctctatacaga

SEQ ID NO: 41 - DNA, 25 bp, Artificial Sequence
  Description of Artificial Sequence Synthetic primer
ctgaaaggtccatggcctgtactga

SEQ ID NO: 42 - DNA, 25 bp, Artificial Sequence
  Description of Artificial Sequence Synthetic primer
ttgtatcgatgcagccaggatccga

SEQ ID NO: 43 - DNA, 25 bp, Artificial Sequence
  Description of Artificial Sequence Synthetic primer
cacagaattagcgatctatgccgga

SEQ ID NO: 44 - DNA, 25 bp, Artificial Sequence
  Description of Artificial Sequence Synthetic primer
cacgcctcatcgtagtgtaggagga

SEQ ID NO: 45 - DNA, 25 bp, Artificial Sequence
  Description of Artificial Sequence Synthetic primer
ataacattgaacgctgccgttgcga

SEQ ID NO: 46 - DNA, 25 bp, Artificial Sequence
  Description of Artificial Sequence Synthetic primer
cagactacggcaatataacgctgga

SEQ ID NO: 47 - DNA, 25 bp, Artificial Sequence
  Description of Artificial Sequence Synthetic primer
acgtaactattacggtgagcgccga

SEQ ID NO: 48 - DNA, 25 bp, Artificial Sequence
  Description of Artificial Sequence Synthetic primer
atggatagccgctgtttaactacga

SEQ ID NO: 49 - DNA, 25 bp, Artificial Sequence
  Description of Artificial Sequence Synthetic primer
ttcggcttccacagagcaaggtaga

SEQ ID NO: 50 - DNA, 25 bp, Artificial Sequence
  Description of Artificial Sequence Synthetic primer
tgtatcagcatctggctcagcgtct

SEQ ID NO: 51 - DNA, 25 bp, Artificial Sequence
  Description of Artificial Sequence Synthetic primer
ctttggggtaagcgaccatcagcct

SEQ ID NO: 52 - DNA, 25 bp, Artificial Sequence
  Description of Artificial Sequence Synthetic primer
tacatagaatctaccgtggtgacct

SEQ ID NO: 53 - DNA, 25 bp, Artificial Sequence
  Description of Artificial Sequence Synthetic primer
acgatggcgttgcaggcgcttacct

SEQ ID NO: 54 - DNA, 25 bp, Artificial Sequence
  Description of Artificial Sequence Synthetic primer
ttgactgagactcctcatgacctct

SEQ ID NO: 55 - DNA, 25 bp, Artificial Sequence
  Description of Artificial Sequence Synthetic primer
gccgtttcatatcgaacaaggcgct

SEQ ID NO: 56 - DNA, 25 bp, Artificial Sequence
  Description of Artificial Sequence Synthetic primer
gggctactcgcaatttcaaattgct

SEQ ID NO: 57 - DNA, 25 bp, Artificial Sequence
  Description of Artificial Sequence Synthetic primer
cgccagcaatcagctttgatacact

SEQ ID NO: 58 - DNA, 51 bp, Artificial Sequence
  Description of Artificial Sequence Synthetic oligonucleotide
tgtatcagcatctggctcagcgtctgctttcattcatatctgcaggttcaa

SEQ ID NO: 59 - DNA, 51 bp, Artificial Sequence
  Description of Artificial Sequence Synthetic oligonucleotide
tacatagaatctaccgtggtgacctgcagtccagagcaccgtggtcctgct

SEQ ID NO: 60 - DNA, 51 bp, Artificial Sequence
  Description of Artificial Sequence Synthetic oligonucleotide
ttgactgagactcctcatgacctctcaccactggggtaaggttttctaggg

SEQ ID NO: 61 - DNA, 51 bp, Artificial Sequence
  Description of Artificial Sequence Synthetic oligonucleotide
gggctactcgcaatttcaaattgctagccagttttccatgggttctactac

SEQ ID NO: 62 - DNA, 51 bp, Artificial Sequence
  Description of Artificial Sequence Synthetic oligonucleotide
tgtatcagcatctggctcagcgtctcttcccggtcagctactcctcttccg

SEQ ID NO: 63 - DNA, 51 bp, Artificial Sequence
  Description of Artificial Sequence Synthetic oligonucleotide
tacatagaatctaccgtggtgaccttgatccattagattcaaatgtagcaa

SEQ ID NO: 64 - DNA, 51 bp, Artificial Sequence
  Description of Artificial Sequence Synthetic oligonucleotide
ttgactgagactcctcatgacctctacctgctggtgccactctggaaaggc

SEQ ID NO: 65 - DNA, 51 bp, Artificial Sequence
  Description of Artificial Sequence Synthetic oligonucleotide
gggctactcgcaatttcaaattgctcttgctgcttccagtaaataaggtga

SEQ ID NO: 66 - DNA, 51 bp, Artificial Sequence
  Description of Artificial Sequence Synthetic oligonucleotide
tgtatcagcatctggctcagcgtctatccttgtccaaggaggctgtttctg

SEQ ID NO: 67 - DNA, 51 bp, Artificial Sequence
  Description of Artificial Sequence Synthetic oligonucleotide
tacatagaatctaccgtggtgaccttccacacgcaaatttccttccactcg

SEQ ID NO: 68 - DNA, 51 bp, Artificial Sequence
  Description of Artificial Sequence Synthetic oligonucleotide
ttgactgagactcctcatgacctctaggagctgctggtgcaggggccacgg

SEQ ID NO: 69 - DNA, 51 bp, Artificial Sequence
  Description of Artificial Sequence Synthetic oligonucleotide
gggctactcgcaatttcaaattgctattcatcggacatgttactgtttttc

SEQ ID NO: 70 - DNA, 51 bp, Artificial Sequence
  Description of Artificial Sequence Synthetic oligonucleotide
tgtatcagcatctggctcagcgtctgtgtcatcaacttggtccacagtcgt

SEQ ID NO: 71 - DNA, 51 bp, Artificial Sequence
  Description of Artificial Sequence Synthetic oligonucleotide
tacatagaatctaccgtggtgacctggccctctagggactcgaacagagat

SEQ ID NO: 72 - DNA, 51 bp, Artificial Sequence
  Description of Artificial Sequence Synthetic oligonucleotide
ttgactgagactcctcatgacctctcagagggaggacgagctgaccttcat

SEQ ID NO: 73 - DNA, 51 bp, Artificial Sequence
  Description of Artificial Sequence Synthetic oligonucleotide
gggctactcgcaatttcaaattgctccaactcgaaattccccgtgaccaga

SEQ ID NO: 74 - DNA, 51 bp, Artificial Sequence
  Description of Artificial Sequence Synthetic oligonucleotide
tgtatcagcatctggctcagcgtctaccagcagatactcagccggaggata

SEQ ID NO: 75 - DNA, 51 bp, Artificial Sequence
  Description of Artificial Sequence Synthetic oligonucleotide
tacatagaatctaccgtggtgacctgaggaccccaagtcccatagggaccc

SEQ ID NO: 76 - DNA, 51 bp, Artificial Sequence
  Description of Artificial Sequence Synthetic oligonucleotide
ttgactgagactcctcatgacctctgcagcgcaccacgggacccaagcccg

SEQ ID NO: 77 - DNA, 51 bp, Artificial Sequence
  Description of Artificial Sequence Synthetic oligonucleotide
gggctactcgcaatttcaaattgctcagagtctgaggtagctgccctggca

SEQ ID NO: 78 - DNA, 51 bp, Artificial Sequence
  Description of Artificial Sequence Synthetic oligonucleotide
tgtatcagcatctggctcagcgtcttgctgttttcttccttcaggcataca

SEQ ID NO: 79 - DNA, 51 bp, Artificial Sequence
  Description of Artificial Sequence Synthetic oligonucleotide
tacatagaatctaccgtggtgacctcctgaacagctcgcggctcagcaggg

SEQ ID NO: 80 - DNA, 51 bp, Artificial Sequence
  Description of Artificial Sequence Synthetic oligonucleotide
ttgactgagactcctcatgacctctatcttcaaagttgcagtaaaaaccca

SEQ ID NO: 81 - DNA, 51 bp, Artificial Sequence
  Description of Artificial Sequence Synthetic oligonucleotide
gggctactcgcaatttcaaattgctcttgattcatgatattttactccaag

SEQ ID NO: 82 - DNA, 51 bp, Artificial Sequence
  Description of Artificial Sequence Synthetic oligonucleotide
tgtatcagcatctggctcagcgtcttccctcattgcactgtactcctcttg

SEQ ID NO: 83 - DNA, 51 bp, Artificial Sequence
  Description of Artificial Sequence Synthetic oligonucleotide
tacatagaatctaccgtggtgacctgtccgaggacaacgatgaggcggcgc

SEQ ID NO: 84 - DNA, 51 bp, Artificial Sequence
  Description of Artificial Sequence Synthetic oligonucleotide
ttgactgagactcctcatgacctctttccgcctggtgttggaagagacagg

SEQ ID NO: 85 - DNA, 51 bp, Artificial Sequence
  Description of Artificial Sequence Synthetic oligonucleotide
gggctactcgcaatttcaaattgcttggtctttcagtgcctccactatgac

SEQ ID NO: 86 - DNA, 51 bp, Artificial Sequence
  Description of Artificial Sequence Synthetic oligonucleotide
tgtatcagcatctggctcagcgtcttgaagagaaatataagaaggctatgg

SEQ ID NO: 87 - DNA, 51 bp, Artificial Sequence
  Description of Artificial Sequence Synthetic oligonucleotide
tacatagaatctaccgtggtgacctcctccaggtgcaggagttcatgctca

SEQ ID NO: 88 - DNA, 51 bp, Artificial Sequence
  Description of Artificial Sequence Synthetic oligonucleotide
ttgactgagactcctcatgacctctaaaggcaatgtgggatcctgaattgc

SEQ ID NO: 89 - DNA, 51 bp, Artificial Sequence
  Description of Artificial Sequence Synthetic oligonucleotide
gggctactcgcaatttcaaattgctgcccgaacagccgctggatatgggac

SEQ ID NO: 90 - DNA, 51 bp, Artificial Sequence
  Description of Artificial Sequence Synthetic oligonucleotide
tgtatcagcatctggctcagcgtctctaaaaaggaccctgaaggttgtgac

SEQ ID NO: 91 - DNA, 51 bp, Artificial Sequence
  Description of Artificial Sequence Synthetic oligonucleotide
tacatagaatctaccgtggtgaccttcatatggatgataatgatggagaac

SEQ ID NO: 92 - DNA, 51 bp, Artificial Sequence
  Description of Artificial Sequence Synthetic oligonucleotide
ttgactgagactcctcatgacctctcttggctgtgctcctgctgctggccg

SEQ ID NO: 93 - DNA, 51 bp, Artificial Sequence
  Description of Artificial Sequence Synthetic oligonucleotide
gggctactcgcaatttcaaattgctatcaactataggttgctttggtggtg

SEQ ID NO: 94 - DNA, 51 bp, Artificial Sequence
  Description of Artificial Sequence Synthetic oligonucleotide
tgtatcagcatctggctcagcgtctaaatttctgaataactgaagttggtc

SEQ ID NO: 95 - DNA, 51 bp, Artificial Sequence
  Description of Artificial Sequence Synthetic oligonucleotide
tacatagaatctaccgtggtgacctggtagcagacaaacctgtggttgatc

SEQ ID NO: 96 - DNA, 51 bp, Artificial Sequence
  Description of Artificial Sequence Synthetic oligonucleotide
ttgactgagactcctcatgacctctgagctttgggttgttccttaggaccc

SEQ ID NO: 97 - DNA, 51 bp, Artificial Sequence
  Description of Artificial Sequence Synthetic oligonucleotide
gggctactcgcaatttcaaattgctggaattacggcagcccttctttccca

SEQ ID NO: 98 - DNA, 51 bp, Artificial Sequence
  Description of Artificial Sequence Synthetic oligonucleotide
tgtatcagcatctggctcagcgtctcggggcctctgcttggatgtgatgac

SEQ ID NO: 99 - DNA, 51 bp, Artificial Sequence
  Description of Artificial Sequence Synthetic oligonucleotide
tacatagaatctaccgtggtgacctggcggccgtggtggcggcagtggtgg

SEQ ID NO: 100 - DNA, 51 bp, Artificial Sequence
  Description of Artificial Sequence Synthetic oligonucleotide
ttgactgagactcctcatgacctctgcagaagtcatatttaggatgtgtac

SEQ ID NO: 101 - DNA, 51 bp, Artificial Sequence
  Description of Artificial Sequence Synthetic oligonucleotide
gggctactcgcaatttcaaattgctggactttttttccaaggctattcagt

SEQ ID NO: 102 - DNA, 51 bp, Artificial Sequence
  Description of Artificial Sequence Synthetic oligonucleotide
tgtatcagcatctggctcagcgtctgagatgtgtaagcgcagccttgagtc

SEQ ID NO: 103 - DNA, 51 bp, Artificial Sequence
  Description of Artificial Sequence Synthetic oligonucleotide
tacatagaatctaccgtggtgacctatttatgctatacatgatgaaacatc

SEQ ID NO: 104 - DNA, 51 bp, Artificial Sequence
  Description of Artificial Sequence Synthetic oligonucleotide
ttgactgagactcctcatgacctctgaaattgatagaagcagaagatcggc

SEQ ID NO: 105 - DNA, 51 bp, Artificial Sequence
  Description of Artificial Sequence Synthetic oligonucleotide
gggctactcgcaatttcaaattgctctcacctcccatgttgctcaaagaac

SEQ ID NO: 106 - DNA, 51 bp, Artificial Sequence
  Description of Artificial Sequence Synthetic oligonucleotide
tgtatcagcatctggctcagcgtctagcattctctgcagtacatcaaccgt

SEQ ID NO: 107 - DNA, 51 bp, Artificial Sequence
  Description of Artificial Sequence Synthetic oligonucleotide
tacatagaatctaccgtggtgacctactttactcacgtttttcccatctag

SEQ ID NO: 108 - DNA, 51 bp, Artificial Sequence
  Description of Artificial Sequence Synthetic oligonucleotide
ttgactgagactcctcatgacctctggcatggtggtggatgtagtggtggt

SEQ ID NO: 109 - DNA, 51 bp, Artificial Sequence
  Description of Artificial Sequence Synthetic oligonucleotide
gggctactcgcaatttcaaattgctatggtggtggatgtagtggtggtgga

SEQ ID NO: 110 - DNA, 51 bp, Artificial Sequence
  Description of Artificial Sequence Synthetic oligonucleotide
tgtatcagcatctggctcagcgtctaacatgagttttttatggcgggaggt

SEQ ID NO: 111 - DNA, 51 bp, Artificial Sequence
  Description of Artificial Sequence Synthetic oligonucleotide
tacatagaatctaccgtggtgacctggacaccggcaaggccaccctgacct

SEQ ID NO: 112 - DNA, 51 bp, Artificial Sequence
  Description of Artificial Sequence Synthetic oligonucleotide
ttgactgagactcctcatgacctctctgacccactcatcccaagacacacc

SEQ ID NO: 113 - DNA, 51 bp, Artificial Sequence
  Description of Artificial Sequence Synthetic oligonucleotide
gggctactcgcaatttcaaattgctgaaagtaacagcttgactatatccac

SEQ ID NO: 114 - DNA, 51 bp, Artificial Sequence
  Description of Artificial Sequence Synthetic oligonucleotide
tgtatcagcatctggctcagcgtctgtgtcctggaatggggcccatgagat

SEQ ID NO: 115 - DNA, 51 bp, Artificial Sequence
  Description of Artificial Sequence Synthetic oligonucleotide
tacatagaatctaccgtggtgaccttggaatttcctcctcgagtctgaacc

SEQ ID NO: 116 - DNA, 51 bp, Artificial Sequence
  Description of Artificial Sequence Synthetic oligonucleotide
ttgactgagactcctcatgacctctttccctccagccccaggttacccctg

SEQ ID NO: 117 - DNA, 51 bp, Artificial Sequence
  Description of Artificial Sequence Synthetic oligonucleotide
gggctactcgcaatttcaaattgctcggggggtcttggatgtgccggcttg

SEQ ID NO: 118 - DNA, 51 bp, Artificial Sequence
  Description of Artificial Sequence Synthetic oligonucleotide
tgtatcagcatctggctcagcgtcttggcctgttggccgtatctgctaaca

SEQ ID NO: 119 - DNA, 51 bp, Artificial Sequence
  Description of Artificial Sequence Synthetic oligonucleotide
tacatagaatctaccgtggtgacctctgttttgttccgaatgtctgaggac

SEQ ID NO: 120 - DNA, 51 bp, Artificial Sequence
  Description of Artificial Sequence Synthetic oligonucleotide
ttgactgagactcctcatgacctctgtgtcaacaattctaaggaggaagat

SEQ ID NO: 121 - DNA, 51 bp, Artificial Sequence
  Description of Artificial Sequence Synthetic oligonucleotide
gggctactcgcaatttcaaattgctgagatccagatgttttggaatattac

SEQ ID NO: 122 - DNA, 51 bp, Artificial Sequence
  Description of Artificial Sequence Synthetic oligonucleotide
tgtatcagcatctggctcagcgtcttggactgtgtatgaaacctggtttta

SEQ ID NO: 123 - DNA, 51 bp, Artificial Sequence
  Description of Artificial Sequence Synthetic oligonucleotide
tacatagaatctaccgtggtgacctcaatctttttaaccattttgtcatcg

SEQ ID NO: 124 - DNA, 51 bp, Artificial Sequence
  Description of Artificial Sequence Synthetic oligonucleotide
ttgactgagactcctcatgacctctccaggggagaaaagtacattggaaac

SEQ ID NO: 125 - DNA, 51 bp, Artificial Sequence
  Description of Artificial Sequence Synthetic oligonucleotide
gggctactcgcaatttcaaattgctctttattcaggtggatgcccctgacc

SEQ ID NO: 126 - DNA, 51 bp, Artificial Sequence
  Description of Artificial Sequence Synthetic oligonucleotide
tgtatcagcatctggctcagcgtctcaacatctcttttccctggaagtttc

SEQ ID NO: 127 - DNA, 51 bp, Artificial Sequence
  Description of Artificial Sequence Synthetic oligonucleotide
tacatagaatctaccgtggtgacctggacatggatcttgtttttctctttg

SEQ ID NO: 128 - DNA, 51 bp, Artificial Sequence
  Description of Artificial Sequence Synthetic oligonucleotide
ttgactgagactcctcatgacctctctcaatctgtagtgctcctggtcggc

SEQ ID NO: 129 - DNA, 51 bp, Artificial Sequence
  Description of Artificial Sequence Synthetic oligonucleotide
gggctactcgcaatttcaaattgctgttttttcaggaggccatctttctcc

SEQ ID NO: 130 - DNA, 51 bp, Artificial Sequence
  Description of Artificial Sequence Synthetic oligonucleotide
tgtatcagcatctggctcagcgtctagaatgagcctgttctgttgacattg

SEQ ID NO: 131 - DNA, 51 bp, Artificial Sequence
  Description of Artificial Sequence Synthetic oligonucleotide
tacatagaatctaccgtggtgacctcaggggagggtgtgggcaggcggttc

SEQ ID NO: 132 - DNA, 51 bp, Artificial Sequence
  Description of Artificial Sequence Synthetic oligonucleotide
ttgactgagactcctcatgacctctgactattcagacatcaatgaggtggc

SEQ ID NO: 133 - DNA, 51 bp, Artificial Sequence
  Description of Artificial Sequence Synthetic oligonucleotide
gggctactcgcaatttcaaattgctctgttcctctacagggccaaaacact

SEQ ID NO: 134 - DNA, 51 bp, Artificial Sequence
  Description of Artificial Sequence Synthetic oligonucleotide
tgtatcagcatctggctcagcgtctgatgatactcactgtccatcagcctc

SEQ ID NO: 135 - DNA, 51 bp, Artificial Sequence
  Description of Artificial Sequence Synthetic oligonucleotide
tacatagaatctaccgtggtgacctagtatcctcacctgtagccaggtatc

SEQ ID NO: 136 - DNA, 51 bp, Artificial Sequence
  Description of Artificial Sequence Synthetic oligonucleotide
ttgactgagactcctcatgacctctgttaattcagcatccagcaggtccct

SEQ ID NO: 137 - DNA, 51 bp, Artificial Sequence
  Description of Artificial Sequence Synthetic oligonucleotide
gggctactcgcaatttcaaattgctacaccaacattcccagctgctggaac

SEQ ID NO: 138 - DNA, 51 bp, Artificial Sequence
  Description of Artificial Sequence Synthetic oligonucleotide
tgtatcagcatctggctcagcgtctcctgggccaggtgtgcatcaaagcgc

SEQ ID NO: 139 - DNA, 51 bp, Artificial Sequence
  Description of Artificial Sequence Synthetic oligonucleotide
tacatagaatctaccgtggtgacctgtgtcaagctactctcaggactgctc

SEQ ID NO: 140 - DNA, 51 bp, Artificial Sequence
  Description of Artificial Sequence Synthetic oligonucleotide
ttgactgagactcctcatgacctctcaaggtgccaggtgcaagacccacca

SEQ ID NO: 141 - DNA, 51 bp, Artificial Sequence
  Description of Artificial Sequence Synthetic oligonucleotide
gggctactcgcaatttcaaattgcttgtcgcgatgaatgtgaaatcctgga

SEQ ID NO: 142 - DNA, 51 bp, Artificial Sequence
  Description of Artificial Sequence Synthetic oligonucleotide
tgtatcagcatctggctcagcgtcttccacaaactcgtcactcatcctccg

SEQ ID NO: 143 - DNA, 51 bp, Artificial Sequence
  Description of Artificial Sequence Synthetic oligonucleotide
tacatagaatctaccgtggtgacctagacatggaagccagtgattatgagc

SEQ ID NO: 144 - DNA, 51 bp, Artificial Sequence
  Description of Artificial Sequence Synthetic oligonucleotide
ttgactgagactcctcatgacctctccacagccaggcagtctgtatcttgc

SEQ ID NO: 145 - DNA, 51 bp, Artificial Sequence
  Description of Artificial Sequence Synthetic oligonucleotide
gggctactcgcaatttcaaattgctatatgtggaggcccaacaaaagagac

SEQ ID NO: 146 - DNA, 51 bp, Artificial Sequence
  Description of Artificial Sequence Synthetic oligonucleotide
tgtatcagcatctggctcagcgtcttggaagttgcgtattgtaagctattc

SEQ ID NO: 147 - DNA, 51 bp, Artificial Sequence
  Description of Artificial Sequence Synthetic oligonucleotide
tacatagaatctaccgtggtgacctggtcagaacaggagtgcacggatagc

SEQ ID NO: 148 - DNA, 51 bp, Artificial Sequence
  Description of Artificial Sequence Synthetic oligonucleotide
ttgactgagactcctcatgacctcttgctttcaatcccaaattatgtgttt

SEQ ID NO: 149 - DNA, 51 bp, Artificial Sequence
  Description of Artificial Sequence Synthetic oligonucleotide
gggctactcgcaatttcaaattgctcatcagtgtgtctgaacatgtggtcc

SEQ ID NO: 150 - DNA, 51 bp, Artificial Sequence
  Description of Artificial Sequence Synthetic oligonucleotide
tgtatcagcatctggctcagcgtctctgccagcctgccctggaggaagaca

SEQ ID NO: 151 - DNA, 51 bp, Artificial Sequence
  Description of Artificial Sequence Synthetic oligonucleotide
tacatagaatctaccgtggtgacctactggaactatctgtaatactggaac

SEQ ID NO: 152 - DNA, 51 bp, Artificial Sequence
  Description of Artificial Sequence Synthetic oligonucleotide
ttgactgagactcctcatgacctctaactctttcacttttacatattaaag

SEQ ID NO: 153 - DNA, 51 bp, Artificial Sequence
  Description of Artificial Sequence Synthetic oligonucleotide
gggctactcgcaatttcaaattgctgcagccagagtggttttttcagggga

SEQ ID NO: 154 - DNA, 51 bp, Artificial Sequence
  Description of Artificial Sequence Synthetic oligonucleotide
ctttggggtaagcgaccatcagcctgctttcattcatatctgcaggttcag

SEQ ID NO: 155 - DNA, 51 bp, Artificial Sequence
  Description of Artificial Sequence Synthetic oligonucleotide
acgatggcgttgcaggcgcttacctgcagtccagagcaccgtggtcctgcc

SEQ ID NO: 156 - DNA, 51 bp, Artificial Sequence
  Description of Artificial Sequence Synthetic oligonucleotide
gccgtttcatatcgaacaaggcgctcaccactggggtaaggttttctagga

SEQ ID NO: 157 - DNA, 51 bp, Artificial Sequence
  Description of Artificial Sequence Synthetic oligonucleotide
cgccagcaatcagctttgatacactagccagttttccatgggttctactat

SEQ ID NO: 158 - DNA, 51 bp, Artificial Sequence
  Description of Artificial Sequence Synthetic oligonucleotide
ctttggggtaagcgaccatcagcctcttcccggtcagctactcctcttcca

SEQ ID NO: 159 - DNA, 51 bp, Artificial Sequence
  Description of Artificial Sequence Synthetic oligonucleotide
acgatggcgttgcaggcgcttaccttgatccattagattcaaatgtagcac

SEQ ID NO: 160 - DNA, 51 bp, Artificial Sequence
  Description of Artificial Sequence Synthetic oligonucleotide
gccgtttcatatcgaacaaggcgctacctgctggtgccactctggaaaggg

SEQ ID NO: 161 - DNA, 51 bp, Artificial Sequence
  Description of Artificial Sequence Synthetic oligonucleotide
cgccagcaatcagctttgatacactcttgctgcttccagtaaataaggtgg

SEQ ID NO: 162 - DNA, 51 bp, Artificial Sequence
  Description of Artificial Sequence Synthetic oligonucleotide
ctttggggtaagcgaccatcagcctatccttgtccaaggaggctgtttcta

SEQ ID NO: 163 - DNA, 51 bp, Artificial Sequence
  Description of Artificial Sequence Synthetic oligonucleotide
acgatggcgttgcaggcgcttaccttccacacgcaaatttccttccactca

SEQ ID NO: 164 - DNA, 51 bp, Artificial Sequence
  Description of Artificial Sequence Synthetic oligonucleotide
gccgtttcatatcgaacaaggcgctaggagctgctggtgcaggggccacgc

SEQ ID NO: 165 - DNA, 51 bp, Artificial Sequence
  Description of Artificial Sequence Synthetic oligonucleotide
cgccagcaatcagctttgatacactattcatcggacatgttactgtttttg

SEQ ID NO: 166 - DNA, 51 bp, Artificial Sequence
  Description of Artificial Sequence Synthetic oligonucleotide
ctttggggtaagcgaccatcagcctgtgtcatcaacttggtccacagtcgg

SEQ ID NO: 167 - DNA, 51 bp, Artificial Sequence
  Description of Artificial Sequence Synthetic oligonucleotide
acgatggcgttgcaggcgcttacctggccctctagggactcgaacagagac

SEQ ID NO: 168 - DNA, 51 bp, Artificial Sequence
  Description of Artificial Sequence Synthetic oligonucleotide
gccgtttcatatcgaacaaggcgctcagagggaggacgagctgaccttcac

SEQ ID NO: 169 - DNA, 51 bp, Artificial Sequence
  Description of Artificial Sequence Synthetic oligonucleotide
cgccagcaatcagctttgatacactccaactcgaaattccccgtgaccagt

SEQ ID NO: 170 - DNA, 51 bp, Artificial Sequence
  Description of Artificial Sequence Synthetic oligonucleotide
ctttggggtaagcgaccatcagcctaccagcagatactcagccggaggatg

SEQ ID NO: 171 - DNA, 51 bp, Artificial Sequence
  Description of Artificial Sequence Synthetic oligonucleotide
acgatggcgttgcaggcgcttacctgaggaccccaagtcccatagggacct

SEQ ID NO: 172 - DNA, 51 bp, Artificial Sequence
  Description of Artificial Sequence Synthetic oligonucleotide
gccgtttcatatcgaacaaggcgctgcagcgcaccacgggacccaagcccc

SEQ ID NO: 173 - DNA, 51 bp, Artificial Sequence
  Description of Artificial Sequence Synthetic oligonucleotide
cgccagcaatcagctttgatacactcagagtctgaggtagctgccctggcg

SEQ ID NO: 174 - DNA, 51 bp, Artificial Sequence
  Description of Artificial Sequence Synthetic oligonucleotide
ctttggggtaagcgaccatcagccttgctgttttcttccttcaggcatacc

SEQ ID NO: 175 - DNA, 51 bp, Artificial Sequence
  Description of Artificial Sequence Synthetic oligonucleotide
acgatggcgttgcaggcgcttacctcctgaacagctcgcggctcagcagga

SEQ ID NO: 176 - DNA, 51 bp, Artificial Sequence
  Description of Artificial Sequence Synthetic oligonucleotide
gccgtttcatatcgaacaaggcgctatcttcaaagttgcagtaaaaacccg

SEQ ID NO: 177 - DNA, 51 bp, Artificial Sequence
  Description of Artificial Sequence Synthetic oligonucleotide
cgccagcaatcagctttgatacactcttgattcatgatattttactccaaa

SEQ ID NO: 178 - DNA, 51 bp, Artificial Sequence
  Description of Artificial Sequence Synthetic oligonucleotide
ctttggggtaagcgaccatcagccttccctcattgcactgtactcctcttt

SEQ ID NO: 179 - DNA, 51 bp, Artificial Sequence
  Description of Artificial Sequence Synthetic oligonucleotide
acgatggcgttgcaggcgcttacctgtccgaggacaacgatgaggcggcgt

SEQ ID NO: 180 - DNA, 51 bp, Artificial Sequence
  Description of Artificial Sequence Synthetic oligonucleotide
gccgtttcatatcgaacaaggcgctttccgcctggtgttggaagagacaga

SEQ ID NO: 181 - DNA, 51 bp, Artificial Sequence
  Description of Artificial Sequence Synthetic oligonucleotide
cgccagcaatcagctttgatacacttggtctttcagtgcctccactatgat

SEQ ID NO: 182 - DNA, 51 bp, Artificial Sequence
  Description of Artificial Sequence Synthetic oligonucleotide
ctttggggtaagcgaccatcagccttgaagagaaatataagaaggctatgt

SEQ ID NO: 183 - DNA, 51 bp, Artificial Sequence
  Description of Artificial Sequence Synthetic oligonucleotide
acgatggcgttgcaggcgcttacctcctccaggtgcaggagttcatgctcg

SEQ ID NO: 184 - DNA, 51 bp, Artificial Sequence
  Description of Artificial Sequence Synthetic oligonucleotide
gccgtttcatatcgaacaaggcgctaaaggcaatgtgggatcctgaattga

SEQ ID NO: 185 - DNA, 51 bp, Artificial Sequence
  Description of Artificial Sequence Synthetic oligonucleotide
cgccagcaatcagctttgatacactgcccgaacagccgctggatatgggaa

SEQ ID NO: 186 - DNA, 51 bp, Artificial Sequence
  Description of Artificial Sequence Synthetic oligonucleotide
ctttggggtaagcgaccatcagcctctaaaaaggaccctgaaggttgtgaa

SEQ ID NO: 187 - DNA, 51 bp, Artificial Sequence
  Description of Artificial Sequence Synthetic oligonucleotide
acgatggcgttgcaggcgcttaccttcatatggatgataatgatggagaaa

SEQ ID NO: 188 - DNA, 51 bp, Artificial Sequence
  Description of Artificial Sequence Synthetic oligonucleotide
gccgtttcatatcgaacaaggcgctcttggctgtgctcctgctgctggcca

SEQ ID NO: 189 - DNA, 51 bp, Artificial Sequence
  Description of Artificial Sequence Synthetic oligonucleotide
cgccagcaatcagctttgatacactatcaactataggttgctttggtggta

SEQ ID NO: 190 - DNA, 51 bp, Artificial Sequence
  Description of Artificial Sequence Synthetic oligonucleotide
ctttggggtaagcgaccatcagcctaaatttctgaataactgaagttggtt

SEQ ID NO: 191 - DNA, 51 bp, Artificial Sequence
  Description of Artificial Sequence Synthetic oligonucleotide
acgatggcgttgcaggcgcttacctggtagcagacaaacctgtggttgata

SEQ ID NO: 192 - DNA, 51 bp, Artificial Sequence
  Description of Artificial Sequence Synthetic oligonucleotide
gccgtttcatatcgaacaaggcgctgagctttgggttgttccttaggacct

SEQ ID NO: 193 - DNA, 51 bp, Artificial Sequence
  Description of Artificial Sequence Synthetic oligonucleotide
cgccagcaatcagctttgatacactggaattacggcagcccttctttcccc

SEQ ID NO: 194 - DNA, 51 bp, Artificial Sequence
  Description of Artificial Sequence Synthetic oligonucleotide
ctttggggtaagcgaccatcagcctcggggcctctgcttggatgtgatgat

SEQ ID NO: 195 - DNA, 51 bp, Artificial Sequence
  Description of Artificial Sequence Synthetic oligonucleotide
acgatggcgttgcaggcgcttacctggcggccgtggtggcggcagtggtgt

SEQ ID NO: 196 - DNA, 51 bp, Artificial Sequence
  Description of Artificial Sequence Synthetic oligonucleotide
gccgtttcatatcgaacaaggcgctgcagaagtcatatttaggatgtgtaa

SEQ ID NO: 197 - DNA, 51 bp, Artificial Sequence
  Description of Artificial Sequence Synthetic oligonucleotide
cgccagcaatcagctttgatacactggactttttttccaaggctattcagg

SEQ ID NO: 198 - DNA, 51 bp, Artificial Sequence
  Description of Artificial Sequence Synthetic oligonucleotide
ctttggggtaagcgaccatcagcctgagatgtgtaagcgcagccttgagtt

SEQ ID NO: 199 - DNA, 51 bp, Artificial Sequence
  Description of Artificial Sequence Synthetic oligonucleotide
acgatggcgttgcaggcgcttacctatttatgctatacatgatgaaacata

SEQ ID NO: 200 - DNA, 51 bp, Artificial Sequence
  Description of Artificial Sequence Synthetic oligonucleotide
gccgtttcatatcgaacaaggcgctgaaattgatagaagcagaagatcgga

SEQ ID NO: 201 - DNA, 51 bp, Artificial Sequence
  Description of Artificial Sequence Synthetic oligonucleotide
cgccagcaatcagctttgatacactctcacctcccatgttgctcaaagaaa

SEQ ID NO: 202 - DNA, 51 bp, Artificial Sequence
  Description of Artificial Sequence Synthetic oligonucleotide
ctttggggtaagcgaccatcagcctagcattctctgcagtacatcaaccgc

SEQ ID NO: 203 - DNA, 51 bp, Artificial Sequence
  Description of Artificial Sequence Synthetic oligonucleotide
acgatggcgttgcaggcgcttacctactttactcacgtttttcccatctat

SEQ ID NO: 204 - DNA, 51 bp, Artificial Sequence
  Description of Artificial Sequence Synthetic oligonucleotide
gccgtttcatatcgaacaaggcgctggcatggtggtggatgtagtggtggg

SEQ ID NO: 205 - DNA, 51 bp, Artificial Sequence
  Description of Artificial Sequence Synthetic oligonucleotide
cgccagcaatcagctttgatacactatggtggtggatgtagtggtggtggg

SEQ ID NO: 206 - DNA, 51 bp, Artificial Sequence
  Description of Artificial Sequence Synthetic oligonucleotide
ctttggggtaagcgaccatcagcctaacatgagttttttatggcgggaggg

SEQ ID NO: 207 - DNA, 51 bp, Artificial Sequence
  Description of Artificial Sequence Synthetic oligonucleotide
acgatggcgttgcaggcgcttacctggacaccggcaaggccaccctgaccg

SEQ ID NO: 208 - DNA, 51 bp, Artificial Sequence
  Description of Artificial Sequence Synthetic oligonucleotide
gccgtttcatatcgaacaaggcgctctgacccactcatcccaagacacact

SEQ ID NO: 209 - DNA, 51 bp, Artificial Sequence
  Description of Artificial Sequence Synthetic oligonucleotide
cgccagcaatcagctttgatacactgaaagtaacagcttgactatatccat

SEQ ID NO: 210 - DNA, 51 bp, Artificial Sequence
  Description of Artificial Sequence Synthetic oligonucleotide
ctttggggtaagcgaccatcagcctgtgtcctggaatggggcccatgagac

SEQ ID NO: 211 - DNA, 51 bp, Artificial Sequence
  Description of Artificial Sequence Synthetic oligonucleotide
acgatggcgttgcaggcgcttaccttggaatttcctcctcgagtctgaaca

SEQ ID NO: 212 - DNA, 51 bp, Artificial Sequence
  Description of Artificial Sequence Synthetic oligonucleotide
gccgtttcatatcgaacaaggcgctttccctccagccccaggttacccctc

SEQ ID NO: 213 - DNA, 51 bp, Artificial Sequence
  Description of Artificial Sequence Synthetic oligonucleotide
cgccagcaatcagctttgatacactcggggggtcttggatgtgccggcttt

SEQ ID NO: 214 - DNA, 51 bp, Artificial Sequence
  Description of Artificial Sequence Synthetic oligonucleotide
ctttggggtaagcgaccatcagccttggcctgttggccgtatctgctaact

SEQ ID NO: 215 - DNA, 51 bp, Artificial Sequence
  Description of Artificial Sequence Synthetic oligonucleotide
acgatggcgttgcaggcgcttacctctgttttgttccgaatgtctgaggaa

SEQ ID NO: 216 - DNA, 51 bp, Artificial Sequence
  Description of Artificial Sequence Synthetic oligonucleotide
gccgtttcatatcgaacaaggcgctgtgtcaacaattctaaggaggaagaa

SEQ ID NO: 217 - DNA, 51 bp, Artificial Sequence
  Description of Artificial Sequence Synthetic oligonucleotide
cgccagcaatcagctttgatacactgagatccagatgttttggaatattaa

SEQ ID NO: 218 - DNA, 51 bp, Artificial Sequence
  Description of Artificial Sequence Synthetic oligonucleotide
ctttggggtaagcgaccatcagccttggactgtgtatgaaacctggttttg

SEQ ID NO: 219 - DNA, 51 bp, Artificial Sequence
  Description of Artificial Sequence Synthetic oligonucleotide
acgatggcgttgcaggcgcttacctcaatctttttaaccattttgtcatct

SEQ ID NO: 220 - DNA, 51 bp, Artificial Sequence
  Description of Artificial Sequence Synthetic oligonucleotide
gccgtttcatatcgaacaaggcgctccaggggagaaaagtacattggaaaa

SEQ ID NO: 221 - DNA, 51 bp, Artificial Sequence
  Description of Artificial Sequence Synthetic oligonucleotide
cgccagcaatcagctttgatacactctttattcaggtggatgcccctgaca

SEQ ID NO: 222 - DNA, 51 bp, Artificial Sequence
  Description of Artificial Sequence Synthetic oligonucleotide
ctttggggtaagcgaccatcagcctcaacatctcttttccctggaagttta

SEQ ID NO: 223 - DNA, 51 bp, Artificial Sequence
  Description of Artificial Sequence Synthetic oligonucleotide
acgatggcgttgcaggcgcttacctggacatggatcttgtttttctctttt

SEQ ID NO: 224 - DNA, 51 bp, Artificial Sequence
  Description of Artificial Sequence Synthetic oligonucleotide
gccgtttcatatcgaacaaggcgctctcaatctgtagtgctcctggtcgga

SEQ ID NO: 225 - DNA, 51 bp, Artificial Sequence
  Description of Artificial Sequence Synthetic oligonucleotide
cgccagcaatcagctttgatacactgttttttcaggaggccatctttctca

SEQ ID NO: 226 - DNA, 51 bp, Artificial Sequence
  Description of Artificial Sequence Synthetic oligonucleotide
ctttggggtaagcgaccatcagcctagaatgagcctgttctgttgacattt

SEQ ID NO: 227 - DNA, 51 bp, Artificial Sequence
  Description of Artificial Sequence Synthetic oligonucleotide
acgatggcgttgcaggcgcttacctcaggggagggtgtgggcaggcggtta

SEQ ID NO: 228 - DNA, 51 bp, Artificial Sequence
  Description of Artificial Sequence Synthetic oligonucleotide
gccgtttcatatcgaacaaggcgctgactattcagacatcaatgaggtgga

SEQ ID NO: 229 - DNA, 51 bp, Artificial Sequence
  Description of Artificial Sequence Synthetic oligonucleotide
cgccagcaatcagctttgatacactctgttcctctacagggccaaaacacc

SEQ ID NO: 230 - DNA, 51 bp, Artificial Sequence
  Description of Artificial Sequence Synthetic oligonucleotide
ctttggggtaagcgaccatcagcctgatgatactcactgtccatcagcctt

SEQ ID NO: 231 - DNA, 51 bp, Artificial Sequence
  Description of Artificial Sequence Synthetic oligonucleotide
acgatggcgttgcaggcgcttacctagtatcctcacctgtagccaggtatt

SEQ ID NO: 232 - DNA, 51 bp, Artificial Sequence
  Description of Artificial Sequence Synthetic oligonucleotide
gccgtttcatatcgaacaaggcgctgttaattcagcatccagcaggtccca

SEQ ID NO: 233 - DNA, 51 bp, Artificial Sequence
  Description of Artificial Sequence Synthetic oligonucleotide
cgccagcaatcagctttgatacactacaccaacattcccagctgctggaaa

SEQ ID NO: 234 - DNA, 51 bp, Artificial Sequence
  Description of Artificial Sequence Synthetic oligonucleotide
ctttggggtaagcgaccatcagcctcctgggccaggtgtgcatcaaagcga

SEQ ID NO: 235 - DNA, 51 bp, Artificial Sequence
  Description of Artificial Sequence Synthetic oligonucleotide
acgatggcgttgcaggcgcttacctgtgtcaagctactctcaggactgcta

SEQ ID NO: 236 - DNA, 51 bp, Artificial Sequence
  Description of Artificial Sequence Synthetic oligonucleotide
gccgtttcatatcgaacaaggcgctcaaggtgccaggtgcaagacccacct

SEQ ID NO: 237 - DNA, 51 bp, Artificial Sequence
  Description of Artificial Sequence Synthetic oligonucleotide
cgccagcaatcagctttgatacacttgtcgcgatgaatgtgaaatcctggg

SEQ ID NO: 238 - DNA, 51 bp, Artificial Sequence
  Description of Artificial Sequence Synthetic oligonucleotide
ctttggggtaagcgaccatcagccttccacaaactcgtcactcatcctcca

SEQ ID NO: 239 - DNA, 51 bp, Artificial Sequence
  Description of Artificial Sequence Synthetic oligonucleotide
acgatggcgttgcaggcgcttacctagacatggaagccagtgattatgaga

SEQ ID NO: 240 - DNA, 51 bp, Artificial Sequence
  Description of Artificial Sequence Synthetic oligonucleotide
gccgtttcatatcgaacaaggcgctccacagccaggcagtctgtatcttga

SEQ ID NO: 241 - DNA, 51 bp, Artificial Sequence
  Description of Artificial Sequence Synthetic oligonucleotide
cgccagcaatcagctttgatacactatatgtggaggcccaacaaaagagaa

SEQ ID NO: 242 - DNA, 51 bp, Artificial Sequence
  Description of Artificial Sequence Synthetic oligonucleotide
ctttggggtaagcgaccatcagccttggaagttgcgtattgtaagctatta

SEQ ID NO: 243 - DNA, 51 bp, Artificial Sequence
  Description of Artificial Sequence Synthetic oligonucleotide
acgatggcgttgcaggcgcttacctggtcagaacaggagtgcacggataga

SEQ ID NO: 244 - DNA, 51 bp, Artificial Sequence
  Description of Artificial Sequence Synthetic oligonucleotide
gccgtttcatatcgaacaaggcgcttgctttcaatcccaaattatgtgttc

SEQ ID NO: 245 - DNA, 51 bp, Artificial Sequence
  Description of Artificial Sequence Synthetic oligonucleotide
cgccagcaatcagctttgatacactcatcagtgtgtctgaacatgtggtct

SEQ ID NO: 246 - DNA, 51 bp, Artificial Sequence
  Description of Artificial Sequence Synthetic oligonucleotide
ctttggggtaagcgaccatcagcctctgccagcctgccctggaggaagact

SEQ ID NO: 247 - DNA, 51 bp, Artificial Sequence
  Description of Artificial Sequence Synthetic oligonucleotide
acgatggcgttgcaggcgcttacctactggaactatctgtaatactggaaa

SEQ ID NO: 248 - DNA, 51 bp, Artificial Sequence
  Description of Artificial Sequence Synthetic oligonucleotide
gccgtttcatatcgaacaaggcgctaactctttcacttttacatattaaat

SEQ ID NO: 249 - DNA, 51 bp, Artificial Sequence
  Description of Artificial Sequence Synthetic oligonucleotide
cgccagcaatcagctttgatacactgcagccagagtggttttttcaggggg

SEQ ID NO: 250 - DNA, 50 bp, Artificial Sequence
  Description of Artificial Sequence Synthetic oligonucleotide
ttttcacatggttttccaggcttgctccggtaagttccagagcgcagagt

SEQ ID NO: 251 - DNA, 50 bp, Artificial Sequence
  Description of Artificial Sequence Synthetic oligonucleotide
gcgccagctccagcaaagccagcactccggtaagttccagagcgcagagt

SEQ ID NO: 252 - DNA, 50 bp, Artificial Sequence
  Description of Artificial Sequence Synthetic oligonucleotide
ttggcttcgacaactttgctgcttgtccggtaagttccagagcgcagagt

SEQ ID NO: 253 - DNA, 50 bp, Artificial Sequence
  Description of Artificial Sequence Synthetic oligonucleotide
taaactagaaaacatacaaaataggtccggtaagttccagagcgcagagt

SEQ ID NO: 254 - DNA, 50 bp, Artificial Sequence
  Description of Artificial Sequence Synthetic oligonucleotide
gtgcccgccggccctcgctggactctcataggacgacttcgtccaagatc

SEQ ID NO: 255 - DNA, 50 bp, Artificial Sequence
  Description of Artificial Sequence Synthetic oligonucleotide
atcagaagccctttgagagtggaagtcataggacgacttcgtccaagatc

SEQ ID NO: 256 - DNA, 50 bp, Artificial Sequence
  Description of Artificial Sequence Synthetic oligonucleotide
ccaagactctctccccagggaagaatcataggacgacttcgtccaagatc

SEQ ID NO: 257 - DNA, 50 bp, Artificial Sequence
  Description of Artificial Sequence Synthetic oligonucleotide
ggtactgtactttaaagaggtcacttcataggacgacttcgtccaagatc

SEQ ID NO: 258 - DNA, 50 bp, Artificial Sequence
  Description of Artificial Sequence Synthetic oligonucleotide
tctgcaaaggagtaagtcgatttggtctgtatagaggtcttcatgcaaac

SEQ ID NO: 259 - DNA, 50 bp, Artificial Sequence
  Description of Artificial Sequence Synthetic oligonucleotide
gataagatgctgaggaggggccagatctgtatagaggtcttcatgcaaac

SEQ ID NO: 260 - DNA, 50 bp, Artificial Sequence
  Description of Artificial Sequence Synthetic oligonucleotide
ggggagcagcctctggcattctgggtctgtatagaggtcttcatgcaaac

SEQ ID NO: 261 - DNA, 50 bp, Artificial Sequence
  Description of Artificial Sequence Synthetic oligonucleotide
ctccctgatgtaccaccaactttactctgtatagaggtcttcatgcaaac

SEQ ID NO: 262 - DNA, 50 bp, Artificial Sequence
  Description of Artificial Sequence Synthetic oligonucleotide
gtcaggaggggcatcaggcgctaagtcagtacaggccatggacctttcag

SEQ ID NO: 263 - DNA, 50 bp, Artificial Sequence
  Description of Artificial Sequence Synthetic oligonucleotide
ctctgcagctgtgggtttctttgcatcagtacaggccatggacctttcag

SEQ ID NO: 264 - DNA, 50 bp, Artificial Sequence
  Description of Artificial Sequence Synthetic oligonucleotide
caagagcgccatcatccagaatgtgtcagtacaggccatggacctttcag

SEQ ID NO: 265 - DNA, 50 bp, Artificial Sequence
  Description of Artificial Sequence Synthetic oligonucleotide
cttttggacaccaggttggtgaatctcagtacaggccatggacctttcag

SEQ ID NO: 266 - DNA, 50 bp, Artificial Sequence
  Description of Artificial Sequence Synthetic oligonucleotide
tttcagaggtgagagtagggcaatttcggatcctggctgcatcgatacaa

SEQ ID NO: 267 - DNA, 50 bp, Artificial Sequence
  Description of Artificial Sequence Synthetic oligonucleotide
ctcgaataggcacagttacccccagtcggatcctggctgcatcgatacaa

SEQ ID NO: 268 - DNA, 50 bp, Artificial Sequence
  Description of Artificial Sequence Synthetic oligonucleotide
ccgagctcgcgccagcccgcgccactcggatcctggctgcatcgatacaa

SEQ ID NO: 269 - DNA, 50 bp, Artificial Sequence
  Description of Artificial Sequence Synthetic oligonucleotide
tatttaacaacatcagccgagacgttcggatcctggctgcatcgatacaa

SEQ ID NO: 270 - DNA, 50 bp, Artificial Sequence
  Description of Artificial Sequence Synthetic oligonucleotide
gaggatgaccccaaagatagtggattccggcatagatcgctaattctgtg

SEQ ID NO: 271 - DNA, 50 bp, Artificial Sequence
  Description of Artificial Sequence Synthetic oligonucleotide
cgtcccagagctggtccacctgcagtccggcatagatcgctaattctgtg

SEQ ID NO: 272 - DNA, 50 bp, Artificial Sequence
  Description of Artificial Sequence Synthetic oligonucleotide
caggcagtttccctatggagagagctccggcatagatcgctaattctgtg

SEQ ID NO: 273 - DNA, 50 bp, Artificial Sequence
  Description of Artificial Sequence Synthetic oligonucleotide
atacaaatgaatcatggagaaatcttccggcatagatcgctaattctgtg

SEQ ID NO: 274 - DNA, 50 bp, Artificial Sequence
  Description of Artificial Sequence Synthetic oligonucleotide
acctgctgtgtcgagaatatccaagtcctcctacactacgatgaggcgtg

SEQ ID NO: 275 - DNA, 50 bp, Artificial Sequence
  Description of Artificial Sequence Synthetic oligonucleotide
ccgggccctgggcggtggcaacggctcctcctacactacgatgaggcgtg

SEQ ID NO: 276 - DNA, 50 bp, Artificial Sequence
  Description of Artificial Sequence Synthetic oligonucleotide
catgggtttggtgacctggcccttgtcctcctacactacgatgaggcgtg

SEQ ID NO: 277 - DNA, 50 bp, Artificial Sequence
  Description of Artificial Sequence Synthetic oligonucleotide
gttgtaggtggcacctctggtgaggtcctcctacactacgatgaggcgtg

SEQ ID NO: 278 - DNA, 50 bp, Artificial Sequence
  Description of Artificial Sequence Synthetic oligonucleotide
tttccaatgctcagctagacaatgatcgcaacggcagcgttcaatgttat

SEQ ID NO: 279 - DNA, 50 bp, Artificial Sequence
  Description of Artificial Sequence Synthetic oligonucleotide
gcttcctccgagaccccttacgagatcgcaacggcagcgttcaatgttat

SEQ ID NO: 280 - DNA, 50 bp, Artificial Sequence
  Description of Artificial Sequence Synthetic oligonucleotide
aaaaaccttcacaacgaccaggccttcgcaacggcagcgttcaatgttat

SEQ ID NO: 281 - DNA, 50 bp, Artificial Sequence
  Description of Artificial Sequence Synthetic oligonucleotide
gaacagccgcaagtttgagtttgaatcgcaacggcagcgttcaatgttat

SEQ ID NO: 282 - DNA, 50 bp, Artificial Sequence
  Description of Artificial Sequence Synthetic oligonucleotide
aaaagtgatgacaaaaacactgtaatccagcgttatattgccgtagtctg

SEQ ID NO: 283 - DNA, 50 bp, Artificial Sequence
  Description of Artificial Sequence Synthetic oligonucleotide
tagatacaccaataaattatagtcttccagcgttatattgccgtagtctg

SEQ ID NO: 284 - DNA, 50 bp, Artificial Sequence
  Description of Artificial Sequence Synthetic oligonucleotide
ggctgtatcgagggcaggcgctccatccagcgttatattgccgtagtctg

SEQ ID NO: 285 - DNA, 50 bp, Artificial Sequence
  Description of Artificial Sequence Synthetic oligonucleotide
ttgccaacacagcctctgcttcttctccagcgttatattgccgtagtctg

SEQ ID NO: 286 - DNA, 50 bp, Artificial Sequence
  Description of Artificial Sequence Synthetic oligonucleotide
ctgaattctatgaaaagtaggtctttcggcgctcaccgtaatagttacgt

SEQ ID NO: 287 - DNA, 50 bp, Artificial Sequence
  Description of Artificial Sequence Synthetic oligonucleotide
ctaaattagtgaaaagaaaaatgtatcggcgctcaccgtaatagttacgt

SEQ ID NO: 288 - DNA, 50 bp, Artificial Sequence
  Description of Artificial Sequence Synthetic oligonucleotide
ggtagggggtgtgcttataaggtaatcggcgctcaccgtaatagttacgt

SEQ ID NO: 289 - DNA, 50 bp, Artificial Sequence
  Description of Artificial Sequence Synthetic oligonucleotide
cccacatggggcccatcaaactccgtcggcgctcaccgtaatagttacgt

SEQ ID NO: 290 - DNA, 50 bp, Artificial Sequence
  Description of Artificial Sequence Synthetic oligonucleotide
ttgcaaagacggtgctatggactgatcgtagttaaacagcggctatccat

SEQ ID NO: 291 - DNA, 50 bp, Artificial Sequence
  Description of Artificial Sequence Synthetic oligonucleotide
cgttggtgatgttggccccgctggctcgtagttaaacagcggctatccat

SEQ ID NO: 292 - DNA, 50 bp, Artificial Sequence
  Description of Artificial Sequence Synthetic oligonucleotide
tatctgtataaataagaaaaaaaggtcgtagttaaacagcggctatccat

SEQ ID NO: 293 - DNA, 50 bp, Artificial Sequence
  Description of Artificial Sequence Synthetic oligonucleotide
gtgcgaggtaatctaatctcttttttcgtagttaaacagcggctatccat

SEQ ID NO: 294 - DNA, 50 bp, Artificial Sequence
  Description of Artificial Sequence Synthetic oligonucleotide
tgtgtattcgctctatcccacactttctaccttgctctgtggaagccgaa

SEQ ID NO: 295 - DNA, 50 bp, Artificial Sequence
  Description of Artificial Sequence Synthetic oligonucleotide
ttataaaggaaaaaaaataccgaaatctaccttgctctgtggaagccgaa

SEQ ID NO: 296 - DNA, 50 bp, Artificial Sequence
  Description of Artificial Sequence Synthetic oligonucleotide
tataaaaaagataatggaaagggattctaccttgctctgtggaagccgaa

SEQ ID NO: 297 - DNA, 50 bp, Artificial Sequence
  Description of Artificial Sequence Synthetic oligonucleotide
catatagtaagtatttaatttatgctctaccttgctctgtggaagccgaa

SEQ ID NO: 298 - DNA, 50 bp, Artificial Sequence
  Description of Artificial Sequence Synthetic oligonucleotide
gacctgtcaaaatagaatgtgagtttccggtaagttccagagcgcagagt

SEQ ID NO: 299 - DNA, 50 bp, Artificial Sequence
  Description of Artificial Sequence Synthetic oligonucleotide
caattccatgcacttctcatttctgtccggtaagttccagagcgcagagt

SEQ ID NO: 300 - DNA, 50 bp, Artificial Sequence
  Description of Artificial Sequence Synthetic oligonucleotide
ggacatgcttcgtcgtctgcttggttccggtaagttccagagcgcagagt

SEQ ID NO: 301 - DNA, 50 bp, Artificial Sequence
  Description of Artificial Sequence Synthetic oligonucleotide
catgcttcgtcgtctgcttggtcactccggtaagttccagagcgcagagt

SEQ ID NO: 302 - DNA, 50 bp, Artificial Sequence
  Description of Artificial Sequence Synthetic oligonucleotide
agactgaccctttttggacttcaggtcataggacgacttcgtccaagatc

SEQ ID NO: 303 - DNA, 50 bp, Artificial Sequence
  Description of Artificial Sequence Synthetic oligonucleotide
cgagcccactgggtgcatcctgagatcataggacgacttcgtccaagatc

SEQ ID NO: 304 - DNA, 50 bp, Artificial Sequence
  Description of Artificial Sequence Synthetic oligonucleotide
tgctacggagaagttgtttaaggggtcataggacgacttcgtccaagatc

SEQ ID NO: 305 - DNA, 50 bp, Artificial Sequence
  Description of Artificial Sequence Synthetic oligonucleotide
atgcccattcttggctgcatcgtgatcataggacgacttcgtccaagatc

SEQ ID NO: 306 - DNA, 50 bp, Artificial Sequence
  Description of Artificial Sequence Synthetic oligonucleotide
ggttgtctgagagagagcttcttgttctgtatagaggtcttcatgcaaac

SEQ ID NO: 307 - DNA, 50 bp, Artificial Sequence
  Description of Artificial Sequence Synthetic oligonucleotide
aaaactgccaggaacaatacacaactctgtatagaggtcttcatgcaaac

SEQ ID NO: 308 - DNA, 50 bp, Artificial Sequence
  Description of Artificial Sequence Synthetic oligonucleotide
tcgtgtggctccttctttgctatagtctgtatagaggtcttcatgcaaac

SEQ ID NO: 309 - DNA, 50 bp, Artificial Sequence
  Description of Artificial Sequence Synthetic oligonucleotide
tcagattggtgagctcccatctgtttctgtatagaggtcttcatgcaaac

SEQ ID NO: 310 - DNA, 50 bp, Artificial Sequence
  Description of Artificial Sequence Synthetic oligonucleotide
ggctgtgccactgctggggaaggcctcagtacaggccatggacctttcag

SEQ ID NO: 311 - DNA, 50 bp, Artificial Sequence
  Description of Artificial Sequence Synthetic oligonucleotide
aagccacaagattacaagaaacggctcagtacaggccatggacctttcag

SEQ ID NO: 312 - DNA, 50 bp, Artificial Sequence
  Description of Artificial Sequence Synthetic oligonucleotide
cctccaaggatgtactgcagtacagtcagtacaggccatggacctttcag

SEQ ID NO: 313 - DNA, 50 bp, Artificial Sequence
  Description of Artificial Sequence Synthetic oligonucleotide
aaaaatgatcatgccaagaagcctatcagtacaggccatggacctttcag

SEQ ID NO: 314 - DNA, 50 bp, Artificial Sequence
  Description of Artificial Sequence Synthetic oligonucleotide
ttacttttcttctcttgatgtgcaatcggatcctggctgcatcgatacaa

SEQ ID NO: 315 - DNA, 50 bp, Artificial Sequence
  Description of Artificial Sequence Synthetic oligonucleotide
tcactttctaagaacttctttatggtcggatcctggctgcatcgatacaa

SEQ ID NO: 316 - DNA, 50 bp, Artificial Sequence
  Description of Artificial Sequence Synthetic oligonucleotide
taaaaagatagaatctgaaagtaaatcggatcctggctgcatcgatacaa

SEQ ID NO: 317 - DNA, 50 bp, Artificial Sequence
  Description of Artificial Sequence Synthetic oligonucleotide
aaaaaggaactgagataaaaccaggtcggatcctggctgcatcgatacaa

SEQ ID NO: 318 - DNA, 50 bp, Artificial Sequence
  Description of Artificial Sequence Synthetic oligonucleotide
aaacggccactgcagacttcaccgatccggcatagatcgctaattctgtg

SEQ ID NO: 319 - DNA, 50 bp, Artificial Sequence
  Description of Artificial Sequence Synthetic oligonucleotide
acaacagtaaaatcacctatgagactccggcatagatcgctaattctgtg

SEQ ID NO: 320 - DNA, 50 bp, Artificial Sequence
  Description of Artificial Sequence Synthetic oligonucleotide
aaaagacaacattctctattttaggtccggcatagatcgctaattctgtg

SEQ ID NO: 321 - DNA, 50 bp, Artificial Sequence
  Description of Artificial Sequence Synthetic oligonucleotide
aacctgccatataaatctaagatcttccggcatagatcgctaattctgtg

SEQ ID NO: 322 - DNA, 50 bp, Artificial Sequence
  Description of Artificial Sequence Synthetic oligonucleotide
tgcgcaccacatcaatcacttcccatcctcctacactacgatgaggcgtg

SEQ ID NO: 323 - DNA, 50 bp, Artificial Sequence
  Description of Artificial Sequence Synthetic oligonucleotide
aagccgttggctggagacacctatttcctcctacactacgatgaggcgtg

SEQ ID NO: 324 - DNA, 50 bp, Artificial Sequence
  Description of Artificial Sequence Synthetic oligonucleotide
agaagatgaaagccgaagataccagtcctcctacactacgatgaggcgtg

SEQ ID NO: 325 - DNA, 50 bp, Artificial Sequence
  Description of Artificial Sequence Synthetic oligonucleotide
cattgactcagatctctcaatccattcctcctacactacgatgaggcgtg

SEQ ID NO: 326 - DNA, 50 bp, Artificial Sequence
  Description of Artificial Sequence Synthetic oligonucleotide
cagttcagcaaggggtcatagacaatcgcaacggcagcgttcaatgttat

SEQ ID NO: 327 - DNA, 50 bp, Artificial Sequence
  Description of Artificial Sequence Synthetic oligonucleotide
aatctggatggctttcaccccctcctcgcaacggcagcgttcaatgttat

SEQ ID NO: 328 - DNA, 50 bp, Artificial Sequence
  Description of Artificial Sequence Synthetic oligonucleotide
ggccttgtcaatgcactagaagagatcgcaacggcagcgttcaatgttat

SEQ ID NO: 329 - DNA, 50 bp, Artificial Sequence
  Description of Artificial Sequence Synthetic oligonucleotide
aaaaactgaaatggacaagaggtcatcgcaacggcagcgttcaatgttat

SEQ ID NO: 330 - DNA, 50 bp, Artificial Sequence
  Description of Artificial Sequence Synthetic oligonucleotide
tcgtccagggacgccaagacacagttccagcgttatattgccgtagtctg

SEQ ID NO: 331 - DNA, 50 bp, Artificial Sequence
  Description of Artificial Sequence Synthetic oligonucleotide
aactaaaacaaaacgatgacaaatttccagcgttatattgccgtagtctg

SEQ ID NO: 332 - DNA, 50 bp, Artificial Sequence
  Description of Artificial Sequence Synthetic oligonucleotide
tgagcagatagcctcccaccacacgtccagcgttatattgccgtagtctg

SEQ ID NO: 333 - DNA, 50 bp, Artificial Sequence
  Description of Artificial Sequence Synthetic oligonucleotide
gaatgtcctgtgtcaaacagagtactccagcgttatattgccgtagtctg

SEQ ID NO: 334 - DNA, 50 bp, Artificial Sequence
  Description of Artificial Sequence Synthetic oligonucleotide
gagctcgcggccatagcgctgtgcttcggcgctcaccgtaatagttacgt

SEQ ID NO: 335 - DNA, 50 bp, Artificial Sequence
  Description of Artificial Sequence Synthetic oligonucleotide
ttgaagatgaaacaagacctgctaatcggcgctcaccgtaatagttacgt

SEQ ID NO: 336 - DNA, 50 bp, Artificial Sequence
  Description of Artificial Sequence Synthetic oligonucleotide
aaaaacatccactctgcctcgaatctcggcgctcaccgtaatagttacgt

SEQ ID NO: 337 - DNA, 50 bp, Artificial Sequence
  Description of Artificial Sequence Synthetic oligonucleotide
tagaagccttattcactaaaattcatcggcgctcaccgtaatagttacgt

SEQ ID NO: 338 - DNA, 50 bp, Artificial Sequence
  Description of Artificial Sequence Synthetic oligonucleotide
aaaaaaagaaaaagattcaggtaagtcgtagttaaacagcggctatccat

SEQ ID NO: 339 - DNA, 50 bp, Artificial Sequence
  Description of Artificial Sequence Synthetic oligonucleotide
aaaacaactagaaaatgatacaagatcgtagttaaacagcggctatccat

SEQ ID NO: 340 - DNA, 50 bp, Artificial Sequence
  Description of Artificial Sequence Synthetic oligonucleotide
ccgaaatttaccgcatggaggaagttcgtagttaaacagcggctatccat

SEQ ID NO: 341 - DNA, 50 bp, Artificial Sequence
  Description of Artificial Sequence Synthetic oligonucleotide
gcaggtagcgggactgtcgggtgggtcgtagttaaacagcggctatccat

SEQ ID NO: 342 - DNA, 50 bp, Artificial Sequence
  Description of Artificial Sequence Synthetic oligonucleotide
gtacagcatcacacccacgctgagatctaccttgctctgtggaagccgaa

SEQ ID NO: 343 - DNA, 50 bp, Artificial Sequence
  Description of Artificial Sequence Synthetic oligonucleotide
ctaaataaaacaaagcagccaaaaatctaccttgctctgtggaagccgaa

SEQ ID NO: 344 - DNA, 50 bp, Artificial Sequence
  Description of Artificial Sequence Synthetic oligonucleotide
cctcatgaggatcactggccagtaatctaccttgctctgtggaagccgaa

SEQ ID NO: 345 - DNA, 50 bp, Artificial Sequence
  Description of Artificial Sequence Synthetic oligonucleotide
gtcttatataagtaatttaaaaaaatctaccttgctctgtggaagccgaa

SEQ ID NO: 346 - DNA, 25 bp, Artificial Sequence
  Description of Artificial Sequence Synthetic primer
ttatcccgagaattcagacagtcac

SEQ ID NO: 347 - DNA, 25 bp, Artificial Sequence
  Description of Artificial Sequence Synthetic primer
cacgggagttgatcctggttttcac

SEQ ID NO: 348 - DNA, 25 bp, Artificial Sequence
  Description of Artificial Sequence Synthetic primer
tatagccgcttaagtctacactcac

SEQ ID NO: 349 - DNA, 25 bp, Artificial Sequence
  Description of Artificial Sequence Synthetic primer
gtttcgtagcgtcctggagtatcac

SEQ ID NO: 350 - DNA, 25 bp, Artificial Sequence
  Description of Artificial Sequence Synthetic primer
aaaatgttctatatgaccgttccac

SEQ ID NO: 351 - DNA, 25 bp, Artificial Sequence
  Description of Artificial Sequence Synthetic primer
atcaagagtttagcacttcgcgcac

SEQ ID NO: 352 - DNA, 25 bp, Artificial Sequence
  Description of Artificial Sequence Synthetic primer
ggcgatgatagattcccctcgtcac

SEQ ID NO: 353 - DNA, 25 bp, Artificial Sequence
  Description of Artificial Sequence Synthetic primer
tatgcgctggcaacatcgacaccac

SEQ ID NO: 354 - DNA, 25 bp, Artificial Sequence
  Description of Artificial Sequence Synthetic primer
cagagatcatccgaaggcttctcac

SEQ ID NO: 355 - DNA, 25 bp, Artificial Sequence
  Description of Artificial Sequence Synthetic primer
attatgagactccccgacgtcccac

SEQ ID NO: 356 - DNA, 25 bp, Artificial Sequence
  Description of Artificial Sequence Synthetic primer
tgtgatcgacggcctttcaaatcac

SEQ ID NO: 357 - DNA, 25 bp, Artificial Sequence
  Description of Artificial Sequence Synthetic primer
aacccaactctggcaagcgttacac

SEQ ID NO: 358 - DNA, 25 bp, Artificial Sequence
  Description of Artificial Sequence Synthetic primer
gcgaaggatttgctgacttaagcac

SEQ ID NO: 359 - DNA, 25 bp, Artificial Sequence
  Description of Artificial Sequence Synthetic primer
atattcatgtgcaaaagcctcccac

SEQ ID NO: 360 - DNA, 25 bp, Artificial Sequence
  Description of Artificial Sequence Synthetic primer
cgtaaccccagacataggccttcac

SEQ ID NO: 361 - DNA, 25 bp, Artificial Sequence
  Description of Artificial Sequence Synthetic primer
ctgaaagcggtcgactaacgggcac

SEQ ID NO: 362 - DNA, 25 bp, Artificial Sequence
  Description of Artificial Sequence Synthetic primer
aattggcgtatacggccccaagcac

SEQ ID NO: 363 - DNA, 25 bp, Artificial Sequence
  Description of Artificial Sequence Synthetic primer
cgtctcaacttaagccagccgacac

SEQ ID NO: 364 - DNA, 25 bp, Artificial Sequence
  Description of Artificial Sequence Synthetic primer
aatagcccggctttatacgctgcac

SEQ ID NO: 365 - DNA, 25 bp, Artificial Sequence
  Description of Artificial Sequence Synthetic primer
cgcttgcgacctcttaaaacgtcac

SEQ ID NO: 366 - DNA, 25 bp, Artificial Sequence
  Description of Artificial Sequence Synthetic primer
gtcatacataactcttgagatccac

SEQ ID NO: 367 - DNA, 25 bp, Artificial Sequence
  Description of Artificial Sequence Synthetic primer
tcgatcgcttcagactatttcgcac

SEQ ID NO: 368 - DNA, 25 bp, Artificial Sequence
  Description of Artificial Sequence Synthetic primer
acgatggtttgtttcaggaaaccac

SEQ ID NO: 369 - DNA, 25 bp, Artificial Sequence
  Description of Artificial Sequence Synthetic primer
acacacttccaggcgatggaaacac

SEQ ID NO: 370 - DNA, 25 bp, Artificial Sequence
  Description of Artificial Sequence Synthetic primer
aaccgctacaaggcggggcaccaca

SEQ ID NO: 371 - DNA, 25 bp, Artificial Sequence
  Description of Artificial Sequence Synthetic primer
accaaacctagtagcgctatccaca

SEQ ID NO: 372 - DNA, 25 bp, Artificial Sequence
  Description of Artificial Sequence Synthetic primer
tgcaggaccagagaattcgaataca

SEQ ID NO: 373 - DNA, 25 bp, Artificial Sequence
  Description of Artificial Sequence Synthetic primer
actcaacatcggcatcgggcctaca

SEQ ID NO: 374 - DNA, 25 bp, Artificial Sequence
  Description of Artificial Sequence Synthetic primer
atttctacaaacgctcgccacaaca

SEQ ID NO: 375 - DNA, 25 bp, Artificial Sequence
  Description of Artificial Sequence Synthetic primer
aaaaatccaagttttaggcgttaca

SEQ ID NO: 376 - DNA, 25 bp, Artificial Sequence
  Description of Artificial Sequence Synthetic primer
tctatctatggccatggtctaaaca

SEQ ID NO: 377 - DNA, 25 bp, Artificial Sequence
  Description of Artificial Sequence Synthetic primer
gattgcgcggtaatagcgccctaca

SEQ ID NO: 378 - DNA, 25 bp, Artificial Sequence
  Description of Artificial Sequence Synthetic primer
tcttacgtgatgatatggcaacaca

SEQ ID NO: 379 - DNA, 25 bp, Artificial Sequence
  Description of Artificial Sequence Synthetic primer
tcccctagcaccctagggtatgaca

SEQ ID NO: 380 - DNA, 25 bp, Artificial Sequence
  Description of Artificial Sequence Synthetic primer
taagtattccatgcacccctaaaca

SEQ ID NO: 381 - DNA, 25 bp, Artificial Sequence
  Description of Artificial Sequence Synthetic primer
ccttacctcgtaactaactaagaca

SEQ ID NO: 382 - DNA, 25 bp, Artificial Sequence
  Description of Artificial Sequence Synthetic primer
tctggacaagattagcttaccaaca

SEQ ID NO: 383 - DNA, 25 bp, Artificial Sequence
  Description of Artificial Sequence Synthetic primer
taaccgatacgtacgagaggcaaca

SEQ ID NO: 384 - DNA, 25 bp, Artificial Sequence
  Description of Artificial Sequence Synthetic primer
cctggacgaggattgactctacaca

SEQ ID NO: 385 - DNA, 25 bp, Artificial Sequence
  Description of Artificial Sequence Synthetic primer
ttcggttaggtcctaccgtacaaca

SEQ ID NO: 386 - DNA, 25 bp, Artificial Sequence
  Description of Artificial Sequence Synthetic primer
attcgaacgctatcgaaaggttaca

SEQ ID NO: 387 - DNA, 25 bp, Artificial Sequence
  Description of Artificial Sequence Synthetic primer
aaggattgagtcacatggcgcaaca

SEQ ID NO: 388 - DNA, 25 bp, Artificial Sequence
  Description of Artificial Sequence Synthetic primer
tcagctaagcccttatgatccgaca

SEQ ID NO: 389 - DNA, 25 bp, Artificial Sequence
  Description of Artificial Sequence Synthetic primer
cataagcgagtcatactgacgaaca

SEQ ID NO: 390 - DNA, 25 bp, Artificial Sequence
  Description of Artificial Sequence Synthetic primer
cgaatggatcagtaactcgagaaca

SEQ ID NO: 391 - DNA, 25 bp, Artificial Sequence
  Description of Artificial Sequence Synthetic primer
gaaagcaggcaggccactgactaca

SEQ ID NO: 392 - DNA, 25 bp, Artificial Sequence
  Description of Artificial Sequence Synthetic primer
tagaaactcgaccagaggagctaca

SEQ ID NO: 393 - DNA, 25 bp, Artificial Sequence
  Description of Artificial Sequence Synthetic primer
gctatcggggaatccgcatcacaca

SEQ ID NO: 394 - DNA, 51 bp, Artificial Sequence
  Description of Artificial Sequence Synthetic oligonucleotide
ctgggggtaactgtgcctattcgaggggtccctatgggacttggggtcctc

SEQ ID NO: 395 - DNA, 51 bp, Artificial Sequence
  Description of Artificial Sequence Synthetic oligonucleotide
ctgggggtaactgtgcctattcgagaggtccctatgggacttggggtcctc

SEQ ID NO: 396 - DNA, 51 bp, Artificial Sequence
  Description of Artificial Sequence Synthetic oligonucleotide
gtgctggctttgctggagctggcgcagcaggaccacggtgctctggactgc

SEQ ID NO: 397 - DNA, 51 bp, Artificial Sequence
  Description of Artificial Sequence Synthetic oligonucleotide
gtgctggctttgctggagctggcgcggcaggaccacggtgctctggactgc

SEQ ID NO: 398 - DNA, 51 bp, Artificial Sequence
  Description of Artificial Sequence Synthetic oligonucleotide
tccagcactctgtcatgaggctgtacattctgggtgggcagtcttcagagc

SEQ ID NO: 399 - DNA, 51 bp, Artificial Sequence
  Description of Artificial Sequence Synthetic oligonucleotide
tccagcactctgtcatgaggctgtagattctgggtgggcagtcttcagagc

SEQ ID NO: 400 - DNA, 51 bp, Artificial Sequence
  Description of Artificial Sequence Synthetic oligonucleotide
caacatcgactttggcgagcccggggcccgcctgtcgccgcccgcgcctcc

SEQ ID NO: 401 - DNA, 51 bp, Artificial Sequence
  Description of Artificial Sequence Synthetic oligonucleotide
caacatcgactttggcgagcccgggccccgcctgtcgccgcccgcgcctcc

SEQ ID NO: 402 - DNA, 51 bp, Artificial Sequence
  Description of Artificial Sequence Synthetic oligonucleotide
gcgagtattactgctactcgaaatgcaaaagccactccaaggctccggaaa

SEQ ID NO: 403 - DNA, 51 bp, Artificial Sequence
  Description of Artificial Sequence Synthetic oligonucleotide
gcgagtattactgctactcgaaatgaaaaagccactccaaggctccggaaa

SEQ ID NO: 404 - DNA, 51 bp, Artificial Sequence
  Description of Artificial Sequence Synthetic oligonucleotide
ccaaatcgacttactcctttgcagacagaaacagcctccttggacaaggat

SEQ ID NO: 405 - DNA, 51 bp, Artificial Sequence
  Description of Artificial Sequence Synthetic oligonucleotide
ccaaatcgacttactcctttgcagatagaaacagcctccttggacaaggat

SEQ ID NO: 406 - DNA, 51 bp, Artificial Sequence
  Description of Artificial Sequence Synthetic oligonucleotide
tctcaggatgcacccagtgggctcgaggtcagggtggccttgccggtgtcc

SEQ ID NO: 407 - DNA, 51 bp, Artificial Sequence
  Description of Artificial Sequence Synthetic oligonucleotide
tctcaggatgcacccagtgggctcgcggtcagggtggccttgccggtgtcc

SEQ ID NO: 408 - DNA, 51 bp, Artificial Sequence
  Description of Artificial Sequence Synthetic oligonucleotide
cctcaccagaggtgccacctacaacgtcatagtggaggcactgaaagacca

SEQ ID NO: 409 - DNA, 51 bp, Artificial Sequence
  Description of Artificial Sequence Synthetic oligonucleotide
cctcaccagaggtgccacctacaacatcatagtggaggcactgaaagacca

SEQ ID NO: 410 - DNA, 51 bp, Artificial Sequence
  Description of Artificial Sequence Synthetic oligonucleotide
aaccgctacaaggcggggcaccacagaggaccccaagtcccatagggaccc

SEQ ID NO: 411 - DNA, 51 bp, Artificial Sequence
  Description of Artificial Sequence Synthetic oligonucleotide
accaaacctagtagcgctatccacagaggaccccaagtcccatagggacct

SEQ ID NO: 412 - DNA, 51 bp, Artificial Sequence
  Description of Artificial Sequence Synthetic oligonucleotide
tgcaggaccagagaattcgaatacagcagtccagagcaccgtggtcctgct

SEQ ID NO: 413 - DNA, 51 bp, Artificial Sequence
  Description of Artificial Sequence Synthetic oligonucleotide
actcaacatcggcatcgggcctacagcagtccagagcaccgtggtcctgcc

SEQ ID NO: 414 - DNA, 51 bp, Artificial Sequence
  Description of Artificial Sequence Synthetic oligonucleotide
atttctacaaacgctcgccacaacagctctgaagactgcccacccagaatg

SEQ ID NO: 415 - DNA, 51 bp, Artificial Sequence
  Description of Artificial Sequence Synthetic oligonucleotide
aaaaatccaagttttaggcgttacagctctgaagactgcccacccagaatc

SEQ ID NO: 416 - DNA, 51 bp, Artificial Sequence
  Description of Artificial Sequence Synthetic oligonucleotide
tctatctatggccatggtctaaacaggaggcgcgggcggcgacaggcgggc

SEQ ID NO: 417 - DNA, 51 bp, Artificial Sequence
  Description of Artificial Sequence Synthetic oligonucleotide
gattgcgcggtaatagcgccctacaggaggcgcgggcggcgacaggcgggg

SEQ ID NO: 418 - DNA, 51 bp, Artificial Sequence
  Description of Artificial Sequence Synthetic oligonucleotide
aaccgctacaaggcggggcaccacatttccggagccttggagtggcttttg

SEQ ID NO: 419 - DNA, 51 bp, Artificial Sequence
  Description of Artificial Sequence Synthetic oligonucleotide
accaaacctagtagcgctatccacatttccggagccttggagtggcttttt

SEQ ID NO: 420 - DNA, 51 bp, Artificial Sequence
  Description of Artificial Sequence Synthetic oligonucleotide
tgcaggaccagagaattcgaatacaatccttgtccaaggaggctgtttctg

SEQ ID NO: 421 - DNA, 51 bp, Artificial Sequence
  Description of Artificial Sequence Synthetic oligonucleotide
actcaacatcggcatcgggcctacaatccttgtccaaggaggctgtttcta

SEQ ID NO: 422 - DNA, 51 bp, Artificial Sequence
  Description of Artificial Sequence Synthetic oligonucleotide
atttctacaaacgctcgccacaacaggacaccggcaaggccaccctgacct

SEQ ID NO: 423 - DNA, 51 bp, Artificial Sequence
  Description of Artificial Sequence Synthetic oligonucleotide
aaaaatccaagttttaggcgttacaggacaccggcaaggccaccctgaccg

SEQ ID NO: 424 - DNA, 51 bp, Artificial Sequence
  Description of Artificial Sequence Synthetic oligonucleotide
tctatctatggccatggtctaaacatggtctttcagtgcctccactatgac

SEQ ID NO: 425 - DNA, 51 bp, Artificial Sequence
  Description of Artificial Sequence Synthetic oligonucleotide
gattgcgcggtaatagcgccctacatggtctttcagtgcctccactatgat

SEQ ID NO: 426 - DNA, 50 bp, Artificial Sequence
  Description of Artificial Sequence Synthetic oligonucleotide
ctcgaataggcacagttacccccaggtgaaaaccaggatcaactcccgtg

SEQ ID NO: 427 - DNA, 50 bp, Artificial Sequence
  Description of Artificial Sequence Synthetic oligonucleotide
gcgccagctccagcaaagccagcacgtgaaaaccaggatcaactcccgtg

SEQ ID NO: 428 - DNA, 50 bp, Artificial Sequence
  Description of Artificial Sequence Synthetic oligonucleotide
tacagcctcatgacagagtgctggagtgaaaaccaggatcaactcccgtg

SEQ ID NO: 429 - DNA, 50 bp, Artificial Sequence
  Description of Artificial Sequence Synthetic oligonucleotide
cccgggctcgccaaagtcgatgttggtgaaaaccaggatcaactcccgtg

SEQ ID NO: 430 - DNA, 50 bp, Artificial Sequence
  Description of Artificial Sequence Synthetic oligonucleotide
catttcgagtagcagtaatactcgcgtgatactccaggacgctacgaaac

SEQ ID NO: 431 - DNA, 50 bp, Artificial Sequence
  Description of Artificial Sequence Synthetic oligonucleotide
tctgcaaaggagtaagtcgatttgggtgatactccaggacgctacgaaac

SEQ ID NO: 432 - DNA, 50 bp, Artificial Sequence
  Description of Artificial Sequence Synthetic oligonucleotide
cgagcccactgggtgcatcctgagagtgatactccaggacgctacgaaac

SEQ ID NO: 433 - DNA, 50 bp, Artificial Sequence
  Description of Artificial Sequence Synthetic oligonucleotide
gttgtaggtggcacctctggtgagggtgatactccaggacgctacgaaac

SEQ ID NO: 434 - DNA, 25 bp, Artificial Sequence
  Description of Artificial Sequence Synthetic primer
gatctggctaggtgccacaacaaaa

SEQ ID NO: 435 - DNA, 25 bp, Artificial Sequence
  Description of Artificial Sequence Synthetic primer
gacatgctaaccacgttgcaggaaa

SEQ ID NO: 436 - DNA, 25 bp, Artificial Sequence
  Description of Artificial Sequence Synthetic primer
gacctcgtaaaagggggtatagaaa

SEQ ID NO: 437 - DNA, 25 bp, Artificial Sequence
  Description of Artificial Sequence Synthetic primer
aaaataccatcttggccattataaa

SEQ ID NO: 438 - DNA, 25 bp, Artificial Sequence
  Description of Artificial Sequence Synthetic primer
gagtgactgcaactaaaatgctaaa

SEQ ID NO: 439 - DNA, 25 bp, Artificial Sequence
  Description of Artificial Sequence Synthetic primer
tgtatcagaggattgcgttcgaaaa

SEQ ID NO: 440 - DNA, 25 bp, Artificial Sequence
  Description of Artificial Sequence Synthetic primer
gttcggggatacattctgagtaaaa

SEQ ID NO: 441 - DNA, 25 bp, Artificial Sequence
  Description of Artificial Sequence Synthetic primer
tgcaactagattgaggcctctaaaa

SEQ ID NO: 442 - DNA, 25 bp, Artificial Sequence
  Description of Artificial Sequence Synthetic primer
ctatatgtaggggctctaaccgaaa

SEQ ID NO: 443 - DNA, 25 bp, Artificial Sequence
  Description of Artificial Sequence Synthetic primer
catctgctgcgtttggaatacgaaa

SEQ ID NO: 444 - DNA, 25 bp, Artificial Sequence
  Description of Artificial Sequence Synthetic primer
ataccagccggctgatgatcgtaaa

SEQ ID NO: 445 - DNA, 25 bp, Artificial Sequence
  Description of Artificial Sequence Synthetic primer
agccactctgtagcactgatggaaa

SEQ ID NO: 446 - DNA, 25 bp, Artificial Sequence
  Description of Artificial Sequence Synthetic primer
taccctagttggcagttcatcgaaa

SEQ ID NO: 447 - DNA, 25 bp, Artificial Sequence
  Description of Artificial Sequence Synthetic primer
ataatagtcgctggtatggtacaaa

SEQ ID NO: 448 - DNA, 25 bp, Artificial Sequence
  Description of Artificial Sequence Synthetic primer
atttggaacaccgcagctcggtaaa

SEQ ID NO: 449 - DNA, 25 bp, Artificial Sequence
  Description of Artificial Sequence Synthetic primer
gaccccgtgcacggatgcatgaaaa

SEQ ID NO: 450 - DNA, 25 bp, Artificial Sequence
  Description of Artificial Sequence Synthetic primer
gtcgggcagcacccaagttctgaaa

SEQ ID NO: 451 - DNA, 25 bp, Artificial Sequence
  Description of Artificial Sequence Synthetic primer
agctgtggttaaggatagttcgaaa

SEQ ID NO: 452 - DNA, 25 bp, Artificial Sequence
  Description of Artificial Sequence Synthetic primer
agtgcaaattcgacacttgacgaaa

SEQ ID NO: 453 - DNA, 25 bp, Artificial Sequence
  Description of Artificial Sequence Synthetic primer
ggccctccttattaaacatccgaaa

SEQ ID NO: 454 - DNA, 25 bp, Artificial Sequence
  Description of Artificial Sequence Synthetic primer
actcactctgggcagacgcagaaaa

SEQ ID NO: 455 - DNA, 25 bp, Artificial Sequence
  Description of Artificial Sequence Synthetic primer
ttcgggcgttctgaagacctgtaaa

SEQ ID NO: 456 - DNA, 25 bp, Artificial Sequence
  Description of Artificial Sequence Synthetic primer
ccgggggagtcattgtattacgaaa

SEQ ID NO: 457 - DNA, 25 bp, Artificial Sequence
  Description of Artificial Sequence Synthetic primer
gtagaccgtagcgaacaccggaaaa

SEQ ID NO: 458 - DNA, 25 bp, Artificial Sequence
  Description of Artificial Sequence Synthetic primer
agtctcggttccgcatgcgtcgaaa

SEQ ID NO: 459 - DNA, 25 bp, Artificial Sequence
  Description of Artificial Sequence Synthetic primer
cataccgtcaactaatattctcaaa

SEQ ID NO: 460 - DNA, 25 bp, Artificial Sequence
  Description of Artificial Sequence Synthetic primer
gtgggatggagtccacgaaattaaa

SEQ ID NO: 461 - DNA, 25 bp, Artificial Sequence
  Description of Artificial Sequence Synthetic primer
ttggagtttagcgacacgcattaaa

SEQ ID NO: 462 - DNA, 25 bp, Artificial Sequence
  Description of Artificial Sequence Synthetic primer
atctatcttgaacccgggcgataaa

SEQ ID NO: 463 - DNA, 25 bp, Artificial Sequence
  Description of Artificial Sequence Synthetic primer
ggcactcgggtcttatccgttgaaa

SEQ ID NO: 464 - DNA, 25 bp, Artificial Sequence
  Description of Artificial Sequence Synthetic primer
gcttatacgcaactgtgtctggaaa

SEQ ID NO: 465 - DNA, 25 bp, Artificial Sequence
  Description of Artificial Sequence Synthetic primer
caaaagaggttgtcgtagctcgaaa

SEQ ID NO: 466 - DNA, 25 bp, Artificial Sequence
  Description of Artificial Sequence Synthetic primer
gtggatgtccaggttaactcaaaaa

SEQ ID NO: 467 - DNA, 25 bp, Artificial Sequence
  Description of Artificial Sequence Synthetic primer
aaggtgcttgagccatgggatcaaa

SEQ ID NO: 468 - DNA, 25 bp, Artificial Sequence
  Description of Artificial Sequence Synthetic primer
gtaacttcatacactccacattaaa

SEQ ID NO: 469 - DNA, 25 bp, Artificial Sequence
  Description of Artificial Sequence Synthetic primer
tttcgtgcaagtcaacaattgaaaa

SEQ ID NO: 470 - DNA, 25 bp, Artificial Sequence
  Description of Artificial Sequence Synthetic primer
gcatgaggccctgatgcagtgaccc

SEQ ID NO: 471 - DNA, 25 bp, Artificial Sequence
  Description of Artificial Sequence Synthetic primer
aacggtgatgtcgtcaaagattccc

SEQ ID NO: 472 - DNA, 25 bp, Artificial Sequence
  Description of Artificial Sequence Synthetic primer
cagacatctcctagcgagtcagccc

SEQ ID NO: 473 - DNA, 25 bp, Artificial Sequence
  Description of Artificial Sequence Synthetic primer
attggtgtctccccgagctgtaccc

SEQ ID NO: 474 - DNA, 25 bp, Artificial Sequence
  Description of Artificial Sequence Synthetic primer
tcgccatatcgtaccgatgtctccc

SEQ ID NO: 475 - DNA, 25 bp, Artificial Sequence
  Description of Artificial Sequence Synthetic primer
cgtagactaaccgactcatcgaccc

SEQ ID NO: 476 - DNA, 25 bp, Artificial Sequence
  Description of Artificial Sequence Synthetic primer
actgaccgtttaagggtccaagccc

SEQ ID NO: 477 - DNA, 25 bp, Artificial Sequence
  Description of Artificial Sequence Synthetic primer
tacgttcaccatcgtcaataggccc

SEQ ID NO: 478 - DNA, 25 bp, Artificial Sequence
  Description of Artificial Sequence Synthetic primer
gtcgccacgaacgctgaagaagccc

SEQ ID NO: 479 - DNA, 25 bp, Artificial Sequence
  Description of Artificial Sequence Synthetic primer
gctgcacgttgtctcacagcttccc

SEQ ID NO: 480 - DNA, 25 bp, Artificial Sequence
  Description of Artificial Sequence Synthetic primer
gctacgcgtcctccaatatgcgccc

SEQ ID NO: 481 - DNA, 25 bp, Artificial Sequence
  Description of Artificial Sequence Synthetic primer
gagtagggtaatacgttctacaccc

SEQ ID NO: 482 - DNA, 25 bp, Artificial Sequence
  Description of Artificial Sequence Synthetic primer
gaaccctttagctccacaattgccc

SEQ ID NO: 483 - DNA, 25 bp, Artificial Sequence
  Description of Artificial Sequence Synthetic primer
aataacgcatgcgttatcccacccc

SEQ ID NO: 484 - DNA, 25 bp, Artificial Sequence
  Description of Artificial Sequence Synthetic primer
aatgatcaacgaacgtcgctggccc

SEQ ID NO: 485 - DNA, 25 bp, Artificial Sequence
  Description of Artificial Sequence Synthetic primer
tatagcaatgagggccagtgatccc

SEQ ID NO: 486 - DNA, 25 bp, Artificial Sequence
  Description of Artificial Sequence Synthetic primer
tagctaagcttgtgctagattaccc

SEQ ID NO: 487 - DNA, 25 bp, Artificial Sequence
  Description of Artificial Sequence Synthetic primer
acggcgtcagttgtaaggatatccc

SEQ ID NO: 488 - DNA, 25 bp, Artificial Sequence
  Description of Artificial Sequence Synthetic primer
tatgataacccacttccaagttccc

SEQ ID NO: 489 - DNA, 25 bp, Artificial Sequence
  Description of Artificial Sequence Synthetic primer
ccataaccttagtatgtagtcgccc

SEQ ID NO: 490 - DNA, 25 bp, Artificial Sequence
  Description of Artificial Sequence Synthetic primer
cgtctgtggcaataacgcttcaccc

SEQ ID NO: 491 - DNA, 25 bp, Artificial Sequence
  Description of Artificial Sequence Synthetic primer
tatgcttcctggagctgcaagcccc

SEQ ID NO: 492 - DNA, 25 bp, Artificial Sequence
  Description of Artificial Sequence Synthetic primer
cggcattctgaacaactatatgccc

SEQ ID NO: 493 - DNA, 25 bp, Artificial Sequence
  Description of Artificial Sequence Synthetic primer
cgcatctgcacgtaaaacggcgccc

SEQ ID NO: 494 - DNA, 25 bp, Artificial Sequence
  Description of Artificial Sequence Synthetic primer
tcagggctacgcgacctcgtacccc

SEQ ID NO: 495 - DNA, 25 bp, Artificial Sequence
  Description of Artificial Sequence Synthetic primer
atgccgagattcgaatatcggaccc

SEQ ID NO: 496 - DNA, 25 bp, Artificial Sequence
  Description of Artificial Sequence Synthetic primer
ccaaattccgcgggccttgaacccc

SEQ ID NO: 497 - DNA, 25 bp, Artificial Sequence
  Description of Artificial Sequence Synthetic primer
acttgcgtacccatacatgtatccc

SEQ ID NO: 498 - DNA, 25 bp, Artificial Sequence
  Description of Artificial Sequence Synthetic primer
tagaagcgcgaagtataggatgccc

SEQ ID NO: 499 - DNA, 25 bp, Artificial Sequence
  Description of Artificial Sequence Synthetic primer
tagtaccggcaattccttgttgccc

SEQ ID NO: 500 - DNA, 25 bp, Artificial Sequence
  Description of Artificial Sequence Synthetic primer
aaccacgagtcgtcactgaccgccc

SEQ ID NO: 501 - DNA, 25 bp, Artificial Sequence
  Description of Artificial Sequence Synthetic primer
gtaaatggtctagaggttacggccc

SEQ ID NO: 502 - DNA, 25 bp, Artificial Sequence
  Description of Artificial Sequence Synthetic primer
cgtcggattgtgctatgtaaaaccc

SEQ ID NO: 503 - DNA, 25 bp, Artificial Sequence
  Description of Artificial Sequence Synthetic primer
gacagttcatctacacattgcaccc

SEQ ID NO: 504 - DNA, 25 bp, Artificial Sequence
  Description of Artificial Sequence Synthetic primer
aagggaaccggcacgaatcagtccc

SEQ ID NO: 505 - DNA, 25 bp, Artificial Sequence
  Description of Artificial Sequence Synthetic primer
tcattgctagcacctaccagacccc

(19) United States
(12) Patent Application Publication
(10) Pub. No.: US 2011/0003301 A1
(43) Pub. Date: Jan. 6, 2011

(54) METHODS FOR DETECTING GENETIC VARIATIONS IN DNA SAMPLES

(75) Inventors: Christopher K. RAYMOND, Seattle, WA (US); Jill F. MAGNUS, Seattle, WA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/776,356

(22) Filed: May 7, 2010

Related U.S. Application Data
(60) Provisional application No. 61/176,806, filed on May 8, 2009.
(60) Provisional application No. 61/241,352, filed on Sep. 10, 2009.

(51) Int. Cl. C12Q 1/68

(52) U.S. Cl. 435/6

(57) ABSTRACT

The invention provides methods, compositions and kits for detecting genetic variation in a DNA sample at one or more polymorphic loci of interest. In some embodiments, the invention provides methods, compositions, and kits for determining the nucleotide present at a single nucleotide variant position of interest in a test sample.

This application claims the filing date benefit of U.S. Provisional Application Nos.: 61/176,806, filed on May 8, 2009, and 61/241,352, filed on Sep. 10, 2009. The contents of each foregoing patent applications are incorporated by reference in their entirety.

Throughout this application various publications are referenced. The disclosures of these publications in their entireties are hereby incorporated by reference into this application in order to more fully describe the state of the art to which this invention pertains.

### BACKGROUND

Molecular profiling will be a key technology in achieving personalized medicine, such as personalized oncology health therapies. The genomes of all mammalian subjects, including humans, undergo spontaneous mutations during the course of evolution. The majority of such mutations create polymorphisms, such that the mutated sequence and the initial sequence co-exist in the species population. The majority of DNA base differences are functionally inconsequential because they do not affect the amino acid sequence of encoded proteins and/or they do not affect the expression levels of the encoded proteins. However, some polymorphisms that lie within genes or their promoters do have a phenotypic effect, such as physical appearance, disease susceptibility, disease resistance, and responsiveness to drug treatments. Single nucleotide polymorphisms (SNPs) represent the most frequent type of human population DNA variation. Other forms of variation include copy number variation (CNVs), as well as short tandem repeats (e.g., microsatellites), long tandem repeats (e.g., minisatellite), and other insertions and deletions.

The study of complex genomes, and in particular, the search for the genetic basis of disease in humans, requires genotyping on a massive scale, which is demanding in terms of cost, time, and labor. Such costly demands are even greater when the methodology employed involves serial analysis of individual DNA samples, i.e., separate reactions for individual samples. Resequencing of polymorphic areas in the genome that are linked to disease development will contribute greatly to the understanding of diseases, such as cancer, and therapeutic development. While high-throughput sequencing platforms (e.g., a flow cell for massively parallel sequencing) provide a vast quantity of data with regard to disease-associated patterns of genetic variation on a genome-wide scale, this capability comes at the cost of a higher error rate than has been associated with traditional DNA sequencing platforms. Therefore, follow-on validation of primary sequencing results is often carried out using labor intensive technologies such as locus-by-locus PCR and capillary resequencing in order to validate potential mutations, such as single nucleotide variants (SNVs).

Thus, there is a need for accurate, high-throughput, and cost-effective methods for high-throughput genotyping of target regions of the genome and/or transcriptome for pharmacogenetics applications, genetic disease association studies, and for validation of cell mutations detected in sequencing.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present invention provides a method of determining the genotype of a test sample at one or more polymorphic loci of interest, the method comprising: (a) contacting in a reaction mixture, a test sample comprising one or more polymorphic loci of interest within one or more target nucleic acid region(s) of interest with one or more set(s) of query oligonucleotides, wherein each set of query oligonucleotides comprises: (i) at least one 5′ ligation oligonucleotide comprising, from the 5′ to 3′ end, a first PCR primer binding region, a target-specific binding region selected to hybridize 5′ of a polymorphic locus of interest, and a 3′ region chosen to hybridize to either a consensus or variant nucleotide sequence at the polymorphic locus of interest; and (ii) a phosphorylated 3′ ligation oligonucleotide comprising, from the 5′ to 3′ end, a target-specific binding region selected to hybridize 3′ of the polymorphic locus of interest and a second PCR primer binding region, under conditions that allow hybridization between the query oligonucleotides and the target nucleic acid region(s) of interest; (b) contacting the reaction mixture of step (a) with DNA ligase under conditions suitable to ligate the 5′ ligation oligonucleotides having a 3′ region that hybridizes to the nucleotide sequence present at the polymorphic locus of interest in the test sample and the adjacent 3′ phosphorylated ligation oligonucleotides, thereby generating a plurality of ligation products indicative of the genotype of the test sample at the one or more polymorphic loci of interest; and (c) measuring the amount of the ligation products in the reaction mixture of step (b). In some embodiments, the one or more polymorphic loci of interest comprise one or more SNV position(s) of interest. In some embodiments, the test sample comprising one or more polymorphic loci of interest within one or more target nucleic acid region(s) of interest is contacted with a thermostable DNA ligase and one or more set(s) of query oligonucleotides.

In another aspect, the present invention provides a method of genotyping a test sample at one or more single nucleotide variant(s) (SNVs) position(s) of interest, the method comprising: (a) for each SNV position of interest, contacting in three separate reaction mixtures: (i) a synthetic template comprising the target region of interest having a consensus nucleotide at the SNV position of interest; (ii) a synthetic template comprising the target region of interest having a variant nucleotide at the SNV position of interest; and (iii) a test sample comprising the target region of interest comprising the SNV position of interest to be genotyped; with one or more set(s) of SNV query oligonucleotides, each set comprising: (i) a pair of allele-specific 5′ ligation oligonucleotides, the pair comprising a first 5′ ligation oligonucleotide comprising, from the 5′ to 3′ end, a first PCR primer binding region, a target-specific binding region selected to hybridize 5′ of the SNV nucleotide position of interest, and a 3′ region chosen to hybridize to the consensus nucleotide sequence at the SNV position of interest and a second 5′ ligation oligonucleotide comprising, from the 5′ to 3′ end, a first PCR primer binding region, a target-specific binding region selected to hybridize 5′ of the SNV nucleotide position of interest, and a 3′ region chosen to hybridize to the variant nucleotide sequence at the SNV position of interest; and (ii) a phosphorylated 3′ ligation oligonucleotide comprising from the 5′ to 3′ end, a target-specific binding region selected to hybridize 3′ of the SNV position of interest and a second PCR primer binding region, under conditions that allow hybridization between the SNV query oligonucleotides and the nucleic acid target regions of interest; (b) contacting the three separate reaction mixtures of step (a) with DNA ligase under conditions suitable to ligate the 5′ ligation oligonucleotides having a 3′ region that hybridizes to the nucleotide sequence present at the SNV nucleotide position of interest in the synthetic templates and test samples and the adjacent 3′ phosphorylated ligation oligonucleotides, thereby generating three separate ligation mixtures; and (c) measuring the amount of the ligation products in each of the three ligation mixtures of step (b). In some embodiments, the synthetic template comprising the target region of interest having a consensus nucleotide at the SNV position of interest, the synthetic template comprising the target region of interest having a variant nucleotide at the SNV position of interest, and the test sample comprising the target nucleic acid region(s) of interest comprising the SNV position of interest to be genotyped, are separately contacted with a thermostable DNA ligase and the one or more set(s) of query oligonucleotides. In some embodiments, step (c) comprises amplification of the ligation products with a plurality of detection primer pairs, each pair comprising a forward PCR primer that binds to the first PCR primer binding region in the 5′ ligation oligonucleotide and a reverse PCR primer that binds to the second PCR primer binding region in the 3′ ligation oligonucleotide.

In another aspect, the present invention provides a method of producing a multi-well container comprising a matrix of detection primer pairs for decoding a multiplexed assay, the method comprising: (a) designing a plurality of detection primer pairs, each pair comprising a forward primer and a reverse primer for amplifying a target nucleic acid molecule of interest comprising a 5′ primer binding region and a 3′ primer binding region, wherein each forward primer comprises a 5′ region that hybridizes to the 5′ primer binding region of the target nucleic acid molecule of interest and a 3′ region selected to avoid primer-dimer formation with the reverse primer; and wherein each reverse primer comprises a 5′ region that hybridizes to the 3′ primer binding region of the target nucleic acid molecule of interest and a 3′ region selected to avoid primer-dimer formation with the forward PCR primer; and (b) dispensing each of the plurality of detection primer pairs into a well in a multi-well container comprising an ordered array of wells arranged in a matrix comprising a plurality of perpendicular rows distributed along the vertical axis of the container and a plurality of columns distributed along the longitudinal axis of the container, such that each well in the matrix is positionally addressable.

In another aspect, the present invention provides a kit for genotyping a test sample at one or more polymorphic loci of interest, the kit comprising at least one set of query oligonucleotides for genotyping a polymorphic loci of interest, the set comprising: (i) at least one 5′ ligation oligonucleotide comprising, from the 5′ to 3′ end, a first PCR primer binding region, a target-specific binding region selected to hybridize 5′ of the polymorphic loci of interest, and a 3′ region chosen to hybridize to either a consensus or variant nucleotide sequence at the polymorphic loci of interest; and (ii) a phosphorylated 3′ ligation oligonucleotide comprising from the 5′ to 3′ end, a target-specific binding region selected to hybridize 3′ of the polymorphic loci of interest and a second PCR primer binding region.

The methods and kits of the invention can be used to genotype a haploid or diploid test sample for the presence or absence of one or more genetic variations, such as an insertion of one or more nucleotides, a deletion of one or more nucleotides, one or more single nucleotide variants (SNVs), one or more duplications, one or more inversions, one or more translocations, one or more repeat sequence expansions or contractions (i.e., changes in microsatellite sequences) at one or more polymorphic loci of interest within a target region of interest. The multi-well containers (e.g., assay plates) of the present invention can be used to measure the presence or amount of one or more target nucleic acid molecules of interest, such as ligation products generated from a multiplexed ligation-dependent genotyping assay according to various embodiments of the methods of the invention.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a method for determining the genotype of a test sample at a single nucleotide variant (SNV) position of interest using a single allele-specific 5′ ligation oligonucleotide, in accordance with an embodiment of the methods of the invention;

FIG. 2 illustrates a method for determining the genotype of a test sample at a single nucleotide variant (SNV) position of interest using a pair of 5′ allele-specific ligation oligonucleotides, in accordance with an embodiment of the methods of the invention, as described in Examples 1 and 3;

FIG. 3 illustrates representative allele-specific 5′ ligation oligonucleotides, 3′ ligation oligonucleotides, and detection PCR primers for use in the methods, multi-well containers and kits of the invention;

FIG. 4 illustrates exemplary reagents for use in a multiplexed ligation-dependent genotyping assay for genotyping a plurality of SNV positions of interest, wherein each assay for each target region of interest (e.g., Gene 1) is carried out with a pair of allele-specific 5′ ligation oligos (300, 400), and a common 3′ ligation oligo (500), and the quantitative PCR detection assay is carried out using corresponding detection PCR primer pairs (600, 700) disposed in a multi-well assay plate at discrete well locations (e.g., plate location A1, B1);

FIG. 5A shows a perspective view of a representative multi-well container of the present invention comprising pairs of detection PCR primers arranged in a matrix for decoding a multiplexed assay comprising query oligonucleotides having regions complementary to the detection PCR primer pairs;

FIG. 5B shows a portion of a transverse cross-section of the representative multi-well container shown in FIG. 5A;

FIG. 6 illustrates the decoding results obtained after carrying out a quantitative PCR assay in three separate, identical assay plates comprising detection PCR primer pairs arranged in a matrix, wherein (A) shows the assay results from a ligation mixture comprising a plurality of consensus synthetic templates and a pool of SNV query ligation oligos, (B) shows the assay results from a ligation mixture comprising a plurality of variant synthetic templates and the pool of SNV query ligation oligos, and (C) shows the assay results from a ligation mixture comprising a test sample and the pool of SNV query ligation oligos, wherein each well (826) on the assay plate (800) contains a unique pair of PCR primers corresponding to a set of target-specific SNV query oligonucleotides, such that adjacent wells (e.g., A1 and B1) provide the qPCR results for a pair of alleles at a particular SNV position of interest, as further illustrated in FIG. 4 and described in Examples 2 and 3;

FIG. 7 illustrates a method of enriching a population of DNA molecules for target regions of interest using capture probes (1200), in accordance with an embodiment of the methods of the invention, as described in Example 3;

FIG. 8 is a flow chart showing the steps of a method for enriching a population of DNA molecules for target regions of interest with solution-based capture using target capture probes (1200), in accordance with an embodiment of the methods of the invention;

FIG. 9 is a flow chart showing the steps of a ligation-dependent genotyping assay in accordance with various embodiments of the invention; and

FIG. 10 is a flow chart showing the steps of a multiplexed ligation-dependent genotyping assay for simultaneously genotyping a plurality of SNV positions of interest, in accordance with various embodiments of the invention.

### DETAILED DESCRIPTION

This section presents a detailed description of the many different aspects and embodiments that are representative of the inventions disclosed herein. This description is by way of several exemplary illustrations of varying detail and specificity. Other features and advantages of these embodiments are apparent from the additional descriptions provided herein, including the different examples. The provided examples illustrate different components and methodology useful in practicing various embodiments of the invention. The examples are not intended to limit the claimed invention. Based on the present disclosure, the ordinary skilled artisan can identify and employ other components and methodology useful for practicing the present invention.

### I. Definitions

Unless defined otherwise, all technical and scientific terms used herein have the meaning commonly understood by one of ordinary skill in the art to which this invention belongs. Practitioners are particularly directed to Sambrook, et al., Molecular Cloning: A Laboratory Manual, 2d ed., Cold Spring Harbor Press, Plainsview, N.Y. (1989); and Ausubel, et al., Current Protocols in Molecular Biology (Supplement 47), John Wiley & Sons, New York (1999), for definitions and terms of the art.

It is contemplated that the use of the term “about” in the context of the present invention is to connote inherent problems with precise measurement of a specific element, characteristic, or other trait. Thus, the term “about,” as used herein in the context of the claimed invention, simply refers to an amount or measurement that takes into account single or collective calibration and other standardized errors generally associated with determining that amount or measurement. For example, a concentration of “about” 100 mM of Tris can encompass an amount of 100 mM±0.5 mM, if 0.5 mM represents the collective error bars in arriving at that concentration. Thus, any measurement or amount referred to in this application can be used with the term “about,” if that measurement or amount is susceptible to errors associated with calibration or measuring equipment, such as a scale, pipetteman, pipette, graduated cylinder, etc.

The use of the words “a” or “an” when used in conjunction with the term “comprising” in the claims and/or the specification may mean “one,” but it is also consistent with the meaning of “one or more,” “at least one,” and “one or more than one.”

As used herein, the term “nucleic acid molecule” encompasses both deoxyribonucleotides and ribonucleotides and refers to a polymeric form of nucleotides including two or more nucleotide monomers. The nucleotides can be naturally occurring, artificial, and/or modified nucleotides.

As used herein, the term “oligonucleotide” refers to a single-stranded multimer of nucleotides of from about 10 to 200 nucleotides that is usually synthetic.

As used herein, an “isolated nucleic acid” is a nucleic acid molecule that exists in a physical form that is non-identical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or intemucleoside bonds not found in nature. When, instead, composed of natural nucleosides in phosphodiester linkage, a nucleic acid can be said to be “isolated” when it exists at a purity not found in nature, where purity can be adjudged with respect to the presence of nucleic acids of other sequences, with respect to the presence of proteins, with respect to the presence of lipids, or with respect to the presence of any other component of a biological cell, or when the nucleic acid lacks a sequence that flanks an otherwise identical sequence in an organism's genome, or when the nucleic acid possesses a sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes, or as integrated into a host cell chromosome.

As used herein, “subject” refers to an organism or to a cell sample, tissue sample, or organ sample derived therefrom, including, for example, cultured cell line, biopsy, blood sample, or fluid sample containing a cell. For example, an organism may be an animal, including but not limited to, an animal such as a cow, a pig, a mouse, a rat, a chicken, a cat, a dog, etc., and is usually a mammal, such as a human.

As used herein, the term “specifically bind” refers to two components (e.g., target-specific binding region and target) that are bound (e.g., hybridized, annealed, complexed) to one another sufficiently that the intended capture and enrichment steps can be conducted. As used herein, the term “specific” refers to the selective binding of two components (e.g., target-specific binding region and target) and not generally to other components unintended for binding to the subject components.

As used herein, the term “high stringency hybridization conditions” means any condition in which hybridization will occur when there is at least 95%, preferably about 97% to 100% nucleotide complementarity (identity) between the nucleic acid sequences of the nucleic acid molecule and its binding partner. However, depending upon the desired purpose, the hybridization conditions may be “medium stringency hybridization,” which can be selected that require less complementarity, such as from about 50% to about 90% (e.g., 60%, 70%, 80%, 85%). The comparison of sequences and determination of percent identity between two sequences can be accomplished using a mathematical algorithm of Karlin and Altschul (Proc. Natl. Acad. Sci. USA 87:2264-2268 (1990)), modified as in Karlin and Altschul (Proc. Natl. Acad. Sci. USA 90:5873-5877 (1993)). Such an algorithm is incorporated into the NBLAST and XBLAST programs of Altschul et al. (J. Mol. Biol. 215:403-410 (1990)).

As used herein, the term “complementary” refers to nucleic acid sequences that are capable of base-pairing according to the standard Watson-Crick complementary rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA.

As used herein, the term “target nucleotide” refers to a nucleic acid molecule or polynucleotide in a starting population of nucleic acid molecules having a target sequence whose presence and/or amount and/or nucleotide sequence is desired to be determined and which has an affinity for a given ligation oligonucleotide. Examples of targets include regions of genomic DNA, PCR amplified products derived from RNA or DNA, DNA derived from RNA or DNA, ESTs, cDNA, and mutations, variants or modifications thereof.

As used herein, the term “target sequence” refers generally to a nucleic acid sequence on a single strand of nucleic acid. The target sequence may be a portion of a gene, a regulatory sequence, genomic DNA, cDNA, RNA including mRNA and rRNA, or others. The target sequence may be a target sequence from a sample, or a secondary target, such as a product of an amplification reaction.

As used herein, the term “predetermined nucleic acid sequence” means that the nucleic acid sequence of a nucleic acid probe is known and was chosen before synthesis of the nucleic acid molecule in accordance with the invention disclosed herein.

As used herein, the term “essentially identical” as applied to synthesized and/or amplified nucleic acid molecules refers to nucleic acid molecules that are designed to have identical nucleic acid sequences, but that may occasionally contain minor sequence variations in comparison to a desired sequence due to base changes introduced during the nucleic acid molecule synthesis process, amplification process, or due to other processes in the method. As used herein, essentially identical nucleic acid molecules are at least 95% identical to the desired sequence, such as at least 96%, such as at least 97%, such as at least 98%, such as at least 99% identical, or absolutely identical, to the desired sequence.

As used herein, the term “resequencing” refers to a technique that determines the sequence of a genome of an organism using a reference sequence that has already been determined. It should be understood that resequencing may be performed on both the entire genome/transcriptome of an organism or a portion of the genome/transcriptome large enough to include the genetic change of the organism as a result of selection. Resequencing may be carried out using various sequencing methods, such as any sequencing platform amenable to producing DNA sequencing reads that can be aligned back to a reference genome, and is typically based on highly parallel technologies such as, for example, dideoxy “Sanger” sequencing, pyrosequencing on beads (e.g., as described in U.S. Pat. No. 7,211,390, assigned to 454 Life Sciences Corporation, Brandord, Conn.), ligation based sequencing on beads (e.g., Applied Biosystems Inc./Invitrogen), sequencing on glass slides (e.g., Illumina Genome Analyzer System, based on technology described in WO 98/44151 (Mayer, P., and Farinelli, L.)), microarrays, or fluorescently labeled micro-beads.

As used herein, the term “polymorphism” refers to the occurrence of two or more genetically determined alternative sequences or alleles in a population. A “polymorphic locus” refers to the locus at which genetic variation occurs. A polymorphic locus can include any type of genetic variation, such as an insertion of one or more nucleotides, a deletion of one or more nucleotides, one or more single nucleotide variants (SNVs), one or more duplications, one or more inversions, one or more translocations, one or more repeat sequence expansions or contractions (i.e., changes in microsatellite sequences). In some embodiments, a polymorphic locus can be as small as one base pair (single nucleotide variant (SNV), which encompasses a single nucleotide polymorphism (SNP)). The first identified allele of a polymorphic locus is arbitrarily designated as the “consensus” allele and the other allele is designated as the “variant” (also sometimes referred as a “mutant”) allele. Typically, a polymorphic locus has at least two alleles, each occurring at a frequency of greater than 1% of a selected population.

The allele occurring most frequently in a selected population is sometimes referred to as the “wild-type” or “consensus” allele. Diploid organisms may be homozygous or heterozygous for the variant allele. The variant allele may or may not produce an observable physical or biochemical characteristic (“phenotype”) in an individual carrying the variant allele. For example, a variant allele may alter the enzymatic activity of a protein encoded by a gene of interest.

As used herein, the term “genetic variation” refers to genotypic differences among individuals in a population, at one or more polymorphic loci, and includes an insertion of one or more nucleotides, a deletion of one or more nucleotides, one or more single nucleotide sequence variations (SNVs), such as SNPs, copy number variation, such as one or more duplications, sequence rearrangements, such as one or more inversions, one or more translocations, or one or more repeat sequence expansions or contractions (i.e., changes in microsatellite sequences) at one or more polymorphic loci of interest within a target region of interest as compared to known reference sequences.

As used herein, the term “single nucleotide variant” or “SNV” refers to a DNA base within an established nucleotide sequence that differs from the known reference sequences. SNVs may be found within a patient sample (e.g., a tumor), they may or may not be present in unperturbed populations, and they include naturally occurring single nucleotide polymorphisms, also referred to as “SNPs”.

As used herein, the term “single nucleotide polymorphism” or “SNP” refers to a single nucleotide position in a genomic sequence for which two or more alternative alleles are present at an appreciable frequency (e.g., at least 1%) in a population of organisms.

As used herein, the term “genotype” broadly refers to the genetic composition of an organism, including, for example, whether a diploid organism is heterozygous or homozygous for one or more single nucleotide variant alleles (SNVs) at a position of interest.

As used herein, the term “haplotype” refers to the identity of the nucleotide(s) that are present at a polymorphic position in the genome of a cell. For example, if the haplotype is bivariant (e.g., “A” and C,” then the haplotypes are AA, CC and AC).

### II. Aspects and Embodiments of the Invention

In accordance with the foregoing, in one aspect, the invention provides a method of determining the genotype of a test sample at one or more polymorphic loci of interest, the method comprising: (a) contacting in a reaction mixture, a test sample comprising one or more polymorphic loci of interest within one or more target nucleic acid region(s) of interest with one or more set(s) of query oligonucleotides, wherein each set of query oligonucleotides comprises: (i) at least one 5′ ligation oligonucleotide comprising, from the 5′ to 3′ end, a first PCR primer binding region, a target-specific binding region selected to hybridize 5′ of a polymorphic locus of interest, and a 3′ region chosen to hybridize to either a consensus or variant nucleotide sequence at the polymorphic locus of interest, and (ii) a phosphorylated 3′ ligation oligonucleotide comprising, from the 5′ to 3′ end, a target-specific binding region selected to hybridize 3′ of the polymorphic locus of interest and a second PCR primer binding region, under conditions that allow hybridization between the query oligonucleotides and the target nucleic acid region(s) of interest; (b) contacting the reaction mixture of step (a) with DNA ligase under conditions suitable to ligate the 5′ ligation oligonucleotides having a 3′ region that hybridizes to the nucleotide sequence present at the polymorphic locus of interest in the test sample and the adjacent 3′ phosphorylated ligation oligonucleotides, thereby generating a plurality of ligation products indicative of the genotype of the test sample at the one or more polymorphic loci of interest; and (c) measuring the amount of the ligation products in the reaction mixture of step (b).

In some embodiments of the method, the hybridization and ligation steps are combined (i.e., coupled), wherein a test sample comprising one or more polymorphic loci of interest within one or more target nucleic acid region(s) of interest is contacted with a thermostable DNA ligase and one or more set(s) of query oligonucleotides. In other embodiments of the method, the hybridization and ligation reactions are carried out sequentially under separate reaction conditions (i.e., uncoupled), and may utilize either thermostable or non-thermostable DNA ligase.

The methods described herein may be used to detect any type of genetic variation, such as an insertion of one or more nucleotides, a deletion of one or more nucleotides, one or more single nucleotide sequence variations (SNVs), such as SNPs, copy number variation, such as one or more duplications, sequence rearrangements, such as one or more inversions, one or more translocations, or one or more repeat sequence expansions or contractions (i.e., changes in microsatellite sequences) at the polymorphic loci of interest in either a haploid or diploid sample of interest as compared to known reference sequences.

In some embodiments, the genetic variation detected is a single nucleotide variation (SNV) at an SNV position of interest. As described in Examples 1 and 3, it has been determined that the sensitivity of the assay methods described herein for a single mismatch adjacent the ligation site can be used to distinguish between two sequences that differ only with respect to the single nucleotide at the SNV position of interest. Accordingly, it will be understood by those of skill in the art that the methods described and demonstrated herein for use in detecting single nucleotide variations can also be used to detect larger regions of genetic variation, such as insertions, deletions, and sequence rearrangements in a haploid or diploid sample of interest. It will therefore be understood by those of skill in the art that while the descriptions herein of methods, kits, and compositions are described with reference to the detection of single nucleotide variants (SNVs), the methods, compositions, and kits are not intended to be limited to detection of SNVs, and are generally applicable to the detection of any type of genetic variation at one or more polymorphic loci of interest. Non-limiting examples of polymorphic loci of interest that may be detected using the methods described herein include nucleotide insertions, deletions, duplications, inversions, translocations, and changes in microsatellite sequences (i.e., sequence expansions and contractions) wherein the methods described herein are suitable for detecting various types of DNA rearrangements in addition to detecting changes in a nucleotide base sequence.

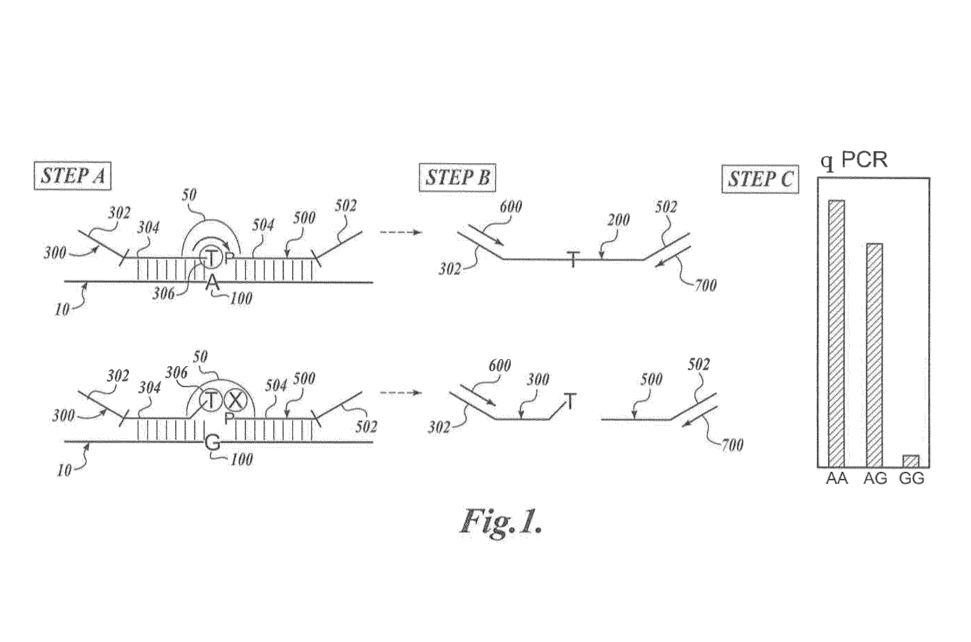

FIG. 1 illustrates an embodiment of this aspect of the method of the invention. As shown in FIG. 1 at Step A, an assay is carried out using a set of query oligonucleotides (e.g., SNV query oligonucleotides) comprising two oligos per polymorphic locus of interest (e.g., SNV position of interest): an allele specific 5′ ligation oligo and a common phosphorylated 3′ ligation oligo, based on the following steps. As shown in Step A, the test diploid genome 10 has a first allele with an “A” nucleotide at the SNV position of interest 100, and a second allele with a “G” nucleotide at the SNV position of interest 100. At Step A, an allele-specific 5′ ligation oligo 300 and 3′ ligation oligo 500 are each hybridized to the target region of the test diploid genome 10 that contains the SNV position of interest 100 under conditions that allow hybridization between the SNV query oligos and the target nucleic acid region of interest. As shown in FIG. 1, the 5′ ligation oligo 300 comprises, from the 5′ to 3′ end, a first PCR primer binding region 302, a target-specific binding region 304 selected to hybridize immediately 5′ of the SNV position of interest 100, and a 3′ region 306 (shown as comprising nucleotide “T”) that is complementary to the wild-type sequence “A” in the test genome (and, therefore, not complementary to the SNV sequence “G”).

As further shown in FIG. 1, the phosphorylated (P) 3′ ligation oligo 500 comprises, from the 5′ to 3′ end, a target-specific binding region 504 selected to hybridize immediately 3′ of the SNV position of interest 100, and a region 502 at the 3′ end that contains a second PCR primer binding region.

As shown in FIG. 1, Step A, a DNA ligase enzyme 50 is contacted with the annealed mixture. For example, a thermostable DNA ligase enzyme may be present in the annealed mixture, or a non-thermostable DNA ligase enzyme may be added after the mixture is annealed. As further shown in FIG. 1, Step B, as a result of the ligation reaction, the adjacent oligos 300 and 500 that are annealed to the test genome with an “A” at SNV position 100, a ligation product 200 is formed that is indicative of the genotype of the test sample, with a “T” at SNV position 100, flanked by 5′ primer binding region 302 and 3′ primer binding region 502. In contrast, the oligo 300 that is annealed to the test genome with a “G” at SNV position 100, resulting in a mismatch, does not form a ligation product with the adjacent oligo 500, due to the mismatch.

As shown in FIG. 1, Step C, the ligation product formed in Step B may be assayed by a quantitative PCR (qPCR) assay using a forward PCR primer 600 that binds to the 5′ primer binding region 302 and a reverse PCR primer 700 that binds to the 3′ primer binding region 502 on the ligation product 200. However, as illustrated in the representative graph shown in Step C, it is more difficult to distinguish between a homozygote (e.g., AA or GG) and a heterozygote (e.g., AG) using the two oligo per SNV assay approach, with one 5′ ligation oligo specific for a given allele in a diploid organism. In haploid organisms, the expected genotype will only be consensus or variant, and not potentially a heterozygous blend of the two as is found in a diploid organism such as a human.

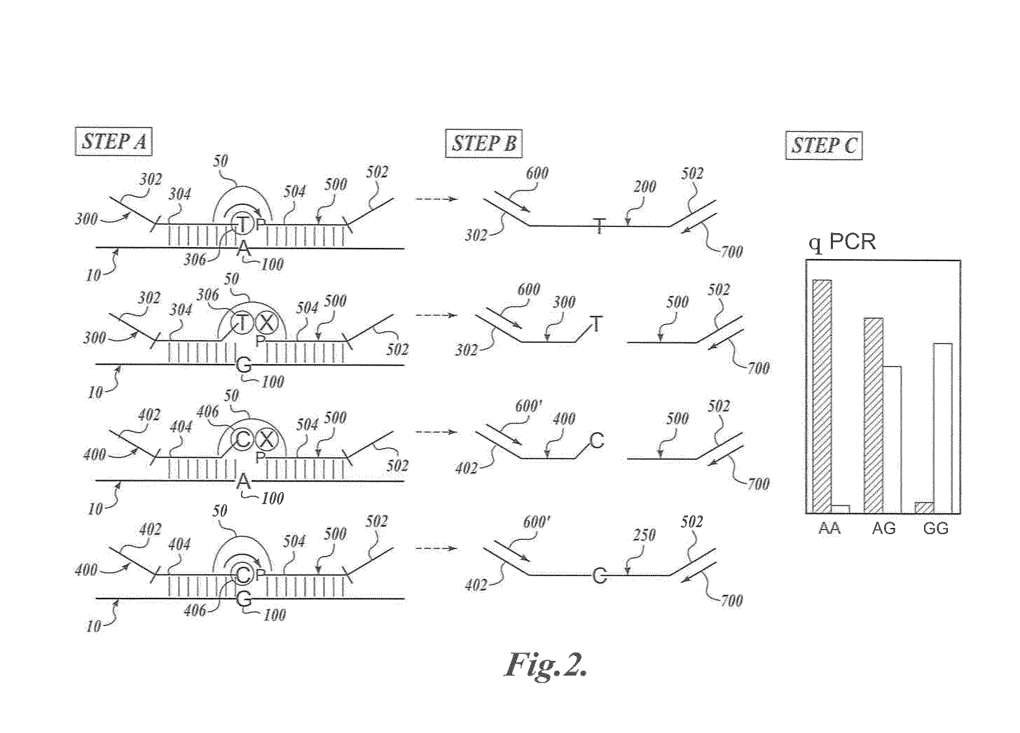

In another embodiment of the method, each set of query oligonucleotides (e.g., SNV query oligonucleotides) according to step (a) comprises a pair of allele-specific 5′ ligation oligonucleotides for each SNV position of interest, the pair comprising a first 5′ ligation oligonucleotide comprising a 3′ region chosen to hybridize to the consensus nucleotide sequence at the SNV position of interest and a second 5′ ligation oligonucleotide comprising a 3′ region chosen to hybridize to the variant nucleotide sequence at the SNV position of interest. In accordance with this embodiment, as shown in FIG. 2, an assay is carried out using a set of SNV query oligonucleotides comprising three primers per SNV position of interest: an allele specific 5′ ligation oligo that binds to the consensus sequence at the SNV position of interest 100, an allele specific 5′ ligation oligo that binds to the variant sequence at the SNV position of interest 100, and a common phosphorylated 3′ ligation oligo. As shown in FIG. 2, Step A, the test diploid genome 10 has a first allele with an “A” nucleotide at the SNV position of interest 100, and a second allele with a “G” nucleotide at the SNV position of interest 100. At Step A, the set of SNV oligonucleotides comprising the three ligation oligos: a 5′ ligation oligo 300 that binds to the consensus sequence “A” at position 100, a 5′ ligation oligo 400 that binds to the variant sequence “G” at position 100, and a common 3′ ligation primer 500 are annealed to the region of the test diploid genome 10 that contains the SNV position 100.

The 5′ ligation oligo 300 comprises, from the 5′ to 3′ end, a first PCR primer binding region 302, a target-specific binding region 304 selected to hybridize immediately 5′ of the SNV position of interest 100, and a 3′ region 306 (shown as comprising nucleotide “T”) that is complementary to the wild-type sequence “A” in the test genome (and, therefore, not complementary to the SNV sequence “G”).

The 5′ ligation oligo 400 comprises, from the 5′ to 3′ end, a first PCR primer binding region 402, a target-specific binding region 404 selected to hybridize immediately 5′ of the SNV position of interest 100, and a 3′ region 406 (shown as comprising nucleotide “C”) that is complementary to the variant sequence “G” in the test genome (and, therefore, not complementary to the wild-type sequence “A”).

As further shown in FIG. 2, Step A, the phosphorylated (P) 3′ common ligation oligo 500 comprises, from the 5′ to 3′ end, a target-specific binding region 504 selected to hybridize immediately 3′ of the SNV position of interest 100, and a region 502 at the 3′ end that contains a second PCR primer binding region.

As shown in FIG. 2, Step A, ligase enzyme 50 is either present in the annealed mixture, or added to the annealed mixture, and as shown in FIG. 2, Step B, as a result of the ligation reaction, a ligation product 200 is formed by ligating the oligo 300 that is annealed to the test genome with an “A” at SNV position 100 and the adjacent common oligo 500, with a “T” at SNV position 100, flanked by 5′ primer binding region 302 and 3′ primer binding region 502. In contrast, the oligo 300 that is annealed to the test genome with a “G” at SNV position 100, resulting in a mismatch, does not form a ligation product with the adjacent oligo 500, due to the mismatch. As further shown in FIG. 2, Step B, a ligation product 250 is formed by ligating the oligo 400 that is annealed to the test genome with a “G” at position 100 and the adjacent common oligo 500, with a “C” at SNV position 100, flanked by 5′ primer binding region 402 and 3′ primer binding region 502.

As shown in FIG. 2, Step C, the amount of the ligation products 200 and 250 formed in Step B may be measured by performing an assay, such as a quantitative PCR (qPCR) assay using a first set of primers: forward PCR primer 600 that has a region 602 that binds to the 5′ primer binding region 302 and a reverse PCR primer 700 that binds to the 3′ primer binding region 502 on the ligation product 200, and a second set of primers: forward PCR primer 600′ that has a region 602 that binds to the 5′ primer binding region 402 and a reverse PCR primer 700 that binds to the 3′ primer binding region 502 on the ligation product 250. As illustrated in the representative graph shown in Step C, the use of the three query oligos per SNV assay allows a read-out of both alleles at a polymorphic site in a diploid sample. Therefore, homozygotes (e.g., AA or GG) are read out in one or the other PCR assays, while heterozygotes (e.g., AG), are read out in both PCR assays, thereby allowing for unambiguous identification of homozygotes and heterozygotes.

Query Oligonucleotides

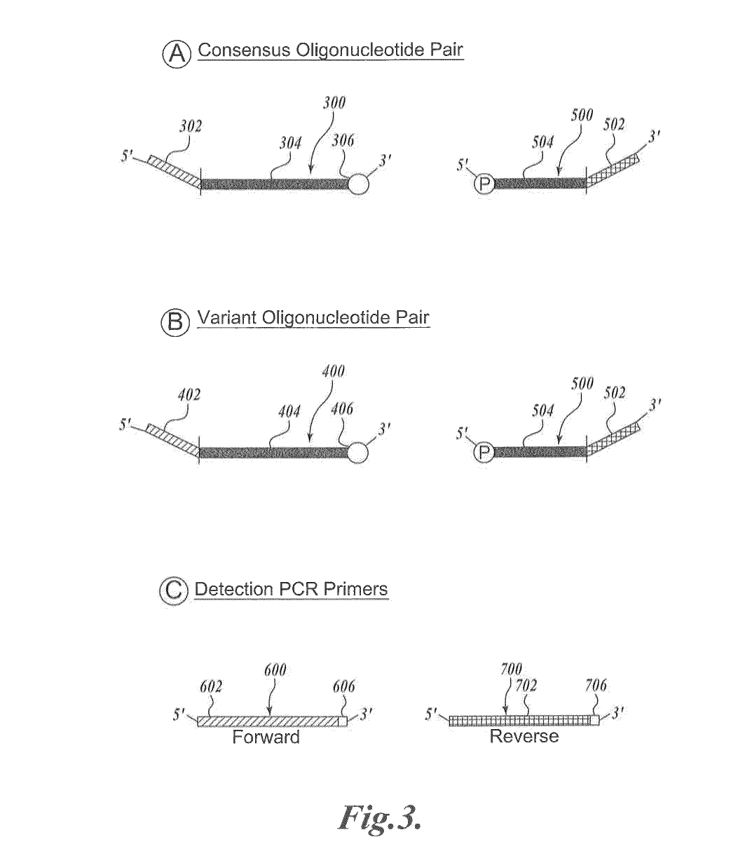

As shown in FIG. 3, the query oligonucleotides for use in the various embodiments of the ligation-dependent genotyping methods described herein (e.g., SNV query oligonucleotides), include a 5′ ligation oligonucleotide (300), a variant 5′ ligation oligonucleotide (400), and a common phosphorylated 3′ ligation oligonucleotide (500).

5′ Ligation Oligonucleotides (300, 400)

As shown in FIG. 3A, the 5′ ligation consensus oligo 300 comprises, from the 5′ to 3′ end, a first PCR primer binding region 302, a target-specific binding region 304 selected to hybridize to the target nucleic acid region immediately 5′ of the SNV position of interest 100, and a 3′ region 306 that is complementary to the wild-type (i.e., consensus) sequence at the SNV position of interest.

As shown in FIG. 3B, the 5′ ligation variant oligonucleotide 400 comprises, from the 5′ to 3′ end, a first PCR primer binding region 402, a target-specific binding region 404 selected to hybridize immediately 5′ of the SNV position of interest 100, and a 3′ region 406 that is complementary to the variant (e.g., mutant) sequence at the SNV position of interest.

The length of each 5′ ligation oligo (300, 400) is typically at least 40 nucleotides, such as at least 45 nucleotides, at least 50 nucleotides, at least 55 nucleotides, at least 60 nucleotides, at least 65 nucleotides, at least 70 nucleotides, up to a maximum length of about 200 nucleotides. In some embodiments, the 5′ ligation oligos are each from about 45 nucleotides to about 70 nucleotides in length.

The target-specific binding region 304, 404, selected to hybridize immediately 5′ of the SNV position of interest 100, is typically at least 10 nucleotides in length, such as at least 15 nucleotides, at least 20 nucleotides, at least 25 nucleotides, at least 30 nucleotides, at least 35 nucleotides, at least 40 nucleotides, at least 45 nucleotides, at least 50 nucleotides, up to 150 nucleotides in length. In some embodiments, the target-specific binding region 304, 404 is from about 20 to 30 nucleotides in length. Typically, the target-specific binding region 304, 404 is designed to have a sequence that is complementary, or substantially complementary, to the nucleic acid sequence contained in a region of interest immediately 5′ of an SNV position of interest 100. In one embodiment, the target-specific binding region (304, 404) comprises a sequence that is 100% complementary to the target region 5′ of the SNV position of interest.

In another embodiment, the target-specific binding region (304, 404) comprises a first region comprising the 20 nucleotides 5′ (upstream) to the SNV position of interest that is 100% complementary to the target region, and a second region comprising from 21 nucleotides 5′ (further upstream) of the SNV position of interest to the 5′ end of the target-specific region (304, 404), wherein the second region comprises a sequence that is substantially complementary (i.e., at least 90% identical, at least 95% identical, at least 96% identical, at least 97% identical, at least 98% identical or at least 99% identical) to the target region 5′ of the SNV position of interest.

One of skill in the art can use art-recognized methods to determine the features of a target-specific binding region (304, 404) that will hybridize to the target region 5′ of the SNV position of interest with minimal non-specific hybridization. For example, one of skill can determine experimentally the features such as length, base composition, and degree of complementarity that will enable a nucleic acid molecule (e.g., the target-specific binding region of a ligation oligo) to specifically hybridize to another nucleic acid molecule (e.g., the nucleic acid target) under conditions of selected stringency, while minimizing non-specific hybridization to other substances or molecules. The target-specific binding region may be designed to take into account genomic features of the target region, such as genetic variation (other than at the SNV position of interest), G:C content, predicted oligo Tm, and the like.

As shown in FIGS. 3A and 3B, the 5′ ligation oligos further comprise a region 306, 406 at the 3′ end of the oligo that has a sequence selected to hybridize to either the consensus (306) or variant (406) nucleotide present at the SNV position of interest. Typically, the region 306, 406 is a single nucleotide in length located at the 3′ end of the ligation oligo 300, 400.

In some embodiments, the region 306, 406 is larger than a single nucleotide in length (e.g., from 2 nt to 1000 nt, 10,000 nt, 100,000 nt or larger), and is selected to detect a genetic variation, such as an insertion, a deletion, or a rearrangement (e.g., inversion, translocation) in the nucleotide sequence at the polymorphic position of interest, such as an SNV position of interest. It is noted that the methods described herein for detecting genetic variation at an SNV position of interest are not limited by the size of the polymorphic locus of interest, and may be used, for example, to detect the presence or absence of a rearrangement, such as a translocation event, between chromosomes in a haploid or diploid sample. In such embodiments, all that is required is the precise knowledge of the nucleotide sequence of the translocation break points.

3′ Ligation Oligonucleotides (500)

As shown in FIGS. 3A and 3B, the phosphorylated (P) 3′ common ligation oligo 500 comprises, from the 5′ to 3′ end, a target-specific binding region 504 selected to hybridize immediately 3′ of the SNV position of interest 100, and a region 502 at the 3′ end that contains a second PCR primer binding region. In operation, the 3′ ligation primer 500 is typically phosphorylated at the 5′ end prior to annealing to the test genome 10.

The length of each 3′ ligation oligo (500) is typically at least 40 nucleotides, such as at least 45 nucleotides, at least 50 nucleotides, at least 55 nucleotides, at least 60 nucleotides, at least 65 nucleotides, at least 70 nucleotides, up to a maximum length of about 200 nucleotides. In some embodiments, the 3′ ligation oligos are each from about 45 nucleotides to about 70 nucleotides in length.

The target-specific binding region 504, selected to hybridize to the target nucleic acid region starting at the nucleotide position immediately 3′ of the SNV position of interest 100, is typically at least 10 nucleotides in length, such as at least 15 nucleotides, at least 20 nucleotides, at least 25 nucleotides, at least 30 nucleotides, at least 35 nucleotides, at least 40 nucleotides, at least 45 nucleotides, at least 50 nucleotides, up to 150 nucleotides in length. In some embodiments, the target-specific binding region 504 is from about 20 to 30 nucleotides in length. The target-specific binding region 504 is designed to have a sequence that is complementary, or substantially complementary, to the nucleic acid sequence contained in a region of interest immediately 3′ of an SNV position of interest 100. In one embodiment, the target-specific binding region (504) comprises a sequence that is 100% complementary to the target region 3′ of the SNV position of interest. In another embodiment, the target-specific binding region (504) comprises a sequence that is substantially complementary (i.e., at least 90% identical, at least 95% identical, at least 96% identical, at least 97% identical, at least 98% identical or at least 99% identical) to the target region 5′ of the SNV position of interest.

In another embodiment, the target-specific binding region (504) comprises a first region comprising the 20 nucleotides downstream (3′) of the SNV position of interest that is 100% complementary to the target region, and a second region comprising from 21 nucleotides further 3′ of the SNV position of interest to the 3′ end of the target-specific region (504), wherein the second region comprises a sequence that is substantially complementary (i.e., at least 90% identical, at least 95% identical, at least 96% identical, at least 97% identical, at least 98% identical, or at least 99% identical) to the target region 3′ of the SNV position of interest.

The 5′ ligation oligonucleotides (300) and variant 5′ ligation oligonucleotides (400), each include PCR primer binding regions 302, 402 (also referred to as “primer tails”) located at the 5′ end of the oligos, for binding to forward PCR primers for use in a quantitative PCR assay. Similarly, the 3′ ligation oligonucleotides (500) each include a PCR primer binding region 502 (primer tail) located at the 3′ end of the oligo, for binding to reverse PCR primers.

The PCR primer binding regions 302, 402, and 502, are typically from about 10 to 50 nucleotides in length, such as at least 10 nucleotides in length, such as at least 15 nucleotides, at least 20 nucleotides, at least 25 nucleotides, at least 30 nucleotides, at least 35 nucleotides, at least 40 nucleotides, at least 45 nucleotides, or at least 50 nucleotides in length. In some embodiments, the PCR primer binding regions 302, 402, and 502 are from about 20 to 30, such as about 25 nucleotides in length.

In some embodiments, the 5′ consensus ligation oligo 300 has a different primer binding region 302 than the primer binding region 402 of the 5′ variant ligation oligo 400, to allow for detection of the presence or amount of the consensus ligation product 200 and the variant ligation product 250 in a single ligation reaction using two different sets of detection PCR primers, each set designed to detect either the consensus ligation product 200 or the variant ligation product 250.

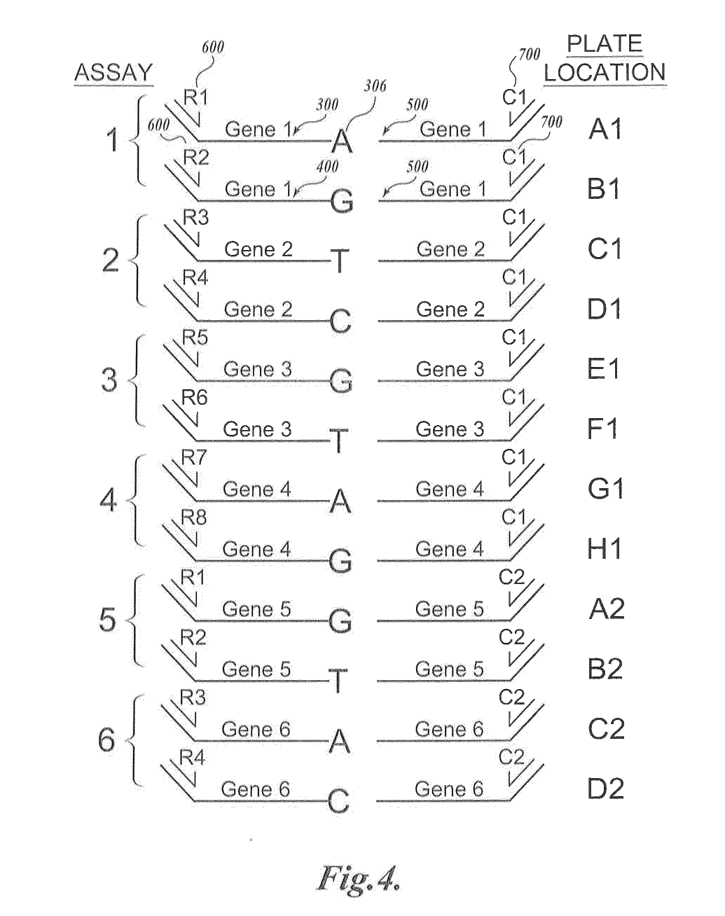

In some embodiments, the ligation-dependent genotyping assay is a multiplexed assay comprising a plurality of sets of SNV query oligos for detecting a plurality of SNV positions of interest, such as at least 5, at least 10, at least 20, at least 40, at least 50, at least 80, at least 100, at least 200, at least 300, at least 500, at least 1000, at least 2,500, at least 5,000, at least 7,500 up to 10,000 more SNV positions of interest in a single ligation reaction. As illustrated in FIG. 4, many different sets of SNV query oligos may be added to a genomic test sample, annealed, ligated, and assayed by qPCR. The results of each independent ligation are read out by unique, tail-specific PCR primer pairs designed to detect a particular ligation product. The advantage to this multiplexing approach is that very small amounts of precious starting material can be interrogated at many different potential mutation locations simultaneously.

For example, as shown in FIG. 4, exemplary genes 1-6 in a target sample, each including a SNV position of interest, are assayed in a single reaction by pooling six sets of SNV query oligos, each set comprising a 5′ consensus ligation oligo (300), a 5′ variant ligation oligo (400), and a 3′ ligation oligo (500), for each gene of interest. As further shown in FIG. 4, each ligation oligo has a tail-specific PCR primer, for example, the forward PCR primers designated R1 to R8 (R stands for “row”), are designed to bind to the tail regions of 5′ ligation primers (consensus and variant) for genes 1-4, with a common reverse PCR primer, designated C1 (C stands for “column”) designed to bind to the tail region of the 3′ ligation primers for genes 1-4. As further shown in FIG. 4, a unique combination of PCR primers (e.g., R1+C1) at a particular location in an assay plate (e.g., A1) is used to amplify a particular ligation product (e.g., Gene 1 consensus) contained in the multiplexed ligation mixture. The PCR primer pairs can be dispensed into individual wells of a multi-well container, thereby allowing for ease of detection of the presence and/or amount of each ligation product in the multiplexed ligation reaction at a designated location.

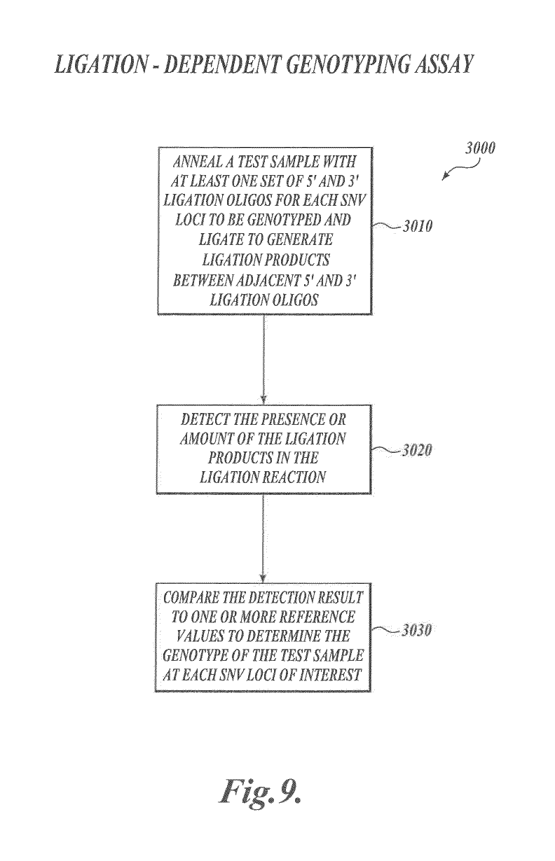

As shown in FIG. 9, the ligation-dependent genotyping assay 3000 includes the step 3010 of annealing a test sample with a least one set of SNV query oligonucleotides comprising at least one of a 5′ ligation oligonucleotide (300) and/or (400), and a 3′ ligation oligonucleotide (500) for each SNV position to be genotyped and ligating the adjacent query oligos annealed to the test sample, detecting the presence or amount of the ligation products at step 3020, and optionally, comparing the detection result to one or more reference values at step 3030 to determine the genotype of the test sample at each SNV loci of interest.

Test Samples

The methods of the invention are useful in any situation in which it is desired to detect one or more SNVs in a target nucleic acid sample (i.e., a haploid or diploid sample), such as, for example, to genotype a particular diploid subject, such as a human, with respect to one or more particular SNV positions of interest (e.g., in the context of determining whether the subject is likely to benefit from a particular therapeutic agent), to confirm the presence or absence of a variant nucleotide at a SNV position of interest that was initially detected during high-throughput sequence analysis, to compare a plurality of subjects of a particular species with respect to a particular target region of interest in order to identify new SNVs within the target region, or to monitor a subject with respect to a particular SNV position of interest over time (e.g., in the context of a therapeutic treatment regime and/or for prognosis or progression of a particular disease, such as cancer).

Examples of a test sample containing one or more target nucleic acid sequence(s) of interest for use in the methods of the invention include genomic DNA, mRNA, tRNA, rRNA, cRNA, oligonucleotides, DNA derived from RNA or DNA, ESTs, cDNA, PCR amplified products derived from RNA or DNA, microRNA, shRNA, siRNA, and mutations, variants or modifications thereof. The starting sample containing nucleic acid molecules may be isolated from a subject, such as a cell sample, tissue sample, or organ sample derived therefrom, including, for example, cultured cell lines, biopsy, blood sample, or fluid sample containing cells. The subject may be an animal, including, but not limited to, an animal such as a cow, a pig, a mouse, a rat, a chicken, a cat, a dog, etc., and is usually a mammal, such as a human. The methods of the invention are also useful to genotype SNV locations of interest in a test sample containing a haploid genome, such as a yeast strain, as demonstrated in Example 7.

Samples containing a target nucleic acid sequence of interest to be genotyped, such as genomic DNA or RNA (e.g., mRNA, rRNA, tRNA, total RNA, microRNA), can be prepared by any of a variety of procedures. In some embodiments, the starting sample comprises genomic DNA. The genomic DNA sample may contain total genomic DNA, intact, fragmented, or enzymatically amplified portions of the same. Genomic DNA can be prepared using routine methods known in the art, (see, e.g., Sambrook, et al., Molecular Cloning: A Laboratory Manual, 2d ed., Cold Spring Harbor Press, Plainsview, N.Y. (1989); and Ausubel, et al., Current Protocols in Molecular Biology (Supplement 47), John Wiley & Sons, New York (1999)).

In some embodiments, the starting sample comprises genomic DNA that has been amplified by whole genome amplification, using multiple displacement amplification, for example as described in Pan et al., PNAS 40(105):15499-15504, incorporated herein by reference.

Target Enrichment

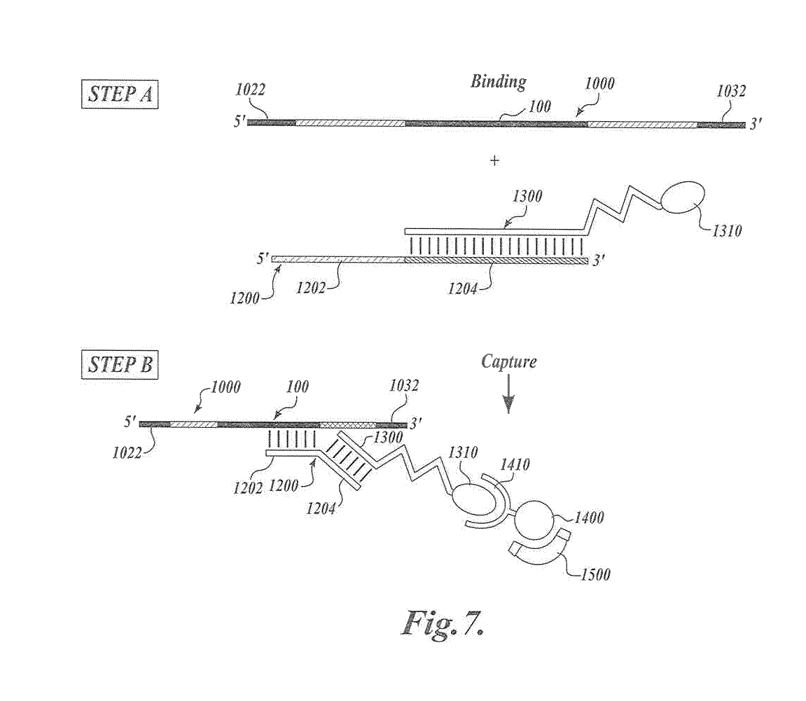

In another embodiment, the starting sample comprises a population of nucleic acid molecules that has been enriched for one or more target regions of interest. In one embodiment, the enriched sample comprises PCR products amplified from a plurality of target-specific amplicons from a nucleic acid containing sample. In another embodiment, as illustrated in FIG. 7, the sample is enriched for a target sequence containing an SNV position of interest 100, using solution-based capture methods from a library of DNA molecules 1000 comprising a subpopulation of nucleic acid target insert sequences of interest flanked by a first primer binding region 1022 and a second primer binding region 1032 within a larger population of nucleic acid insert sequences flanked by the first primer binding region and the second primer binding region.

The step of enriching a library for target sequences 100 with the population of DNA molecules 1000 may be carried out as illustrated in FIG. 7. As shown in FIG. 7, at step A, solution-based capture is carried out by first annealing a library of single-stranded capture probes 1200, each capture probe comprising a target specific region 1202 that hybridizes to a target sequence 100 contained in a library insert, with a library of nucleic acid molecules 1000 comprising nucleic acid target insert sequences of interest 100 flanked by a first primer binding region 1022 on one end and a second primer binding region 1032 on the other end. As further shown in FIG. 7, step A, in one embodiment, the library of nucleic acid molecules 1000 is annealed with a combination of a library of single-stranded capture probes 1200 each comprising a region 1204 that hybridizes to a universal adaptor oligo 1300 and an equimolar amount of universal adaptor oligos 1300 comprising a moiety 1310 for binding to a capture reagent 1400.

The annealing step for solution-based capture is typically carried out by mixing a molar excess of capture probes (or capture probes plus universal adaptor oligos) with the library in a high salt solution comprising from 100 mM to 2 M NaCl (osmolarity=200 to 4000 molar). An exemplary high salt solution for annealing is 10 mM Tris pH 7.6, 0.1 mM EDTA, 1 M NaCl (osmolarity=2000 molar). The nucleic acid molecules in the mixture are then denatured (i.e., by heating to 94 degrees) and allowed to cool to room temperature. In one embodiment, the annealing step is carried out in a high salt solution comprising from 100 mM to 2 M NaCl with the addition of 0.1% triton X100 (or Tween or NP40) nonionic detergent.

An amount of capture reagent 1400 is added to the annealed mixture sufficient to generate a plurality of complexes each containing a nucleic acid molecule, a capture probe (or a capture probe and a universal adaptor oligo), and a capture reagent. This step is carried out in a high salt solution comprising from 100 mM to 2 M NaCl (osmolarity=200 to 4000 molar). An exemplary high salt solution for anneal is 10 mM Tris pH 7.6, 0.1 mM EDTA, 1 M NaCl (osmolarity=2000 molar). The mixture is incubated at room temperature with mixing for about 15 minutes.

The complexes formed are then isolated or separated from solution with a sorting device 1500 (e.g., a magnet) that pulls or sorts the capture reagent 1400 out of solution.

The sorted complexes bound to the capture reagent 1400 are washed with a low salt wash buffer (less than 10 mM NaCl, and more preferably no NaCl) to remove non-target nucleic acids. An exemplary low salt wash buffer is 10 mM Tris pH 7.6, 0.1 mM EDTA (osmolarity=10 millimolar). In some embodiments, the low salt wash optionally contains from 15% to 30% formamide, such as 25% formamide (osmolarity=6.3 molar). For each wash step, the capture reagent 1400 bound to the complexes (e.g., magnetic beads) are resuspended in the low salt wash buffer and rocked for 5 minutes, then sorted again with the sorting device (magnet). The wash step may be repeated 2 to 4 times.

The nucleic acid molecules containing the target sequences are then eluted from the complexes bound to the capture reagent as follows. The washed complexes bound to the capture reagent 1400 are resuspended in water, or in a low salt buffer (i.e., osmolarity less than 100 millimolar), heated to 94° C. for 30 seconds, the capture reagent (e.g., magnetic beads) is pulled out using a sorting device (e.g., magnet), and the supernatant (eluate) containing the target nucleic acid molecules is collected.

The eluate may optionally be amplified in a PCR reaction with a first PCR primer that binds to the first primer binding site 1022 in the first linker and a second PCR primer that binds to the second primer binding site 1032 in the second linker, producing an enriched library which can be optionally sequenced.

The capture oligonucleotides 1200 may be designed to bind to a target region at selected positions spaced across the target region at various intervals. The capture oligo design and target selection process may also take into account genomic features of the target region such as genetic variation, G:C content, predicted oligo Tm, and the like. The length of a capture probe 1200 is typically in the range of from 10 nucleotides to about 200 nucleotides, such as from about 20 nucleotides to about 150 nucleotides, such as from about 30 nucleotides to about 100 nucleotides, such as from about 40 nucleotides to about 80 nucleotides.

The target-specific binding region 1202 of the target capture probe 1200 is typically from about 25 to about 150 nucleotides in length (e.g., 50 nucleotides, 100 nucleotides) and is chosen to specifically hybridize to a target sequence of interest. In one embodiment, the target-specific binding region 1202 comprises a sequence that is substantially complementary (i.e., at least 90% identical, at least 95% identical, at least 96% identical, at least 97% identical, at least 98% identical, at least 99% identical, or 100% identical) to a target sequence of interest.

In one embodiment, the capture probe 1200 is about 70 nucleotides in length, comprising a target-specific region of about 35 nucleotides in length.

One of skill in the art can use art-recognized methods to determine the features of a target binding region 1202 that will hybridize to the target region comprising the SNV position of interest 100 with minimal non-specific hybridization. For example, one of skill can determine experimentally the features such as length, base composition, and degree of complementarity that will enable a nucleic acid molecule (e.g., the target-specific binding region of a target capture probe) to specifically hybridize to another nucleic acid molecule (e.g., the nucleic acid target) under conditions of selected stringency, while minimizing non-specific hybridization to other substances or molecules. For example, for an exon target of interest, a target gene sequence is retrieved from a public database such as GenBank, and the sequence is searched for stretches of from 25 to 150 bp with a complementary sequence having a GC content in the range of 45% to 55%. The identified sequence may also be scanned to ensure the absence of potential secondary structure and may also be searched against a public database (e.g., a BLAST search) to ensure a lack of complementarity to other genes.

In some embodiments, solution-based capture is used to enrich a population of nucleic acid molecules for one or more target polymorphic position(s) of interest, in order to determine the presence of a particular SNV, SNP, or deletion, addition, or other modification using the ligation-dependent genotyping assay described herein. In accordance with such embodiments, the set of target capture probes 1200 are typically designed such that there is a very dense array of capture probes that are closely spaced together such that a single target sequence, which may contain a mutation, will be bound by multiple capture probes that overlap the target sequence. For example, capture probes may be designed that cover every base of a target region, on one or both strands (i.e., head to tail) or that are spaced at intervals of every 2, 3, 4, 5, 10, 15, 20, 40, 50, 90, 100, or more bases across a sequence region.

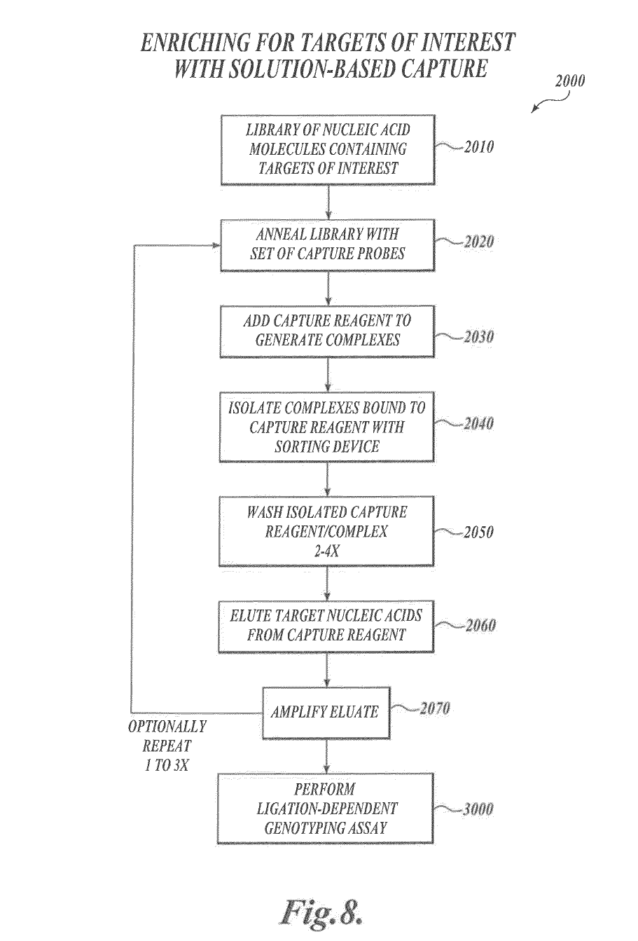

As shown in FIG. 8, the methods of solution-based capture 2000 include the step 2010 of providing a library of nucleic acid molecules comprising nucleic acid target insert sequences of interest flanked by a first primer binding region on one end and a second primer binding region on the other end. At step 2020, the library of nucleic acid molecules 1000 is annealed with a set of capture probes 1200, each capture probe comprising a region that hybridizes to a target sequence contained in a library insert. In one embodiment, the capture probes 1200 comprise a moiety 1310 (e.g., biotinylated) for binding to a capture reagent 1400 (e.g., streptavidin coated beads). In another embodiment, the library of nucleic acid molecules 1000 is annealed with a combination of a set of capture probes 1200, each comprising a region 1204 that hybridizes to a universal adaptor oligo 1300 and an equimolar amount of universal adaptor oligos 1300 comprising a moiety 1310 for binding to a capture reagent 1400.

The annealing step 2020 for solution-based capture is carried out by mixing a molar excess of capture probes (or capture probes plus universal adaptor oligos) with the library in a high salt solution comprising from 100 mM to 2 M NaCl (osmolarity=200 to 4000 molar). An exemplary high salt solution for annealing is 10 mM Tris pH 7.6, 0.1 mM EDTA, 1 M NaCl (osmolarity=2000 molar). The nucleic acid molecules in the mixture are then denatured (i.e., by heating to 94 degrees) and allowed to cool to room temperature. In one embodiment, the annealing step is carried out in a high salt solution comprising from 100 mM to 2 M NaCl with the addition of 0.1% triton X100 (or Tween or NP40) nonionic detergent.

At step 2030, an amount of capture reagent is added to the annealed mixture sufficient to generate a plurality of complexes each containing a nucleic acid molecule, a capture probe (or a capture probe and a universal adaptor oligo), and a capture reagent. This step is carried out in a high salt solution comprising from 100 mM to 2 M NaCl (osmolarity=200 to 4000 molar). An exemplary high salt solution for anneal is 10 mM Tris pH 7.6, 0.1 mM EDTA, 1 M NaCl (osmolarity=2000 molar). The mixture is incubated at room temperature with mixing for about 15 minutes.

At step 2040, the complexes formed in step 2030 are isolated or separated from solution with a sorting device 1500 (e.g., a magnet) that pulls or sorts the capture reagent 1400 out of solution.

At step 2050, the sorted complexes bound to the capture reagent 1400 are washed with a low salt wash buffer (less than 10 mM NaCl, and more preferably no NaCl) to remove non-target nucleic acids. An exemplary low salt wash buffer is 10 mM Tris pH 7.6, 0.1 mM EDTA (osmolarity=10 millimolar). In some embodiments, the low salt wash optionally contains from 15% to 30% formamide, such as 25% formamide (osmolarity=6.3 molar). For each wash step, the capture reagent 1400 bound to the complexes (i.e., magnetic beads) are resuspended in the low salt wash buffer and rocked for 5 minutes, then sorted again with the sorting device (magnet). The wash step may be repeated 2 to 4 times.

At step 2060, the nucleic acid molecules containing the target sequences are eluted from the complexes bound to the capture reagent as follows. The washed complexes bound to the capture reagent 1400 are resuspended in water, or in a low salt buffer (i.e., osmolarity less than 100 millimolar), heated to 94° C. for 30 seconds, the capture reagent (i.e., magnetic beads) are pulled out using a sorting device (i.e., magnet), and the supernatant (eluate) containing the target nucleic acid molecules is collected.

At step 2070, the eluate is amplified in a PCR reaction with a first PCR primer that binds to the first primer binding site in the first linker and a second PCR primer that binds to the second primer binding site in the second linker, producing a once-enriched library which can be optionally genotyped at step 3000.

Alternatively, as shown in FIG. 8, the once-enriched library may be further processed according to steps 2020-2070 using the same set of capture probes in each round of enrichment to generate a library that is twice-enriched, or three-times enriched, etc., for the target sequences of interest prior to performing a ligation-dependent genotyping assay 3000.

In one embodiment, the ratio of the concentration of the DNA target in the first and second round of enrichment to the concentration of capture oligo is a concentration of about 500 ng/ml DNA target to a concentration in the range of from about 1 nM to 10 nM of capture oligo. In one embodiment, the ratio of the concentration of DNA target in the third round of enrichment to concentration of capture oligo is a concentration of about 500 ng/ml of the twice-enriched library to a concentration of about 1 nM of capture oligo.

In one embodiment, the first round of enrichment (steps 2020-2070 shown in FIG. 8) are carried out with a first set of capture probes designed to target a first set of targets, followed by a second round of enrichment that is carried out with a second set of capture probes designed to target a second set of targets.

In one embodiment, the capture reagent (1400) comprises streptavidin coated magnetic beads, each bead having a binding capacity of approximately 50 pmol of biotinylated double-stranded DNA/50 μl of beads. In one embodiment, at step 2030, about 50 μl of the streptavidin coated magnetic beads are added to about 5 μg of the annealed nucleic acids (e.g., in the first and second rounds of enrichment). In one embodiment, at step 2030, about 5 μl of the streptavidin coated magnetic beads are added to about 5 μg of the annealed nucleic acids (e.g., in the third round of enrichment).

Annealing and Ligation for the Ligation-Dependent Genotyping Assay

With reference to FIG. 9, in one embodiment of the method, the annealing and ligation step 3010 of the ligation-dependent genotyping assay 3000 is carried out by mixing a set of SNV query oligonucleotides with the test sample comprising nucleic acids containing the target region of interest under conditions that allow hybridization between the SNV query oligonucleotides and the target nucleic acid region(s) of interest in the presence of a thermostable DNA ligase, and under conditions suitable to ligate the 5′ ligation oligonucleotides having a 3′ region that hybridizes to the nucleotide sequence present at the polymorphic locus of interest in the test sample and the adjacent 3′ phosphorylated ligation oligonucleotides, thereby generating a plurality of ligation products indicative of the genotype of the test sample at the one or more polymorphic loci of interest.

In another embodiment of the method, the annealing and ligation step 3010 of the ligation-dependent genotyping assay 3000 is carried out by first mixing a set of SNV query oligonucleotides with the test sample comprising nucleic acids containing the target region of interest under conditions that allow hybridization between the SNV query oligonucleotides and the target nucleic acid region(s) of interest, then contacting the annealed mixture with either a thermostable, or non-thermostable DNA ligase under conditions suitable to ligate the 5′ ligation oligonucleotides having a 3′ region that hybridizes to the nucleotide sequence present at the polymorphic locus of interest in the test sample and the adjacent 3′ phosphorylated ligation oligonucleotides, thereby generating a plurality of ligation products indicative of the genotype of the test sample at the one or more polymorphic loci of interest.

Hybridizing conditions for hybridizing the SNV query oligos to the target nucleic acid molecules in the test sample are selected at a suitable stringency to achieve specific hybridization and are chosen based on the length of the target-specific binding region and the level of identity between the binding region and the target. The hybridization parameters that can be varied include salt concentration, buffer, pH, temperature, time of incubation, amount and type of denaturant, such as formamide, etc. (see, e.g., Sambrook et al., Molecular Cloning: A Laboratory Manual, 2d ed., Vols. 1-3, Cold Spring Harbor Press, New York, 1989; Hames et al., Nucleic Acid Hybridization, IL Press, 1985; Davis et al., Basic Methods in Molecular Biology, Elsevier Sciences Publishing, Inc., New York, 1986). The reaction conditions required to achieve specific interactions of the SNV query oligos and target nucleic acid molecules are routine and conventional in the art (e.g., as described in Niemeyer et al., Nucleic Acid Res. 22:5530-5539, 1994; Fodor et al., U.S. Pat. No. 5,510,270; Pirrung et al., U.S. Pat. No. 5,143,854, incorporated herein by reference).

In some embodiments, the hybridization step of a hybridization reaction followed by a ligation reaction, or a coupled hybridization/ligation reaction is carried out in a suitable reaction mixture comprising at least one monovalent cationic salt selected from the group consisting of KCl, NaCl and NH₄Cl, in order to stimulate annealing of the genotyping primers to the complementary genotyping primers, for example, as described in Example 5.

In some embodiments, the hybridization step of a hybridization reaction followed by a ligation reaction, or a coupled hybridization/ligation reaction is carried out in a suitable reaction mixture by incubating the mixture at an initial temperature greater than 90° C. to denature the nucleic acids and gradually cooling to room temperature over a time period ranging from 30 minutes to 2 hours or longer, such as for at least 30 minutes, at least 60 minutes, at least 120 minutes, at least 170 minutes, or longer.

For example, hybridization of two binding partners may be carried out in a buffer such as, for example, 6× SSPE-T (0.9 M NaCl, 60 mM MaH₂PO₄, 6 mM EDTA and 0.05% Triton-X-100) for a time period from 10 minutes to at least 3 hours, at a temperature from about 4° to about 37°. In some embodiments of the invention, the reaction conditions can approximate physiological conditions. An exemplary solution for annealing is 10 mM Tris pH 7.6, 0.1 mM EDTA, 20 mM NaCl, as described in Example 1.

The amount of SNV query oligos added to the test sample per genotyping reaction is typically from about 1 pM to about 50 nM, such as from about 10 pM to about 5 nM, such as about 50 pM to about 1000 pM, such as from about 100 pM to about 500 pM. As described in Example 3, it was determined that SNV oligo concentrations in the range of 100 pM improved assay sensitivity by increasing the signal-to-noise ratio. The nucleic acids in the mixture are then denatured (i.e., by heating to 94 degrees) and allowed to cool to room temperature.

In one embodiment, a thermostable DNA ligase, such as, for example, Taq DNA ligase or 9° N DNA ligase, is utilized in the methods of the invention. The use of a thermostable ligase is advantageous because the enzyme activity is retained at the high temperatures needed for DNA melting and reannealing. In another embodiment, either a thermostable (such as Taq DNA ligase) or a non-thermostable DNA ligase (such as T4 DNA ligase) is added to the annealed mixture, as described in Example 5.

In accordance with some embodiments, a ligation reaction comprising a non-thermostable DNA ligase is typically incubated at a temperature ranging from about 15° C. to about 45° C. for a time period ranging from at least one minute to 30 minutes or longer (e.g., at least about 1 minute, at least 5 minutes, at least 10 minutes, at least 20 minutes, or at least 30 minutes).

In accordance with some embodiments, a ligation reaction comprising a thermostable DNA ligase is typically incubated at a temperature ranging from about 37° C. to about 75° C. for a time period ranging from at least one minute to 30 minutes or longer (e.g., at least about 1 minute, at least 5 minutes, at least 10 minutes, at least 20 minutes, or at least 30 minutes). In addition to the fact that thermostable ligases may be utilized at high temperatures, it has been determined that thermostable DNA ligases have greater specificity and preference for ligating nicks in dsDNA and have little ssDNA joining activity (i.e., randomly joining oligos together in the absence of template, such as a target nucleic acid of interest), whereas it has been determined that T4 DNA ligase, a non-thermostable ligase, joins oligos in the absence of template at a significant rate.

Detection of Ligation Products

At step 3020, the presence and/or amount of the ligation products in the ligation reaction are detected. The presence and/or amount of the ligation products in the ligation reaction may be determined using any suitable method of measurement. As used herein, the terms “determining,” “measuring,” “evaluating,” “assessing,” and “assaying” are used interchangeably to refer to any form of measurement, and include determining if an element, (e.g., such as the variant or consensus nucleotide, or the ligation product indicative of presence of the variant or consensus nucleotide), is present or not. These terms include both quantitative and/or qualitative determinations, which may be relative or absolute.

In one embodiment, the amount of the ligation products in the ligation reaction are measured using quantitative PCR (qPCR) comprising amplification of the ligation products with one or more pair(s) of detection primers with a DNA polymerase, each primer pair comprising a forward PCR primer that binds to the first PCR primer binding region in the 5′ ligation oligonucleotide and a reverse PCR primer that binds to the second PCR primer binding region in the 3′ ligation oligonucleotide. In such embodiments, it is noted that the tails 302, 402, 502 on the ligation primers 300, 400, and 500, respectively, containing primer binding sites for primers used for subsequent real-time quantitative PCR, can, in principle, be many different sequences. This allows for multiplexing of numerous assays to detect different SNVs in a single ligation reaction, as further described in Examples 3, 5, and 7.

In one embodiment, a fluorescent dye, such as SYBR green, is included in the qPCR reaction that intercalates with double-stranded DNA, causing fluorescence of the dye. An increase in DNA product during PCR therefore leads to an increase in fluorescence intensity and is measured at each cycle, thus allowing DNA concentration to be quantified. In order to reduce background levels due to the binding of the dye to non-specific PCR products, such as primer-dimers, in one embodiment, a paired set of primers is used for each PCR reaction, wherein the penultimate two or three nucleotides at the 3′ end of the forward and reverse primers are selected to avoid primer-dimer formation.

In another embodiment, the qPCR reaction is carried out using a set of fluorescent reporter probes. An increase in the product targeted by the reporter probe occurs during each PCR cycle, therefore, causes a proportional increase in fluorescence.

Fluorescence is detected and measured in the real-time PCR thermocycler and its geometric increase corresponding to exponential increase of the product is used to determine the threshold cycle (Ct) in each reaction. Relative concentrations of DNA present during the exponential phase of the reaction are determined by plotting fluorescence against cycle number on a logarithmic scale. A threshold for detection of fluorescence above background is determined. The cycle at which the fluorescence from a sample crosses the threshold is called the cycle threshold (Ct). Since the quantity of DNA doubles every cycle during the exponential phase, relative amounts of DNA can be calculated. For example, a sample whose Ct is 3 cycles earlier than another sample has 2³=8 times more template.

In some embodiments of the genotyping methods, as shown in FIG. 9 at step 3030, the detection result is compared to one or more reference values obtained from one or more reference standards to determine the genotype of the test sample at each SNV position of interest, for example, using the methods described supra. The one or more reference values may be obtained by carrying out the ligation-dependent genotyping assay with one or more reference standards, such as a set of SNV query oligos and a pair of synthetic double-stranded templates comprising a target specific sequence region including either a consensus or variant nucleotide at the SNV position of interest, as described herein. The synthetic double-stranded templates containing an SNV position of interest are typically at least 30 to 200 nucleotides in length and may be generated by annealing complementary synthesized oligos, as described in Example 1. The SNV position of interest is typically located at or within 10 nucleotides of the middle of the synthetic template.

In another aspect, the present invention provides a method of genotyping a test sample at one or more single nucleotide variant(s) (SNVs) position(s) of interest, the method comprising: (a) for each SNV position of interest, contacting in three separate reaction mixtures: (i) a synthetic template comprising the target region of interest having a consensus nucleotide at the SNV position of interest; (ii) a synthetic template comprising the target region of interest having a variant nucleotide at the SNV position of interest; and (iii) a test sample comprising the target region of interest comprising the SNV position of interest to be genotyped; with one or more set(s) of SNV query oligonucleotides, each set comprising: (i) a pair of allele-specific 5′ ligation oligonucleotides, the pair comprising a first 5′ ligation oligonucleotide comprising, from the 5′ to 3′ end, a first PCR primer binding region, a target-specific binding region selected to hybridize 5′ of the SNV nucleotide position of interest, and a 3′ region chosen to hybridize to the consensus nucleotide sequence at the SNV position of interest and a second 5′ ligation oligonucleotide comprising, from the 5′ to 3′ end, a first PCR primer binding region, a target-specific binding region selected to hybridize 5′ of the SNV nucleotide position of interest, and a 3′ region chosen to hybridize to the variant nucleotide sequence at the SNV position of interest and (ii) a phosphorylated 3′ ligation oligonucleotide comprising from the 5′ to 3′ end, a target-specific binding region selected to hybridize 3′ of the SNV position of interest and a second PCR primer binding region, under conditions that allow hybridization between the SNV query oligonucleotides and the nucleic acid target regions of interest; (b) contacting the three separate reaction mixtures of step (a) with DNA ligase under conditions suitable to ligate the 5′ ligation oligonucleotides having a 3′ region that hybridizes to the nucleotide sequence present at the SNV nucleotide position of interest in the synthetic templates and test samples and the adjacent 3′ phosphorylated ligation oligonucleotides, thereby generating three separate ligation mixtures; and (c) measuring the amount of the ligation products in each of the three ligation mixtures of step (b).

In some embodiments of the method, the hybridization and ligation steps are combined (i.e., coupled), wherein a test sample comprising one or more SNV positions of interest within one or more target nucleic acid region(s) of interest is contacted with one or more set(s) of query oligonucleotides in the presence of a thermostable DNA ligase. In other embodiments of the method, the hybridization and ligation reactions are carried out sequentially under separate reaction conditions (i.e., uncoupled), and may utilize either thermostable or non-thermostable DNA ligase.

The synthetic templates and SNV query oligos for a SNV position of interest may be generated as previously described herein.

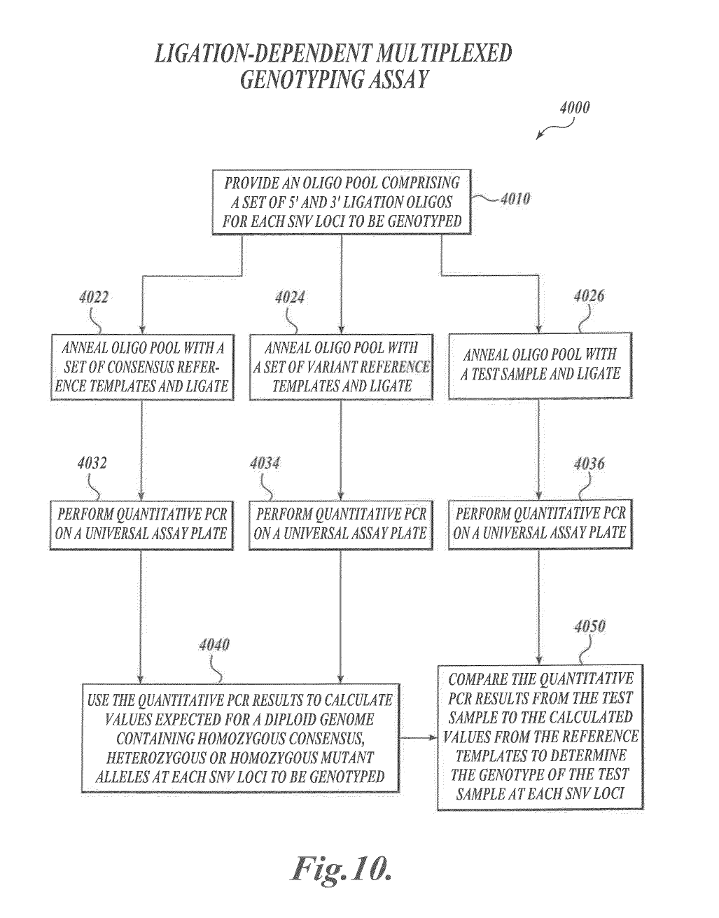

As shown in FIG. 10, an embodiment of the ligation-dependent multiplexed genotyping assay 4000 is carried out for a set of SNV positions of interest by providing a pool of one or more sets of SNV query oligos (i.e., an oligo pool) at step 4010, each set of SNV query oligos comprising 5′ and 3′ ligation oligos for each SNV position of interest to be genotyped. The pool at step 4010 may comprise at least 5 sets of SNV query oligos, at least 10, at least 20, at least 40, at least 50, at least 80, at least 100, up to 500 or more, wherein each set is designed to genotype an SNV position of interest.

At step 4022, the oligo pool according to step 4010 is annealed with a set of consensus reference templates corresponding to the SNV positions of interest and ligated in a first reaction vessel. At step 4024, the oligo pool according to step 4010 is annealed with a set of variant reference templates corresponding to the SNV positions of interest in the presence of DNA ligase and ligated in a second reaction vessel. At step 4026, the oligo pool according to step 4010 is annealed with a test sample comprising nucleic acid molecules having the SNV positions of interest and ligated in a third reaction vessel. The annealing and ligation steps may be carried out as previously described herein.

At step 4032, the ligation mixture from step 4022 (consensus templates) is distributed over a multi-well container (e.g., a universal assay plate) comprising PCR detection primer pairs arranged in a matrix such that each well in the matrix is positionally addressable and contains a different detection primer pair, and a quantitative PCR assay is carried out in the multi-well container.

At step 4034, the ligation mixture from step 4024 (variant templates) is distributed over a multi-well container (e.g., a universal assay plate) comprising PCR detection primer pairs arranged in a matrix such that each well in the matrix is positionally addressable and contains a different detection primer pair, and a quantitative PCR assay is carried out in the multi-well container. In some embodiments, the PCR detection primer pairs in the matrix are minimally-interacting primer pairs, as described herein.

At step 4036, the ligation mixture from step 4026 (test sample) is distributed over a multi-well container (e.g., a universal assay plate) comprising PCR detection primer pairs arranged in a matrix such that each well in the matrix is positionally addressable and contains a different detection primer pair, and a quantitative PCR assay is carried out in the multi-well container. The multi-well containers used in steps 4032, 4034, and 4036, are separate, but substantially identical containers (i.e., each container contains the same primer pairs, arranged in the same grid pattern, so that the results of each assay can be compared side by side).

At step 4040, the quantitative PCR results obtained from step 4032 (consensus templates) and from step 4034 (variant templates) are used to calculate the reference values expected for a diploid genome containing homozygous consensus nucleotides, heterozygous nucleotides, or homozygous variant nucleotides, at each SNV position of interest. The quantitative PCR results may be raw cycle threshold (Ct) results (i.e., the cycle at which the fluorescence from a sample crosses the threshold), or may be processed results (such as those obtained by subtracting a background measurement, or by rejecting a reading for a feature which is below a predetermined threshold, normalizing the results, or the average Ct value of replicate samples, and the like). An exemplary method of calculating the reference values expected for a diploid genome using quantitative PCR results obtained from a pair of reference templates (consensus and variant) for each SNV position of interest is provided in Example 3.

At step 4050, the quantitative PCR results obtained from step 4036 (the test sample) are compared to the calculated reference values from step 4040 to determine the genotype of the test sample at each SNV position of interest, and assigning the genotype based on the closest pairing between the experimental value from the test sample and the calculated reference values for each potential genotype at each SNV position of interest. For example, genotyping with the consensus template may yield a Ct value of “25” in the consensus assay (assay with SNV consensus query oligos), and a Ct value of “30” in the variant assay (assay with SNV variant query oligos). The genotyping results are calculated as a result of the Ct of the variant (Ct(var)) minus the Ct of the consensus (Ct(cons)). Therefore, in the above example, the consensus template yields a Ct(var)−Ct(cons) of “30”−“25”=5. The variant template in the above example yields a Ct(var)−Ct(cons) value of “25”−“30”=−5. Finally, a mixed template would be inferred to give a Ct(var)−Ct(cons) value of “25”−“25”=0. Assuming the sample is a diploid, then a sample with a homozygous consensus base at the SNV position would be expected to yield a Ct(var)−Ct(cons) value of approximately “30”−“25”≈5. Similarly, a homozygous variant base would be expected to yield a value of ≈−5, and a heterozygous consensus plus variant would be expected to return a Ct(var)−Ct(cons) value of approximately zero. By comparing the actual Ct(var)−Ct(cons) value of the test sample in the genotyping assay to the reference templates, genotypes are assigned based on the closest numerical similarity to the homozygous consensus, homozygous variant or heterozygous consensus and variant values produced with the templates and the Ct(var)−Ct(cons) calculation.

Assessing the Performance of the Ligation-Dependent Genotyping Assay

The performance of the ligation-dependent genotyping assay carried out using quantitative PCR may be evaluated by calculating the dynamic range of the assay as follows. The average Cts across replicate samples (e.g., quadruplicate wells) in the qPCR assay for each consensus and variant pair of an SNV assay set is calculated, wherein a Ct value below 30 is indicative of an informative qPCR assay. The Ct(variant)−Ct(consensus)=Δ consensus is calculated for each of the consensus template assays and the Ct(consensus)−Ct(variant)=Δ variant is calculated for each of the variant template assays. The sum of Δ consensus for the consensus template assays plus Δ variant for the variant template assays is then calculated. As described in Example 7, it was experimentally determined that if the sum of A consensus for the consensus template assays plus Δ variant for the variant template assays is ≧3, then genotyping calls can be made with confidence in diploid organisms.

Matrix of Detection Primer Pairs

In another aspect, the present invention provides a method of producing a multi-well container comprising a matrix of detection primer pairs for decoding a multiplexed assay, the method comprising: (a) designing a plurality of detection primer pairs, each pair comprising a forward primer and a reverse primer for amplifying a target nucleic acid molecule of interest comprising a 5′ primer binding region and a 3′ primer binding region, wherein each forward primer comprises a 5′ region that hybridizes to the 5′ primer binding region of the target nucleic acid molecule of interest and a 3′ region selected to avoid primer-dimer formation with the reverse primer; and wherein each reverse primer comprises a 5′ region that hybridizes to the 3′ primer binding region of the target nucleic acid molecule of interest and a 3′ region selected to avoid primer-dimer formation with the forward PCR primer; and (b) dispensing each of the plurality of detection primer pairs into a well in a multi-well container comprising an ordered array of wells arranged in a matrix comprising a plurality of perpendicular rows distributed along the vertical axis of the container and a plurality of columns distributed along the longitudinal axis of the container, such that each well in the matrix is positionally addressable. In one embodiment, the present invention provides multi-well containers comprising a matrix of detection primer pairs for decoding a multiplexed assay. In some embodiments, the detection primer pairs in the matrix are designed to be minimally-interacting primer pairs (i.e., primer pairs each comprising a 3′ region selected to avoid primer-dimer formation), as described herein.

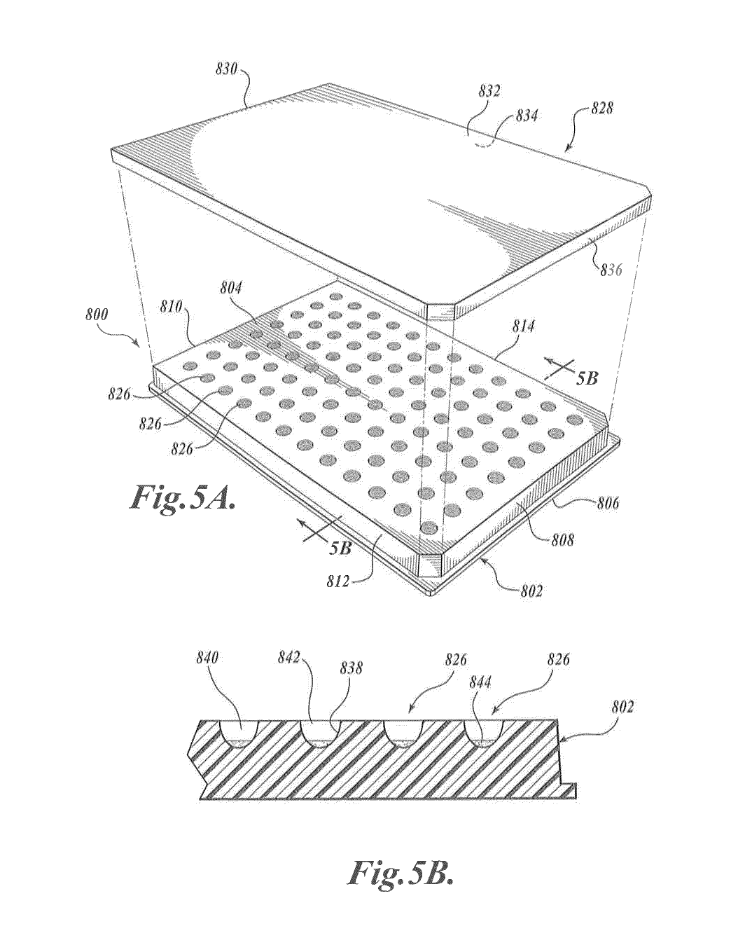

An exemplary multi-well container useful for carrying out the detection step of the genotyping assay is shown in FIG. 5A. FIG. 5A shows a perspective view of a representative multi-well container 800 of the present invention. The multiwell container 800 includes a body 802 including an upper surface 804, a lower surface 806 disposed opposite the upper surface 804, a right side 808, a left side 810 disposed opposite the right side 808, a top 814, and a bottom 812 disposed opposite the top 814. The container body 802 defines multiple wells 826. A lid 828 may optionally cover the upper surface 804. As shown in FIG. 5A, the lid 828 includes a lid body 830 defining an outer surface 832, an inner surface 834 and a lip 836 that extends around the perimeter of lid body 830. In the embodiment shown in FIG. 5A, the multi-well container 800 comprises an ordered array of individual wells 826. In the embodiment shown in FIG. 5A, the ordered array of individual wells 826 is arranged in a matrix of a plurality of perpendicular rows distributed along the vertical axis (i.e., from the top 814 to the bottom 812) of the multi-well container 800 and a plurality of columns distributed along the longitudinal axis (i.e., from the left side 810 to the right side 808). In exemplary embodiments, the multi-well container 800 includes a matrix having a dimension of 8 columns×12 rows=96 wells, or 16 columns×24 rows=384 wells, or 32 columns×48 rows=1536 wells.

As shown more clearly in FIG. 5B, in the representative embodiment of the multi-well container 800, each well 826 is generally hemispherical and includes a well wall 838 defining a well lumen 840 that opens onto upper surface 804 of the container body 802 through the opening 842. The multiple wells 826 are sized for receiving and retaining aliquots (e.g., aliquots that each have a volume of from 1 μl to 1000 μl) of a liquid composition, such as a PCR reaction mixture.

In the exemplary embodiment shown in FIG. 5A, the multi-well container 800 has a generally rectangular shape, but the multi-well container 800 can have any shape, such as square or circular. Similarly, the wells 826 can have any desired shape provided that they are capable of containing a liquid composition 844. The lid 828 is suitably dimensioned to fit over upper surface 814 of container body 802. The container body 802 and the lid 828 may be made from any suitable material, or mixtures of suitable materials. Typically, the container body 802 and the lid 828 are made from a material, such as plastic, that can withstand freezing and thawing at least once, as well as multiple cycling to temperatures up to at least 95° C. (e.g., in a thermocycler). Exemplary containers include a 96 well assay plate, or a 384 well assay plate, such as commercially-available 96-well plastic plates manufactured by Island Scientific (7869 NE Day Rd West, Bainbridge Island, Wash. 98110), or by MWG Biotech (4170 Mendenhall Oaks Parkway, Suite 160, High Point, N.C. 27265), or optical plates from ABI for real time PCR analysis with the ABI 7900 (Applied Biosystems, Foster City, Calif.), or any other multi-well assay plate suitable for quantitative PCR assay analysis.

In one embodiment of the invention, the present invention provides a multi-well container 800 comprising a matrix of a plurality of compositions 844, each composition 844 comprising detection primer pairs dispensed into individual wells for decoding a multiplexed assay. The multi-well containers 800 are preferably produced en masse, easily stored, and reproducible, allowing multiple genotyping assays to be assayed and easily compared with each other.

In some embodiments, at least 20% of the wells 826 (e.g., at least 20% (e.g., at least 25%, at least 30%, at least 40%, at least 50%, at least 60%, or at least 70%, or at least 80%, or at least 90%, or all of the wells in the container) comprise a composition 844 comprising a PCR detection primer pair, each pair comprising a forward PCR primer and a reverse PCR primer for amplifying a target nucleic acid molecule of interest flanked by a 5′ primer binding region and a 3′ primer binding region, wherein each forward PCR primer comprises a 5′ region that hybridizes to the 5′ primer binding region of the target nucleic acid molecule of interest and a 3′ region selected to avoid primer-dimer formation with the reverse PCR primer; and wherein each reverse PCR primer comprises a 5′ region that hybridizes to the 3′ primer binding region of the target nucleic acid molecule of interest and a 3′ region selected to avoid primer-dimer formation with the forward PCR primer, also referred to as “minimally interacting primer pairs”.

In some embodiments, the 3′ region of the minimally interacting forward and reverse primer pairs selected to avoid primer-dimer formation consists of from two to nine 3′ terminal nucleotides (e.g., the last 2 nucleotides, the last 3 nucleotides, the last 4 nucleotides, the last 5 nucleotides, the last 6 nucleotides, the last 7 nucleotides, the last 8 nucleotides, or the last 9 nucleotides as measured from the 3′ end) wherein the 3′ terminal nucleotide sequence is selected to reduce background signal and provide the greatest possible dynamic range for genotyping assays.

In some embodiments, the 3′ region of the forward and reverse primer pairs consists of the last two or three nucleotides at the 3′ end of the respective oligonucleotides. In accordance with such embodiments, as described in Examples 2, 4, 6, and 7, the last two or three nucleotides at the 3′ end of the primer pairs are designed with sequences that cannot pair with one another nor can they self anneal. For example, in one representative embodiment, each forward primer in a primer matrix is designed to end in the sequence “CT” and each reverse primer in the primer matrix is designed to end in the sequence “GA,” as described in Example 2. In another representative embodiment, a set of forward primers in a primer matrix is designed to end in “ACA” and a set of reverse primers in the primer matrix is designed to end in “CAC,” as described in Example 4. In some embodiments, candidate primers for use as minimally interactive primer pairs are further screened to eliminate primers containing sequences present within 9 nucleotides of the 3′ end of the primer that would hybridize to the 3′ terminal sequences, such as primers containing the sequence “GTG” or “TGT” within the last 9 nucleotides of the 3′ end, as described in Example 4.

In another representative embodiment, a set of minimally interacting primer pairs is selected by first generating a set of candidate random 22-mer DNA sequences, screening the sequences for the presence of either “TTT” or “GGG” in the 3′ terminal 6 nucleotides, and removing such candidate primers to generate a subset of candidate primers, adding the 3′ terminal sequence “CCC” to a first group of the subset of primers and adding the 3′ terminal sequence “AAA” to a second group of the subset primers, to generate a set of candidate primer pairs, and performing a control assay with no template with the set of candidate primer pairs to identify primer pairs that generated a Ct value indicative of a low background level, such as a Ct value of greater than 35 (such as a Ct value greater than 36, a Ct value greater than 37, a Ct value greater than 38, a Ct value greater than 39, or a Ct value greater than 40). In some embodiments, the 3′ terminal sequence “ACA” is added to the first group of the subset primers and the 3′ terminal sequence “CAC” is added to the second group of primers in order to provide primer sets with closely matched Tm values.

A primer matrix is then generated that includes only the primer pairs with the desired low background level (e.g., all primer pairs generated a Ct value of greater than 35 in a no template control assay), as described in Examples 6 and 7.

In some embodiments, the PCR detection primer pairs are dispensed into a plurality of individual wells 826 (also referred to as “features”) in the multi-well container such that each pair of PCR detection primers in each well 826 of the matrix is positionally addressable, i.e., is localized to a known, defined well 826 in the container 800 such that the identity (i.e., the sequence) of each amplified ligation product can be determined from its position on the matrix.

In some embodiments, at least 20% (e.g., at least 25%, at least 30%, at least 40%, at least 50%, at least 60%, at least 70%, at least 80%, at least 90%, or all of the wells in the container) of the wells 826 in the multi-well container 800 comprise a composition 844 comprising a pair of PCR detection primer pairs that is different from the PCR detection primer pairs contained in the other wells of the multi-well container 800.

In some embodiments, the composition 844 further comprises reagents for carrying out an enzyme reaction, such as a polymerase, such as a DNA polymerase, or such as a reverse transcriptase.

In some embodiments, the composition 844 further comprises one or more reagents for carrying out a PCR amplification reaction. PCR amplification methods are well known in the art and are described, for example, in Innis et al., eds., 1990, PCR Protocols: A Guide to Methods and Applications, Academic Press Inc., San Diego, Calif., and Ausubel et al., Short Protocols in Molecular Biology, Wiley, 1995; and Innis et al., PCR Protocols, Academic Press, 1990. An amplification reaction typically includes the DNA that is to be amplified, a thermostable DNA polymerase, two oligonucleotide primers, deoxynucleotide triphosphates (dNTPs), reaction buffer and magnesium.

In some embodiments, the composition 844 comprises a pair of PCR detection primers, DNA polymerase, and reagents for carrying out a quantitative PCR reaction, such as one or more of the following: a Tris buffer, a potassium salt (e.g., potassium chloride), a magnesium salt (e.g., magnesium chloride), nucleotides (e.g., adenine, cytosine, guanine and thymidine), or derivatives thereof, and a detection reagent, such as a fluorescent dye (e.g., SYBR green) or other qPCR reagents known in the art, such as TaqMan, or molecular beacons. In some embodiments, the composition 844 comprises 2× SYBR master mix, commercially available from Applied Biosystems, Foster City, Calif.

In some embodiments, the method of making a matrix for decoding the results of a multiplexed assay further comprises aliquoting the liquid composition 844 into multiple wells 826 of the multi-well container 800 and freezing the liquid composition 844 or freezing and drying (i.e., lyophilizing) the composition 844, wherein each dried aliquot comprises an amount of water that is less than 0.1% by weight of the dried aliquot. Aliquots of the liquid composition 844 can be frozen by any means, such as by placing the container containing the aliquots of the liquid composition 844 into a freezer where the container is incubated at a temperature below the freezing point of the liquid mixture until the aliquots of the mixture freeze.

In some embodiments, the method further comprises storing the frozen liquid or lyophilized aliquots at a temperature below minus 15° C. In some embodiments, the method comprises packaging the multi-well container 800 comprising the aliquoted composition 844 into a packaging material, such as a plastic wrapper, or other suitable protective outer packaging material.

Kits for Ligation-Dependent Genotyping Assays

In another aspect, the invention provides a kit for genotyping a test sample at one or more polymorphic loci of interest, such as at one or more single nucleotide variant(s) (SNVs) position(s) of interest. The kit in accordance with this aspect of the invention comprises at least one set of query oligonucleotides for genotyping at least one polymorphic locus of interest, the set comprising (i) at least one 5′ ligation oligonucleotide comprising, from the 5′ to 3′ end, a first PCR primer binding region, a target-specific binding region selected to hybridize 5′ of the polymorphic locus of interest, and a 3′ region chosen to hybridize to either a consensus or variant nucleotide sequence at the polymorphic locus of interest, and (ii) a phosphorylated 3′ ligation oligonucleotide comprising from the 5′ to 3′ end, a target-specific binding region selected to hybridize 3′ of the polymorphic locus of interest and a second PCR primer binding region. The query ligation oligonucleotides (e.g., SNV query ligation oligonucleotides) may be generated as described herein.

In some embodiments, the kit may further comprise a thermostable DNA ligase, such as Taq DNA ligase or 9° N DNA ligase. In some embodiments, the kit may further comprise at least one synthetic template comprising the target region of interest having a consensus or variant nucleotide at the SNV position of interest. The synthetic templates may be generated as described herein.

In some embodiments, the kit may further comprise one or more detection primer pairs for quantitative PCR analysis of the ligation mixture. In some embodiments, the kit may comprise a multi-well container comprising a plurality of detection primer pairs arranged in a matrix (i.e., a universal plate for decoding a multiplex assay), as described herein. In some embodiments, the kit may further comprise one or more reagents for carrying out a quantitative PCR reaction, such as one or more of the following: a Tris buffer, a potassium salt (e.g., potassium chloride), a magnesium salt (e.g., magnesium chloride), nucleotides (e.g., adenine, cytosine, guanine and thymidine), or derivatives thereof, and a detection reagent, such as a fluorescent dye (e.g., SYBR green) or other qPCR reagents known in the art, such as TaqMan, or molecular beacons.

Oligonucleotide Synthesis

DNA synthesis of the various oligonucleotides of the invention (e.g., SNV query oligos, synthetic templates, PCR detection primer linkers, and capture probes) can be carried out by any art-recognized chemistry, including phosphodiester, phosphotriester, phosphate triester, or N-phosphonate and phosphoramidite chemistries (see, e.g., Froehler et al., Nucleic Acid Res. 14:5399-5407, 1986; McBride et al., Tetrahedron Lett. 24:246-248, 1983). Methods of oligonucleotide synthesis are well known in the art and generally involve coupling an activated phosphorous derivative on the 3′ hydroxyl group of a nucleotide with the 5′ hydroxyl group of the nucleic acid molecule (see, e.g., Gait, Oligonucleotide Synthesis: A Practical Approach, IRL Press, 1984).

Suitable nucleotides useful in the synthesis of the various oligonucleotides of the invention include nucleotides that contain activated phosphorus-containing groups such as phosphodiester, phosphotriester, phosphate triester, H-phosphonate and phosphoramidite groups. In some embodiments, oligonucleotides can be synthesized using modified nucleotides, or nucleotide derivatives, such as, for example, combinations of modified phosphodiester linkages such as phosphorothiate, phosphorodithioate, and methylphosphonate, as well as nucleotides having modified bases such as inosine, 5′-nitroindole, and 3′ nitropyrrole. Additionally, it is possible to vary the charge on the phosphate backbone of the nucleic acid molecule, for example, by thiolation or methylation, or to use a peptide rather than a phosphate backbone. In some embodiments, oligonucleotides may be synthesized for use in the methods described herein that include one or more nucleotide analogs at one or more positions, wherein the nucleotide analogs enhance oligonucleotide binding affinity, such as 2-O-ethyl modified nucleotides or locked nucleic acid molecules. As used herein, the term “locked nucleic acid molecule” (abbreviated as LNA molecule) refers to a nucleic acid molecule that includes a 2′-O,4′-C-methylene-β-D-ribofuranosyl moiety. Exemplary 2′-O,4′-C-methylene-β-D-ribofuranosyl moieties, and exemplary LNAs including such moieties, are described, for example, in Petersen, M. and Wengel, J., Trends in Biotechnology 21(2):74-81 (2003) which publication is incorporated herein by reference in its entirety. The making of such modifications is within the skill of one trained in the art.

A population of nucleic acid molecules can be synthesized on a substrate by any art-recognized means including, for example, photolithography (see, Lipshutz et al., Nat. Genet. 21(1 Suppl):20-24, 1999) and piezoelectric printing (see, Blanchard et al., Biosensors and Bioelectronics 11:687-690, 1996). In some embodiments, nucleic acid molecules are synthesized in a defined pattern on a solid substrate to form a high-density microarray. Techniques are known for producing arrays containing thousands of oligonucleotides comprising defined sequences at defined locations on a substrate (see, e.g., Pease et al., Proc. Nat'l. Acad. Sci. 91:5022-5026, 1994; Lockhart et al., Nature Biotechnol. 14:1675-80, 1996; and Lipshutz et al., Nat. Genet. 21 (1 Suppl):20-4, 1999).

In some embodiments, populations of nucleic acid molecules are synthesized on a substrate, to form a high density microarray, by means of an ink jet printing device for oligonucleotide synthesis, such as described by Blanchard in U.S. Pat. No. 6,028,189; Blanchard et al., Biosensors and Bioelectrics 11:687-690 (1996); Blanchard, Synthetic DNA Arrays in Genetic Engineering, Vol. 20, J. K. Setlow, Ed. Plenum Press, New York at pages 111-123; and U.S. Pat. No. 6,028,189 issued to Blanchard. The nucleic acid sequences in such microarrays are typically synthesized in arrays, for example, on a glass slide, by serially depositing individual nucleotide bases in “microdroplets” of a high surface tension solvent such as propylene carbonate. The microdroplets have small volumes (e.g., 100 picoliters (pL) or less, or 50 pL or less) and are separated from each other on the microarray (e.g., by hydrophobic domains) to form surface tension wells which define the areas containing the array elements (i.e., the different populations of nucleic acid molecules). Microarrays manufactured by this ink-jet method are typically of high density, typically having a density of at least about 2,000 different nucleic acid molecules per 1 cm². The nucleic acid molecules may be covalently attached directly to the substrate, or to a linker attached to the substrate at either the 3′ or 5′ end of the polynucleotide. Exemplary chain lengths of the synthesized nucleic acid molecules suitable for use in the present methods are in the range of about 20 to about 200 nucleotides in length, such as 50 to 100, 60 to 100, 70 to 100, 80 to 100, or 90 to 100 nucleotides in length. In some embodiments, the nucleic acid molecules are in the range of 40 to 100 nucleotides in length.

Exemplary ink jet printing devices suitable for oligonucleotide synthesis in the practice of the present invention contain microfabricated ink-jet pumps, or nozzles, which are used to deliver specified volumes of synthesis reagents to an array of surface tension wells (see, Kyser et al., J. Appl. Photographic Eng. 7:73-79, 1981).

In some embodiments, a population of nucleic acid molecules is synthesized to form a high-density microarray. A DNA microarray, or chip, is an array of nucleic acid molecules, such as synthetic oligonucleotides, disposed in a defined pattern onto defined areas of a solid support (see, Schena, BioEssays 18:427, 1996). The arrays are preferably reproducible, allowing multiple copies of a given array to be produced and easily compared with each other. Microarrays are typically made from materials that are stable under nucleic acid molecule hybridization conditions. In some embodiments, the nucleic acid molecules on the array are single-stranded DNA sequences. Exemplary microarrays and methods for their manufacture and use are set forth in T. R. Hughes et al., Nature Biotechnology 19:342-347, April 2001, which publication is incorporated herein by reference.

In some embodiments, the methods of the invention utilize oligonucleotides that are synthesized on a multiplex parallel DNA synthesis system based on an integrated microfluidic microarray platform for parallel production of oligonucleotides, wherein the DNA synthesis system utilizes photogenerated acid chemistry, parallel microfluidics and a programmable digital light controlled synthesizer, as described in U.S. Patent Publication No. 2007/0059692; Gao et al., Biopolymers 73:579-596 (2004); and Zhou et al., Nucleic Acids Research 32(18):5409-5417 (2004), each of which is incorporated herein by reference.

In some embodiments, the methods of the invention utilize synthesized oligonucleotides that are cleaved off a substrate, such as a microarray. The synthesized nucleic acid molecules can be harvested from the substrate by any useful means. In some embodiments, the portion of the nucleic acid molecule that is directly attached to the substrate, or attached to a linker that is attached to the substrate, is attached to the substrate or linker by an ester bond which is susceptible to hydrolysis by exposure to a hydrolyzing agent, such as hydroxide ions, for example, an aqueous solution of sodium hydroxide or ammonium hydroxide. The entire substrate can be treated with a hydrolyzing agent, or alternatively, a hydrolyzing agent can be applied to a portion of the substrate. For example, a silane linker can be cleaved by exposure of the silica surface to ammonium hydroxide, yielding various silicate salts and releasing the nucleic acid molecules with the silane linker into solution. In some embodiments, ammonium hydroxide can be applied to the portion of a substrate that is covalently attached to the nucleic acid molecules, thereby releasing the nucleic acid molecules into the solution (see, Scott and McLean, Innovations and Perspectives in Solid Phase Synthesis, 3^{rd} International Symposium, 1994, Mayflower Worldwide, pp. 115-124).

The following examples merely illustrate the best mode now contemplated for practicing the invention, but should not be construed to limit the invention.

### Example 1

This Example describes a method for validating single nucleotide variants (SNV) using oligonucleotide ligation and detection of the ligation product by PCR to confirm the presence of a panel of potential SNVs identified during massively parallel sequencing analysis.

Rationale: One of the unforeseen issues that has emerged with SNV and/or mutation detection in the context of massively parallel sequencing platforms is that allele calls are often ambiguous. A combination of factors including sequence read depth, sequence quality, misaligned reads, alignment algorithms, etc., all likely contribute to the error rate associated with high throughput sequence analysis. None of the current methods for single nucleotide variant (SNV) validation are simple, economical, and orthogonal solutions that are suitable to validate thousands of potential SNVs. Therefore, it is important to have a follow-on validation assay that unambiguously detects polymorphisms in a high-throughput manner.

This Example describes a high throughput assay for SNV detection for genotyping genomic DNA samples in which ligation primers are annealed directly to a genomic DNA template in the presence of DNA ligase, followed by a real-time PCR assay for the ligation product. The oligonucleotide ligation occurs when query 5′ and 3′ ligation oligonucleotides bind with perfect complementarity to adjacent sites on target DNA, leaving a gap that can be sealed by DNA ligase. The joining of upstream (5′) and downstream (3′) query ligation oligonucleotides creates a ligation product that serves as a PCR template. A single nucleotide mismatch at the site of the gap significantly impairs ligation efficiency, and therefore decreases the amount of ligation product (i.e., PCR template) that is created. The amount of ligation product generated, which is read out by quantitative PCR, is indicative of the genotype of the target DNA. As further described and demonstrated in Example 3, because only minute quantities of query ligation oligonucleotides are added to the ligation reaction and each annealing event is independent of other annealing events, hundreds of SNV validation assays can be multiplexed in a single reaction vessel for any given test sample.

Methods:

In this Example, three target synthetic templates containing an SNV of interest were designed and investigated in a total of 18 different assays. The first two synthetic template sets were based on actual human SNPs, and the third synthetic template set was based on an actual mouse SNP. Each double-stranded synthetic SNP template was 61 bp in length, which was generated by synthesizing complementary oligos, in which the SNP base (polymorphic site) was located precisely in the center of the synthetic template (i.e., 30 bp on either side of the SNP). All genotypes described herein are oriented to the forward strand, (e.g., A/G) with the first nucleotide (e.g., “A”) listed as the SNV position of interest.

Template set #1 (hSNP1:A/G) contains a synthetic template corresponding to a wild-type (consensus) human allele (SEQ ID NO:1/SEQ ID NO:2) (A/T), and a synthetic template corresponding to a variant human allele (SEQ ID NO:3/SEQ ID NO:4) (G/C), for use as control templates in an assay to distinguish between the presence or absence of the human SNP1 (A/G).

Template set #2 (hSNP2:G/T) contains a synthetic template corresponding to a wild-type human allele (SEQ ID NO:5/SEQ ID NO:6) (G/C), and a synthetic template corresponding to a variant human allele (SEQ ID NO:7/SEQ ID NO:8) (T/A), for use as control templates in an assay to distinguish between the presence or absence of the human SNP (G/T).

Template set #3 (mSNP: A/G) contains a synthetic template corresponding to a wild-type mouse allele (SEQ ID NO:9/SEQ ID NO:10) (A/T), and a synthetic template corresponding to a variant mouse allele (SEQ ID NO:11/SEQ ID NO:12) (G/C), for use as control templates in an assay to distinguish between the presence or absence of the mouse SNP (A/G).

TABLE 1
OLIGONUCLEOTIDES FOR SYNTHETIC TEMPLATES:

| | | | |
|---|---|---|---|
| | | | SEQ |
| | Template | | ID |
| Ref No. | Set | Sequence | NO: |

| | | | |
|---|---|---|---|
| rs949895FA | 1 (FA) | 5′CTTCTGGCAATTGAAGAAAAAAAATTGAGCAGCTGTAACT | 1 |
| | | GCATGCACATTATGCAAATTT3′ | |
| rs949895RT | 1 (RT) | 5′AAATTTGCATAATGTGCATGCAGTTACAGCTGCTCAATTT | 2 |
| | | TTTTTCTTCAATTGCCAGAAG3′ | |
| rs949895FG | 1 (FG) | 5′CTTCTGGCAATTGAAGAAAAAAAATTGAGCGGCTGTAACT | 3 |
| | | GCATGCACATTATGCAAATTT3′ | |
| rs949895RC | 1 (RC) | 5′AAATTTGCATAATGTGCATGCAGTTACAGCCGCTCAATTT | 4 |
| | | TTTTTCTTCAATTGCCAGAAG3′ | |
| rs11042937FG | 2 (FG) | 5′TGCAGCACAAGGGCTGGCACACAGCAGGCCGCCATATTC | 5 |
| | | ATGTGCTGTTCTGCCAGACGTT3′ | |
| rs11042937RC | 2 (RC) | 5′AACGTCTGGCAGAACAGCACATGAATATGGCGGCCTGCT | 6 |
| | | GTGTGCCAGCCCTTGTGCTGCA3′ | |
| rs11042937FT | 2 (FT) | 5′TGCAGCACAAGGGCTGGCACACAGCAGGCCTCCATATTC | 7 |
| | | ATGTGCTGTTCTGCCAGACGTT3′ | |
| rs11042937RA | 2 (RA) | 5′AACGTCTGGCAGAACAGCACATGAATATGGAGGCCTGCT | 8 |
| | | GTGTGCCAGCCCTTGTGCTGCA3′ | |
| FTA | 3 (FA) | 5′GGAGGCCTCGGTGAAGGGCATGCTGGGACGACTCACTAG | 9 |
| | | CACATTGGGTGGCTCAGCTTCC3′ | |
| RTT | 3 (RT) | 5′GGAAGCTGAGCCACCCAATGTGCTAGTGAGTCGTCCCAGC | 10 |
| | | ATGCCCTTCACCGAGGCCTCC3′ | |
| FTG | 3 (FG) | 5′GGAGGCCTCGGTGAAGGGCATGCTGGGACGGCTCACTAG | 11 |
| | | CACATTGGGTGGCTCAGCTTCC3′ | |
| RTC | 3 (RC) | 5′GGAAGCTGAGCCACCCAATGTGCTAGTGAGCCGTCCCAG | 12 |
| | | CATGCCCTTCACCGAGGCCTCC3′ | |

Pooling the Templates

Because each template shown in TABLE 1 has a length of 61 bp, and a molecular weight (MW) of ˜40,000 amu, therefore, a 250 nM solution is 10 ng/μl. Complementary oligonucleotides at a concentration of 10 μM were mixed in buffer containing TEzero (10 mM Tris pH 7.6, 0.1 mM EDTA) plus 20 mM NaCl, diluted to 250 nM, and then diluted 10-fold in TEzero plus 20 mM NaCl that contained 10 ng/μl of human genomic DNA (hgDNA obtained from Clontech). Because hgDNA (diploid) is 6×10⁹ bases, and the templates are 6×10¹ bases, the template was present in 100,000,000-fold excess. The templates were then diluted in buffered hgDNA 10⁶-fold to produce a solution that had 100-fold excess of template over hgDNA. Controls were set up that contained no template (TEzero plus 20 mM NaCl) and the 10 ng/μl hgDNA diluted in TEzero plus 20 mM NaCl.

Ligation Oligonucleotides:

Each assay described in this Example was carried out with two different 5′ allele-specific ligation oligos 300, 400, and one common, phosphorylated 3′ ligation oligo 500 (e.g., as illustrated in FIG. 2). For this Example, three sets of 5′ ligation oligos were designed for each synthetic template, wherein each set had a different primer binding tail sequence 302, in order to determine whether different tail sequences influence the ability to detect a ligation product by PCR amplification. The 5′ ligation oligonucleotides for the human synthetic templates (template sets 1 and 2) were each designed to have 30 nt of complementarity to the target template, and the 5′ ligation oligos for the mouse synthetic templates (template set 3) were designed to have 25 nt of complementarity to the target template. The sequences for the ligation oligos are provided below in TABLE 2.

For each 5′ ligation allele-specific oligo (SEQ ID NO:13-30) the tail sequence 302 containing the PCR primer binding site is underlined, and the 3′ allele-specific region 306 is shown as underlined in bold. For each 3′ common phosphorylated [P] ligation oligo (SEQ ID NO:31-33), the tail sequence 502 containing the PCR binding site is underlined.

TABLE 2
5′ AND 3′ LIGATION OLIGONUCLEOTIDES

| | | | |
|---|---|---|---|
| | | | SEQ |
| | Template | | ID |
| Ref. number | Target | Sequence | NO: |

5′ ligation oligos

| | | | |
|---|---|---|---|
| rs949895_FP1_5′LPA | Template Set | 5′ AGTATAGCCCCAGCGTGTCTACGAGCTTCT | 13 |
| | 1: consensus | GGCAATTGAAGAAAAAAAATTGAGCA 3′ | |
| rs949895_FP1_5′LPG | Template Set | 5′ AGTATAGCCCCAGCGTGTCTACGAGCTTCT | 14 |
| | 1: variant | GGCAATTGAAGAAAAAAAATTGAGCG 3′ | |
| rs949895_FP2_5′LPA | Template Set | 5′ AATCGCTACTGTCGCAAGGGGTCCTCTTCT | 15 |
| | 1: consensus | GGCAATTGAAGAAAAAAAATTGAGCA 3′ | |
| rs949895_FP2_5′LPG | Template Set | 5′ AATCGCTACTGTCGCAAGGGGTCCTCTTCT | 16 |
| | 1: variant | GGCAATTGAAGAAAAAAAATTGAGCG 3′ | |
| rs949895_FP4_5′LPA | Template Set | 5′ GGCTGTAGTCATACCATAGTGCATCCTTCT | 17 |
| | 1: consensus | GGCAATTGAAGAAAAAAAATTGAGCA 3′ | |
| rs949895_FP4_5′LPG | Template Set | 5′ GGCTGTAGTCATACCATAGTGCATCCTTCT | 18 |
| | 1: variant | GGCAATTGAAGAAAAAAAATTGAGCG 3′ | |
| rs11042937_FP1_5′LPA | Template Set | 5′ AGTATAGCCCCAGCGTGTCTACGAGTGCA | 19 |
| | 2: consensus | GCACAAGGGCTGGCACACAGCAGGCCG 3′ | |
| rs11042937_FP1_5′LPG | Template Set | 5′ AGTATAGCCCCAGCGTGTCTACGAGTGCA | 20 |
| | 2: variant | GCACAAGGGCTGGCACACAGCAGGCCT 3′ | |
| rs11042937_FP2_5′LPA | Template Set | 5′ AATCGCTACTGTCGCAAGGGGTCCTTGCAG | 21 |
| | 2: consensus | CACAAGGGCTGGCACACAGCAGGCCG 3′ | |
| rs11042937_FP2_5′LPG | Template Set | 5′ AATCGCTACTGTCGCAAGGGGTCCTTGCAG | 22 |
| | 2: variant | CACAAGGGCTGGCACACAGCAGGCCT 3′ | |
| rs11042937_FP4_5′LPA | Template Set | 5′ GGCTGTAGTCATACCATAGTGCATCTGCAG | 23 |
| | 2: consensus | CACAAGGGCTGGCACACAGCAGGCCG 3′ | |
| rs11042937_FP4_5′LPG | Template Set | 5′ GGCTGTAGTCATACCATAGTGCATCTGCAG | 24 |
| | 2: variant | CACAAGGGCTGGCACACAGCAGGCCT 3′ | |
| mouse_1PEA | Template Set | 5′ AGTATAGCCCCAGCGTGTCTACGAGCTCGG | 25 |
| | 3: consensus | TGAAGGGCATGCTGGGACGA 3′ | |
| mouse_1PEG | Template Set | 5′ AGTATAGCCCCAGCGTGTCTACGAGCTCGG | 26 |
| | 3: variant | TGAAGGGCATGCTGGGACGG 3′ | |
| mouse_2PEA | Template Set | 5′ AATCGCTACTGTCGCAAGGGGTCCTCTCGG | 27 |
| | 3: consensus | TGAAGGGCATGCTGGGACGA 3′ | |
| mouse_2PEG | Template Set | 5′ AATCGCTACTGTCGCAAGGGGTCCTCTCGG | 28 |
| | 3: variant | TGAAGGGCATGCTGGGACGG 3′ | |
| mouse_4PEA | Template Set | 5′ GGCTGTAGTCATACCATAGTGCATCCTCGG | 29 |
| | 3: consensus | TGAAGGGCATGCTGGGACGA 3′ | |
| mouse_4PEG | Template Set | 5′ GGCTGTAGTCATACCATAGTGCATCCTCGG | 30 |
| | 3: variant | TGAAGGGCATGCTGGGACGG 3′ | |

3′ ligation oligos

| | | | |
|---|---|---|---|
| rs949895_FP3_3′LP | Template set 1 | 5′ [P]GCTGTAACTGCATGCACATTATGCAAAT | 31 |
| | | TTTTCCAGCTATCCTGTAAGGCAACGT 3′ | |
| rs11042937_FP3_3′LP | Template set 2 | 5′ [P]CCATATTCATGTGCTGTTCTGCCAGACG | 32 |
| | | TTTTCCAGCTATCCTGTAAGGCAACGT 3′ | |
| mouse_SNP_FP3_3′LP | Template set 3 | 5′ [P]CTCACTAGCACATTGGGTGGCTCAGCTT | 33 |
| | | CCTTCCAGCTATCCTGTAAGGCAACGT 3′ | |

Annealing and Ligation Reaction

The annealing/ligation reactions were carried out under very dilute conditions (5 fmol). A 5 nM stock solution was prepared for each ligation primer in water.

Two different thermo-stable ligases were tested in this Example: Taq DNA ligase, and 9° N DNA Ligase (both obtained from New England Biolabs).

Ligation Reactions:

1 μl 10× ligase buffer (New England Biolabs)

5 μl H₂O

0.1 μl of 5 M NaCl (to increase salt for annealing)

2 μl of annealed template (25 fM)

1 μl of 5′ ligation oligo (5 nM) (allele specific)

1 μl of 3′ ligation primer (5 nM) (phosphorylated common primer)

0.2 μl (40 U/μl) ligase (Taq DNA ligase or 9° N DNA Ligase, New England Biolabs)

A ligation cocktail was prepared that contained the following: each ligase enzyme type (Taq DNA ligase or 9° N DNA Ligase), water, salt, buffer and the common 3′ phosphorylated ligation oligo. 7 μl of the ligation cocktail was aliquoted into wells of a 96 well plate. 2 μl of annealed template and 1 μl of 5′ allele-specific ligation oligo was then added.

The following assays were carried out:

Set #1 (hSNP1:A/G)

Templates tested: SEQ ID NO:1/SEQ ID NO:2 (A) and SEQ ID NO:3/SEQ ID NO:4 (G)

5′ allele-specific ligation oligos tested: SEQ ID NO:13-18

3′ common ligation oligo tested: SEQ ID NO:31

Set #2 (hSNP2:G/T)

Templates tested: SEQ ID NO:5/SEQ ID NO:6 (G) and SEQ ID NO:7/SEQ ID NO:8 (T)

5′ allele-specific ligation oligos tested: SEQ ID NOS:19-24

3′ common ligation oligo tested: SEQ ID NO:32

Set #3 (mSNP:A/G)

Templates tested: SEQ ID NO:9/SEQ ID NO:10 (A) and SEQ ID NO:11/SEQ ID NO:12 (G).

5′ allele-specific ligation oligos tested: SEQ ID NOS:25-30

3′ common ligation oligo tested: SEQ ID NO:33

The ligation reactions were aliquoted into a grid pattern in a 96-well assay plate as shown below in TABLE 3 and incubated in a thermal cycler across the following temperatures:

95° C. for 5 minutes;

75° C. for 15 minutes;

70° C. for 15 minutes;

65° C. for 15 minutes;

60° C. for 15 minutes;

55° C. for 15 minutes;

50° C. for 15 minutes;

45° C. for 15 minutes;

4° C. rest;

The ligation reactions were then diluted to 100 μl with 90 μl of TEzero (10 mM Tris pH 7.6, 0.1 mM EDTA), and quantitative PCR assays were carried out as described below.

TABLE 3
QPCR DETECTION SCHEME FOR LIGATED TEMPLATES

| | | Template | Template | Template | Template | Template | Template |
|---|---|---|---|---|---|---|---|
| Sample | qPCR | Set 1 | Set 1 | Set 2 | Set 2 | Set 3 | Set 3 |
| Series # | Primers | (hSNP1-A) | (hSNP1-G) | (hSNP2-G) | (hSNP2-T) | (mSNP-A) | (mSNP-G) |
| 1 | FP1 + RP3 | SEQ ID | SEQ ID | SEQ ID | SEQ ID | SEQ ID | SEQ ID |
| | | NO: 13 | NO: 13 | NO: 19 | NO: 19 | NO: 25 | NO: 25 |
| 2 | FP1 + RP3 | SEQ ID | SEQ ID | SEQ ID | SEQ ID | SEQ ID | SEQ ID |
| | | NO: 14 | NO:14 | NO: 20 | NO: 20 | NO: 26 | NO: 26 |
| 3 | FP2 + RP3 | SEQ ID | SEQ ID | SEQ ID | SEQ ID | SEQ ID | SEQ ID |
| | | NO: 15 | NO: 15 | NO: 21 | NO: 21 | NO: 27 | NO: 27 |
| 4 | FP2 + RP3 | SEQ ID | SEQ ID | SEQ ID | SEQ ID | SEQ ID | SEQ ID |
| | | NO: 16 | NO: 16 | NO: 22 | NO: 22 | NO: 28 | NO: 28 |
| 5 | FP4 + RP3 | SEQ ID | SEQ ID | SEQ ID | SEQ ID | SEQ ID | SEQ ID |
| | | NO: 17 | NO: 17 | NO: 23 | NO: 23 | NO: 29 | NO: 29 |
| 6 | FP4 + RP3 | SEQ ID | SEQ ID | SEQ ID | SEQ ID | SEQ ID | SEQ ID |
| | | NO: 18 | NO: 18 | NO: 24 | NO: 24 | NO: 30 | NO: 30 |

Quantitative PCR Assays

PCR Primers

Three forward PCR primers were designed to hybridize to the tail regions of the 5′ allele-specific ligation oligos (300, 400) containing three different PCR Primer Binding sites (302, 402) as follows:

| | | |
|---|---|---|
| FP1 | 5′ AGTATAGCCCCAGCGTGTCTACGAG3′ | (SEQ ID NO: 34) |
| FP2 | 5′ AATCGCTACTGTCGCAAGGGGTCCT3′ | (SEQ ID NO: 35) |
| FP4 | 5′ GGCTGTAGTCATACCATAGTGCATC3′ | (SEQ ID NO: 36) |

One reverse PCR Primer was designed to hybridize to the tail region of the 3′ common ligation oligo (500):

| | | |
|---|---|---|
| RP3 | 5′ ACGTTGCCTTACAGGATAGCTGGAA3′ | (SEQ ID NO: 37) |

Power SYBR master mix (Applied Biosystems): a premix of all the components (SYBR Green Dye, AmpliTaq Gold® DNA Polymerase, dNTPs, and buffer components) except primers, template, and water, necessary to perform real-time PCR. The SYBR Green dye, which binds to double-stranded DNA, provides a fluorescent signal that reflects the amount of dsDNA product generated during PCR. The master mix includes AmpliTaq Gold® DNA Polymerase, provided in an inactive state to allow pre-mixing of PCR reagents at room temperate and allows for an automated, hot start. Upon thermal activation, the enzyme is activated.

qPCR Assay:

2 μl of each diluted ligation reaction was used as a template in four 10 μl qPCR reactions as follows:

A PCR reaction cocktail was prepared so that each sample would contain:

5 μl of 2× master mix (Power SYBR master mix, manufactured by Applied Biosystems)

1.4 μl H₂O

0.8 μl of Forward PCR primer (10 μM)

0.8 μl of Reverse PCR primer (10 μM)

8 μl total volume was aliquoted into wells of the 96 well plate, and 2 μl of diluted ligation template was added.

qPCR was run for 40 cycles with a denaturation step at the end to assess product integrity.

The qPCR abundance ratios for correct over mismatched calls for all 36 assays that were run is shown below in TABLE 4.

TABLE 4
QPCR RATIOS OF TARGET/OFF-TARGET FOR LIGATION
DEPENDENT GENOTYPING ASSAYS

| qPCR primer | ligation template | 9° N data | Taq data |
|---|---|---|---|
| FP1 | hSNP1-A | 19 | 77 |
| FP1 | hSNP1-G | 93 | 17 |
| FP2 | hSNP1-A | 8* | 126 |
| FP2 | hSNP1-G | 71 | 20 |
| FP4 | hSNP1-A | 10 | 111 |
| FP4 | hSNP1-G | 59 | 20 |
| FP1 | hSNP2-G | 211 | 311 |
| FP1 | hSNP2-T | 195 | 34 |
| FP2 | hSNP2-G | 53 | 309 |
| FP2 | hSNP2-T | 312 | 95 |
| FP4 | hSNP2-G | 489 | 288 |
| FP4 | hSNP2-T | 217 | 0* |
| FP1 | mSNP-A | 29 | 154 |
| FP1 | mSNP-G | 50 | 42 |
| FP2 | mSNP-A | 41 | 72 |
| FP2 | mSNP-G | 62 | 29 |
| FP4 | mSNP-A | 58 | 101 |
| FP4 | mSNP-G | 31 | 20 |

Results:

The data shown above in TABLE 4 is a measure of the specificity of the SNV detection assay, with all but two assay sets (shown with *) registering ratios of correct versus mismatch >10. The magnitude of this differential detection is an adequate foundation for a genotyping assay because the ten-fold and greater difference in absolute abundances between correctly matched assays and mismatched assays translates into a correct-allele-to-incorrect allele Ct difference in qPCR measurements of >3, which is a threshold value well above random deviations that are observed within an experiment.

These data demonstrate that all three of the target synthetic SNV templates were accurately detected with the total of 18 different assays using at least one of the two tested thermostable DNA ligase enzymes. DNA ligase and, in particular, the thermostable Taq DNA ligase, are ideal enzymes for interrogating nucleotide polymorphisms because they can only seal nicks at sites of perfect base pairing. The thermostable nature of the DNA ligase is advantageous because the enzyme activity is retained at the high temperatures needed for DNA melting and reannealing. It is noted that the ligation oligos worked at very dilute concentrations (5 fmol), and all the tested arbitrary PCR binding tails all appeared to work; therefore, the multiplexing aspect of the assays is likely to be successfully implemented.

This Example demonstrates the successful use of a ligation-dependent assay to detect SNVs in synthetic templates mixed with genomic DNA. However, the allele calls were not perfect in this experiment, likely due to the fact that the mismatch generated significant background, which is typical of many genotyping assays. In order to improve the accuracy of this detection assay, each set of ligation oligos was calibrated against a control set of synthetic reference and variant templates, as described in Example 3.

### Example 2

This Example describes the manufacture of a 96-well assay plate comprising a 12 column by 8 row primer matrix of detection primer pairs (also referred to as a “universal PCR decoding matrix”), which can be pre-made and stored in a freezer, for decoding a multiplex assay, such as a multiplex ligation-dependent genotyping assay for genotyping a test sample at a plurality of SNV positions of interest.

PCR Primer Matrix Design

As described in Example 1, the 5′ and 3′ ligation oligos (300, 400, and 500) for each genotyping assay are tailed with unique PCR primer binding regions (302, 402, 502) that correspond to a pair of PCR detection primers that are present in a particular well (also referred to as an “address”) in the universal assay matrix. Therefore, each address (for example, a well in a 96-well plate) “decodes” the result from an individual genotyping assay.

An important element of the universal PCR decoding matrix is that the last two or three (penultimate) 3′ bases of the PCR primers are chosen to reduce and preferably eliminate primer-dimer formation, and the remaining bases are specificity tags chosen to provide a unique address at an intersection position (well) in the matrix, disposed into one or more assay plates.

A matrix comprising 20 “universal” paired decoding PCR primers (provided in TABLE 5) was produced for use in a universal detection assay carried out on a 96-well plate 800 (e.g., as shown in FIGS. 4-6), as follows.

Each of the 12 column “C” PCR primers were aliquoted into a separate well 826 along the horizontal axis of the 96 well assay plate (columns 1-12).

Each of the 8 row “R” PCR primers were aliquoted into a separate well 826 along the vertical axis of the 96 well assay plate (rows A-H).

As shown below in TABLE 6, each well 826 located at the intersection of a row and column of the 96 well assay plate contained a unique PCR primer pair, thereby providing a unique “address” at a designated physical location on the matrix (i.e., a positionally addressable array). The universal PCR plate containing the 96 unique pairs of PCR primers was then used to “decode” the results of a multiplexed ligation-dependent genotyping assay. The allele-specific ligation oligonucleotides in the genotyping assay were designed with tail sequences that are complementary to the PCR primers at a specific well location in the assay plate.

The PCR Primer Design for the Universal PCR Decoding Plate:

It was previously determined that almost any two 25 mer oligonucleotides having DNA sequences with a balanced A, C, G, and T content can serve as quantitative PCR primer pairs, provided that they terminate in a di- or tri-nucleotide sequence that inhibits primer-dimer formation (data not shown). In this Example, each PCR primer was 25 nucleotides in length, with the 23 bases at the 5′ end of the primer 602, 702 serving as specificity “addresses,” due to the fact that each well of the matrix contained a unique pair of primers which would bind to and amplify the ligation product resulting from an individual genotyping assay.

As shown in FIG. 3C, each forward PCR primer 600 has a 5′ region 602 that binds to a primer binding region in the 5′ tail of a 5′ ligation oligo, and a region 606 at the 3′ end having a sequence selected to inhibit primer-dimer interactions with the reverse PCR primer. For example, as shown in FIG. 2B, forward primer 600 has a region 602 that binds to primer binding region 302 in the 5′ tail of the 5′ ligation oligo 300, and forward primer 600′ has a region 602 that binds to primer region 402 in the 5′ tail of the 5′ ligation oligo 400. Similarly, each reverse PCR primer 700 has a 5′ region 702 that binds to a primer binding region in the 3′ tail of a 3′ ligation oligo, and a region 706 at the 3′ end having a sequence selected to inhibit primer-dimer interactions with the forward PCR primer.

The “C” series was designed as the reverse primer set 700 to bind to the 3′ common tail region 502 on the ligation products 200, 250.

The “R” series was designed as the forward primer set 600, each forward primer having a region 606 designed to specifically bind to the 5′ tail region 302, 402 on the ligation products 200, 250.

To alleviate primer dimer formation, each “R” PCR primer sequence ended in “CT” and each “C” PCR primer sequence ended in “GA.” These terminal dinucleotides cannot pair with one another nor can they self anneal, hence they prevent the formation of primer dimers. It will be understood by those of skill in the art that other di- or tri-nucleotide sequences could be chosen to avoid primer-dimer formation. Exemplary tri-nucleotide sequences chosen to avoid the formation of primer-dimers are provided in Example 4 herein.

The PCR primers were synthesized by MWG/Operon, Huntsville, Ala., resuspended in water to a concentration of 100 μM and a 1 ml of a 10 μM working stock was made for each primer.

The sequences of the 20 universal PCR primers used to generate the universal PCR decoding matrix are provided below in TABLE 5.

TABLE 5
UNIVERSAL DECODING PCR PRIMERS

| Reference | | SEQ ID |
|---|---|---|
| number | Sequence | NO: |
| C1 | 5′ ACTCTGCGCTCTGGAACTTACCGGA 3′ | 38 |
| C2 | 5′GATCTTGGACGAAGTCGTCCTATGA 3′ | 39 |
| C3 | 5′GTTTGCATGAAGACCTCTATACAGA 3′ | 40 |
| C4 | 5′CTGAAAGGTCCATGGCCTGTACTGA 3′ | 41 |
| C5 | 5′TTGTATCGATGCAGCCAGGATCCGA 3′ | 42 |
| C6 | 5′CACAGAATTAGCGATCTATGCCGGA 3′ | 43 |
| C7 | 5′CACGCCTCATCGTAGTGTAGGAGGA 3′ | 44 |
| C8 | 5′ATAACATTGAACGCTGCCGTTGCGA 3′ | 45 |
| C9 | 5′CAGACTACGGCAATATAACGCTGGA 3′ | 46 |
| C10 | 5′ACGTAACTATTACGGTGAGCGCCGA 3′ | 47 |
| C11 | 5′ATGGATAGCCGCTGTTTAACTACGA 3′ | 48 |
| C12 | 5′TTCGGCTTCCACAGAGCAAGGTAGA 3′ | 49 |
| R1 | 5′TGTATCAGCATCTGGCTCAGCGTCT 3′ | 50 |
| R2 | 5′CTTTGGGGTAAGCGACCATCAGCCT 3′ | 51 |
| R3 | 5′TACATAGAATCTACCGTGGTGACCT 3′ | 52 |
| R4 | 5′ACGATGGCGTTGCAGGCGCTTACCT 3′ | 53 |
| R5 | 5′TTGACTGAGACTCCTCATGACCTCT 3′ | 54 |
| R6 | 5′GCCGTTTCATATCGAACAAGGCGCT 3′ | 55 |
| R7 | 5′GGGCTACTCGCAATTTCAAATTGCT 3′ | 56 |
| R8 | 5′CGCCAGCAATCAGCTTTGATACACT 3′ | 57 |

Design of Universal Assay Matrix

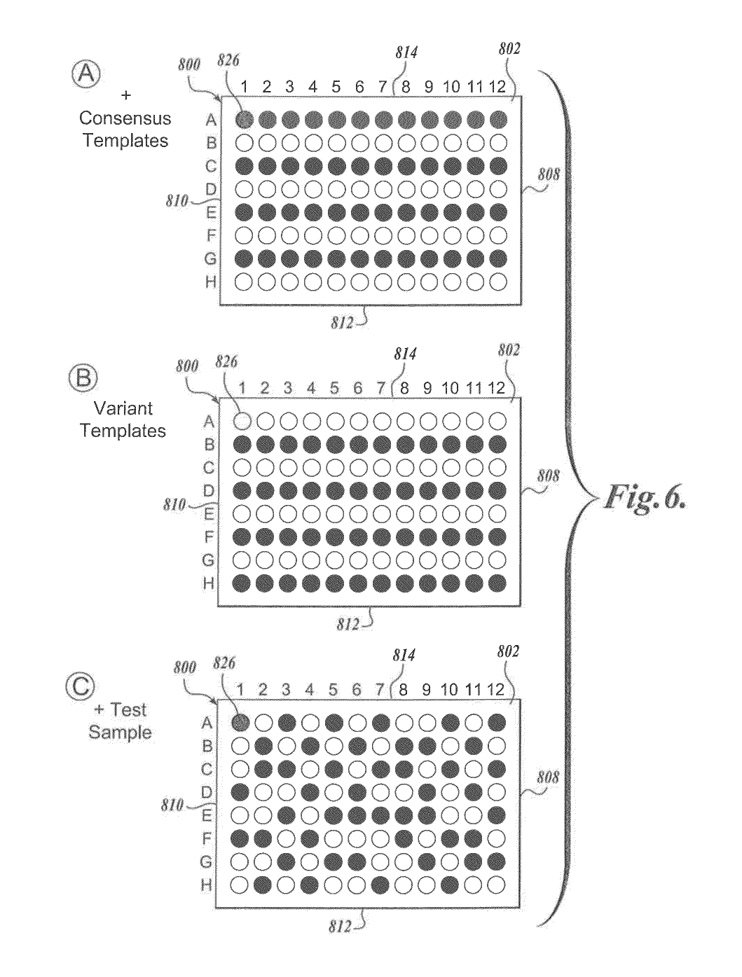

The layout of the universal assay matrix for qPCR to detect ligation products in a multiplexed ligation-dependent genotyping assay for multiple SNV positions of interest, was a matrix of wells (i.e., features), the matrix comprising a plurality of columns and rows. For example, with reference to FIG. 6A, wells A1 and B1 represent the qPCR assay result detecting the ligation products resulting from ligation of a 5′ consensus ligation oligo (at A1) or a 5′ variant ligation oligo (at B1) with the 3′ ligation oligo in an assay for a determining the nucleotide present at a particular SNV position of interest.

For example, as shown in more detail in FIG. 4, the tail region of the 5′ consensus allele-specific ligation oligo 300 for SNV (Gene #1) had a first primer binding sequence (e.g., for binding to PCR primer R1=SEQ ID NO:50), and the tail region of the 5′ variant allele-specific ligation oligo 400 for SNV (Gene #1) had a second primer binding sequence (e.g., for binding to PCR primer R2=SEQ ID NO:51). The 3′ phosphorylated common primer 500, common to both assays for SNV (Gene #1), had a common primer binding sequence (e.g., for binding to PCR primer C1=SEQ ID NO:38). As further shown in FIG. 4, assay #1 is decoded in the universal assay matrix dispensed into a 96-well plate at well A1 (containing PCR primers R1+C1) for measuring the amount of consensus ligation product 200, and at well B1 (containing PCR primers R2 and C1), for measuring the amount of variant ligation product 250 present in the multiplexed ligation mixture. Thus, a 96 well assay plate can accommodate a universal matrix for carrying out consensus versus variant assays for 48 SNV positions of interest.

As shown in FIG. 6, a set of three identical 96-well assay plates 800, each comprising a universal matrix can be used to generate a set of data representing: a pool of synthetic consensus templates (FIG. 6A); a pool of synthetic variant templates (FIG. 6B); and a test sample (FIG. 6C), each assayed with the same pool of SNV query consensus and variant allele-specific ligation oligos for a particular SNV having PCR tails corresponding to a particular location on the assay plate.

Preparation of the Assay Plate(s) Containing the Universal Matrix

The assay plates were prepared for quantitative PCR (qPCR) assays as follows:

35 mls of 2× Power SYBR master mix (Applied Biosystems, Foster City, Calif.) was combined with 10 mls of H₂O. 450 μl of the mixture was aliquoted into each well of a 96 well assay plate. 55 μl of the 12 “C” (reverse) primers (10 μM) were aliquoted into the wells of the columns (C) of the assay plate, and 55 μl of the 8 “R” (forward) primers (10 μM) were aliquoted into the wells of the rows (R) of the 96 well assay plate, as shown below in TABLE 6. The assay plate can be run in a 96 well plate format. Alternatively, for an assay done in quadruplicate, the reagents were mixed, then 8 μl per well was aliquoted in quadruplicate into a 384 qPCR plate, in order to carry out 4 identical reactions for each qPCR primer pair, as described in Example 3.

For example, as described in Example 3, 2 μl aliquots were dispensed into all wells of the prepared 384 well qPCR plate (4×96). The samples were mixed, and the qPCR assay was run on an ABI 7900 instrument set on SYBR detection channel.

TABLE 6
96-WELL ASSAY PLATE CONTAINING
A UNIVERSAL PCR PRIMER MATRIX

| | C1 | C2 | C3 | C4 | C5 | C6 | C7 | C8 | C9 | C10 | C11 | C12 |
|---|---|---|---|---|---|---|---|---|---|---|---|---|
| | (SEQ | (SEQ | (SEQ | (SEQ | (SEQ | (SEQ | (SEQ | (SEQ | (SEQ | (SEQ | (SEQ | (SEQ |
| | ID | ID | ID | ID | ID | ID | ID | ID | ID | ID | ID | ID |
| | NO: 38) | NO: 39) | NO: 40) | NO: 41) | NO: 42) | NO: 43) | NO: 44) | NO: 45) | NO: 46) | NO: 47) | NO: 48) | NO: 49) |
| A: R1 | R1 × | R1 × | R1 × | R1 × | R1 × | R1 × | R1 × | R1 × | R1 × | R1 × | R1 × | R1 × |
| (SEQ ID | C1 | C2 | C3 | C4 | C5 | C6 | C7 | C8 | C9 | C10 | C11 | C12 |
| NO: 50) | | | | | | | | | | | | |
| B: R2 | R2 × | R2 × | R2 × | R2 × | R2 × | R2 × | R2 × | R2 × | R2 × | R2 × | R2 × | R2 × |
| (SEQ ID | C1 | C2 | C3 | C4 | C5 | C6 | C7 | C8 | C9 | C10 | C11 | C12 |
| NO: 51) | | | | | | | | | | | | |
| C: R3 | R3 × | R3 × | R3 × | R3 × | R3 × | R3 × | R3 × | R3 × | R3 × | R3 × | R3 × | R3 × |
| (SEQ ID | C1 | C2 | C3 | C4 | C5 | C6 | C7 | C8 | C9 | C10 | C11 | C12 |
| NO: 52) | | | | | | | | | | | | |
| D: R4 | R4 × | R4 × | R4 × | R4 × | R4 × | R4 × | R4 × | R4 × | R4 × | R4 × | R4 × | R4 × |
| (SEQ ID | C1 | C2 | C3 | C4 | C5 | C6 | C7 | C8 | C9 | C10 | C11 | C12 |
| NO: 53) | | | | | | | | | | | | |
| E: R5 | R5 × | R5 × | R5 × | R5 × | R5 × | R5 × | R5 × | R5 × | R5 × | R5 × | R5 × | R5 × |
| (SEQ ID | C1 | C2 | C3 | C4 | C5 | C6 | C7 | C8 | C9 | C10 | C11 | C12 |
| NO: 54) | | | | | | | | | | | | |
| F: R6 | R6 × | R6 × | R6 × | R6 × | R6 × | R6 × | R6 × | R6 × | R6 × | R6 × | R6 × | R6 × |
| (SEQ ID | C1 | C2 | C3 | C4 | C5 | C6 | C7 | C8 | C9 | C10 | C11 | C12 |
| NO: 55) | | | | | | | | | | | | |
| G: R7 | R7 × | R7 × | R7 × | R7 × | R7 × | R7 × | R7 × | R7 × | R7 × | R7 × | R7 × | R7 × |
| (SEQ ID | C1 | C2 | C3 | C4 | C5 | C6 | C7 | C8 | C9 | C10 | C11 | C12 |
| NO: 56) | | | | | | | | | | | | |
| H: R8 | R8 × | R8 × | R8 × | R8 × | R8 × | R8 × | R8 × | R8 × | R8 × | R8 × | R8 × | R8 × |
| (SEQ ID | C1 | C2 | C3 | C4 | C5 | C6 | C7 | C8 | C9 | C10 | C11 | C12 |
| NO: 57) | | | | | | | | | | | | |

Results:

In order to validate the universal PCR decoding matrix, an initial experiment was carried out in which TEzero (no template control) was added to a universal PCR decoding matrix, prepared as described above, and a qPCR assay was carried out with the 8 (“R”) forward PCR primers (SEQ ID NOS:50-57) and the 12 (“C”) reverse PCR primers (SEQ ID NOS:38-49). The qPCR data was analyzed at the level of raw Cts. In this initial experiment, only three wells in column C10 gave background Ct values less than 30 (lower Ct values represent higher amounts of product), and this background did not significantly impact assay performance. Therefore, the concept of a universal matrix of PCR primers was validated by this initial experiment. It will be understood by those of skill in the art that the design of the universal PCR matrix described herein, and the design principles of the PCR primers can be expanded to accommodate approximately 1000 or more samples, which can be assayed on an appropriately sized multi-well assay plate. For example, a primer matrix with >1000 addresses can be constructed from as few as 32 row and 36 column primers (32×36=1152 unique addresses, otherwise referred to as “unique features”).

### Example 3

This Example describes a multiplexed, high throughput assay for SNV genotyping using oligonucleotide ligation and detection of the ligation product by PCR to validate the presence or absence of a panel of 96 potential SNVs that were initially detected during high-throughput sequence analysis.

Rationale:

In order to further develop the ligation-dependent SNV detection assay described in Example 1 for high-throughput analysis, an experiment was set up to genotype 96 potential SNVs that were initially identified during massively-parallel sequencing of a genomic DNA library representing 139 cancer-related genes from a Calu6 cell line. This assay, referred to as “oligonucleotide ligation validation of potential SNVs” or “OLIVES” combines single-tube multiplexing with assay read-out in a universal PCR decoding plate (described in Example 2) to provide both validated genotypes and assay reagents for follow-on genotyping studies.

In the first step of the assay, 5′ allele-specific oligos and 3′ phosphorylated common ligation oligonucleotides (up to 1000 or more) are annealed to the test DNA and ligated. In the second step, the ligation mixture is distributed across universal PCR “decoding” plates, as described in Example 2, which can be pre-made and stored in a freezer prior to use.

Methods:

1. Prepare DNA Samples for Genotyping:

Genotyping signal-to-noise ratios typically improve when DNA samples are enriched in target sequences. Therefore, in this Example a comparison was made between genotyping total genomic DNA and genotyping a genomic DNA library enriched for target sequences.

A. Total genomic DNA 85 ng/μl was isolated from the Calu6 cell line from cells grown in culture using a standard genomic DNA purification kit (Qiagen, Valencia, Calif.).

B. A genomic DNA library was generated from a panel of 139 cancer-related genes from the Calu6 cell line and was enriched using solution-based capture as follows.

Preparation of Capture Probes

All the exons of the set of 139 genes were identified. An algorithm was then applied for picking alternating sense and antisense strand chimeric oligos with a 5′ target-specific region (35 nt) with a sequence that hybridizes to either the sense or antisense strand of each of these exons, and a 3′ region that hybridizes to the biotinylated adaptor capture oligo.

These capture oligonucleotides were chosen as follows. For exons less than 69 nucleotides in length, two oligonucleotides, both targeting the same strand and oriented in the same direction, and not overlapping one another in sequence by more than 10 nucleotides were chosen. In some cases where exons were very short (i.e., <60 nucleotides), these capture oligonucleotides included flanking exon sequences.

For exons between 70 and 115 nucleotides in length, two oligonucleotides targeting opposite Watson and Crick strands and oriented in the opposite orientations were selected. The first oligonucleotide covered exon base positions 1-35 and the second oligonucleotide was positioned from base positions 80-115, which often included flanking intron sequences, so that the oligos were each about 35 nt in length, and spaced about 45 nt apart.

For exonic sequences greater than 115 nucleotides in length, the first capture oligonucleotide was placed at exon positions 1-35 and successive oligos were placed in alternating orientations with a spacing of 45 nucleotides between oligonucleotides.

The oligos designed as described above were synthesized by Operon and provided in a plate at 100 μM and pooled into a single 50 ml sample using a Biomek robot. The pooled 3229 capture oligos were then diluted to 10 μM and 1 μM.

TaqMan assays were developed for the 139 target genes. TaqMan assays were also developed for off-target genes for use as negative controls. These genes were not targeted by capture oligonucleotides, and it was shown that their representation diminished during the course of target library enrichment.

Library Generation:

Genomic DNA libraries were generated by fragmenting Calu6 genomic DNA and ligating on linkers containing a first and second primer binding site, followed by PCR amplification for 20 cycles with PCR forward primer and PCR reverse primer, then the PCR product was purified over a Qiaquick column

Solution-Based Capture and Enrichment of Libraries for Target Sequences

Capture reagents: 10 μM of the capture oligos for the 139 candidate genes described were mixed with 10 μM of the biotinylated adaptor oligo.

Capture Mixture: 125 μl of 2× binding buffer (2 M NaCl, 20 mM Tris pH 7.6, 0.2 mM EDTA), 60 μl (4.3 μg) of gDNA library, 5 μl capture oligo pool (50 pM of 10 μM of oligo pool+adaptor oligo), and 60 μl water, for a total volume of 250 μl.

The reaction mixture was annealed as follows:

94° C. for 1 minute

90° C. for 1 minute

85° C. for 1 minute

80° C. for 1 minute

75° C. for 1 minute

70° C. for 1 minute

65° C. for 1 minute

60° C. for 1 minute

55° C. for 1 minute

50° C. for 1 minute

45° C. for 1 minute

40° C. for 1 minute

25° C.—hold

Capture Reagents: Washed beads were prepared by combining six aliquots of 50 μl beads (in principle, each 50 μl of beads is capable of binding 50 pmol of dsDNA complex), 500 μl 2× binding buffer and 440 μl water. The beads were pulled over with a magnet and washed twice with 1 ml 1× binding buffer.

1st Round of Capture/Enrichment: The aliquots of washed oligos were combined with the annealed oligos into a total volume of 1 ml of 1× binding buffer and mixed gently for 15 minutes.

Wash Solutions:

A series of wash buffers with increasing formamide were tested, each with 100 mM Tris pH 7.6, 1 mM EDTA, and a range of formamide from 15%, 20%, 25%, 30%, and 50%.

It was previously determined that the presence of 20 mM NaCl in the 10 mM Tris pH 7.6, 1 mM EDTA buffer enhanced non-specific binding (data not shown), therefore the NaCl was eliminated in the wash buffer in this experiment.

The capture oligos/library/bead complexes were washed four times with the above-described wash buffers including formamide, 1 ml each wash for 5 minutes.

Elution: The DNA bound to the beads was eluted with two aliquots of 50 μl of water by incubation at 94° C. for 1 minute each, pulling over the beads and removing the eluate, for a total eluate volume of 100 μl.

Amplification of Eluate (Once Enriched Library):

PCR Reaction Mixture (5% DMSO)

29 μl H₂O

20 μl 5× buffer (supplied by manufacturer with the EXPAND^{plus}® kit, Roche)

10 μl 25 mM MgCl₂

10 μl template ( 1/10th eluate from once enriched fragment library)

5 μl dNTPs (10 nM each dNTP)

5 μl DMSO

10 μl 10 μM Forward PCR primer

10 μl 10 μM Reverse PCR primer

1 μl Expand^{PLUS}® polymerase (Roche)

100 μl total volume

PCR Cycling Conditions

1 cycle:
94° C. for 2 minutes

10 cycles:
94° C. for 30 sec
60° C. for 30 sec
72° C. for 1 minute

10 or 15 cycles:
94° C. for 30 sec
60° C. for 30 sec
72° C. for 1 minute plus 10 sec/cycle

1 cycle:
72° C. for 7 minutes
4° C. hold

The PCR reaction products were purified over a Qiaquick column and quantified.

1 μl of PCR product was analyzed on a 2% agarose gel.

2. Design of SNV Query Oligos for Ligation-Dependent Genotyping Assay

A set of SNV query oligos were designed to determine the presence or absence of a panel of potential SNVs that had been identified during the sequencing of 139 genes from the Calu6 cell line using massively-parallel sequencing techniques (data not shown). From this initial sequencing analysis, 96 non-synonymous SNV calls were identified, whose confidence was ranked from high to low based on the degree of overlapping bioinformatic evidence. Assays 1 to 96 listed in TABLE 11 correspond to the 96 distinct putative SNVs that were initially detected as potential polymorphic loci during massively parallel sequencing. The lowest numbered assays in TABLE 11 (starting at assay #1), correspond to the highest confidence ranking, based on the degree of overlapping bioinformatic evidence (e.g., the presence of the SNV in dbSNP). As shown in TABLE 11, many known SNPs from the dbSNP database and two known mutations identified in the Wellcome Trust COSMIC database were included in the set of 96 non-synonymous SNV calls.

For each of the 96 SNV Positions of Interest, the Following Reagents were Generated:
A 5′ allele-specific consensus ligation oligo (51 mer) (TABLE 7)
A 5′ allele-specific variant ligation oligo (51 mer) (TABLE 8)
A 3′ common ligation oligo (50 mer) (TABLE 9)
A pair of oligos to generate an annealed reference template with the consensus SNV sequence (51 mers) (not shown)
A pair of oligos to generate an annealed reference template with the variant SNV sequence (51 mers) (not shown)

Ligation Oligonucleotides

The paired set of allele-specific (consensus and variant) 5′ and 3′ ligation oligonucleotide pairs for each SNV target of interest were designed as follows:

Each 5′ ligation oligo 300, 400 had a total length of 51 nucleotides, with a target-specific complementary region 304, 404 of 25 nucleotides, an allele-specific region 306, 406 of 1 nucleotide, and a primer-binding tail region 302, 402 of 25 nucleotides in length, including 2 nt at the 3′ end corresponding to the forward PCR primer region 606 selected to avoid primer dimer (e.g., “CT”).

The target-specific binding region 304, 404 of the 5′ ligation oligos was designed to have a length of 25 nt that were 100% complementary to the target region of interest immediately 5′ of each of the panel of 96 SNV loci of interest. The allele-specific binding region 306, 406 of the 5′ ligation oligos were designed to have a length of 1 nt that was complementary to the consensus or variant allele for a particular SNV of interest. The sequence of the tail region 302, 402 was selected to bind to a forward PCR primer 600 in the universal assay plate 800 made as described in Example 2. The 5′ consensus ligation oligos for SNV assays 1-96 are provided in TABLE 7. The 5′ variant ligation oligos for SNV assays 1-96 are provided in TABLE 8.

Each 3′ ligation oligo 500 had a total length of 50 nucleotides, with a target-specific complementary region 504 of 25 nucleotides, and a primer-binding tail region 502 of 25 nucleotides in length. The target-specific complementary region of the 3′ ligation oligos was designed to have a length of 25 nt that was 100% complementary to the target region of interest starting at the nucleotide immediately 3′ to the SNV position of interest. The sequence of the tail region 502 was selected to bind to a reverse PCR primer 700 in the universal assay plate 800 made as described in Example 2. The 3′ ligation oligos were phosphorylated prior to use in the assay.

The 3′ ligation sequences for SNV assays 1-96 are provided below in TABLE 9.

Ligation Primers:

6 plates of 96 well plates were ordered for synthesis as follows:

Plate 1: 5′ consensus and variant ligation oligos for 1-48

Plate 2: 3′ common ligation oligos for 1-48

Plate 3: consensus templates for 1-96

Plate 4: 5′ consensus and variant ligation oligos for 49-96

Plate 5: 3′ common ligation oligos for 49-96

Plate 6: variant templates for 1-96

TABLE 7
CONSENSUS 5′ LIGATION OLIGOS

| | | |
|---|---|---|
| | | SEQ |
| SNV | | ID |
| Assay # | SEQUENCE (5′ to 3′) | NO: |

| | | |
|---|---|---|
| 1 | TGTATCAGCATCTGGCTCAGCGTCTGCTTTCATTCATATCTGCAGGTTCAA | 58 |
| 2 | TACATAGAATCTACCGTGGTGACCTGCAGTCCAGAGCACCGTGGTCCTGCT | 59 |
| 3 | TTGACTGAGACTCCTCATGACCTCTCACCACTGGGGTAAGGTTTTCTAGGG | 60 |
| 4 | GGGCTACTCGCAATTTCAAATTGCTAGCCAGTTTTCCATGGGTTCTACTAC | 61 |
| 5 | TGTATCAGCATCTGGCTCAGCGTCTCTTCCCGGTCAGCTACTCCTCTTCCG | 62 |
| 6 | TACATAGAATCTACCGTGGTGACCTTGATCCATTAGATTCAAATGTAGCAA | 63 |
| 7 | TTGACTGAGACTCCTCATGACCTCTACCTGCTGGTGCCACTCTGGAAAGGC | 64 |
| 8 | GGGCTACTCGCAATTTCAAATTGCTCTTGCTGCTTCCAGTAAATAAGGTGA | 65 |
| 9 | TGTATCAGCATCTGGCTCAGCGTCTATCCTTGTCCAAGGAGGCTGTTTCTG | 66 |
| 10 | TACATAGAATCTACCGTGGTGACCTTCCACACGCAAATTTCCTTCCACTCG | 67 |
| 11 | TTGACTGAGACTCCTCATGACCTCTAGGAGCTGCTGGTGCAGGGGCCACGG | 68 |
| 12 | GGGCTACTCGCAATTTCAAATTGCTATTCATCGGACATGTTACTGTTTTTC | 69 |
| 13 | TGTATCAGCATCTGGCTCAGCGTCTGTGTCATCAACTTGGTCCACAGTCGT | 70 |
| 14 | TACATAGAATCTACCGTGGTGACCTGGCCCTCTAGGGACTCGAACAGAGAT | 71 |
| 15 | TTGACTGAGACTCCTCATGACCTCTCAGAGGGAGGACGAGCTGACCTTCAT | 72 |
| 16 | GGGCTACTCGCAATTTCAAATTGCTCCAACTCGAAATTCCCCGTGACCAGA | 73 |
| 17 | TGTATCAGCATCTGGCTCAGCGTCTACCAGCAGATACTCAGCCGGAGGATA | 74 |
| 18 | TACATAGAATCTACCGTGGTGACCTGAGGACCCCAAGTCCCATAGGGACCC | 75 |
| 19 | TTGACTGAGACTCCTCATGACCTCTGCAGCGCACCACGGGACCCAAGCCCG | 76 |
| 20 | GGGCTACTCGCAATTTCAAATTGCTCAGAGTCTGAGGTAGCTGCCCTGGCA | 77 |
| 21 | TGTATCAGCATCTGGCTCAGCGTCTTGCTGTTTTCTTCCTTCAGGCATACA | 78 |
| 22 | TACATAGAATCTACCGTGGTGACCTCCTGAACAGCTCGCGGCTCAGCAGGG | 79 |
| 23 | TTGACTGAGACTCCTCATGACCTCTATCTTCAAAGTTGCAGTAAAAACCCA | 80 |
| 24 | GGGCTACTCGCAATTTCAAATTGCTCTTGATTCATGATATTTTACTCCAAG | 81 |
| 25 | TGTATCAGCATCTGGCTCAGCGTCTTCCCTCATTGCACTGTACTCCTCTTG | 82 |
| 26 | TACATAGAATCTACCGTGGTGACCTGTCCGAGGACAACGATGAGGCGGCGC | 83 |
| 27 | TTGACTGAGACTCCTCATGACCTCTTTCCGCCTGGTGTTGGAAGAGACAGG | 84 |
| 28 | GGGCTACTCGCAATTTCAAATTGCTTGGTCTTTCAGTGCCTCCACTATGAC | 85 |
| 29 | TGTATCAGCATCTGGCTCAGCGTCTTGAAGAGAAATATAAGAAGGCTATGG | 86 |
| 30 | TACATAGAATCTACCGTGGTGACCTCCTCCAGGTGCAGGAGTTCATGCTCA | 87 |
| 31 | TTGACTGAGACTCCTCATGACCTCTAAAGGCAATGTGGGATCCTGAATTGC | 88 |
| 32 | GGGCTACTCGCAATTTCAAATTGCTGCCCGAACAGCCGCTGGATATGGGAC | 89 |
| 33 | TGTATCAGCATCTGGCTCAGCGTCTCTAAAAAGGACCCTGAAGGTTGTGAC | 90 |
| 34 | TACATAGAATCTACCGTGGTGACCTTCATATGGATGATAATGATGGAGAAC | 91 |
| 35 | TTGACTGAGACTCCTCATGACCTCTCTTGGCTGTGCTCCTGCTGCTGGCCG | 92 |
| 36 | GGGCTACTCGCAATTTCAAATTGCTATCAACTATAGGTTGCTTTGGTGGTG | 93 |
| 37 | TGTATCAGCATCTGGCTCAGCGTCTAAATTTCTGAATAACTGAAGTTGGTC | 94 |
| 38 | TACATAGAATCTACCGTGGTGACCTGGTAGCAGACAAACCTGTGGTTGATC | 95 |
| 39 | TTGACTGAGACTCCTCATGACCTCTGAGCTTTGGGTTGTTCCTTAGGACCC | 96 |
| 40 | GGGCTACTCGCAATTTCAAATTGCTGGAATTACGGCAGCCCTTCTTTCCCA | 97 |
| 41 | TGTATCAGCATCTGGCTCAGCGTCTCGGGGCCTCTGCTTGGATGTGATGAC | 98 |
| 42 | TACATAGAATCTACCGTGGTGACCTGGCGGCCGTGGTGGCGGCAGTGGTGG | 99 |
| 43 | TTGACTGAGACTCCTCATGACCTCTGCAGAAGTCATATTTAGGATGTGTAC | 100 |
| 44 | GGGCTACTCGCAATTTCAAATTGCTGGACTTTTTTTCCAAGGCTATTCAGT | 101 |
| 45 | TGTATCAGCATCTGGCTCAGCGTCTGAGATGTGTAAGCGCAGCCTTGAGTC | 102 |
| 46 | TACATAGAATCTACCGTGGTGACCTATTTATGCTATACATGATGAAACATC | 103 |
| 47 | TTGACTGAGACTCCTCATGACCTCTGAAATTGATAGAAGCAGAAGATCGGC | 104 |
| 48 | GGGCTACTCGCAATTTCAAATTGCTCTCACCTCCCATGTTGCTCAAAGAAC | 105 |
| 49 | TGTATCAGCATCTGGCTCAGCGTCTAGCATTCTCTGCAGTACATCAACCGT | 106 |
| 50 | TACATAGAATCTACCGTGGTGACCTACTTTACTCACGTTTTTCCCATCTAG | 107 |
| 51 | TTGACTGAGACTCCTCATGACCTCTGGCATGGTGGTGGATGTAGTGGTGGT | 108 |
| 52 | GGGCTACTCGCAATTTCAAATTGCTATGGTGGTGGATGTAGTGGTGGTGGA | 109 |
| 53 | TGTATCAGCATCTGGCTCAGCGTCTAACATGAGTTTTTTATGGCGGGAGGT | 110 |
| 54 | TACATAGAATCTACCGTGGTGACCTGGACACCGGCAAGGCCACCCTGACCT | 111 |
| 55 | TTGACTGAGACTCCTCATGACCTCTCTGACCCACTCATCCCAAGACACACC | 112 |
| 56 | GGGCTACTCGCAATTTCAAATTGCTGAAAGTAACAGCTTGACTATATCCAC | 113 |
| 57 | TGTATCAGCATCTGGCTCAGCGTCTGTGTCCTGGAATGGGGCCCATGAGAT | 114 |
| 58 | TACATAGAATCTACCGTGGTGACCTTGGAATTTCCTCCTCGAGTCTGAACC | 115 |
| 59 | TTGACTGAGACTCCTCATGACCTCTTTCCCTCCAGCCCCAGGTTACCCCTG | 116 |
| 60 | GGGCTACTCGCAATTTCAAATTGCTCGGGGGGTCTTGGATGTGCCGGCTTG | 117 |
| 61 | TGTATCAGCATCTGGCTCAGCGTCTTGGCCTGTTGGCCGTATCTGCTAACA | 118 |
| 62 | TACATAGAATCTACCGTGGTGACCTCTGTTTTGTTCCGAATGTCTGAGGAC | 119 |
| 63 | TTGACTGAGACTCCTCATGACCTCTGTGTCAACAATTCTAAGGAGGAAGAT | 120 |
| 64 | GGGCTACTCGCAATTTCAAATTGCTGAGATCCAGATGTTTTGGAATATTAC | 121 |
| 65 | TGTATCAGCATCTGGCTCAGCGTCTTGGACTGTGTATGAAACCTGGTTTTA | 122 |
| 66 | TACATAGAATCTACCGTGGTGACCTCAATCTTTTTAACCATTTTGTCATCG | 123 |
| 67 | TTGACTGAGACTCCTCATGACCTCTCCAGGGGAGAAAAGTACATTGGAAAC | 124 |
| 68 | GGGCTACTCGCAATTTCAAATTGCTCTTTATTCAGGTGGATGCCCCTGACC | 125 |
| 69 | TGTATCAGCATCTGGCTCAGCGTCTCAACATCTCTTTTCCCTGGAAGTTTC | 126 |
| 70 | TACATAGAATCTACCGTGGTGACCTGGACATGGATCTTGTTTTTCTCTTTG | 127 |
| 71 | TTGACTGAGACTCCTCATGACCTCTCTCAATCTGTAGTGCTCCTGGTCGGC | 128 |
| 72 | GGGCTACTCGCAATTTCAAATTGCTGTTTTTTCAGGAGGCCATCTTTCTCC | 129 |
| 73 | TGTATCAGCATCTGGCTCAGCGTCTAGAATGAGCCTGTTCTGTTGACATTG | 130 |
| 74 | TACATAGAATCTACCGTGGTGACCTCAGGGGAGGGTGTGGGCAGGCGGTTC | 131 |
| 75 | TTGACTGAGACTCCTCATGACCTCTGACTATTCAGACATCAATGAGGTGGC | 132 |
| 76 | GGGCTACTCGCAATTTCAAATTGCTCTGTTCCTCTACAGGGCCAAAACACT | 133 |
| 77 | TGTATCAGCATCTGGCTCAGCGTCTGATGATACTCACTGTCCATCAGCCTC | 134 |
| 78 | TACATAGAATCTACCGTGGTGACCTAGTATCCTCACCTGTAGCCAGGTATC | 135 |
| 79 | TTGACTGACTCCTCATGACCTCTGTTAATTCAGCATCCAGCAGGTCCCT | 136 |
| 80 | GGGCTACTCGCAATTTCAAATTGCTACACCAACATTCCCAGCTGCTGGAAC | 137 |
| 81 | TGTATCAGCATCTGGCTCAGCGTCTCCTGGGCCAGGTGTGCATCAAAGCGC | 138 |
| 82 | TACATAGAATCTACCGTGGTGACCTGTGTCAAGCTACTCTCAGGACTGCTC | 139 |
| 83 | TTGACTGAGACTCCTCATGACCTCTCAAGGTGCCAGGTGCAAGACCCACCA | 140 |
| 84 | GGGCTACTCGCAATTTCAAATTGCTTGTCGCGATGAATGTGAAATCCTGGA | 141 |
| 85 | TGTATCAGCATCTGGCTCAGCGTCTTCCACAAACTCGTCACTCATCCTCCG | 142 |
| 86 | TACATAGAATCTACCGTGGTGACCTAGACATGGAAGCCAGTGATTATGAGC | 143 |
| 87 | TTGACTGAGACTCCTCATGACCTCTCCACAGCCAGGCAGTCTGTATCTTGC | 144 |
| 88 | GGGCTACTCGCAATTTCAAATTGCTATATGTGGAGGCCCAACAAAAGAGAC | 145 |
| 89 | TGTATCAGCATCTGGCTCAGCGTCTTGGAAGTTGCGTATTGTAAGCTATTC | 146 |
| 90 | TACATAGAATCTACCGTGGTGACCTGGTCAGAACAGGAGTGCACGGATAGC | 147 |
| 91 | TTGACTGAGACTCCTCATGACCTCTTGCTTTCAATCCCAAATTATGTGTTT | 148 |
| 92 | GGGCTACTCGCAATTTCAAATTGCTCATCAGTGTGTCTGAACATGTGGTCC | 149 |
| 93 | TGTATCAGCATCTGGCTCAGCGTCTCTGCCAGCCTGCCCTGGAGGAAGACA | 150 |
| 94 | TACATAGAATCTACCGTGGTGACCTACTGGAACTATCTGTAATACTGGAAC | 151 |
| 95 | TTGACTGAGACTCCTCATGACCTCTAACTCTTTCACTTTTACATATTAAAG | 152 |
| 96 | GGGCTACTCGCAATTTCAAATTGCTGCAGCCAGAGTGGTTTTTTCAGGGGA | 153 |

TABLE 8
VARIANT 5′ LIGATION OLIGOS

| | | |
|---|---|---|
| | | SEQ |
| SNV | | ID |
| Assay # | SEQUENCE (5′ to 3′) | NO: |

| | | |
|---|---|---|
| 1 | CTTTGGGGTAAGCGACCATCAGCCTGCTTTCATTCATATCTGCAGGTTCAG | 154 |
| 2 | ACGATGGCGTTGCAGGCGCTTACCTGCAGTCCAGAGCACCGTGGTCCTGCC | 155 |
| 3 | GCCGTTTCATATCGAACAAGGCGCTCACCACTGGGGTAAGGTTTTCTAGGA | 156 |
| 4 | CGCCAGCAATCAGCTTTGATACACTAGCCAGTTTTCCATGGGTTCTACTAT | 157 |
| 5 | CTTTGGGGTAAGCGACCATCAGCCTCTTCCCGGTCAGCTACTCCTCTTCCA | 158 |
| 6 | ACGATGGCGTTGCAGGCGCTTACCTTGATCCATTAGATTCAAATGTAGCAC | 159 |
| 7 | GCCGTTTCATATCGAACAAGGCGCTACCTGCTGGTGCCACTCTGGAAAGGG | 160 |
| 8 | CGCCAGCAATCAGCTTTGATACACTCTTGCTGCTTCCAGTAAATAAGGTGG | 161 |
| 9 | CTTTGGGGTAAGCGACCATCAGCCTATCCTTGTCCAAGGAGGCTGTTTCTA | 162 |
| 10 | ACGATGGCGTTGCAGGCGCTTACCTTCCACACGCAAATTTCCTTCCACTCA | 163 |
| 11 | GCCGTTTCATATCGAACAAGGCGCTAGGAGCTGCTGGTGCAGGGGCCACGC | 164 |
| 12 | CGCCAGCAATCAGCTTTGATACACTATTCATCGGACATGTTACTGTTTTTG | 165 |
| 13 | CTTTGGGGTAAGCGACCATCAGCCTGTGTCATCAACTTGGTCCACAGTCGG | 166 |
| 14 | ACGATGGCGTTGCAGGCGCTTACCTGGCCCTCTAGGGACTCGAACAGAGAC | 167 |
| 15 | GCCGTTTCATATCGAACAAGGCGCTCAGAGGGAGGACGAGCTGACCTTCAC | 168 |
| 16 | CGCCAGCAATCAGCTTTGATACACTCCAACTCGAAATTCCCCGTGACCAGT | 169 |
| 17 | CTTTGGGGTAAGCGACCATCAGCCTACCAGCAGATACTCAGCCGGAGGATG | 170 |
| 18 | ACGATGGCGTTGCAGGCGCTTACCTGAGGACCCCAAGTCCCATAGGGACCT | 171 |
| 19 | GCCGTTTCATATCGAACAAGGCGCTGCAGCGCACCACGGGACCCAAGCCCC | 172 |
| 20 | CGCCAGCAATCAGCTTTGATACACTCAGAGTCTGAGGTAGCTGCCCTGGCG | 173 |
| 21 | CTTTGGGGTAAGCGACCATCAGCCTTGCTGTTTTCTTCCTTCAGGCATACC | 174 |
| 22 | ACGATGGCGTTGCAGGCGCTTACCTCCTGAACAGCTCGCGGCTCAGCAGGA | 175 |
| 23 | GCCGTTTCATATCGAACAAGGCGCTATCTTCAAAGTTGCAGTAAAAACCCG | 176 |
| 24 | CGCCAGCAATCAGCTTTGATACACTCTTGATTCATGATATTTTACTCCAAA | 177 |
| 25 | CTTTGGGGTAAGCGACCATCAGCCTTCCCTCATTGCACTGTACTCCTCTTT | 178 |
| 26 | ACGATGGCGTTGCAGGCGCTTACCTGTCCGAGGACAACGATGAGGCGGCGT | 179 |
| 27 | GCCGTTTCATATCGAACAAGGCGCTTTCCGCCTGGTGTTGGAAGAGACAGA | 180 |
| 28 | CGCCAGCAATCAGCTTTGATACACTTGGTCTTTCAGTGCCTCCACTATGAT | 181 |
| 29 | CTTTGGGGTAAGCGACCATCAGCCTTGAAGAGAAATATAAGAAGGCTATGT | 182 |
| 30 | ACGATGGCGTTGCAGGCGCTTACCTCCTCCAGGTGCAGGAGTTCATGCTCG | 183 |
| 31 | GCCGTTTCATATCGAACAAGGCGCTAAAGGCAATGTGGGATCCTGAATTGA | 184 |
| 32 | CGCCAGCAATCAGCTTTGATACACTGCCCGAACAGCCGCTGGATATGGGAA | 185 |
| 33 | CTTTGGGGTAAGCGACCATCAGCCTCTAAAAAGGACCCTGAAGGTTGTGAA | 186 |
| 34 | ACGATGGCGTTGCAGGCGCTTACCTTCATATGGATGATAATGATGGAGAAA | 187 |
| 35 | GCCGTTTCATATCGAACAAGGCGCTCTTGGCTGTGCTCCTGCTGCTGGCCA | 188 |
| 36 | CGCCAGCAATCAGCTTTGATACACTATCAACTATAGGTTGCTTTGGTGGTA | 189 |
| 37 | CTTTGGGGTAAGCGACCATCAGCCTAAATTTCTGAATAACTGAAGTTGGTT | 190 |
| 38 | ACGATGGCGTTGCAGGCGCTTACCTGGTAGCAGACAAACCTGTGGTTGATA | 191 |
| 39 | GCCGTTTCATATCGAACAAGGCGCTGAGCTTTGGGTTGTTCCTTAGGACCT | 192 |
| 40 | CGCCAGCAATCAGCTTTGATACACTGGAATTACGGCAGCCCTTCTTTCCCC | 193 |
| 41 | CTTTGGGGTAAGCGACCATCAGCCTCGGGGCCTCTGCTTGGATGTGATGAT | 194 |
| 42 | ACGATGGCGTTGCAGGCGCTTACCTGGCGGCCGTGGTGGCGGCAGTGGTGT | 195 |
| 43 | GCCGTTTCATATCGAACAAGGCGCTGCAGAAGTCATATTTAGGATGTGTAA | 196 |
| 44 | CGCCAGCAATCAGCTTTGATACACTGGACTTTTTTTCCAAGGCTATTCAGG | 197 |
| 45 | CTTTGGGGTAAGCGACCATCAGCCTGAGATGTGTAAGCGCAGCCTTGAGTT | 198 |
| 46 | ACGATGGCGTTGCAGGCGCTTACCTATTTATGCTATACATGATGAAACATA | 199 |
| 47 | GCCGTTTCATATCGAACAAGGCGCTGAAATTGATAGAAGCAGAAGATCGGA | 200 |
| 48 | CGCCAGCAATCAGCTTTGATACACTCTCACCTCCCATGTTGCTCAAAGAAA | 201 |
| 49 | CTTTGGGGTAAGCGACCATCAGCCTAGCATTCTCTGCAGTACATCAACCGC | 202 |
| 50 | ACGATGGCGTTGCAGGCGCTTACCTACTTTACTCACGTTTTTCCCATCTAT | 203 |
| 51 | GCCGTTTCATATCGAACAAGGCGCTGGCATGGTGGTGGATGTAGTGGTGGG | 204 |
| 52 | CGCCAGCAATCAGCTTTGATACACTATGGTGGTGGATGTAGTGGTGGTGGG | 205 |
| 53 | CTTTGGGGTAAGCGACCATCAGCCTAACATGAGTTTTTTATGGCGGGAGGG | 206 |
| 54 | ACGATGGCGTTGCAGGCGCTTACCTGGACACCGGCAAGGCCACCCTGACCG | 207 |
| 55 | GCCGTTTCATATCGAACAAGGCGCTCTGACCCACTCATCCCAAGACACACT | 208 |
| 56 | CGCCAGCAATCAGCTTTGATACACTGAAAGTAACAGCTTGACTATATCCAT | 209 |
| 57 | CTTTGGGGTAAGCGACCATCAGCCTGTGTCCTGGAATGGGGCCCATGAGAC | 210 |
| 58 | ACGATGGCGTTGCAGGCGCTTACCTTGGAATTTCCTCCTCGAGTCTGAACA | 211 |
| 59 | GCCGTTTCATATCGAACAAGGCGCTTTCCCTCCAGCCCCAGGTTACCCCTC | 212 |
| 60 | CGCCAGCAATCAGCTTTGATACACTCGGGGGGTCTTGGATGTGCCGGCTTT | 213 |
| 61 | CTTTGGGGTAAGCGACCATCAGCCTTGGCCTGTTGGCCGTATCTGCTAACT | 214 |
| 62 | ACGATGGCGTTGCAGGCGCTTACCTCTGTTTTGTTCCGAATGTCTGAGGAA | 215 |
| 63 | GCCGTTTCATATCGAACAAGGCGCTGTGTCAACAATTCTAAGGAGGAAGAA | 216 |
| 64 | CGCCAGCAATCAGCTTTGATACACTGAGATCCAGATGTTTTGGAATATTAA | 217 |
| 65 | CTTTGGGGTAAGCGACCATCAGCCTTGGACTGTGTATGAAACCTGGTTTTG | 218 |
| 66 | ACGATGGCGTTGCAGGCGCTTACCTCAATCTTTTTAACCATTTTGTCATCT | 219 |
| 67 | GCCGTTTCATATCGAACAAGGCGCTCCAGGGGAGAAAAGTACATTGGAAAA | 220 |
| 68 | CGCCAGCAATCAGCTTTGATACACTCTTTATTCAGGTGGATGCCCCTGACA | 221 |
| 69 | CTTTGGGGTAAGCGACCATCAGCCTCAACATCTCTTTTCCCTGGAAGTTTA | 222 |
| 70 | ACGATGGCGTTGCAGGCGCTTACCTGGACATGGATCTTGTTTTTCTCTTTT | 223 |
| 71 | GCCGTTTCATATCGAACAAGGCGCTCTCAATCTGTAGTGCTCCTGGTCGGA | 224 |
| 72 | CGCCAGCAATCAGCTTTGATACACTGTTTTTTCAGGAGGCCATCTTTCTCA | 225 |
| 73 | CTTTGGGGTAAGCGACCATCAGCCTAGAATGAGCCTGTTCTGTTGACATTT | 226 |
| 74 | ACGATGGCGTTGCAGGCGCTTACCTCAGGGGAGGGTGTGGGCAGGCGGTTA | 227 |
| 75 | GCCGTTTCATATCGAACAAGGCGCTGACTATTCAGACATCAATGAGGTGGA | 228 |
| 76 | CGCCAGCAATCAGCTTTGATACACTCTGTTCCTCTACAGGGCCAAAACACC | 229 |
| 77 | CTTTGGGGTAAGCGACCATCAGCCTGATGATACTCACTGTCCATCAGCCTT | 230 |
| 78 | ACGATGGCGTTGCAGGCGCTTACCTAGTATCCTCACCTGTAGCCAGGTATT | 231 |
| 79 | GCCGTTTCATATCGAACAAGGCGCTGTTAATTCAGCATCCAGCAGGTCCCA | 232 |
| 80 | CGCCAGCAATCAGCTTTGATACACTACACCAACATTCCCAGCTGCTGGAAA | 233 |
| 81 | CTTTGGGGTAAGCGACCATCAGCCTCCTGGGCCAGGTGTGCATCAAAGCGA | 234 |
| 82 | ACGATGGCGTTGCAGGCGCTTACCTGTGTCAAGCTACTCTCAGGACTGCTA | 235 |
| 83 | GCCGTTTCATATCGAACAAGGCGCTCAAGGTGCCAGGTGCAAGACCCACCT | 236 |
| 84 | CGCCAGCAATCAGCTTTGATACACTTGTCGCGATGAATGTGAAATCCTGGG | 237 |
| 85 | CTTTGGGGTAAGCGACCATCAGCCTTCCACAAACTCGTCACTCATCCTCCA | 238 |
| 86 | ACGATGGCGTTGCAGGCGCTTACCTAGACATGGAAGCCAGTGATTATGAGA | 239 |
| 87 | GCCGTTTCATATCGAACAAGGCGCTCCACAGCCAGGCAGTCTGTATCTTGA | 240 |
| 88 | CGCCAGCAATCAGCTTTGATACACTATATGTGGAGGCCCAACAAAAGAGAA | 241 |
| 89 | CTTTGGGGTAAGCGACCATCAGCCTTGGAAGTTGCGTATTGTAAGCTATTA | 242 |
| 90 | ACGATGGCGTTGCAGGCGCTTACCTGGTCAGAACAGGAGTGCACGGATAGA | 243 |
| 91 | GCCGTTTCATATCGAACAAGGCGCTTGCTTTCAATCCCAAATTATGTGTTC | 244 |
| 92 | CGCCAGCAATCAGCTTTGATACACTCATCAGTGTGTCTGAACATGTGGTCT | 245 |
| 93 | CTTTGGGGTAAGCGACCATCAGCCTCTGCCAGCCTGCCCTGGAGGAAGACT | 246 |
| 94 | ACGATGGCGTTGCAGGCGCTTACCTACTGGAACTATCTGTAATACTGGAAA | 247 |
| 95 | GCCGTTTCATATCGAACAAGGCGCTAACTCTTTCACTTTTACATATTAAAT | 248 |
| 96 | CGCCAGCAATCAGCTTTGATACACTGCAGCCAGAGTGGTTTTTTCAGGGGG | 249 |

TABLE 9
3′ PHOSPHORYLATED LIGATION OLIGOS

| | | |
|---|---|---|
| | | SEQ |
| SNV | | ID |
| Assay # | SEQUENCE (5′ to 3′) | NO: |

| | | |
|---|---|---|
| 1 | TTTTCACATGGTTTTCCAGGCTTGCTCCGGTAAGTTCCAGAGCGCAGAGT | 250 |
| 2 | GCGCCAGCTCCAGCAAAGCCAGCACTCCGGTAAGTTCCAGAGCGCAGAGT | 251 |
| 3 | TTGGCTTCGACAACTTTGCTGCTTGTCCGGTAAGTTCCAGAGCGCAGAGT | 252 |
| 4 | TAAACTAGAAAACATACAAAATAGGTCCGGTAAGTTCCAGAGCGCAGAGT | 253 |
| 5 | GTGCCCGCCGGCCCTCGCTGGACTCTCATAGGACGACTTCGTCCAAGATC | 254 |
| 6 | ATCAGAAGCCCTTTGAGAGTGGAAGTCATAGGACGACTTCGTCCAAGATC | 255 |
| 7 | CCAAGACTCTCTCCCCAGGGAAGAATCATAGGACGACTTCGTCCAAGATC | 256 |
| 8 | GGTACTGTACTTTAAAGAGGTCACTTCATAGGACGACTTCGTCCAAGATC | 257 |
| 9 | TCTGCAAAGGAGTAAGTCGATTTGGTCTGTATAGAGGTCTTCATGCAAAC | 258 |
| 10 | GATAAGATGCTGAGGAGGGGCCAGATCTGTATAGAGGTCTTCATGCAAAC | 259 |
| 11 | GGGGAGCAGCCTCTGGCATTCTGGGTCTGTATAGAGGTCTTCATGCAAAC | 260 |
| 12 | CTCCCTGATGTACCACCAACTTTACTCTGTATAGAGGTCTTCATGCAAAC | 261 |
| 13 | GTCAGGAGGGGCATCAGGCGCTAAGTCAGTACAGGCCATGGACCTTTCAG | 262 |
| 14 | CTCTGCAGCTGTGGGTTTCTTTGCATCAGTACAGGCCATGGACCTTTCAG | 263 |
| 15 | CAAGAGCGCCATCATCCAGAATGTGTCAGTACAGGCCATGGACCTTTCAG | 264 |
| 16 | CTTTTGGACACCAGGTTGGTGAATCTCAGTACAGGCCATGGACCTTTCAG | 265 |
| 17 | TTTCAGAGGTGAGAGTAGGGCAATTTCGGATCCTGGCTGCATCGATACAA | 266 |
| 18 | CTCGAATAGGCACAGTTACCCCCAGTCGGATCCTGGCTGCATCGATACAA | 267 |
| 19 | CCGAGCTCGCGCCAGCCCGCGCCACTCGGATCCTGGCTGCATCGATACAA | 268 |
| 20 | TATTTAACAACATCAGCCGAGACGTTCGGATCCTGGCTGCATCGATACAA | 269 |
| 21 | GAGGATGACCCCAAAGATAGTGGATTCCGGCATAGATCGCTAATTCTGTG | 270 |
| 22 | CGTCCCAGAGCTGGTCCACCTGCAGTCCGGCATAGATCGCTAATTCTGTG | 271 |
| 23 | CAGGCAGTTTCCCTATGGAGAGAGCTCCGGCATAGATCGCTAATTCTGTG | 272 |
| 24 | ATACAAATGAATCATGGAGAAATCTTCCGGCATAGATCGCTAATTCTGTG | 273 |
| 25 | ACCTGCTGTGTCGAGAATATCCAAGTCCTCCTACACTACGATGAGGCGTG | 274 |
| 26 | CCGGGCCCTGGGCGGTGGCAACGGCTCCTCCTACACTACGATGAGGCGTG | 275 |
| 27 | CATGGGTTTGGTGACCTGGCCCTTGTCCTCCTACACTACGATGAGGCGTG | 276 |
| 28 | GTTGTAGGTGGCACCTCTGGTGAGGTCCTCCTACACTACGATGAGGCGTG | 277 |
| 29 | TTTCCAATGCTCAGCTAGACAATGATCGCAACGGCAGCGTTCAATGTTAT | 278 |
| 30 | GCTTCCTCCGAGACCCCTTACGAGATCGCAACGGCAGCGTTCAATGTTAT | 279 |
| 31 | AAAAACCTTCACAACGACCAGGCCTTCGCAACGGCAGCGTTCAATGTTAT | 280 |
| 32 | GAACAGCCGCAAGTTTGAGTTTGAATCGCAACGGCAGCGTTCAATGTTAT | 281 |
| 33 | AAAAGTGATGACAAAAACACTGTAATCCAGCGTTATATTGCCGTAGTCTG | 282 |
| 34 | TAGATACACCAATAAATTATAGTCTTCCAGCGTTATATTGCCGTAGTCTG | 283 |
| 35 | GGCTGTATCGAGGGCAGGCGCTCCATCCAGCGTTATATTGCCGTAGTCTG | 284 |
| 36 | TTGCCAACACAGCCTCTGCTTCTTCTCCAGCGTTATATTGCCGTAGTCTG | 285 |
| 37 | CTGAATTCTATGAAAAGTAGGTCTTTCGGCGCTCACCGTAATAGTTACGT | 286 |
| 38 | CTAAATTAGTGAAAAGAAAAATGTATCGGCGCTCACCGTAATAGTTACGT | 287 |
| 39 | GGTAGGGGGTGTGCTTATAAGGTAATCGGCGCTCACCGTAATAGTTACGT | 288 |
| 40 | CCCACATGGGGCCCATCAAACTCCGTCGGCGCTCACCGTAATAGTTACGT | 289 |
| 41 | TTGCAAAGACGGTGCTATGGACTGATCGTAGTTAAACAGCGGCTATCCAT | 290 |
| 42 | CGTTGGTGATGTTGGCCCCGCTGGCTCGTAGTTAAACAGCGGCTATCCAT | 291 |
| 43 | TATCTGTATAAATAAGAAAAAAAGGTCGTAGTTAAACAGCGGCTATCCAT | 292 |
| 44 | GTGCGAGGTAATCTAATCTCTTTTTTCGTAGTTAAACAGCGGCTATCCAT | 293 |
| 45 | TGTGTATTCGCTCTATCCCACACTTTCTACCTTGCTCTGTGGAAGCCGAA | 294 |
| 46 | TTATAAAGGAAAAAAAATACCGAAATCTACCTTGCTCTGTGGAAGCCGAA | 295 |
| 47 | TATAAAAAAGATAATGGAAAGGGATTCTACCTTGCTCTGTGGAAGCCGAA | 296 |
| 48 | CATATAGTAAGTATTTAATTTATGCTCTACCTTGCTCTGTGGAAGCCGAA | 297 |
| 49 | GACCTGTCAAAATAGAATGTGAGTTTCCGGTAAGTTCCAGAGCGCAGAGT | 298 |
| 50 | CAATTCCATGCACTTCTCATTTCTGTCCGGTAAGTTCCAGAGCGCAGAGT | 299 |
| 51 | GGACATGCTTCGTCGTCTGCTTGGTTCCGGTAAGTTCCAGAGCGCAGAGT | 300 |
| 52 | CATGCTTCGTCGTCTGCTTGGTCACTCCGGTAAGTTCCAGAGCGCAGAGT | 301 |
| 53 | AGACTGACCCTTTTTGGACTTCAGGTCATAGGACGACTTCGTCCAAGATC | 302 |
| 54 | CGAGCCCACTGGGTGCATCCTGAGATCATAGGACGACTTCGTCCAAGATC | 303 |
| 55 | TGCTACGGAGAAGTTGTTTAAGGGGTCATAGGACGACTTCGTCCAAGATC | 304 |
| 56 | ATGCCCATTCTTGGCTGCATCGTGATCATAGGACGACTTCGTCCAAGATC | 305 |
| 57 | GGTTGTCTGAGAGAGAGCTTCTTGTTCTGTATAGAGGTCTTCATGCAAAC | 306 |
| 58 | AAAACTGCCAGGAACAATACACAACTCTGTATAGAGGTCTTCATGCAAAC | 307 |
| 59 | TCGTGTGGCTCCTTCTTTGCTATAGTCTGTATAGAGGTCTTCATGCAAAC | 308 |
| 60 | TCAGATTGGTGAGCTCCCATCTGTTTCTGTATAGAGGTCTTCATGCAAAC | 309 |
| 61 | GGCTGTGCCACTGCTGGGGAAGGCCTCAGTACAGGCCATGGACCTTTCAG | 310 |
| 62 | AAGCCACAAGATTACAAGAAACGGCTCAGTACAGGCCATGGACCTTTCAG | 311 |
| 63 | CCTCCAAGGATGTACTGCAGTACAGTCAGTACAGGCCATGGACCTTTCAG | 312 |
| 64 | AAAAATGATCATGCCAAGAAGCCTATCAGTACAGGCCATGGACCTTTCAG | 313 |
| 65 | TTACTTTTCTTCTCTTGATGTGCAATCGGATCCTGGCTGCATCGATACAA | 314 |
| 66 | TCACTTTCTAAGAACTTCTTTATGGTCGGATCCTGGCTGCATCGATACAA | 315 |
| 67 | TAAAAAGATAGAATCTGAAAGTAAATCGGATCCTGGCTGCATCGATACAA | 316 |
| 68 | AAAAAGGAACTGAGATAAAACCAGGTCGGATCCTGGCTGCATCGATACAA | 317 |
| 69 | AAACGGCCACTGCAGACTTCACCGATCCGGCATAGATCGCTAATTCTGTG | 318 |
| 70 | ACAACAGTAAAATCACCTATGAGACTCCGGCATAGATCGCTAATTCTGTG | 319 |
| 71 | AAAAGACAACATTCTCTATTTTAGGTCCGGCATAGATCGCTAATTCTGTG | 320 |
| 72 | AACCTGCCATATAAATCTAAGATCTTCCGGCATAGATCGCTAATTCTGTG | 321 |
| 73 | TGCGCACCACATCAATCACTTCCCATCCTCCTACACTACGATGAGGCGTG | 322 |
| 74 | AAGCCGTTGGCTGGAGACACCTATTTCCTCCTACACTACGATGAGGCGTG | 323 |
| 75 | AGAAGATGAAAGCCGAAGATACCAGTCCTCCTACACTACGATGAGGCGTG | 324 |
| 76 | CATTGACTCAGATCTCTCAATCCATTCCTCCTACACTACGATGAGGCGTG | 325 |
| 77 | CAGTTCAGCAAGGGGTCATAGACAATCGCAACGGCAGCGTTCAATGTTAT | 326 |
| 78 | AATCTGGATGGCTTTCACCCCCTCCTCGCAACGGCAGCGTTCAATGTTAT | 327 |
| 79 | GGCCTTGTCAATGCACTAGAAGAGATCGCAACGGCAGCGTTCAATGTTAT | 328 |
| 80 | AAAAACTGAAATGGACAAGAGGTCATCGCAACGGCAGCGTTCAATGTTAT | 329 |
| 81 | TCGTCCAGGGACGCCAAGACACAGTTCCAGCGTTATATTGCCGTAGTCTG | 330 |
| 82 | AACTAAAACAAAACGATGACAAATTTCCAGCGTTATATTGCCGTAGTCTG | 331 |
| 83 | TGAGCAGATAGCCTCCCACCACACGTCCAGCGTTATATTGCCGTAGTCTG | 332 |
| 84 | GAATGTCCTGTGTCAAACAGAGTACTCCAGCGTTATATTGCCGTAGTCTG | 333 |
| 85 | GAGCTCGCGGCCATAGCGCTGTGCTTCGGCGCTCACCGTAATAGTTACGT | 334 |
| 86 | TTGAAGATGAAACAAGACCTGCTAATCGGCGCTCACCGTAATAGTTACGT | 335 |
| 87 | AAAAACATCCACTCTGCCTCGAATCTCGGCGCTCACCGTAATAGTTACGT | 336 |
| 88 | TAGAAGCCTTATTCACTAAAATTCATCGGCGCTCACCGTAATAGTTACGT | 337 |
| 89 | AAAAAAAGAAAAAGATTCAGGTAAGTCGTAGTTAAACAGCGGCTATCCAT | 338 |
| 90 | AAAACAACTAGAAAATGATACAAGATCGTAGTTAAACAGCGGCTATCCAT | 339 |
| 91 | CCGAAATTTACCGCATGGAGGAAGTTCGTAGTTAAACAGCGGCTATCCAT | 340 |
| 92 | GCAGGTAGCGGGACTGTCGGGTGGGTCGTAGTTAAACAGCGGCTATCCAT | 341 |
| 93 | GTACAGCATCACACCCACGCTGAGATCTACCTTGCTCTGTGGAAGCCGAA | 342 |
| 94 | CTAAATAAAACAAAGCAGCCAAAAATCTACCTTGCTCTGTGGAAGCCGAA | 343 |
| 95 | CCTCATGAGGATCACTGGCCAGTAATCTACCTTGCTCTGTGGAAGCCGAA | 344 |
| 96 | GTCTTATATAAGTAATTTAAAAAAATCTACCTTGCTCTGTGGAAGCCGAA | 345 |

Step 1. Pooling of Synthesized Oligos.

All of the oligos from each of the 6 plates were pooled into separate, labeled pools of 100 μM oligos, resulting in a pool of 96 variant templates, a pool of 96 consensus templates, a pool of consensus plus variant 5′ ligation oligos for templates 1-48, a pool of consensus plus variant 5′ ligation oligos for templates 49-96, and a pool of 3′ common ligation oligos for templates 1-48, and a pool of 3′ common ligation oligos for templates 49-96.

100 μl of the Ligation oligos were diluted to a stock solution of 0.5 μM=5 nM in each ligation oligo.

The pooled templates were diluted to a working concentration of 100 pM.

Step 2. Kinase Treatment of the 3′ Common Ligation Oligo Pools.

The 3′ common ligation oligo pools (from plate 2 and plate 5) were kinased as follows:

A 100 μl reaction of 1 μM pooled common oligos=10 μl of 10 μM 3′ oligos=100 pmoles of termini (optimal molarity of ends in a 100 μl kinase reaction).

The kinase reaction was carried out as follows:

10 μl 10× T4 kinase buffer (New England Biolabs, Ipswich, Mass.)

10 μl 10 mM ATP

10 μl of 10 μM 3′ common ligation oligo pool

70 μl H₂O

100 μl total volume, mix, add 2 μl T4 kinase (New England Biolabs, Ipswich, Mass.), mix and incubate at 37° C. for 30 minutes, then incubate at 65° C. for 20 minutes.

The kinase reaction was then diluted by adding 300 μl of H₂O to a 400 μl mixture of 250 nM 3′ common ligation primer that was 5 nM in each primer.

Step 3. The Ligation-Dependent Genotyping Assays were Carried Out as Follows:

For each assay, the ligation mixture contains

1. 96 consensus templates with 500 pM ligation oligos (high) (assays 1-48)

2. 96 variant templates with 500 pM ligation oligos (high) (assays 1-48)

3. No template control with 500 pM ligation oligos (high) (assays 1-48)

4. 96 consensus templates with 100 pM ligation oligos (low) (assays 1-48)

5. 96 variant templates with 100 pM ligation oligos (low) (assays 1-48)

6. No template control with 100 pM ligation oligos (low) (assays 1-48)

7. 96 consensus templates with 500 pM ligation oligos (high) (assays 49-96)

8. 96 variant templates with 500 pM ligation oligos (high) (assays 49-96)

9. Calu6 gDNA library with 500 pM ligation oligos (high) (assays 1-48)

10. Calu6 gDNA library with 500 pM ligation oligos (high) (assays 49-96)

11. Calu6 enriched (E1) library* with 500 pM ligation oligos (high) (assays 1-48)

12. Calu6 enriched (E1) library* with 500 pM ligation oligos (high) (assays 49-96)

The Calu6 enriched (E1) library is a pool of PCR Products from a Calu6 gDNA library that was enriched with a single round of solution-based capture for the Maxwell 139 gene set followed by PCR amplification, as described above.

For each genotyping ligation reaction, the following reagents were combined:

50 μl H₂O

20 μl target DNA (100 pM synthetic templates, or DNA samples: Calu6 gDNA (85 ng/μl); or Calu6 E1 (75 ng/μl))

10 μl (high) or 2 μl (low) of 500 nM 5′ ligation oligo pool (consensus and variant)

10 μl (high) or 2 μl (low) of 250 nM kinased, 3′ common oligo pool

10 μl of 10× Taq DNA ligase buffer (New England Biolabs, Mass.)

1 μ15M NaCl

100 μl total volume, mix and add 2 μl Taq DNA ligase (New England Biolabs)

The ligation mixture was then incubated in a thermal cycler across the following temperatures:

95° C. for 5 minutes;

75° C. for 15 minutes;

70° C. for 15 minutes;

65° C. for 30 minutes;

60° C. for 45 minutes;

55° C. for 30 minutes;

50° C. for 15 minutes;

45° C. for 15 minutes;

4° C. rest.

The ligation reactions were diluted to 1 ml with 900 μl of TEzero.

To measure the performance of the ligation-dependent genotyping assay, the following 6 ligation reactions were carried out on synthetic templates (all using high concentration (500 pM) ligation oligos), followed by qPCR analysis of each ligation reaction on a universal 384 well qPCR plate.

Templates: consensus templates, variant templates, no template control, Calu6 genomic DNA, and Calu6 enriched (E1) library.

Ligation Oligo pools: pool of 5′ consensus and variant ligation oligos for assays 1-48 plus 3′ common ligation primers for assays 1-48; pool of 5′ consensus and variant ligation oligos for assays 49-96, plus 3′ common ligation oligos for assays 49-96.

Therefore, for each set of SNVs of interest (e.g., assays 1-48, represent 48 different potential SNVs), a total of 5 ligation reactions were carried out:

1. ligation oligo pool (5′ consensus, 5′ variant, and 3′ common) plus synthetic consensus templates; and

2. ligation oligo pool (5′ consensus, 5′ variant, and 3′ common) plus synthetic variant templates;

3. ligation oligo pool (5′ consensus, 5′ variant, and 3′ common) plus no template control.

4. ligation oligo pool (5′ consensus, 5′ variant, and 3′ common) plus Calu6 genomic DNA.

5. ligation oligo pool (5′ consensus, 5′ variant, and 3′ common) plus Calu6 enriched (E1) library.

Each ligation reaction was then plated onto a separate prepared universal qPCR plate and assayed, providing a set of qPCR results for ligation reaction #1 consensus template (qPCR plate 1), #2 variant template (qPCR plate 2), #3 no template (qPCR plate 3), #4 Calu6 gDNA (plate 9), and #5 Calu6 E1 library (plate 11).

Step 4: Quantitative PCR (qPCR):

Manufacture of Universal Assay Plate

The assay plates were prepared for quantitative PCR (qPCR) assays using the PCR primers as described in Example 2:

Briefly described, 35 mls of 2× SYBR master mix (ABI) was combined with 10 mls of H₂O. 450 μl of the mixture was aliquoted into each well of a 96 well assay plate. 55 μl of the “C” (reverse) primers (10 μM) were added to the wells along the columns of the assay plate, and 55 μl of the “R” (forward) primers (10 μM) were added to the wells along the rows of the assay plate, as shown above in TABLE 6. The reagents were mixed, then 8 μl per well was aliquoted in quadruplicate into a 384 qPCR plate, in order to carry out 4 identical reactions for each qPCR primer pair.

Quantitative PCR Assay:

120 μl aliquots of each diluted genotyping ligation reaction were distributed into 8 wells of the 384 well qPCR plate. Then, 2 μl aliquots were dispensed into all wells of the prepared 384 well qPCR plate (4×96). The samples were mixed, and the qPCR assay was run on an ABI 7900 instrument set on SYBR detection channel.

qPCR Results of Ligation-Dependent Genotyping Assay

As a measurement of assay performance, the average raw Ct data from each of the qPCR assays was first determined across four wells of each quadruplicate for assays 1-96 (high primer input). The results of the ligation with consensus templates (plates 1 and 4) or variant templates (plates 2 and 5) were measured against a no template control (plates 3 and 6), to obtain a set of raw Ct data (data not shown).

Dynamic Range of the Ligation-Dependent Genotyping Assays

In order to determine the dynamic range of each assay for a SNV position, from the raw Ct data, the Ct spread between consensus and variant ligation assays using consensus templates (e.g., plate 1) was determined. Then, the Ct spreads for variant versus consensus ligation assays when variant templates were measured (e.g., plate 2) was calculated. The sum of the Ct spreads for plate 1 and plate 2 were calculated, which represents the complete dynamic range of the assay.

For example, for assay #1, the Ct spread between consensus and variant ligation assays using a consensus template (Ct(var)−Ct(cons)=3. The Ct spread for variant versus consensus ligation assays when variant template was measured (Ct(cons)−Ct(var)=2. The sum of the Ct spreads (3+2=5) represents the complete dynamic range of assay #1.

It was determined that the sum of the Ct spreads for plate 1 (consensus template) and plate 2 (variant template) was ≧5 Cts for all but two of assays (assay #15 and #17), which is a very tractable Ct spread. Significantly, when the same analysis was performed on ligation reactions using a lower concentration of ligation oligos (100 pM), every single assay registered a dynamic range greater than 5 Cts (data not shown). The average dynamic range for the ligation-dependent genotyping assays carried out with high ligation oligo concentration (500 pM oligos) was 9.1 Cts, whereas the average dynamic range for the ligation-dependent genotyping assays carried out with low ligation oligo concentration (100 pM oligos) was 10.4 Cts. These results demonstrate that the use of ligation oligos in the range of 100 pM improves assay performance by proving a greater dynamic range.

Scoring Scheme for Genotyping

The ligation-dependent genotyping assay results generated using the synthetic template for the consensus and variant versions of the target sequence were then used to generate a calibrating “truth,” or “reference” value for the Ct values that are expected from a test sample (diploid) that contains a homozygous consensus (con/con), heterozygous (con/var), or homozygous variant (var/var) for a particular polymorphic site of interest (e.g., SNV or SNP), as follows.

If the actual test sample contains a diploid homozygous consensus sequence (con/con) at the polymorphic locus of interest, then on average Ct(var)>Ct(cons) and the term [Ct(var)−Ct(cons)] is expected to return a positive integer value.

If the actual test sample contains a diploid heterozygote sequence (con/var) at the polymorphic locus of interest, then on average, Ct(var)≈Ct(cons) and the term [Ct(var)−Ct(cons)] is expected to return a value near zero.

If the actual test sample contains a diploid homozygous variant sequence (var/var) at the polymorphic locus of interest, then on average, Ct(var)<Ct(cons) and the term [Ct(var)−Ct(cons)] is expected to return a negative integer value.

The calibrating consensus and variant synthetic templates are scored as follows:
Value homozygous consensus base=[Ct(var)−Ct(cons)] for consensus template measurements.
Value heterozygous=Ct(var) for variant template−Ct(cons) for consensus template.
Value homozygous variant base=[Ct(var)−Ct(cons)] for variant template.

The above scoring matrix was applied to the ligation-dependent genotyping assays using synthetic templates, and the results are shown below in TABLE 10, Column 2. The key observation is that all ligation-dependent genotyping assays 1-96, with the exception of assays 15 and 26, returned discrete integer values for each of the three genetic states. Importantly, it is noted that assays 15 and 26 did return discrete integer values when repeated with the more dilute ligation oligos (100 pmol) (data not shown).

TABLE 10
QPCR ASSAY RESULTS OF
LIGATION-DEPENDENT GENOTYPING

| | Scoring Matrix | Test Sample: |
|---|---|---|
| Assay # | (based on synthetic template assays): | E1 Calu6 DNA Ct Value |
| 1 | homo-cons: 3 | 0 |
| | hetero: 0 | (hetero) |
| | homo-var: −2 | |
| 2 | homo-cons: 9 | −3 |
| | hetero: 1 | (homo-var) |
| | homo-var: −3 | |
| 3 | homo-cons: 5 | −1 |
| | hetero: 1 | (homo-var) |
| | homo-var: −2 | |
| 4 | homo-cons: 6 | −4 |
| | hetero: 1 | (homo-var) |
| | homo-var: −3 | |
| 5 | homo-cons: 6 | 2 |
| | hetero: 1 | (hetero) |
| | homo-var: −2 | |
| 6 | homo-cons: 9 | −5 |
| | hetero: 1 | (homo-var) |
| | homo-var: −3 | |
| 7 | homo-cons: 9 | −2 |
| | hetero: 1 | (homo-var) |
| | homo-var: −5 | |
| 8 | homo-cons: 5 | −2 |
| | hetero: 1 | (homo-var) |
| | homo-var: −2 | |
| 9 | homo-cons: 7 | 1 |
| | hetero: 3 | (homo-var) |
| | homo-var: 0 | |
| 10 | homo-cons: 9 | −3 |
| | hetero: 2 | (homo-var) |
| | homo-var: −2 | |
| 11 | homo-cons: 11 | −3 |
| | hetero: 1 | (homo-var) |
| | homo-var: −3 | |
| 12 | homo-cons: 7 | 0 |
| | hetero: 0 | (hetero) |
| | homo-var: −4 | |
| 13 | homo-cons: 8 | −3 |
| | hetero: 0 | (homo-var) |
| | homo-var: −4 | |
| 14 | homo-cons: 4 | −1 |
| | hetero: 1 | (homo-var) |
| | homo-var: −1 | |
| 15 | homo-cons: 0 | −3 |
| | hetero: 0 | (homo-var) |
| | homo-var: −3 | |
| 16 | homo-cons: 4 | −5 |
| | hetero: 1 | (homo-var) |
| | homo-var: −4 | |
| 17 | homo-cons: 2 | −1 |
| | hetero: 0 | (homo-var) |
| | homo-var: −2 | |
| 18 | homo-cons: 3 | −3 |
| | hetero: 1 | (homo-var) |
| | homo-var: −3 | |
| 19 | homo-cons: 6 | 2 |
| | hetero: 1 | (hetero) |
| | homo-var: −3 | |
| 20 | homo-cons: 8 | 0 |
| | hetero: 1 | (hetero) |
| | homo-var: −3 | |
| 21 | homo-cons: 7 | −5 |
| | hetero: 1 | (homo-var) |
| | homo-var: −5 | |
| 22 | homo-cons: 5 | −2 |
| | hetero: 1 | (homo-var) |
| | homo-var: −2 | |
| 23 | homo-cons: 7 | 0 |
| | hetero: 0 | (hetero) |
| | homo-var: −4 | |
| 24 | homo-cons: 4 | −2 |
| | hetero: 1 | (homo-var) |
| | homo-var: −3 | |
| 25 | homo-cons: 8 | 0 |
| | hetero: 1 | (hetero) |
| | homo-var: −3 | |
| 26 | homo-cons: 9 | 1 |
| | hetero: 4 | (hetero) |
| | homo-var: 4 | |
| 27 | homo-cons: 8 | −3 (homo-var) |
| | hetero: 2 | |
| | homo-var: −2 | |
| 28 | homo-cons: 5 | −2 (homo-var) |
| | hetero: 1 | |
| | homo-var: −2 | |
| 29 | homo-cons: 6 | 2 |
| | hetero: 2 | (hetero) |
| | homo-var: −2 | |
| 30 | homo-cons: 4 | 1 |
| | hetero: 1 | (hetero) |
| | homo-var: −2 | |
| 31 | homo-cons: 8 | 11 |
| | hetero: 0 | (homo-cons) |
| | homo-var: −3 | |
| 32 | homo-cons: 7 | 8 |
| | hetero: 1 | (homo-cons) |
| | homo-var: −1 | |
| 33 | homo-cons: 6 | 8 |
| | hetero: 1 | (homo-cons) |
| | homo-var: −2 | |
| 34 | homo-cons: 9 | 9 |
| | hetero: 2 | (homo-cons) |
| | homo-var: −2 | |
| 35 | homo-cons: 1 | −3 |
| | hetero: −1 | (hetero-var) |
| | homo-var: −4 | |
| 36 | homo-cons: 7 | −2 |
| | hetero: 1 | (hetero-var) |
| | homo-var: −2 | |
| 37 | homo-cons: 4 | 0 |
| | hetero: 1 | (hetero) |
| | homo-var: −3 | |
| 38 | homo-cons: 5 | 6 |
| | hetero: 1 | (homo-cons) |
| | homo-var: −3 | |
| 39 | homo-cons: 4 | −4 |
| | hetero: 0 | (homo-var) |
| | homo-var: −4 | |
| 40 | homo-cons: 7 | 8 |
| | hetero: 1 | (homo-cons) |
| | homo-var: −2 | |
| 41 | homo-cons: 4 | 3 |
| | hetero: 1 | (homo-cons) |
| | homo-var: −1 | |
| 42 | homo-cons: 7 | 8 |
| | hetero: 1 | (homo-cons) |
| | homo-var: −3 | |
| 43 | homo-cons: 9 | 10 |
| | hetero: 1 | (homo-cons) |
| | homo-var: −4 | |
| 44 | homo-cons: 10 | 11 |
| | hetero: 0 | (homo-cons) |
| | homo-var: −5 | |
| 45 | homo-cons: 5 | 5 |
| | hetero: 0 | (homo-cons) |
| | homo-var: −6 | |
| 46 | homo-cons: 9 | 9 |
| | hetero: 1 | (homo-cons) |
| | homo-var: −4 | |
| 47 | homo-cons: 11 | 12 |
| | hetero: 1 | (homo-cons) |
| | homo-var: −2 | |
| 48 | homo-cons: 10 | 14 |
| | hetero: 2 | (homo-cons) |
| | homo-var: −2 | |
| 49 | homo-cons: 5 | 1 |
| | hetero: 0 | (hetero) |
| | homo-var: −4 | |
| 50 | homo-cons: 6 | 6 |
| | hetero: 2 | (homo-cons) |
| | homo-var: −5 | |
| 51 | homo-cons: 6 | 6 |
| | hetero: 0 | (homo-cons) |
| | homo-var: −4 | |
| 52 | homo-cons: 5 | 5 |
| | hetero: 0 | (homo-cons) |
| | homo-var: −4 | |
| 53 | homo-cons: 11 | 13 |
| | hetero: 2 | (homo-cons) |
| | homo-var: −1 | |
| 54 | homo-cons: 6 | −3 |
| | hetero: 0 | (homo-var) |
| | homo-var: −3 | |
| 55 | homo-cons: 3 | 3 |
| | hetero: 0 | (homo-cons) |
| | homo-var: −5 | |
| 56 | homo-cons: 6 | 0 |
| | hetero: 0 | (hetero) |
| | homo-var: −4 | |
| 57 | homo-cons: 6 | 1 |
| | hetero: 1 | (hetero) |
| | homo-var: −3 | |
| 58 | homo-cons: 7 | 8 |
| | hetero: 2 | (homo-cons) |
| | homo-var: −3 | |
| 59 | homo-cons: 5 | 1 |
| | hetero: 0 | (hetero) |
| | homo-var: −6 | |
| 60 | homo-cons: 6 | 7 |
| | hetero: 1 | (homo-cons) |
| | homo-var: −6 | |
| 61 | homo-cons: 5 | 5 |
| | hetero: 1 | (homo-cons) |
| | homo-var: −5 | |
| 62 | homo-cons: 4 | 6 |
| | hetero: 1 | (homo-cons) |
| | homo-var: −3 | |
| 63 | homo-cons: 5 | 5 |
| | hetero: 0 | (homo-cons) |
| | homo-var: −5 | |
| 64 | homo-cons: 6 | 7 |
| | hetero: 1 | (homo-cons) |
| | homo-var: −6 | |
| 65 | homo-cons: 3 | 3 |
| | hetero: 0 | (homo-cons) |
| | homo-var: −5 | |
| 66 | homo-cons: 5 | 5 |
| | hetero: 2 | (homo-cons) |
| | homo-var: −6 | |
| 67 | homo-cons: 6 | 6 |
| | hetero: 0 | (homo-cons) |
| | homo-var: −6 | |
| 68 | homo-cons: 6 | 7 |
| | hetero: 1 | (homo-cons) |
| | homo-var: −6 | |
| 69 | homo-cons: 6 | 7 |
| | hetero: 1 | (homo-cons) |
| | homo-var: −6 | |
| 70 | homo-cons: 6 | 7 |
| | hetero: 2 | (homo-cons) |
| | homo-var: −6 | |
| 71 | homo-cons: 6 | 8 |
| | hetero: 0 | (homo-cons) |
| | homo-var: −5 | |
| 72 | homo-cons: 6 | 8 |
| | hetero: 0 | (homo-cons) |
| | homo-var: −6 | |
| 73 | homo-cons: 5 | 6 |
| | hetero: 0 | (homo-cons) |
| | homo-var: −4 | |
| 74 | homo-cons: 7 | 7 |
| | hetero: 1 | (homo-cons) |
| | homo-var: −4 | |
| 75 | homo-cons: 5 | 8 |
| | hetero: 0 | (homo-cons) |
| | homo-var: −5 | |
| 76 | homo-cons: 6 | 7 |
| | hetero: 0 | (homo-cons) |
| | homo-var: −4 | |
| 77 | homo-cons: 4 | 4 |
| | hetero: 1 | (homo-cons) |
| | homo-var: −6 | |
| 78 | homo-cons: 4 | 4 |
| | hetero: 1 | (homo-cons) |
| | homo-var: −5 | |
| 79 | homo-cons: 6 | 8 |
| | hetero: 0 | (homo-cons) |
| | homo-var: −5 | |
| 80 | homo-cons: 6 | 8 |
| | hetero: 1 | (homo-cons) |
| | homo-var: −5 | |
| 81 | homo-cons: 4 | 5 |
| | hetero: 0 | (homo-cons) |
| | homo-var: −6 | |
| 82 | homo-cons: 6 | 6 |
| | hetero: 1 | (homo-cons) |
| | homo-var: −9 | |
| 83 | homo-cons: 5 | 5 |
| | hetero: 3 | (homo-cons) |
| | homo-var: −3 | |
| 84 | homo-cons: 3 | 2 |
| | hetero: 0 | (homo-cons) |
| | homo-var: −5 | |
| 85 | homo-cons: 5 | 3 |
| | hetero: 1 | (homo-cons) |
| | homo-var: −3 | |
| 86 | homo-cons: 5 | 6 |
| | hetero: 1 | (homo-cons) |
| | homo-var: −4 | |
| 87 | homo-cons: 3 | 4 |
| | hetero: 0 | (homo-cons) |
| | homo-var: −7 | |
| 88 | homo-cons: 5 | 6 |
| | hetero: 0 | (homo-cons) |
| | homo-var: −5 | |
| 89 | homo-cons: 7 | 7 |
| | hetero: 1 | (homo-cons) |
| | homo-var: −4 | |
| 90 | homo-cons: 8 | 7 |
| | hetero: 1 | (homo-cons) |
| | homo-var: −4 | |
| 91 | homo-cons: 6 | 7 |
| | hetero: 0 | (homo-cons) |
| | homo-var: −5 | |
| 92 | homo-cons: 6 | 6 |
| | hetero: 1 | (homo-cons) |
| | homo-var: −5 | |
| 93 | homo-cons: 3 | 2 |
| | hetero: 0 | (homo-cons) |
| | homo-var: −5 | |
| 94 | homo-cons: 9 | 11 |
| | hetero: 2 | (homo-cons) |
| | homo-var: −5 | |
| 95 | homo-cons: 7 | 7 |
| | hetero: 0 | (homo-cons) |
| | homo-var: −5 | |
| 96 | homo-cons: 6 | 6 |
| | hetero: 1 | (homo-cons) |
| | homo-var: −5 | |

Genotyping of Calu6 Test Samples

There were two samples tested that were derived from Calu6 gDNA. The first was genomic DNA (gDNA), and the second was a population of PCR products that were generated from a library made from Calu6 gDNA (E1 Calu6 DNA) which was enriched for the Maxwell 139 set of genes by solution-based capture, as described above.

In an initial experiment, genomic DNA gave little signal above background when tested in the ligation-dependent genotyping assay. For example, the average decrease in Ct (corresponding to an increase in signal) for Calu6 gDNA versus background for 96 assays (plate 9 versus plate 3) was 1.3 Cts (data not shown). However, it is noted that the 96 assays with Calu6 gDNA were carried out with a high concentration (500 pM) of ligation oligos. As described above, it was determined in experiments with the synthetic templates that reducing the primer concentration to 100 pM increased the dynamic range, thereby improving the sensitivity of the assay (i.e., increased signal-to-noise ratio). Such improved sensitivity with a lower concentration of ligation oligos may allow for genotyping of gDNA using the ligation-dependent assay.

For the enriched (E1) Calu6 DNA test samples, the average decrease in Ct (corresponding to an increase in signal) was 5 Cts, as shown in TABLE 10, Column 3, which was adequate sensitivity for genotyping. As shown in TABLE 10, assignments of homozygous consensus alleles (con/con), heterozygous alleles (var/con), or homozygous variant alleles (var/var) for the E1 Calu6 DNA samples at each of the 96 polymorphic loci of interest were made by comparing the experimental values obtained from the E1 Calu6 DNA to the “truth set” shown as the “scoring matrix” in Column 2 of TABLE 10, based on the genotyping assays carried out using the synthetic templates. Genotypes were then assigned to the test samples based on the closest pairing between the experimental value and the scoring matrix.

TABLE 11 provides a comparison of the results of the ligation-dependent genotyping assay shown in TABLE 10 with the genotype initially determined from massive parallel sequencing. Assays 1 to 96 correspond to 96 distinct putative SNVs that were initially detected as potential polymorphic loci during massively parallel sequencing. The list of 96 assays is sorted by highest (assay #1) to lowest (assay #96) confidence levels, with known database SNPs dominating the top portion of the list. In the situations where the polymorphic locus of interest corresponded to a SNP that is present in the dbSNP (http://www.ncbi.nlm nih gov/projects/SNP/) or COSMIC (http://www.sanger.ac.uk/genetics/CGP/cosmic/), the corresponding SNP reference number is provided in Column 2. A “0” value in Column 2 means that the potential polymorphic loci was not present in the dbSNP or COSMIC.

TABLE 11
LIGATION DEPENDENT GENOTYPING ASSAY RESULTS

| | Target sequence: | Initial Sequencing | Scoring Matrix | | Comparison of |
|---|---|---|---|---|---|
| | dbSNP or | call (massively | (based on | | ligation method to |
| | COSMIC | parallel sequence | synthetic | Test Sample: | sequence method |
| Assay # | reference number | platform) | template assays): | E1 Calu6 DNA | (confirm, differs) |
| 1 | rs6537825 | homo-var | homo-cons: 3 | 0 (hetero) | D |
| | | | hetero: 0 | | |
| | | | homo-var: −2 | | |
| 2 | rs1052576 | homo-var | homo-cons: 9 | −3 | C |
| | | | hetero: 1 | (homo-var) | |
| | | | homo-var: −3 | | |
| 3 | rs2308941 | homo-var | homo-cons: 5 | −1 | C |
| | | | hetero: 1 | (homo-var) | |
| | | | homo-var: −2 | | |
| 4 | rs2230804 | homo-var | homo-cons: 6 | −4 | C |
| | | | hetero: 1 | (homo-var) | |
| | | | homo-var: −3 | | |
| 5 | rs1799939 | hetero | homo-cons: 6 | 2 | C |
| | | | hetero: 1 | (hetero) | |
| | | | homo-var: −2 | | |
| 6 | rs144848 | homo-var | homo-cons: 9 | −5 | C |
| | | | hetero: 1 | (homo-var) | |
| | | | homo-var: −3 | | |
| 7 | rs1058808 | homo-var | homo-cons: 9 | −2 | C |
| | | | hetero: 1 | (homo-var) | |
| | | | homo-var: −5 | | |
| 8 | rs4986764 | homo-var | homo-cons: 5 | −2 | C |
| | | | hetero: 1 | (homo-var) | |
| | | | homo-var: −2 | | |
| 9 | rs6504459 | homo-var | homo-cons: 7 | 1 | C |
| | | | hetero: 3 | (homo-var) | |
| | | | homo-var: 0 | | |
| 10 | Cosmic | homo-var | homo-cons: 9 | −3 | C |
| | | | hetero: 2 | (homo-var) | |
| | | | homo-var: −2 | | |
| 11 | rs1042522 | homo-var | homo-cons: 11 | −3 | C |
| | | | hetero: 1 | (homo-var) | |
| | | | homo-var: −3 | | |
| 12 | rs2229571 | homo-var | homo-cons: 7 | 0 | D |
| | | | hetero: 0 | (hetero) | |
| | | | homo-var: −4 | | |
| 13 | rs2577301 | homo-var | homo-cons: 8 | −3 | C |
| | | | hetero: 0 | (homo-var) | |
| | | | homo-var: −4 | | |
| 14 | rs1670283 | homo-var | homo-cons: 4 | −1 | C |
| | | | hetero: 1 | (homo-var) | |
| | | | homo-var: −1 | | |
| 15 | rs753381 | homo-var | homo-cons: 0 | −3 | C |
| | | | hetero: 0 | (homo-var) | |
| | | | homo-var: −3 | | |
| 16 | rs235768 | homo-var | homo-cons: 4 | −5 | C |
| | | | hetero: 1 | (homo-var) | |
| | | | homo-var: −4 | | |
| 17 | rs20551 | homo-var | homo-cons: 2 | −1 | C |
| | | | hetero: 0 | (homo-var) | |
| | | | homo-var: −2 | | |
| 18 | rs376618 | homo-var | homo-cons: 3 | −3 | C |
| | | | hetero: 1 | (homo-var) | |
| | | | homo-var: −3 | | |
| 19 | rs2854746 | hetero | homo-cons: 6 | 2 | C |
| | | | hetero: 1 | (hetero) | |
| | | | homo-var: −3 | | |
| 20 | rs1073123 | homo-var | homo-cons: 8 | 0 | D |
| | | | hetero: 1 | (hetero) | |
| | | | homo-var: −3 | | |
| 21 | rs682632 | homo-var | homo-cons: 7 | −5 | C |
| | | | hetero: 1 | (homo-var) | |
| | | | homo-var: −5 | | |
| 22 | rs1052571 | homo-var | homo-cons: 5 | −2 | C |
| | | | hetero: 1 | (homo-var) | |
| | | | homo-var: −2 | | |
| 23 | rs35093491 | hetero | homo-cons: 7 | 0 | C |
| | | | hetero: 0 | (hetero) | |
| | | | homo-var: −4 | | |
| 24 | rs1801516 | homo-var | homo-cons: 4 | −2 | C |
| | | | hetero: 1 | (homo-var) | |
| | | | homo-var: −3 | | |
| 25 | Cosmic | hetero | homo-cons: 8 | 0 | C |
| | | | hetero: 1 | (hetero) | |
| | | | homo-var: −3 | | |
| 26 | rs1805097 | homo-var | homo-cons: 9 | 1 | D |
| | | | hetero: 4 | (hetero) | |
| | | | homo-var: 4 | | |
| 27 | rs2240308 | homo-var | homo-cons: 8 | −3 (homo-var) | C |
| | | | hetero: 2 | | |
| | | | homo-var: −2 | | |
| 28 | rs1250209 | homo-var | homo-cons: 5 | −2 (homo-var) | C |
| | | | hetero: 1 | | |
| | | | homo-var: −2 | | |
| 29 | 0 | hetero | homo-cons: 6 | 2 | C |
| | | | hetero: 2 | (hetero) | |
| | | | homo-var: −2 | | |
| 30 | rs2228246 | hetero | homo-cons: 4 | 1 (hetero) | C |
| | | | hetero: 1 | | |
| | | | homo-var: −2 | | |
| 31 | 0 | hetero | homo-cons: 8 | 11 | D |
| | | | hetero: 0 | (homo-cons) | |
| | | | homo-var: −3 | | |
| 32 | 0 | hetero | homo-cons: 7 | 8 | D |
| | | | hetero: 1 | (homo-cons) | |
| | | | homo-var: −1 | | |
| 33 | 0 | hetero | homo-cons: 6 | 8 | D |
| | | | hetero: 1 | (homo-cons) | |
| | | | homo-var: −2 | | |
| 34 | 0 | hetero | homo-cons: 9 | 9 | D |
| | | | hetero: 2 | (homo-cons) | |
| | | | homo-var: −2 | | |
| 35 | rs351855 | homo-var | homo-cons: 1 | −3 (hetero-var) | C |
| | | | hetero: −1 | | |
| | | | homo-var: −4 | | |
| 36 | 0 | homo-var | homo-cons: 7 | −2 (hetero-var) | C |
| | | | hetero: 1 | | |
| | | | homo-var: −2 | | |
| 37 | rs529038 | hetero | homo-cons: 4 | 0 | C |
| | | | hetero: 1 | (hetero) | |
| | | | homo-var: −3 | | |
| 38 | 0 | homo-var | homo-cons: 5 | 6 | D |
| | | | hetero: 1 | (homo-cons) | |
| | | | homo-var: −3 | | |
| 39 | rs502209 | homo-var | homo-cons: 4 | −4 (homo-var) | C |
| | | | hetero: 0 | | |
| | | | homo-var: −4 | | |
| 40 | 0 | homo-var | homo-cons: 7 | 8 | D |
| | | | hetero: 1 | (homo-cons) | |
| | | | homo-var: −2 | | |
| 41 | 0 | hetero | homo-cons: 4 | 3 | D |
| | | | hetero: 1 | (homo-cons) | |
| | | | homo-var: −1 | | |
| 42 | 0 | hetero | homo-cons: 7 | 8 | D |
| | | | hetero: 1 | (homo-cons) | |
| | | | homo-var: −3 | | |
| 43 | 0 | hetero | homo-cons: 9 | 10 | D |
| | | | hetero: 1 | (homo-cons) | |
| | | | homo-var: −4 | | |
| 44 | 0 | hetero | homo-cons: 10 | 11 | D |
| | | | hetero: 0 | (homo-cons) | |
| | | | homo-var: −5 | | |
| 45 | 0 | homo-var | homo-cons: 5 | 5 | D |
| | | | hetero: 0 | (homo-cons) | |
| | | | homo-var: −6 | | |
| 46 | rs1046984 | hetero | homo-cons: 9 | 9 | D |
| | | | hetero: 1 | (homo-cons) | |
| | | | homo-var: −4 | | |
| 47 | 0 | hetero | homo-cons: 11 | 12 | D |
| | | | hetero: 1 | (homo-cons) | |
| | | | homo-var: −2 | | |
| 48 | 0 | hetero | homo-cons: 10 | 14 | D |
| | | | hetero: 2 | (homo-cons) | |
| | | | homo-var: −2 | | |
| 49 | rs7190823 | hetero | homo-cons: 5 | 1 (hetero) | D |
| | | | hetero: 0 | | |
| | | | homo-var: −4 | | |
| 50 | 0 | hetero | homo-cons: 6 | 6 | D |
| | | | hetero: 2 | (homo-cons) | |
| | | | homo-var: −5 | | |
| 51 | 0 | hetero | homo-cons: 6 | 6 | D |
| | | | hetero: 0 | (homo-cons) | |
| | | | homo-var: −4 | | |
| 52 | 0 | hetero | homo-cons: 5 | 5 | D |
| | | | hetero: 0 | (homo-cons) | |
| | | | homo-var: −4 | | |
| 53 | 0 | hetero | homo-cons: 11 | 13 | D |
| | | | hetero: 2 | (homo-cons) | |
| | | | homo-var: −1 | | |
| 54 | rs243383 | homo-var | homo-cons: 6 | −3 (homo-var) | D |
| | | | hetero: 0 | | |
| | | | homo-var: −3 | | |
| 55 | 0 | hetero | homo-cons: 3 | 3 | D |
| | | | hetero: 0 | (homo-cons) | |
| | | | homo-var: −5 | | |
| 56 | rs2070094 | hetero | homo-cons: 6 | 0 | D |
| | | | hetero: 0 | (hetero) | |
| | | | homo-var: −4 | | |
| 57 | rs17449032 | hetero | homo-cons: 6 | 1 | D |
| | | | hetero: 1 | (hetero) | |
| | | | homo-var: −3 | | |
| 58 | 0 | hetero | homo-cons: 7 | 8 | D |
| | | | hetero: 2 | (homo-cons) | |
| | | | homo-var: −3 | | |
| 59 | rs1881421 | homo-var | homo-cons: 5 | 1 | D |
| | | | hetero: 0 | (hetero) | |
| | | | homo-var: −6 | | |
| 60 | 0 | hetero | homo-cons: 6 | 7 | D |
| | | | hetero: 1 | (homo-cons) | |
| | | | homo-var: −6 | | |
| 61 | 0 | hetero | homo-cons: 5 | 5 | D |
| | | | hetero: 1 | (homo-cons) | |
| | | | homo-var: −5 | | |
| 62 | 0 | homo-var | homo-cons: 4 | 6 | D |
| | | | hetero: 1 | (homo-cons) | |
| | | | homo-var: −3 | | |
| 63 | 0 | hetero | homo-cons: 5 | 5 | D |
| | | | hetero: 0 | (homo-cons) | |
| | | | homo-var: −5 | | |
| 64 | 0 | hetero | homo-cons: 6 | 7 | D |
| | | | hetero: 1 | (homo-cons) | |
| | | | homo-var: −6 | | |
| 65 | 0 | hetero | homo-cons: 3 | 3 | D |
| | | | hetero: 0 | (homo-cons) | |
| | | | homo-var: −5 | | |
| 66 | 0 | homo-var | homo-cons: 5 | 5 | D |
| | | | hetero: 2 | (homo-cons) | |
| | | | homo-var: −6 | | |
| 67 | 0 | homo-var | homo-cons: 6 | 6 | D |
| | | | hetero: 0 | (homo-cons) | |
| | | | homo-var: −6 | | |
| 68 | 0 | hetero | homo-cons: 6 | 7 | D |
| | | | hetero: 1 | (homo-cons) | |
| | | | homo-var: −6 | | |
| 69 | 0 | hetero | homo-cons: 6 | 7 | D |
| | | | hetero: 1 | (homo-cons) | |
| | | | homo-var: −6 | | |
| 70 | 0 | hetero | homo-cons: 6 | 7 | D |
| | | | hetero: 2 | (homo-cons) | |
| | | | homo-var: −6 | | |
| 71 | 0 | hetero | homo-cons: 6 | 8 | D |
| | | | hetero: 0 | (homo-cons) | |
| | | | homo-var: −5 | | |
| 72 | 0 | hetero | homo-cons: 6 | 8 | D |
| | | | hetero: 0 | (homo-cons) | |
| | | | homo-var: −6 | | |
| 73 | 0 | hetero | homo-cons: 5 | 6 | D |
| | | | hetero: 0 | (homo-cons) | |
| | | | homo-var: −4 | | |
| 74 | 0 | hetero | homo-cons: 7 | 7 | D |
| | | | hetero: 1 | (homo-cons) | |
| | | | homo-var: −4 | | |
| 75 | 0 | hetero | homo-cons: 5 | 8 | D |
| | | | hetero: 0 | (homo-cons) | |
| | | | homo-var: −5 | | |
| 76 | 0 | homo-var | homo-cons: 6 | 7 | D |
| | | | hetero: 0 | (homo-cons) | |
| | | | homo-var: −4 | | |
| 77 | 0 | hetero | homo-cons: 4 | 4 | D |
| | | | hetero: 1 | (homo-cons) | |
| | | | homo-var: −6 | | |
| 78 | 0 | homo-var | homo-cons: 4 | 4 | D |
| | | | hetero: 1 | (homo-cons) | |
| | | | homo-var: −5 | | |
| 79 | 0 | hetero | homo-cons: 6 | 8 | D |
| | | | hetero: 0 | (homo-cons) | |
| | | | homo-var: −5 | | |
| 80 | 0 | hetero | homo-cons: 6 | 8 | D |
| | | | hetero: 1 | (homo-cons) | |
| | | | homo-var: −5 | | |
| 81 | 0 | hetero | homo-cons: 4 | 5 | D |
| | | | hetero: 0 | (homo-cons) | |
| | | | homo-var: −6 | | |
| 82 | 0 | hetero | homo-cons: 6 | 6 | D |
| | | | hetero: 1 | (homo-cons) | |
| | | | homo-var: −9 | | |
| 83 | 0 | hetero | homo-cons: 5 | 5 | D |
| | | | hetero: 3 | (homo-cons) | |
| | | | homo-var: −3 | | |
| 84 | 0 | hetero | homo-cons: 3 | 2 | D |
| | | | hetero: 0 | (homo-cons) | |
| | | | homo-var: −5 | | |
| 85 | 0 | hetero | homo-cons: 5 | 3 | D |
| | | | hetero: 1 | (homo-cons) | |
| | | | homo-var: −3 | | |
| 86 | 0 | hetero | homo-cons: 5 | 6 | D |
| | | | hetero: 1 | (homo-cons) | |
| | | | homo-var: −4 | | |
| 87 | 0 | hetero | homo-cons: 3 | 4 | D |
| | | | hetero: 0 | (homo-cons) | |
| | | | homo-var: −7 | | |
| 88 | 0 | hetero | homo-cons: 5 | 6 | D |
| | | | hetero: 0 | (homo-cons) | |
| | | | homo-var: −5 | | |
| 89 | 0 | hetero | homo-cons: 7 | 7 | D |
| | | | hetero: 1 | (homo-cons) | |
| | | | homo-var: −4 | | |
| 90 | 0 | hetero | homo-cons: 8 | 7 | D |
| | | | hetero: 1 | (homo-cons) | |
| | | | homo-var: −4 | | |
| 91 | 0 | hetero | homo-cons: 6 | 7 | D |
| | | | hetero: 0 | (homo-cons) | |
| | | | homo-var: −5 | | |
| 92 | 0 | hetero | homo-cons: 6 | 6 | D |
| | | | hetero: 1 | (homo-cons) | |
| | | | homo-var: −5 | | |
| 93 | 0 | hetero | homo-cons: 3 | 2 | D |
| | | | hetero: 0 | (homo-cons) | |
| | | | homo-var: −5 | | |
| 94 | 0 | hetero | homo-cons: 9 | 11 | D |
| | | | hetero: 2 | (homo-cons) | |
| | | | homo-var: −5 | | |
| 95 | 0 | hetero | homo-cons: 7 | 7 | D |
| | | | hetero: 0 | (homo-cons) | |
| | | | homo-var: −5 | | |
| 96 | 0 | hetero | homo-cons: 6 | 6 | D |
| | | | hetero: 1 | (homo-cons) | |
| | | | homo-var: −5 | | |

As shown above in TABLE 11, all but one dbSNP call and both of the COSMIC SNP calls were validated by the ligation-dependent genotyping assay. Also, in most cases (31/36=86%), the heterozygous versus homozygous assignment from the ligation-dependent genotyping assay agreed with the results from sequence analysis. Two novel missense alleles identified by sequencing were validated in the ligation-dependent genotyping assay. All the other sequencing calls that indicated a potential SNV that were tested in the ligation-dependent genotyping assay proved to be false.

Conclusion: This Example demonstrates that the ligation-dependent genotyping assay can be successfully multiplexed in a single reaction tube and read out on a universal PCR matrix. The use of reference consensus and reference variant templates in a multiplex ligation assay allows for a simple scoring scheme for genotyping a test sample that is amenable to high throughput automation and analysis. The results described in Example 1 and in this Example demonstrate the successful genotyping of 144 of 144 SNV loci of interest, a 100% conversion rate (i.e., the percentage of designed assays that produce meaningful results).

As further described in this Example, it was determined that the use of lower concentrations of ligation primers (e.g., about 100 pM) reduce the background signal in the qPCR assay that was observed at high concentrations of ligation primers (e.g., about 500 pM). A 5-fold decrease in input ligation primer concentration (at fixed template) decreased signal by only 1.5 Cts, but decreased background signal by 3 Cts in real time qPCR measurements. This improved signal at decreased ligation oligo concentration indicates that it will be possible to multiplex hundreds of genotyping assays in a single reaction without compromising assay readout accuracy.

Taken together, the results described herein form the basis for an inexpensive and very high throughput two-step sequence validation/genotyping system. In step one, ligation oligos (potentially 1000 or more at once) are mixed with a sample, annealed and ligated in a single reaction mixture. In step two, the ligation mixture is distributed across a universal PCR “decoding” matrix, which can be dispensed into one or more multi-well assay plates and stored in a freezer prior to use, as described in Examples 2 and 4. The magnitude of the qPCR signal is indicative of the underlying genotype at a given SNV position of interest. As demonstrated herein, the ligation-dependent assay can distinguish between heterozygous and homozygous states in a diploid genome.

### Example 4

This Example describes the manufacture of a 576 feature matrix of detection primers (also referred to as a “universal PCR decoding matrix”), which can be pre-made and stored in a freezer, for decoding a multiplex assay, such as a multiplex ligation-dependent genotyping assay for genotyping a test sample at a plurality of SNV positions of interest.

Rationale:

As described in Example 2, an important element of the universal PCR decoding matrix is that the last (i.e., penultimate) two or three 3′ bases of the PCR primers are chosen to reduce and preferably eliminate primer-dimer formation, and the remaining bases are specificity tags chosen to provide a unique address at an intersection position (also referred to as a “feature”), in the matrix, such as a particular well on a multi-well assay plate. The universal PCR decoding matrix may be disposed into one or more multi-well assay plates.

This Example describes the manufacture of a 576 feature matrix of detection primers (universal PCR decoding matrix), that has minimal primer-dimer background due to the fact that the last three 3′ bases of the PCR primers were chosen to avoid primer-dimer formation. The 576 feature matrix was dispensed into a total of six 384-well assay plates, wherein each plate contained 96 primer pairs (i.e., features) in adjacent quadruplicate wells, and stored in a freezer for use in decoding a multiplex PCR assay.

PCR Primer Matrix Design

The goal of this Example was to design a larger matrix of minimally interacting primer pairs to manufacture a 576 feature matrix of detection primer pairs. A combined bioinformatic and empirical approach was used to create the 576 feature primer matrix that has minimal primer-dimer background and therefore the greatest possible measurement dynamic range for genotyping assays.

Since A residues and C residues cannot base pair with themselves or with C or A, respectively, these sequences were used as trinucleotides on the 3′ ends of primers as the basis of a minimally interactive, non-primer-dimer forming primer matrix. Specifically, one set of 36 potential primers was designed to end in “ACA,” and a second set of 36 primers was designed to end in “CAC”. Both primer sets were composed entirely of 25 nucleotide sequences. The 22 nucleotide “address” portions of each primer that are located at the 5′ end of each primer were screened from a computationally selected randomized list of 22 nt sequences that were specified to contain at least four of each A, C, G, or T DNA residues. Each candidate 22 nt sequence was screened for “GTG” and “TGT” sequences within 9 nt of the 3′ end of the 22 nt sequence, and those terminal 9 nt sequences containing these trinucleotides were eliminated. The rationale for this screening step is that the terminal “ACA” can pair with “TGT” and the terminal “CAC” can pair with “GTG”. Hence by eliminating potential 22 nt address sequences that possess 9 nt terminal “GTG” or “TGT” sequences, the probability of spurious primer-dimer formation is further reduced.

As shown in FIG. 3C, each forward PCR primer 600 has a 5′ region 602 that binds to a primer binding region 302, 403 in the 5′ tail of a 5′ ligation oligo 300, 400, and a region 606 at the 3′ end having a sequence selected to inhibit primer-dimer interactions with the reverse PCR primer 700. In this Example, the forward PCR primers 600 are located in rows in the primer matrix, and the “ACA” series was arbitrarily chosen to occupy these row positions. Similarly, as shown in FIG. 3C, each reverse PCR primer 700 has a 5′ region 702 that binds to a primer binding region 502 in the 3′ tail of a 3′ ligation oligo, and a region 706 at the 3′ end having a sequence selected to inhibit primer-dimer interactions with the forward PCR primer 600. In this Example, the reverse PCR primers 700 are located in columns in the primer matrix, and the “CAC” series was arbitrarily chosen to represent the column primers.

The 36 primer sequences in the “ACA” row series were designed as the forward primer set 600 to bind to the 5′ tail region 302, 402 on the ligation products 200, 250.

The 36 primer sequences in the “CAC” column series were designed as the reverse primer set 700 to bind to the 3′ common tail region 502 on the ligation products 200, 250.

The set of 36 “column” primers (“CAC” series) and 36 “row” primers (“ACA” series) was empirically tested in a complete “all-by-all” matrix for the formation of primer dimers, as follows.

The primers were synthesized by MWG/Operon (Huntsville, Ala.), diluted to a working stock concentration of 10 μM, and 4 μl of “row” primers and 4 μl of “column” primers were added in rows and columns, respectively, to a 96 well plate that contained 42 μl of PCR mix in each well. The PCR mix was composed of 25 μl of 2× Power SYBR master mix (Applied Biosystems, Foster City, Calif.) and 17 μl of water. The entire matrix collection of 36 row primers and 36 column primers occupied fifteen 96 well plates. For each 96 well plate, ten microliters of PCR mix from each unique well was aliquoted in quadruplicate to 384 well optical PCR plates (Applied Biosystems) and these were run for 40 cycles under standard SYBR green PCR cycling conditions on an ABI7900 qPCR instrument (Applied Biosystems).

Results:

Each set of quadruplicate wells was analyzed for the average Ct value with the goal of identifying a primer matrix where all Cts are 35 or higher. While certain addresses in the 36 by 36 primer matrix had Cts lower than this, by eliminating 12 of the “CAC” column primers and 12 of the “ACA” row primers, a matrix where all primer pairs yield background Cts>35 was identified, as shown in TABLE 12 and TABLE 13.

TABLE 12
SET OF 24 ROW “ACA” AND 24 COLUMN “CAC” PRIMERS

| Reference | | SEQ |
|---|---|---|
| No. | Sequence (5′ to 3′) | ID NO: |
| CAC1 | 5′ TTATCCCGAGAATTCAGACAGTCAC 3′ | 346 |
| CAC3 | 5′ CACGGGAGTTGATCCTGGTTTTCAC 3′ | 347 |
| CAC4 | 5′ TATAGCCGCTTAAGTCTACACTCAC 3′ | 348 |
| CAC5 | 5′ GTTTCGTAGCGTCCTGGAGTATCAC 3′ | 349 |
| CAC6 | 5′ AAAATGTTCTATATGACCGTTCCAC 3′ | 350 |
| CAC8 | 5′ ATCAAGAGTTTAGCACTTCGCGCAC 3′ | 351 |
| CAC9 | 5′ GGCGATGATAGATTCCCCTCGTCAC 3′ | 352 |
| CAC13 | 5′ TATGCGCTGGCAACATCGACACCAC 3′ | 353 |
| CAC14 | 5′ CAGAGATCATCCGAAGGCTTCTCAC 3′ | 354 |
| CAC16 | 5′ ATTATGAGACTCCCCGACGTCCCAC 3′ | 355 |
| CAC18 | 5′ TGTGATCGACGGCCTTTCAAATCAC 3′ | 356 |
| CAC19 | 5′ AACCCAACTCTGGCAAGCGTTACAC 3′ | 357 |
| CAC20 | 5′ GCGAAGGATTTGCTGACTTAAGCAC 3′ | 358 |
| CAC21 | 5′ ATATTCATGTGCAAAAGCCTCCCAC 3′ | 359 |
| CAC24 | 5′ CGTAACCCCAGACATAGGCCTTCAC 3′ | 360 |
| CAC25 | 5′ CTGAAAGCGGTCGACTAACGGGCAC 3′ | 361 |
| CAC28 | 5′ AATTGGCGTATACGGCCCCAAGCAC 3′ | 362 |
| CAC29 | 5′ CGTCTCAACTTAAGCCAGCCGACAC 3′ | 363 |
| CAC30 | 5′ AATAGCCCGGCTTTATACGCTGCAC 3′ | 364 |
| CAC31 | 5′ CGCTTGCGACCTCTTAAAACGTCAC 3′ | 365 |
| CAC32 | 5′ GTCATACATAACTCTTGAGATCCAC 3′ | 366 |
| CAC33 | 5′ TCGATCGCTTCAGACTATTTCGCAC 3′ | 367 |
| CAC34 | 5′ ACGATGGTTTGTTTCAGGAAACCAC 3′ | 368 |
| CAC35 | 5′ ACACACTTCCAGGCGATGGAAACAC 3′ | 369 |
| ACA1 | 5′ AACCGCTACAAGGCGGGGCACCACA 3′ | 370 |
| ACA2 | 5′ ACCAAACCTAGTAGCGCTATCCACA 3′ | 371 |
| ACA3 | 5′ TGCAGGACCAGAGAATTCGAATACA 3′ | 372 |
| ACA6 | 5′ ACTCAACATCGGCATCGGGCCTACA 3′ | 373 |
| ACA7 | 5′ ATTTCTACAAACGCTCGCCACAACA 3′ | 374 |
| ACA8 | 5′ AAAAATCCAAGTTTTAGGCGTTACA 3′ | 375 |
| ACA9 | 5′ TCTATCTATGGCCATGGTCTAAACA 3′ | 376 |
| ACA10 | 5′ GATTGCGCGGTAATAGCGCCCTACA 3′ | 377 |
| ACA12 | 5′ TCTTACGTGATGATATGGCAACACA 3′ | 378 |
| ACA13 | 5′ TCCCCTAGCACCCTAGGGTATGACA 3′ | 379 |
| ACA17 | 5′ TAAGTATTCCATGCACCCCTAAACA 3′ | 380 |
| ACA19 | 5′ CCTTACCTCGTAACTAACTAAGACA 3′ | 381 |
| ACA21 | 5′ TCTGGACAAGATTAGCTTACCAACA 3′ | 382 |
| ACA22 | 5′ TAACCGATACGTACGAGAGGCAACA 3′ | 383 |
| ACA24 | 5′ CCTGGACGAGGATTGACTCTACACA 3′ | 384 |
| ACA25 | 5′ TTCGGTTAGGTCCTACCGTACAACA 3′ | 385 |
| ACA26 | 5′ ATTCGAACGCTATCGAAAGGTTACA 3′ | 386 |
| ACA27 | 5′ AAGGATTGAGTCACATGGCGCAACA 3′ | 387 |
| ACA28 | 5′ TCAGCTAAGCCCTTATGATCCGACA 3′ | 388 |
| ACA29 | 5′ CATAAGCGAGTCATACTGACGAACA 3′ | 389 |
| ACA30 | 5′ CGAATGGATCAGTAACTCGAGAACA 3′ | 390 |
| ACA31 | 5′ GAAAGCAGGCAGGCCACTGACTACA 3′ | 391 |
| ACA32 | 5′ TAGAAACTCGACCAGAGGAGCTACA 3′ | 392 |
| ACA33 | 5′ GCTATCGGGGAATCCGCATCACACA 3′ | 393 |

TABLE 13
ASSAY RESULTS OF PCR PRIMER MATRIX
FOR THE PRESENCE OF PRIMER DIMERS.

| Ct values | CAC1 | CAC3 | CAC4 | CAC5 | CAC6 | CAC8 | CAC9 | CAC13 | CAC14 | CAC16 | CAC18 | CAC19 |
|---|---|---|---|---|---|---|---|---|---|---|---|---|
| ACA1 | 40 | 40 | 40 | 40 | 40 | 40 | 40 | 40 | 39 | 39 | 40 | 40 |
| ACA2 | 40 | 40 | 40 | 40 | 40 | 40 | 40 | 40 | 40 | 40 | 40 | 40 |
| ACA3 | 40 | 40 | 40 | 40 | 40 | 40 | 40 | 40 | 40 | 40 | 40 | 40 |
| ACA6 | 40 | 40 | 40 | 39 | 39 | 40 | 40 | 40 | 40 | 40 | 40 | 40 |
| ACA7 | 40 | 40 | 40 | 40 | 40 | 40 | 40 | 40 | 40 | 40 | 40 | 40 |
| ACA8 | 40 | 40 | 40 | 40 | 40 | 40 | 40 | 40 | 40 | 40 | 40 | 40 |
| ACA9 | 40 | 40 | 40 | 40 | 40 | 40 | 40 | 40 | 40 | 40 | 40 | 40 |
| ACA10 | 40 | 40 | 40 | 40 | 40 | 40 | 40 | 40 | 40 | 40 | 40 | 40 |
| ACA12 | 40 | 40 | 40 | 40 | 40 | 40 | 39 | 40 | 40 | 40 | 40 | 40 |
| ACA13 | 40 | 40 | 40 | 40 | 40 | 40 | 40 | 40 | 40 | 40 | 40 | 40 |
| ACA17 | 40 | 40 | 40 | 40 | 40 | 40 | 40 | 40 | 40 | 40 | 40 | 40 |
| ACA19 | 40 | 40 | 40 | 40 | 40 | 40 | 40 | 40 | 40 | 40 | 40 | 40 |
| ACA21 | 40 | 40 | 40 | 40 | 40 | 40 | 40 | 40 | 40 | 40 | 40 | 40 |
| ACA22 | 40 | 40 | 40 | 40 | 40 | 38 | 40 | 40 | 40 | 40 | 40 | 40 |
| ACA24 | 40 | 40 | 40 | 40 | 40 | 40 | 40 | 40 | 40 | 40 | 40 | 40 |
| ACA25 | 40 | 40 | 40 | 40 | 40 | 40 | 40 | 40 | 40 | 40 | 40 | 40 |
| ACA26 | 40 | 40 | 40 | 40 | 40 | 40 | 40 | 40 | 40 | 40 | 40 | 40 |
| ACA27 | 40 | 40 | 40 | 40 | 40 | 40 | 40 | 40 | 40 | 40 | 40 | 40 |
| ACA28 | 39 | 40 | 40 | 40 | 40 | 40 | 40 | 40 | 40 | 40 | 40 | 40 |
| ACA29 | 40 | 40 | 40 | 40 | 40 | 40 | 40 | 40 | 40 | 40 | 40 | 40 |
| ACA30 | 40 | 40 | 40 | 40 | 39 | 40 | 40 | 40 | 40 | 39 | 40 | 40 |
| ACA31 | 40 | 40 | 40 | 40 | 40 | 40 | 40 | 40 | 40 | 40 | 40 | 40 |
| ACA32 | 40 | 40 | 40 | 40 | 40 | 39 | 40 | 40 | 40 | 40 | 40 | 40 |
| ACA33 | 40 | 40 | 40 | 40 | 40 | 40 | 40 | 40 | 40 | 40 | 40 | 40 |
| Ct values | CAC20 | CAC21 | CAC24 | CAC25 | CAC28 | CAC29 | CAC30 | CAC31 | CAC32 | CAC33 | CAC34 | CAC35 |
| ACA1 | 40 | 40 | 40 | 40 | 40 | 40 | 40 | 40 | 40 | 40 | 40 | 40 |
| ACA2 | 40 | 38 | 40 | 40 | 40 | 40 | 39 | 40 | 40 | 40 | 39 | 40 |
| ACA3 | 40 | 40 | 40 | 40 | 40 | 40 | 40 | 40 | 40 | 40 | 40 | 40 |
| ACA6 | 40 | 40 | 40 | 40 | 40 | 40 | 40 | 40 | 40 | 40 | 40 | 40 |
| ACA7 | 40 | 40 | 40 | 40 | 40 | 40 | 40 | 40 | 40 | 40 | 40 | 40 |
| ACA8 | 40 | 40 | 40 | 40 | 40 | 40 | 40 | 40 | 40 | 40 | 40 | 40 |
| ACA9 | 40 | 40 | 40 | 40 | 40 | 40 | 40 | 40 | 40 | 39 | 40 | 40 |
| ACA10 | 40 | 40 | 40 | 40 | 40 | 40 | 40 | 40 | 40 | 40 | 39 | 40 |
| ACA12 | 40 | 39 | 40 | 40 | 40 | 40 | 40 | 40 | 40 | 40 | 40 | 40 |
| ACA13 | 40 | 40 | 40 | 40 | 40 | 40 | 40 | 40 | 40 | 40 | 40 | 40 |
| ACA17 | 40 | 40 | 40 | 40 | 40 | 40 | 40 | 40 | 40 | 40 | 39 | 40 |
| ACA19 | 40 | 40 | 39 | 40 | 40 | 40 | 40 | 40 | 40 | 40 | 40 | 40 |
| ACA21 | 40 | 40 | 40 | 40 | 40 | 40 | 40 | 40 | 40 | 40 | 40 | 40 |
| ACA22 | 40 | 40 | 40 | 40 | 40 | 40 | 39 | 40 | 40 | 40 | 40 | 40 |
| ACA24 | 40 | 40 | 40 | 40 | 40 | 40 | 40 | 40 | 40 | 40 | 40 | 40 |
| ACA25 | 40 | 40 | 40 | 40 | 40 | 40 | 40 | 40 | 40 | 40 | 40 | 40 |
| ACA26 | 40 | 40 | 40 | 39 | 40 | 40 | 40 | 40 | 40 | 40 | 40 | 40 |
| ACA27 | 40 | 40 | 40 | 40 | 40 | 40 | 40 | 40 | 40 | 36 | 40 | 40 |
| ACA28 | 40 | 40 | 40 | 39 | 40 | 40 | 40 | 40 | 40 | 40 | 40 | 40 |
| ACA29 | 40 | 40 | 40 | 40 | 40 | 40 | 40 | 40 | 40 | 40 | 40 | 40 |
| ACA30 | 40 | 40 | 40 | 40 | 40 | 40 | 40 | 40 | 40 | 40 | 40 | 40 |
| ACA31 | 40 | 40 | 40 | 40 | 40 | 40 | 40 | 40 | 40 | 40 | 40 | 40 |
| ACA32 | 40 | 40 | 40 | 39 | 40 | 40 | 40 | 40 | 39 | 40 | 40 | 40 |
| ACA33 | 40 | 40 | 40 | 40 | 40 | 40 | 40 | 40 | 38 | 40 | 40 | 40 |

As shown above in TABLE 13, a matrix of 24 “CAC” column primers and 24 “ACA” row primers was identified where all primer pairs yielded a background level of Cts>35.

As demonstrated in this Example, by using the described combined informatic and empirical approach, a set of 24 “CAC” column primers (SEQ ID NOS:346-369) and 24 “ACA” row primers (SEQ ID NOS:370-393) have been identified that fulfill the criterion of being a minimally interactive, low primer dimer forming matrix. The complete set of primers that comprise this matrix are shown in TABLE 12.

The universal PCR decoding matrix containing 24 column primers and 24 row primers (576 features) was dispensed into a total of six 384 well assay plates, wherein each plate contained 96 primer pairs (features) in adjacent quadruplicate wells. The assay plates containing the universal PCR decoding matrix were stored in a freezer for use in decoding a multiplex PCR assay as described herein.

### Example 5

This Example describes a method of ligation-dependent genotyping using separate annealing and ligation steps, and various other assay modifications that result in improved assay performance.

Rationale:

This Example describes a series of experiments that were carried out to determine the effect of various assay modifications on the performance of the ligation-dependant genotyping assay, including the use of separate annealing and ligation reaction conditions, the effect of different monovalent cations (e.g., Na+, K+, NH4+) on ligation efficiencies, the effect of ligation temperature, the effect of different ligases (TAQ or T4 DNA ligase), and the effect of ligase enzyme concentration and the length of ligation.

Methods:

A set of eight genotyping assays were designed to measure 8 SNV positions of interest under the various assay conditions as follows:

1. Preparation of Reagents for Ligation-Dependent Genotyping Assays

Synthetic Templates: The synthetic templates corresponding to the wild-type (consensus) allele, and the variant allele for each of the 8 SNV positions is provided in TABLE 14 (reverse complement sequences are shown). The length of each synthetic template is 51 nucleotides, with the polymorphic site (shown as underlined) located in the center of the template (i.e., 25 nucleotides on either side of the SNV position of interest).

Ligation Oligonucleotides: Each assay described in this Example was carried out with two different 5′ allele-specific ligation oligos 300, 400 and one common, phosphorylated 3′ ligation oligo 500 (e.g., as illustrated in FIG. 2).

The 5′ ligation oligos 300, 400 for assaying the 8 SNV positions of interest, shown in TABLE 15, were designed to have a total length of 51 nucleotides, with a 25 nt first primer binding tail region 302, 402 (underlined) at the 5′ most end, a 25 nt region of complementarity to the target template 304, 404, and a one nucleotide 3′ allele-specific region 306, 406 shown as underlined in bold.

The 3′ common phosphorylated [P] ligation oligos 500 for assaying the 8 SNV positions of interest, also shown in TABLE 15, were designed to have a total length of 50 nucleotides, with a 5′ target-specific binding region 504 of 25 nucleotides selected to hybridize immediately 3′ of the SNV position of interest, and a region 502 at the 3′ end that contains a second PCR primer binding region that is 25 nucleotides (underlined).

TABLE 14

SYNTHETIC TEMPLATES
(REVERSE COMPLEMENT SEQUENCE IS SHOWN)

| | | | | |
|---|---|---|---|---|
| | | | | SEQ |
| | | | | ID |
| SNV # | Ref. # | Template | Sequence (5′ to 3′) | NO: |

| | | | | |
|---|---|---|---|---|
| 1 | CT5 | consensus | CTGGGGGTAACTGTGCCTATTCGAGGGGTCCCTATGGGACTTGGGGTCCTC | 394 |
| 1 | VT5 | variant | CTGGGGGTAACTGTGCCTATTCGAGAGGTCCCTATGGGACTTGGGGTCCTC | 395 |
| 2 | CT6 | consensus | GTGCTGGCTTTGCTGGAGCTGGCGCAGCAGGACCACGGTGCTCTGGACTGC | 396 |
| 2 | VT6 | variant | GTGCTGGCTTTGCTGGAGCTGGCGCGGCAGGACCACGGTGCTCTGGACTGC | 397 |
| 3 | CT7 | consensus | TCCAGCACTCTGTCATGAGGCTGTACATTCTGGGTGGGCAGTCTTCAGAGC | 398 |
| 3 | VT7 | variant | TCCAGCACTCTGTCATGAGGCTGTAGATTCTGGGTGGGCAGTCTTCAGAGC | 399 |
| 4 | CT8 | consensus | CAACATCGACTTTGGCGAGCCCGGGGCCCGCCTGTCGCCGCCCGCGCCTCC | 400 |
| 4 | VT8 | variant | CAACATCGACTTTGGCGAGCCCGGGCCCCGCCTGTCGCCGCCCGCGCCTCC | 401 |
| 5 | CT13 | consensus | GCGAGTATTACTGCTACTCGAAATGCAAAAGCCACTCCAAGGCTCCGGAAA | 402 |
| 5 | VT13 | variant | GCGAGTATTACTGCTACTCGAAATGAAAAAGCCACTCCAAGGCTCCGGAAA | 403 |
| 6 | CT14 | consensus | CCAAATCGACTTACTCCTTTGCAGACAGAAACAGCCTCCTTGGACAAGGAT | 404 |
| 6 | VT14 | variant | CCAAATCGACTTACTCCTTTGCAGATAGAAACAGCCTCCTTGGACAAGGAT | 405 |
| 7 | CT15 | consensus | TCTCAGGATGCACCCAGTGGGCTCGAGGTCAGGGTGGCCTTGCCGGTGTCC | 406 |
| 7 | VT15 | variant | TCTCAGGATGCACCCAGTGGGCTCGCGGTCAGGGTGGCCTTGCCGGTGTCC | 407 |
| 8 | CT16 | consensus | CCTCACCAGAGGTGCCACCTACAACGTCATAGTGGAGGCACTGAAAGACCA | 408 |
| 8 | VT16 | variant | CCTCACCAGAGGTGCCACCTACAACATCATAGTGGAGGCACTGAAAGACCA | 409 |

TABLE 15
5′ AND 3′ LIGATION OLIGONUCLEOTIDES

| | | | |
|---|---|---|---|
| | | | SEQ |
| | Template | | ID |
| Ref # | Target | Sequence (5′ to 3′) | NO: |

5′ Ligation Olios

| | | | |
|---|---|---|---|
| FC5 | CT5 | AACCGCTACAAGGCGGGGCACCACAGAGGACCCCAAGTCCCATAGGGACCC | 410 |
| FV5 | VT5 | ACCAAACCTAGTAGCGCTATCCACAGAGGACCCCAAGTCCCATAGGGACCT | 411 |
| FC6 | CT6 | TGCAGGACCAGAGAATTCGAATACAGCAGTCCAGAGCACCGTGGTCCTGCT | 412 |
| FV6 | VT6 | ACTCAACATCGGCATCGGGCCTACAGCAGTCCAGAGCACCGTGGTCCTGCC | 413 |
| FC7 | CT7 | ATTTCTACAAACGCTCGCCACAACAGCTCTGAAGACTGCCCACCCAGAATG | 414 |
| FV7 | VT7 | AAAAATCCAAGTTTTAGGCGTTACAGCTCTGAAGACTGCCCACCCAGAATC | 415 |
| FC8 | CT8 | TCTATCTATGGCCATGGTCTAAACAGGAGGCGCGGGCGGCGACAGGCGGGC | 416 |
| FV8 | VT8 | GATTGCGCGGTAATAGCGCCCTACAGGAGGCGCGGGCGGCGACAGGCGGGG | 417 |
| FC13 | CT13 | AACCGCTACAAGGCGGGGCACCACATTTCCGGAGCCTTGGAGTGGCTTTTG | 418 |
| FV13 | VT13 | ACCAAACCTAGTAGCGCTATCCACATTTCCGGAGCCTTGGAGTGGCTTTTT | 419 |
| FC14 | CT14 | TGCAGGACCAGAGAATTCGAATACAATCCTTGTCCAAGGAGGCTGTTTCTG | 420 |
| FV14 | VT14 | ACTCAACATCGGCATCGGGCCTACAATCCTTGTCCAAGGAGGCTGTTTCTA | 421 |
| FC15 | CT15 | ATTTCTACAAACGCTCGCCACAACAGGACACCGGCAAGGCCACCCTGACCT | 422 |
| FV15 | VT15 | AAAAATCCAAGTTTTAGGCGTTACAGGACACCGGCAAGGCCACCCTGACCG | 423 |
| FC16 | CT16 | TCTATCTATGGCCATGGTCTAAACATGGTCTTTCAGTGCCTCCACTATGAC | 424 |
| FV16 | VT16 | GATTGCGCGGTAATAGCGCCCTACATGGTCTTTCAGTGCCTCCACTATGAT | 425 |

3′ Ligation Oligos (5′ to 3′)

| | | | |
|---|---|---|---|
| CP5 | CT5 | [P]CTCGAATAGGCACAGTTACCCCCAGGTGAAAACCAGGATCAACTCCCG | 426 |
| | VT5 | TG | |
| CP6 | CT6 | [P]GCGCCAGCTCCAGCAAAGCCAGCACGTGAAAACCAGGATCAACTCCCG | 427 |
| | VT6 | TG | |
| CP7 | CT7 | [P]TACAGCCTCATGACAGAGTGCTGGAGTGAAAACCAGGATCAACTCCCG | 428 |
| | VT7 | TG | |
| CP8 | CT8 | [P]CCCGGGCTCGCCAAAGTCGATGTTGGTGAAAACCAGGATCAACTCCCG | 429 |
| | VT8 | TG | |
| CP13 | CT13 | [P]CATTTCGAGTAGCAGTAATACTCGCGTGATACTCCAGGACGCTACGAA | 430 |
| | VT13 | AC | |
| CP14 | CT14 | [P]TCTGCAAAGGAGTAAGTCGATTTGGGTGATACTCCAGGACGCTACGAA | 431 |
| | VT14 | AC | |
| CP15 | CT15 | [P]CGAGCCCACTGGGTGCATCCTGAGAGTGATACTCCAGGACGCTACGAA | 432 |
| | VT15 | AC | |
| CP16 | CT16 | [P]GTTGTAGGTGGCACCTCTGGTGAGGGTGATACTCCAGGACGCTACGAA | 433 |
| | VT16 | AC | |

2. Pooling the Template Oligos

For each target SNV position of interest to be assayed, a set of control oligonucleotides were synthesized to generate double-stranded synthetic consensus and variant templates, with the reverse complement template sequences shown in TABLE 14.

Template oligonucleotides (sense and anti-sense template oligonucleotides) were mixed in two separate pools of 8 templates, resulting in a first pool containing 8 synthetic templates containing the consensus alleles for the 8 SNV positions of interest, and a second pool containing 8 synthetic templates containing the variant alleles for the 8 SNV positions of interest, and each pool was diluted to 10 pM.

3. Pooling the Ligation Oligos

The consensus and variant 5′ ligation oligos were combined and diluted to 500 nM (31.25 nM in each individual sequence).

The 3′ common ligation primers were kinased in a 100 μl reaction containing a 1 μM mixture of primers (62.5 nM in each sequence), 1× kinase buffer (New England Biolabs, Ipswich, Mass.), 1 mM ATP, and 20 U of T4 polynucleotide kinase. The reaction mixture was incubated at 37° C. for 30 minutes and 65° C. for 20 minutes. The kinased 3′ common ligation primers were then diluted to a final working concentration of 250 nM.

4. Quantitative PCT Assay (qPCR)

qPCR primers were synthesized as shown below in TABLE 16.

TABLE 16
QPCR PRIMERS

| Ref No | Sequence (5′ to 3′) | SEQ ID NO: |
|---|---|---|
| CAC3 | CACGGGAGTTGATCCTGGTTTTCAC | 347 |
| CAC5 | GTTTCGTAGCGTCCTGGAGTATCAC | 349 |
| ACA1 | AACCGCTACAAGGCGGGGCACCACA | 370 |
| ACA2 | ACCAAACCTAGTAGCGCTATCCACA | 371 |
| ACA3 | TGCAGGACCAGAGAATTCGAATACA | 372 |
| ACA6 | ACTCAACATCGGCATCGGGCCTACA | 373 |
| ACA7 | ATTTCTACAAACGCTCGCCACAACA | 374 |
| ACA8 | AAAAATCCAAGTTTTAGGCGTTACA | 375 |
| ACA9 | TCTATCTATGGCCATGGTCTAAACA | 376 |
| ACA10 | GATTGCGCGGTAATAGCGCCCTACA | 377 |

The qPCR primers were used in qPCR assays at a final concentration of 800 nM in each primer.

The qPCR assay plates used in each experiment described in this Example were configured to test 8 consensus assays and 8 variant assays (16 total), across six different experimental conditions, in an assay plate format shown below in TABLE 17.

TABLE 17
qPCR ASSAY PLATE FORMAT

| | CAC3 | CAC5 | CAC3 | CAC5 | CAC3 | CAC5 | CAC3 | CAC5 | CAC3 | CAC5 | CAC3 | CAC5 |
|---|---|---|---|---|---|---|---|---|---|---|---|---|
| ACA1 | 1 | 1 | 2 | 2 | 3 | 3 | 4 | 4 | 5 | 5 | 6 | 6 |
| ACA2 | 1 | 1 | 2 | 2 | 3 | 3 | 4 | 4 | 5 | 5 | 6 | 6 |
| ACA3 | 1 | 1 | 2 | 2 | 3 | 3 | 4 | 4 | 5 | 5 | 6 | 6 |
| ACA6 | 1 | 1 | 2 | 2 | 3 | 3 | 4 | 4 | 5 | 5 | 6 | 6 |
| ACA7 | 1 | 1 | 2 | 2 | 3 | 3 | 4 | 4 | 5 | 5 | 6 | 6 |
| ACA8 | 1 | 1 | 2 | 2 | 3 | 3 | 4 | 4 | 5 | 5 | 6 | 6 |
| ACA9 | 1 | 1 | 2 | 2 | 3 | 3 | 4 | 4 | 5 | 5 | 6 | 6 |
| ACA10 | 1 | 1 | 2 | 2 | 3 | 3 | 4 | 4 | 5 | 5 | 6 | 6 |

Note:
each number (1-6) indicates a different assay condition that was tested.

Although 96 wells are shown in the assay plate format depicted above in TABLE 17, it will be understood that each of the 96 positions represents a quadruplicate set of assay wells in a 384 well PCR plate.

Each qPCR assay was carried out in quadruplicate, with 10 μl of SYBR green PCR reaction mix (5 μl of 2× power SYBR master mix, Applied Biosystems, Foster City Calif.), 1.4 μl H₂O, 0.8 μl of 10 μM row and column primers and 2 μl of template (e.g., 2 μl of a genotyping assay reaction). The genotyping assay reactions are described below.

5. Annealing and Ligation Reactions

A. Determination of the Effect of Different Monovalent Cations Na⁺, K⁺, and NH₄⁺, on Ligation Efficiencies.

Methods:

A coupled annealing/ligation reaction was performed in which different monovalent cationic salts were added to stimulate annealing of the genotyping primers to the complementary genotyping targets.

Stock solutions of 2.5 M KCl, 2.5 M NH₄Cl, and 2.5 M NaCl were prepared.

Genotyping Reactions:

Consensus synthetic templates or no template controls were assayed using 5′ ligation oligos (consensus and variant) primer pools.

For each genotyping ligation reaction, the following reagents were combined:

75 μl H₂O

10 μl of 10 pM consensus synthetic template or water (no template control)

2 μl of 500 nM combined 5′ consensus and variant primer pools (each individual query oligo was present in the final genotyping mix at a final concentration of 625 pM)

2 μl of 250 nM 3′ kinased common primer pool (each individual query oligo was present in the final genotyping mix at a final concentration of 625 pM)

10 μl of 10× Taq DNA ligase buffer (New England Biolabs, Ipswich, Mass. (NEB))

2 μl of 2.5 M NaCl or 2.5 M KCl or 2.5 M NH₃Cl

100 μl total volume. 2 μl ligase enzyme (40 U/μl Taq DNA ligase, NEB) was added and the ligation mixture was then incubated in a thermal cycler across the following temperatures:

95° C. for 5 minutes;

75° C. for 15 minutes;

70° C. for 15 minutes;

65° C. for 30 minutes;

60° C. for 45 minutes;

55° C. for 30 minutes;

50° C. for 15 minutes;

45° C. for 15 minutes;

4° C. rest.

The ligation reactions were diluted to 1 ml with 900 μl of TEzero (10 mM Tris pH 7.6, 0.1 mM EDTA) and 2 μl of each ligation reaction was assayed in quadruplicate qPCR reactions as described above in Section 4.

Results:

The average raw Ct data from each of the qPCR assays was first determined across four wells of each quadruplicate assay. The results of the ligation with consensus templates were measured against a no template control to obtain a set of raw Ct data (data not shown). The scoring scheme of genotyping was then applied to the Ct data as described in Example 3.

Table 18 below shows the Ct(variant)−Ct(consensus) assay results for each of the eight assays under the three salt conditions tested (NaCl, KCl and NH₄Cl), and the average Ct(consensus), Ct(variant), and Ct(background) for each monovalent cation.

TABLE 18
CONSENSUS TEMPLATE GENOTYPING CALLS FOR SNV
POSITIONS 1-8 AND AVERAGE CTS FROM ASSAYS CARRIED
OUT USING THE THREE DIFFERENT MONOVALENT CATIONS.

| NaCl | | KCL | | NH₄Cl | |
|---|---|---|---|---|---|
| monovalent cation | | monovalent cation | | monovalent cation | |
| Ct(var) − Ct(cons) | | Ct(var) − Ct(cons) | | Ct(var) − Ct(cons) | |
| SNV 1 | 2 | SNV 1 | 1 | SNV 1 | 1 |
| SNV 2 | 4 | SNV 2 | 4 | SNV 2 | 4 |
| SNV 3 | 2 | SNV 3 | 2 | SNV 3 | 2 |
| SNV 4 | 5 | SNV 4 | 5 | SNV 4 | 5 |
| SNV 5 | 3 | SNV 5 | 2 | SNV 5 | 1 |
| SNV 6 | 6 | SNV 6 | 5 | SNV 6 | 3 |
| SNV 7 | 1 | SNV 7 | 2 | SNV 7 | 1 |
| SNV 8 | 5 | SNV 8 | 4 | SNV 8 | 3 |
| Average Ct | 24 | Average Ct | 25 | Average Ct | 25 |
| (cons) | | (cons) | | (cons) | |
| Average Ct | 28 | Average Ct | 28 | Average Ct | 27 |
| (var) | | (var) | | (var) | |
| Average Ct | 31 | Average Ct | 31 | Average Ct | 32 |
| (bgd) | | (bgd) | | (bgd) | |

As shown above in TABLE 18, optimal assay performance is observed with NaCl, however there are relatively minor differences between the three cations tested. This result was unexpected because according to Takahashi et al., J. Biol Chem. 259(16):10041-10047 (1984), Na+ inhibits Taq DNA ligase activity, while K+ and NH₄⁺ stimulate enzyme activity.

B. Determination of the Effect of Separating the Annealing and Ligation Steps, with Either (1) a Shorter Annealing Time, (2) Different Ligation Enzymes, (3) Various Ligation Temperatures, or (4) Various Ligation Concentrations, on the Performance of the Ligation-Dependent Genotyping Assay

Rationale: The genotyping assays described in Examples 1 and 3 above were carried out with coupled annealing/ligation reactions in which the oligonucleotide reagents were added in the presence of thermostable ligase and subjected to conditions that allowed hybridization of the query oligonucleotides to the target templates. The following experiments were carried out to determine whether the annealing of the query oligonucleotides to the target template and subsequent ligation reaction in separate steps would improve the performance of the genotyping assay, and to test the effect of a shorter annealing time, different ligation enzymes, various ligation temperatures, and various ligase concentrations, on the performance of the genotyping assay.

Methods:

Annealing of templates and assay oligos was carried out as follows for each genotyping assay:

10 μl of 10 pM synthetic template (consensus or variant)

2 μl of 500 nM 5′ consensus and variant ligation primers

2 μl of 250 nM kinased 3′ common ligation primers

2 μl of 5 M NaCl

16 μl total (Note: the NaCl concentration in this annealing reaction is twice the concentration used in the monovalent comparison experiment described above)

The annealing mixtures were incubated in a thermal cycler across the following temperatures for the following time periods:

1. Standard Protocol (Total: 170 Minutes)

95° C. for 5 minutes;

75° C. for 15 minutes;

70° C. for 15 minutes;

65° C. for 30 minutes;

60° C. for 45 minutes;

55° C. for 30 minutes;

50° C. for 15 minutes;

45° C. for 15 minutes;

4° C. rest.

2. Rapid Annealing Protocol (Total: 65 Min)

95° C. for 5 minutes;

75° C. for 15 minutes;

70° C. for 15 minutes;

65° C. for 30 minutes;

4° C. rest.

Ligations:

1. Taq DNA Ligase Reactions

A ligation mix “cocktail” was prepared containing:

10 μl of 10× Taq DNA ligase buffer (NEB)

72 μl of H₂O

2 μl of Taq DNA Ligase (40 U/μl, NEB)

84 μl total, which was added to each annealed reaction mixture (16 μl) for a total volume of 100 μl in each ligation reaction. For the Taq DNA ligase reactions, ligations were performed at 37° C., 45° C., 55° C., and 65° C. for 30 minutes.

For the rapid annealing protocol described above, follow on ligation with Taq DNA ligase was performed at 45° C. for 30 minutes.

2. T4 DNA Ligase Reactions

A ligation mix “cocktail” was prepared containing:

10 μl of 10× T4 DNA ligase buffer (NEB)

72 μl of H₂O

2 μl of T4 DNA Ligase (400 U/μl (NEB))

84 μl total, which was added to each annealed reaction mixture (16 μl) for a total volume of 100 μl in each ligation reaction.

For the T4 DNA ligase reactions, ligations were performed at 25° C., 30° C., and 37° C. for 30 minutes.

Following the ligation reaction incubations at the indicated temperatures, each of the 100 μl ligation mixtures was diluted with 900 μl of TEzero (10 mM Tris pH 7.6, 0.1 mM EDTA) and 2 μl was assayed in quadruplicate by SYBR green qPCR as described above in Section 4.

Results:

The average raw Ct data from each of the qPCR assays was first determined across four wells of each quadruplicate assay. The results of the ligation with consensus templates were measured against a no template control to obtain a set of raw Ct data (data not shown). The scoring scheme of genotyping was then applied to the Ct data as described in Example 3.

TABLES 19 to 22 below show the genotyping results for all of the genotyping assays described in this Example.

“HC” stands for “homozygous consensus” genotyping calls, and is calculated as the Ct(variant)-Ct(consensus) for reactions with the consensus templates.

“HET” stands for “heterozygous” genotyping calls, and is calculated as the Ct(variant) for the variant template minus the Ct(consensus) for the consensus template.

“HV” stands for “homozygous variant” genotyping calls, and is calculated as the Ct(variant)−Ct(consensus) for reactions with the variant templates.

The symbol “Δ” represents the overall dynamic range of each assay set, which is calculated as the absolute value of “HC”−“HV.”

The average values across the eight assays are shown for each condition in bold at the bottom of each table.

TABLE 19
HISTORICAL DATA (COUPLED ANNEALING/LIGATION
REACTION WITH TAQ DNA LIGASE AS DESCRIBED
IN EXAMPLE 3)

| | HC | HET | HV | Δ |
|---|---|---|---|---|
| SNV 1 | 1 | −2 | −5 | 6 |
| SNV 2 | 6 | −2 | −5 | 11 |
| SNV 3 | 2 | 0 | −6 | 8 |
| SNV 4 | 4 | 1 | −6 | 10 |
| SNV 5 | 3 | −2 | −9 | 12 |
| SNV 6 | 5 | 1 | −2 | 7 |
| SNV 7 | 2 | −1 | −4 | 6 |
| SNV 8 | 5 | 0 | −3 | 8 |
| Average | 3 | −1 | −5 | 9 |

TABLE 20
T4 DNA LIGASE REACTIONS AT 25° C., 30° C. AND 37° C.
(SEPARATE ANNEALING, LIGATION STEPS)

| | 25° C. ligation | | | | 30° C. ligation | | | | 37° C. ligation | | | |
|---|---|---|---|---|---|---|---|---|---|---|---|---|
| | HC | HET | HV | Δ | HC | HET | HV | Δ | HC | HET | HV | Δ |
| SNV 1 | 3 | 1 | −2 | 4 | 3 | 1 | −2 | 5 | 3 | 0 | −2 | 5 |
| SNV 2 | 2 | 0 | −2 | 4 | 2 | 0 | −2 | 4 | 2 | 0 | −2 | 4 |
| SNV 3 | 2 | 0 | −3 | 5 | 2 | 0 | −3 | 5 | 2 | 0 | −3 | 5 |
| SNV 4 | 3 | 2 | −3 | 6 | 3 | 2 | −3 | 6 | 4 | 2 | −3 | 7 |
| SNV 5 | −1 | −1 | −4 | 3 | 0 | −1 | −4 | 4 | 1 | −1 | −3 | 4 |
| SNV 6 | 3 | 1 | −1 | 4 | 4 | 1 | −1 | 4 | 4 | 1 | −1 | 5 |
| SNV 7 | 3 | 2 | 2 | 1 | 3 | 2 | 1 | 2 | 3 | 1 | 0 | 3 |
| SNV 8 | 4 | 1 | −2 | 6 | 4 | 1 | −2 | 6 | 4 | 1 | −3 | 6 |
| Average | 2 | 1 | −2 | 4 | 3 | 1 | −2 | 5 | 3 | 0 | −2 | 5 |

TABLE 21
TAQ DNA LIGASE REACTIONS AT 37° C., 55° C. AND 65° C.
(SEPARATE ANNEALING, LIGATION STEPS)

| | 37° C. ligation | | | | 55° C. ligation | | | | 65° C. ligation | | | |
|---|---|---|---|---|---|---|---|---|---|---|---|---|
| | HC | HET | HV | Δ | HC | HET | HV | Δ | HC | HET | HV | Δ |
| SNV 1 | 6 | 0 | −8 | 14 | 7 | 0 | −8 | 14 | 6 | 0 | −8 | 14 |
| SNV 2 | 5 | −1 | −5 | 11 | 6 | 0 | −6 | 12 | 6 | 0 | −5 | 11 |
| SNV 3 | 2 | 1 | −6 | 8 | 2 | 1 | −7 | 9 | 3 | 1 | −6 | 8 |
| SNV 4 | 7 | 2 | −4 | 11 | 8 | 2 | −4 | 12 | 7 | 2 | −4 | 11 |
| SNV 5 | 7 | −1 | −11 | 18 | 7 | −1 | −12 | 19 | 7 | 0 | −10 | 18 |
| SNV 6 | 6 | 0 | −5 | 12 | 7 | 0 | −6 | 13 | 7 | 0 | −6 | 13 |
| SNV 7 | 2 | −1 | −6 | 8 | 2 | −1 | −7 | 9 | 2 | −1 | −6 | 8 |
| SNV 8 | 7 | 1 | −8 | 15 | 7 | 1 | −8 | 15 | 7 | 1 | −7 | 14 |
| Average | 5 | 0 | −7 | 12 | 6 | 0 | −7 | 13 | 6 | 0 | −7 | 12 |

TABLE 22
TAQ DNA LIGASE REACTIONS AT 45° C. AFTER RAPID
ANNEALING (65 MINUTES) OR AFTER STANDARD ANNEALING
(170 MINUTES) (SEPARATE ANNEALING, LIGATION STEPS)

| | 45° C. ligation after | | | | 45° C. ligation after | | | |
|---|---|---|---|---|---|---|---|---|
| | standard annealing | | | | rapid annealing | | | |
| | HC | HET | HV | Δ | HC | HET | HV | Δ |
| SNV 1 | 7 | 0 | −7 | 14 | 6 | 0 | −8 | 14 |
| SNV 2 | 6 | −1 | −6 | 12 | 6 | −1 | −5 | 11 |
| SNV 3 | 2 | 1 | −5 | 7 | 2 | 1 | −6 | 8 |
| SNV 4 | 8 | 2 | −3 | 11 | 7 | 1 | −4 | 11 |
| SNV 5 | 7 | −1 | −11 | 18 | 6 | −1 | −11 | 17 |
| SNV 6 | 8 | 0 | −5 | 12 | 7 | 0 | −5 | 12 |
| SNV 7 | 2 | −1 | −7 | 9 | 2 | −1 | −7 | 9 |
| SNV 8 | 8 | 1 | −8 | 15 | 6 | 1 | −8 | 14 |
| Average | 6 | 0 | −6 | 12 | 5 | 0 | −7 | 12 |

Discussion of Results:

Based on the results shown above in TABLES 19 and 20, the ligation-dependent genotyping assays carried out with T4 DNA ligase do not perform as well as those carried out with Taq DNA ligase. It is noted that the greater the Ct spreads between measurements of consensus versus variant genotypes, the better the accuracy in assigning genotypes. In this regard, the dynamic ranges of Taq ligated assays was far greater (i.e., average Δ value of 9) as compared to the dynamic range of the T4 DNA ligase assays (i.e., average Δ value of 4 to 5). It was determined, based on analysis of the raw Ct values, that the reason for this difference in dynamic range is due to the fact that T4 ligase has a tendency to ligate mismatched oligos, therefore the background in the T4 ligase based assay is worse than in the Taq ligase based assay.

Importantly, as shown above in TABLES 19, 21, and 22, it was observed that the genotyping assays carried out with an annealing reaction followed by separate Taq DNA ligase reaction performed better than the coupled annealing/ligation assays with Taq DNA ligase at all ligation temperatures tested. For example, the average dynamic range of the coupled annealing/ligation genotyping assay with Taq DNA ligase had a dynamic range Δ value of 9, whereas the average dynamic range of the uncoupled assay (i.e., separate annealing and ligation steps) with Taq DNA ligase was increased (e.g., 37° C.=Δ value of 12; 45° C.=Δ value of 12; 55° C.=Δ value of 13; and 65° C.=Δ value of 12). Also, the distance between each of the genotyping calls (HC, HET, HV) was greater for the uncoupled Taq DNA ligase assays (e.g., average value for 37° C. assay of 5, 0, −7, respectively), as compared to the distance between each genotyping call for the coupled Taq DNA ligase assays (e.g., average value of 3, 1, −5, respectively).

As shown in TABLES 20 and 21, the genotyping assays carried out with Taq DNA ligase under the various ligation temperatures tested in an uncoupled genotyping assay appear to be more or less equivalent. Therefore, a 45° C. ligation temperature with Taq DNA ligase in an uncoupled annealing and ligation reaction was chosen for future experiments.

TABLE 22 shows the results of the comparison of a rapid annealing time (65 minutes total) to a standard annealing time (170 minutes) in an uncoupled genotyping assay with the ligation step carried out with Taq DNA ligase at 45° C. As shown in TABLE 22, the results are more or less equivalent, with the same dynamic range (Δ value of 12), and a good distance between each genotyping call (HC, HET, HV) for the rapid annealing assay (i.e., average value of 5, 0, −7, respectively), as compared to the distance between each genotyping call for the assay with the longer annealing time (i.e., average value of 6, 0, −6 respectively). These results demonstrate that oligonucleotide annealing times can be shortened from 170 minutes to 65 minutes or less, and the shorter annealing times were used in all subsequent experiments.

Therefore, based on the above results, it was concluded that the decoupled annealing and ligation reaction generally improved the results of the genotyping assays as compared to the coupled annealing/ligation reaction. In particular, it was observed that the optimal conditions for the ligation-dependent genotyping assay involved a rapid annealing step (approximately 60 minutes), followed by ligation with Taq DNA ligase at 45° C.

C. Determination of the Effect of Ligase Enzyme Concentration and Incubation Time on the Performance of the Ligation-Dependent Genotyping Assay

In this series of experiments, the variables of Taq DNA ligase enzyme concentration and time of ligation were measured with respect to the genotyping assay performance. In order to determine the minimum ligase concentration required and the influence of time on ligation efficiency, the set of eight SNV query oligos described above in TABLE 15 were assayed against the consensus templates shown in TABLE 14 in a first experiment and the same query reagents were assayed in a second experiment with the variant templates shown in TABLE 14. The genotyping assays were carried out with the rapid annealing protocol followed by ligation with Taq DNA ligase at 45° C.

Annealing Reaction

For each assay reaction, the following reagents were combined:

10 μl of 10 pM pooled templates (variant or consensus)

2 μl of 500 nM pooled consensus and variant 5′ ligation primers

2 μl of 250 nM kinased 3′ common ligation primers

2 μl of 5 M NaCl

16 μl total volume

Annealing Temperatures:

The rapid annealing protocol was carried out as follows:

95° C. for 5 minutes;

75° C. for 15 minutes;

70° C. for 15 minutes;

65° C. for 30 minutes;

4° C. rest.

Taq DNA Ligation Reactions

A ligation mix “cocktail” was prepared containing:

10 μl of 10× Taq DNA ligase buffer (NEB)

74 μl of H₂O

2 μl, 1 μl, 0.5 μl, 0.1 or 0.02 μl of Taq DNA ligase (40 U/μl, NEB)

85 μl total, which was added to each annealed reaction mixture (16 μl) for a total volume of 100 μl in each ligation reaction.

The ligation reactions were incubated at 45° C. for 30 minutes, 20 minutes, 10 minutes, 5 minutes, or 1 minute. The ligation reactions were terminated by the addition of 900 μl of TE, and 2 μl of each ligation reaction was assayed in quadruplicate 10 μl qPCR reactions as described above in Section 4.

Results:

The average raw Ct data from each of the qPCR assays was first determined across four wells of each quadruplicate assays. The results of the ligation with consensus templates were measured against a no template control to obtain a set of raw Ct data (data not shown). The scoring scheme of genotyping was then applied to the Ct data as described in Example 3. The results are shown below in TABLE 23 and TABLE 24.

TABLE 23
RESULTS OF THE LIGATION REACTIONS CARRIED OUT WITH
VARIOUS TIME AND ENZYME CONCENTRATIONS ON THE PERFORMANCE
OF THE GENOTYPING ASSAYS USING THE CONSENSUS TEMPLATES.
UNLESS OTHERWISE INDICATED, VALUES
SHOWN ARE (CT (VARIANT) − CT (CONSENSUS).

| | 30 min ligation | | | 20 min ligation | | | 10 min ligation | | | 5 min ligation | | |
|---|---|---|---|---|---|---|---|---|---|---|---|---|
| ligase | 2 μl | 1 μl | 0.5 μl | 2 μl | 1 μl | 0.5 μl | 2 μl | 1 μl | 0.5 μl | 2 μl | 1 μl | 0.5 μl |
| SNV 1 | 8 | 9 | 9 | 8 | 9 | 8 | 7 | 9 | 8 | 8 | 9 | 8 |
| SNV 2 | 7 | 6 | 7 | 6 | 6 | 5 | 6 | 6 | 6 | 6 | 6 | 6 |
| SNV 3 | 2 | 2 | 2 | 2 | 2 | 2 | 2 | 2 | 2 | 2 | 2 | 3 |
| SNV 4 | 8 | 7 | 7 | 8 | 7 | 8 | 7 | 7 | 7 | 7 | 8 | 8 |
| SNV 5 | 8 | 8 | 9 | 8 | 7 | 7 | 7 | 7 | 7 | 7 | 8 | 8 |
| SNV 6 | 9 | 8 | 8 | 8 | 8 | 8 | 8 | 8 | 9 | 9 | 7 | 8 |
| SNV 7 | 2 | 3 | 3 | 2 | 2 | 2 | 2 | 3 | 3 | 2 | 2 | 3 |
| SNV 8 | 7 | 7 | 7 | 7 | 6 | 6 | 7 | 6 | 7 | 6 | 7 | 7 |
| Average | 25 | 25 | 25 | 25 | 25 | 26 | 25 | 26 | 26 | 25 | 26 | 27 |
| Ct (cons) | | | | | | | | | | | | |
| Average | 31 | 31 | 32 | 31 | 31 | 31 | 31 | 32 | 32 | 31 | 32 | 33 |
| Ct (var) | | | | | | | | | | | | |
| Average | 6 | 6 | 6 | 6 | 6 | 6 | 6 | 6 | 6 | 6 | 6 | 6 |
| Ct (var) − | | | | | | | | | | | | |
| Ct (cons) | | | | | | | | | | | | |

TABLE 24
RESULTS OF THE LIGATION REACTIONS CARRIED OUT WITH
VARIOUS TIME AND ENZYME CONCENTRATIONS ON THE
PERFORMANCE OF THE GENOTYPING ASSAYS USING THE
VARIANT TEMPLATES. UNLESS OTHERWISE INDICATED,
VALUES SHOWN ARE (CT (CONSENSUS) − CT (VARIANT).

| | 5 min ligation | | | 1 min ligation | | |
|---|---|---|---|---|---|---|
| ligase | 0.5 μl | 0.1 μl | 0.02 μl | 0.5 μl | 0.1 μl | 0.02 μl |
| SNV 1 | 8 | 7 | 7 | 8 | 8 | 7 |
| SNV 2 | 5 | 4 | 2 | 5 | 5 | 4 |
| SNV 3 | 6 | 8 | 5 | 7 | 6 | 6 |
| SNV 4 | 5 | 5 | 6 | 4 | 4 | 3 |
| SNV 5 | 9 | 9 | 8 | 10 | 8 | 8 |
| SNV 6 | 5 | 6 | 8 | 5 | 7 | 7 |
| SNV 7 | 8 | 7 | 6 | 7 | 7 | 5 |
| SNV 8 | 9 | 8 | 11 | 9 | 7 | 7 |
| Average | 26 | 28 | 31 | 26 | 28 | 31 |
| Ct (var) | | | | | | |
| Average | 33 | 35 | 37 | 33 | 35 | 36 |
| Ct (cons) | | | | | | |
| Average | 7 | 7 | 7 | 7 | 6 | 6 |
| Ct | | | | | | |
| (cons) − | | | | | | |
| Ct (var) | | | | | | |

Discussion of Results:

As shown above in TABLE 23 and TABLE 24, the results of the genotyping assay with a ligation reaction carried out for 5 minutes is about equivalent to the results of the genotyping assay with a ligation reaction carried out for longer periods of time (i.e., 10, 20, or 30 minutes), both in terms of Ct(variant)-Ct(consensus) differences and with respect to the absolute Ct values for cognate versus mismatched templates.

As further shown in TABLE 23 and TABLE 24, low concentrations (0.5 μl to 1 μl of 40 U/μl) of Taq DNA ligase appear adequate for driving ligation to the same levels as observed with greater amounts of Taq DNA ligase enzyme.

Therefore, based on the above results, it was determined that the optimal conditions for the 100 μl ligation reaction in the ligation-dependent genotyping assay includes the use of a rapid annealing step (approximately 60 minutes), followed by ligation with Taq DNA ligase at a concentration of from about 0.5 μl to about 1.0 μl of 40 U/μl for 5 minutes at 45° C.

### Example 6

This Example describes the manufacture of a 576-feature matrix of minimally interacting pairs of detection primers (also referred to as a “universal PCR decoding matrix”) for use in decoding a multiplex assay, such as a multiplex ligation-dependent genotyping assay for genotyping a test sample at a plurality of SNV positions of interest.

PCR Primer Matrix Design

The goal of this Example was to design a matrix of minimally interacting primer pairs to manufacture a 576-feature matrix of detection primer pairs.

Rationale:

Since adenine residues cannot base pair with cytosine residues, these sequences were used as trinucleotides on the 3′ ends of primers as the basis of a minimally interactive, non-primer-dimer forming primer matrix. Specifically, one set of 36 potential primers was designed to end in “CCC,” and a second set of 36 primers was designed to end in “AAA” at each of their 3′ ends.

Candidate 25 mer PCR primer sequences were chosen in the following way.

First, a 10,000 list of random 22-mer DNA sequences was generated. The only criterion was that these sequences were required to have at least four of each type of DNA base (A, G, C, T).

A list of 200 of the 10,000 sequences were chosen at random and screened for the presence of either “TTT” or “GGG” in the 3′ terminal 6 nucleotides, which were then removed from the list of candidate primers. The rationale for removal of these primers is that “TTT” can pair with “AAA” and “GGG” can pair with “CCC,” therefore, primers with these 3′ terminal sequences would be susceptible to primer-dimer formation. Approximately 15% of the randomly selected sequences were removed from the list of candidate PCR primers via this filtering process, leaving a total of 170 candidate sequences.

72 of the 170 remaining candidate sequences were randomly chosen as the candidate PCR primer sequences. The 3′ terminal sequence of “CCC” was added to the first set of 36 of these sequences (“row primers”), and the 3′ terminal sequence of “AAA” was added to the second set of 36 of these sequences (“column primers”), thereby creating a 36 by 36 primer matrix, as shown below in TABLE 25.

TABLE 25
SET OF 36 “CCC” AND 36 “AAA” PRIMERS

| | | SEQ | Used in |
|---|---|---|---|
| Reference | | ID | final |
| Number | Sequence (5′ to 3′) | NO: | matrix |
| AAA1 | GATCTGGCTAGGTGCCACAACAAAA | 434 | + |
| AAA2 | GACATGCTAACCACGTTGCAGGAAA | 435 | + |
| AAA3 | GACCTCGTAAAAGGGGGTATAGAAA | 436 | − |
| AAA4 | AAAATACCATCTTGGCCATTATAAA | 437 | + |
| AAA5 | GAGTGACTGCAACTAAAATGCTAAA | 438 | + |
| AAA6 | TGTATCAGAGGATTGCGTTCGAAAA | 439 | + |
| AAA7 | GTTCGGGGATACATTCTGAGTAAAA | 440 | − |
| AAA8 | TGCAACTAGATTGAGGCCTCTAAAA | 441 | + |
| AAA9 | CTATATGTAGGGGCTCTAACCGAAA | 442 | − |
| AAA10 | CATCTGCTGCGTTTGGAATACGAAA | 443 | + |
| AAA11 | ATACCAGCCGGCTGATGATCGTAAA | 444 | + |
| AAA12 | AGCCACTCTGTAGCACTGATGGAAA | 445 | + |
| AAA13 | TACCCTAGTTGGCAGTTCATCGAAA | 446 | + |
| AAA14 | ATAATAGTCGCTGGTATGGTACAAA | 447 | + |
| AAA15 | ATTTGGAACACCGCAGCTCGGTAAA | 448 | + |
| AAA16 | GACCCCGTGCACGGATGCATGAAAA | 449 | + |
| AAA17 | GTCGGGCAGCACCCAAGTTCTGAAA | 450 | + |
| AAA18 | AGCTGTGGTTAAGGATAGTTCGAAA | 451 | + |
| AAA19 | AGTGCAAATTCGACACTTGACGAAA | 452 | + |
| AAA20 | GGCCCTCCTTATTAAACATCCGAAA | 453 | + |
| AAA21 | ACTCACTCTGGGCAGACGCAGAAAA | 454 | − |
| AAA22 | TTCGGGCGTTCTGAAGACCTGTAAA | 455 | − |
| AAA23 | CCGGGGGAGTCATTGTATTACGAAA | 456 | − |
| AAA24 | GTAGACCGTAGCGAACACCGGAAAA | 457 | + |
| AAA25 | AGTCTCGGTTCCGCATGCGTCGAAA | 458 | − |
| AAA26 | CATACCGTCAACTAATATTCTCAAA | 459 | + |
| AAA27 | GTGGGATGGAGTCCACGAAATTAAA | 460 | + |
| AAA28 | TTGGAGTTTAGCGACACGCATTAAA | 461 | + |
| AAA29 | ATCTATCTTGAACCCGGGCGATAAA | 462 | + |
| AAA30 | GGCACTCGGGTCTTATCCGTTGAAA | 463 | − |
| AAA31 | GCTTATACGCAACTGTGTCTGGAAA | 464 | − |
| AAA32 | CAAAAGAGGTTGTCGTAGCTCGAAA | 465 | + |
| AAA33 | GTGGATGTCCAGGTTAACTCAAAAA | 466 | + |
| AAA34 | AAGGTGCTTGAGCCATGGGATCAAA | 467 | − |
| AAA35 | GTAACTTCATACACTCCACATTAAA | 468 | + |
| AAA36 | TTTCGTGCAAGTCAACAATTGAAAA | 469 | + |
| CCC1 | GCATGAGGCCCTGATGCAGTGACCC | 470 | + |
| CCC2 | AACGGTGATGTCGTCAAAGATTCCC | 471 | − |
| CCC3 | CAGACATCTCCTAGCGAGTCAGCCC | 472 | − |
| CCC4 | ATTGGTGTCTCCCCGAGCTGTACCC | 473 | + |
| CCC5 | TCGCCATATCGTACCGATGTCTCCC | 474 | − |
| CCC6 | CGTAGACTAACCGACTCATCGACCC | 475 | + |
| CCC7 | ACTGACCGTTTAAGGGTCCAAGCCC | 476 | + |
| CCC8 | TACGTTCACCATCGTCAATAGGCCC | 477 | + |
| CCC9 | GTCGCCACGAACGCTGAAGAAGCCC | 478 | + |
| CCC10 | GCTGCACGTTGTCTCACAGCTTCCC | 479 | − |
| CCC11 | GCTACGCGTCCTCCAATATGCGCCC | 480 | + |
| CCC12 | GAGTAGGGTAATACGTTCTACACCC | 481 | − |
| CCC13 | GAACCCTTTAGCTCCACAATTGCCC | 482 | − |
| CCC14 | AATAACGCATGCGTTATCCCACCCC | 483 | + |
| CCC15 | AATGATCAACGAACGTCGCTGGCCC | 484 | + |
| CCC16 | TATAGCAATGAGGGCCAGTGATCCC | 485 | + |
| CCC17 | TAGCTAAGCTTGTGCTAGATTACCC | 486 | + |
| CCC18 | ACGGCGTCAGTTGTAAGGATATCCC | 487 | + |
| CCC19 | TATGATAACCCACTTCCAAGTTCCC | 488 | − |
| CCC20 | CCATAACCTTAGTATGTAGTCGCCC | 489 | + |
| CCC21 | CGTCTGTGGCAATAACGCTTCACCC | 490 | + |
| CCC22 | TATGCTTCCTGGAGCTGCAAGCCCC | 491 | + |
| CCC23 | CGGCATTCTGAACAACTATATGCCC | 492 | + |
| CCC24 | CGCATCTGCACGTAAAACGGCGCCC | 493 | − |
| CCC25 | TCAGGGCTACGCGACCTCGTACCCC | 494 | + |
| CCC26 | ATGCCGAGATTCGAATATCGGACCC | 495 | + |
| CCC27 | CCAAATTCCGCGGGCCTTGAACCCC | 496 | + |
| CCC28 | ACTTGCGTACCCATACATGTATCCC | 497 | − |
| CCC29 | TAGAAGCGCGAAGTATAGGATGCCC | 498 | − |
| CCC30 | TAGTACCGGCAATTCCTTGTTGCCC | 499 | + |
| CCC31 | AACCACGAGTCGTCACTGACCGCCC | 500 | + |
| CCC32 | GTAAATGGTCTAGAGGTTACGGCCC | 501 | − |
| CCC33 | CGTCGGATTGTGCTATGTAAAACCC | 502 | + |
| CCC34 | GACAGTTCATCTACACATTGCACCC | 503 | + |
| CCC35 | AAGGGAACCGGCACGAATCAGTCCC | 504 | + |
| CCC36 | TCATTGCTAGCACCTACCAGACCCC | 505 | − |

Screening for Minimally Interacting Primers for Use in the 24×24 Primer Matrix

The 72 candidate PCR primers shown above in TABLE 25 were screened as described below in order to identify a subset of 24 column primers and 24 row primers that would collectively define a primer matrix with low levels of primer-dimer formation.

The 72 candidate PCR primers for use in a primer matrix were resuspended to a working concentration of 10 μM in water. A grid of 36 by 36 wells containing PCR master mix was prepared by aliquoting 25 μl of 2× power SYBR master mix (Applied Biosystems, Foster City, Calif.) and 17 μl of water in each well of a set of 384 well optical PCR plates as follows.

4 μl of column primers (“AAA”:SEQ ID NOS:434-469) were added to each well along each column and 4 μl of row primers (“CCC”:SEQ ID NOS:470-505) were added to each well along each row. Following mixing, four 10 μl aliquots from each well were distributed in quadruplicate into 384 optical PCR plates as shown below in TABLE 26 and analyzed for qPCR using 40 cycles on an ABI 7900 qPCR instrument following the standard cycling protocol. The average Ct of each set of quadruplicate wells was then calculated and wells with Ct values of less than 38 were identified (as shown in bold in TABLE 26) as wells in which primer pairs were able to interact to form detectable PCR products at unacceptably low Cts. The primer pairs with Ct values>38 are indicated with a “+” symbol, indicating that the primer pairs are useful for inclusion in the final matrix.

TABLE 26
PRIMER PAIRS THAT PRODUCED CT VALUES LESS THAN 38 ARE SHOWN IN BOLD

| Ct values | AAA1 | AAA2 | AAA3 | AAA4 | AAA5 | AAA6 | AAA7 | AAA8 | AAA9 | AAA10 | AAA11 | AAA12 |
|---|---|---|---|---|---|---|---|---|---|---|---|---|
| CCC1 | + | + | + | + | + | + | + | + | + | + | + | + |
| CCC2 | + | 32 | + | + | + | + | + | + | + | + | + | 33 |
| CCC3 | + | + | + | + | + | + | + | + | + | + | 35 | + |
| CCC4 | + | + | 38 | + | + | + | + | + | + | + | + | + |
| CCC5 | + | + | + | + | + | + | 36 | + | + | + | + | + |
| CCC6 | + | + | + | + | + | + | + | + | + | + | + | + |
| CCC7 | + | + | + | + | + | + | + | + | + | + | + | + |
| CCC8 | + | + | + | + | + | + | + | + | + | + | + | + |
| CCC9 | + | + | + | + | + | + | + | + | + | + | + | + |
| CCC10 | + | 25 | + | + | + | + | + | + | + | + | + | 27 |
| CCC11 | + | + | + | + | + | + | + | + | + | + | + | + |
| CCC12 | + | + | 35 | + | + | + | + | + | + | + | + | + |
| CCC13 | + | + | + | + | 36 | + | + | + | + | + | + | + |
| CCC14 | + | + | + | + | + | + | + | + | + | + | + | + |
| CCC15 | + | + | + | + | + | + | + | + | + | + | + | + |
| CCC16 | + | + | + | + | + | + | + | + | + | + | + | + |
| CCC17 | + | + | + | + | + | + | + | + | + | + | + | + |
| CCC18 | + | + | + | + | + | + | + | + | + | + | + | + |
| CCC19 | + | 34 | + | + | + | + | + | + | + | + | + | 35 |
| CCC20 | + | + | + | + | + | + | + | + | + | + | + | + |
| CCC21 | + | + | + | + | + | + | + | + | + | + | + | + |
| CCC22 | + | + | + | + | + | + | + | + | + | + | + | + |
| CCC23 | + | + | + | + | + | + | + | + | + | + | + | + |
| CCC24 | + | + | + | + | + | + | 36 | + | 38 | + | 37 | + |
| CCC25 | + | + | + | + | + | + | + | + | + | + | + | + |
| CCC26 | + | + | + | + | + | + | + | + | + | + | + | + |
| CCC27 | + | + | + | + | + | + | 38 | + | + | + | + | + |
| CCC28 | + | + | + | + | + | + | 30 | + | + | + | + | + |
| CCC29 | + | + | + | + | + | + | + | + | + | + | + | + |
| CCC30 | + | + | + | + | + | + | + | + | + | + | + | + |
| CCC31 | + | + | + | + | + | + | + | + | + | + | + | + |
| CCC32 | + | + | + | + | + | + | + | + | 30 | + | + | + |
| CCC33 | + | + | + | + | + | + | + | + | + | + | + | + |
| CCC34 | + | + | + | + | + | + | + | + | + | + | + | + |
| CCC35 | + | + | + | + | + | + | + | + | + | + | + | + |
| CCC36 | + | + | 33 | + | + | + | + | + | + | + | + | + |
| Ct values | AAA13 | AAA14 | AAA15 | AAA16 | AAA17 | AAA18 | AAA19 | AAA20 | AAA21 | AAA22 | AAA23 | AAA24 |
| CCC1 | + | + | + | + | + | + | + | + | + | + | + | + |
| CCC2 | + | + | + | + | + | + | 36 | + | + | + | + | + |
| CCC3 | + | + | + | + | 36 | + | + | + | 27 | + | + | + |
| CCC4 | + | + | + | + | + | + | + | + | + | + | + | + |
| CCC5 | + | + | + | + | + | + | + | + | 37 | + | 37 | + |
| CCC6 | + | + | + | + | + | + | + | + | + | + | + | + |
| CCC7 | + | + | + | + | + | + | + | + | + | 38 | + | + |
| CCC8 | + | + | + | + | + | + | + | + | + | + | + | + |
| CCC9 | + | + | + | + | + | + | + | + | + | 35 | + | + |
| CCC10 | + | + | + | + | 33 | + | + | + | 37 | + | + | + |
| CCC11 | + | + | + | + | 37 | + | + | + | + | 38 | + | + |
| CCC12 | + | + | + | + | + | + | + | + | + | + | + | + |
| CCC13 | + | + | + | + | 37 | 36 | + | + | + | + | + | + |
| CCC14 | + | + | + | + | + | + | + | + | + | + | + | + |
| CCC15 | + | + | + | + | 34 | + | + | + | 37 | + | + | + |
| CCC16 | + | + | + | + | + | + | + | + | + | + | + | + |
| CCC17 | + | + | + | + | + | + | + | + | + | + | + | + |
| CCC18 | + | + | + | + | + | + | + | + | + | + | + | + |
| CCC19 | + | + | + | + | + | + | + | + | + | + | + | + |
| CCC20 | + | + | + | + | + | + | + | + | 34 | 35 | + | + |
| CCC21 | + | + | + | + | + | + | + | + | + | + | + | + |
| CCC22 | + | + | + | + | + | + | + | + | + | + | + | + |
| CCC23 | + | + | + | + | + | + | + | + | + | + | + | + |
| CCC24 | + | + | 37 | + | + | + | + | + | 36 | 35 | + | + |
| CCC25 | + | + | + | + | + | + | + | + | + | + | + | + |
| CCC26 | + | + | + | + | + | + | + | + | + | + | + | + |
| CCC27 | + | + | + | + | + | + | + | + | + | + | + | + |
| CCC28 | + | + | + | + | + | + | + | + | + | + | + | + |
| CCC29 | + | + | + | + | 32 | + | + | + | 29 | 35 | + | + |
| CCC30 | + | + | + | + | 35 | + | + | + | + | + | + | + |
| CCC31 | + | + | + | + | 36 | + | + | + | 34 | 33 | + | + |
| CCC32 | + | + | + | + | 37 | + | + | + | + | 38 | + | + |
| CCC33 | + | + | + | + | + | + | + | + | + | + | + | + |
| CCC34 | + | + | + | + | + | + | + | + | + | + | + | + |
| CCC35 | + | + | + | + | + | + | + | + | + | + | + | + |
| CCC36 | + | + | + | + | + | + | + | + | + | + | + | + |
| Ct values | AAA25 | AAA26 | AAA27 | AAA28 | AAA29 | AAA30 | AAA31 | AAA32 | AAA33 | AAA34 | AAA35 | AAA36 |
| CCC1 | + | + | + | + | + | + | + | + | + | + | + | + |
| CCC2 | + | + | + | + | 34 | + | + | + | + | + | + | + |
| CCC3 | + | + | + | + | + | + | + | + | + | + | + | + |
| CCC4 | + | + | + | + | + | + | + | + | + | + | + | + |
| CCC5 | + | + | + | + | + | + | + | + | + | + | + | + |
| CCC6 | 37 | + | + | + | + | 37 | + | + | + | + | + | + |
| CCC7 | + | + | + | + | + | + | + | + | + | + | + | + |
| CCC8 | + | + | + | + | + | + | 38 | + | + | 30 | + | + |
| CCC9 | + | + | + | + | + | + | + | + | + | + | + | + |
| CCC10 | + | + | + | + | 29 | + | + | + | + | + | + | + |
| CCC11 | + | + | + | + | + | 31 | + | + | + | + | + | + |
| CCC12 | + | + | + | + | + | + | + | + | + | + | + | + |
| CCC13 | + | + | + | + | + | 35 | + | + | + | + | + | + |
| CCC14 | + | + | + | + | + | + | + | + | + | + | + | + |
| CCC15 | + | + | + | + | + | + | + | + | + | + | + | + |
| CCC16 | + | + | + | + | + | 38 | + | + | + | 30 | + | + |
| CCC17 | + | + | + | + | + | + | + | + | + | + | + | + |
| CCC18 | + | + | + | + | + | + | + | + | + | + | + | + |
| CCC19 | + | + | + | + | 36 | + | + | + | + | + | + | + |
| CCC20 | + | + | + | + | + | 26 | + | + | + | + | + | + |
| CCC21 | + | + | + | + | + | + | + | + | + | + | + | + |
| CCC22 | + | + | + | + | + | + | 36 | + | + | + | + | + |
| CCC23 | + | + | + | + | + | + | + | + | + | + | + | + |
| CCC24 | + | + | + | + | + | 34 | 36 | + | + | + | + | + |
| CCC25 | + | + | + | + | + | + | + | + | + | + | + | + |
| CCC26 | + | + | + | + | + | + | + | + | + | + | + | + |
| CCC27 | + | + | + | + | + | + | + | + | + | 34 | + | + |
| CCC28 | + | + | + | + | + | + | + | + | + | + | + | + |
| CCC29 | + | + | + | + | + | + | + | + | + | + | + | + |
| CCC30 | + | + | + | + | + | 37 | + | + | + | + | + | + |
| CCC31 | + | + | + | + | + | 30 | + | + | + | + | + | + |
| CCC32 | + | + | + | + | + | + | + | + | + | + | + | + |
| CCC33 | + | + | + | + | + | + | 34 | + | + | + | + | + |
| CCC34 | + | + | + | + | + | + | + | + | + | + | + | + |
| CCC35 | + | + | + | + | + | + | + | + | + | + | + | + |
| CCC36 | + | + | + | + | + | + | 38 | + | + | + | + | + |

TABLE 27
FINAL CONFIGURATION OF 24 × 24 PRIMER MATRIX”

| | AAA1 | AAA2 | AAA4 | AAA5 | AAA6 | AAA8 | AAA10 | AAA11 | AAA12 | AAA13 | AAA15 | AAA16 |
|---|---|---|---|---|---|---|---|---|---|---|---|---|
| CCC1 | + | + | + | + | + | + | + | + | + | + | + | + |
| CCC4 | + | + | + | + | + | + | + | + | + | + | + | + |
| CCC6 | + | + | + | + | + | + | + | + | + | + | + | + |
| CCC7 | + | + | + | + | + | + | + | + | + | + | + | + |
| CCC8 | + | + | + | + | + | + | + | + | + | + | + | + |
| CCC9 | + | + | + | + | + | + | + | + | + | + | + | + |
| CCC11 | + | + | + | + | + | + | + | + | + | + | + | + |
| CCC14 | + | + | + | + | + | + | + | + | + | + | + | + |
| CCC15 | + | + | + | + | + | + | + | + | + | + | + | + |
| CCC16 | + | + | + | + | + | + | + | + | + | + | + | + |
| CCC17 | + | + | + | + | + | + | + | + | + | + | + | + |
| CCC18 | + | + | + | + | + | + | + | + | + | + | + | + |
| CCC20 | + | + | + | + | + | + | + | + | + | + | + | + |
| CCC21 | + | + | + | + | + | + | + | + | + | + | + | + |
| CCC22 | + | + | + | + | + | + | + | + | + | + | + | + |
| CCC23 | + | + | + | + | + | + | + | + | + | + | + | + |
| CCC25 | + | + | + | + | + | + | + | + | + | + | + | + |
| CCC26 | + | + | + | + | + | + | + | + | + | + | + | + |
| CCC27 | + | + | + | + | + | + | + | + | + | + | + | + |
| CCC30 | + | + | + | + | + | + | + | + | + | + | + | + |
| CCC31 | + | + | + | + | + | + | + | + | + | + | + | + |
| CCC33 | + | + | + | + | + | + | + | + | + | + | + | + |
| CCC34 | + | + | + | + | + | + | + | + | + | + | + | + |
| CCC35 | + | + | + | + | + | + | + | + | + | + | + | + |
| | AAA18 | AAA19 | AAA20 | AAA24 | AAA26 | AAA27 | AAA28 | AAA29 | AAA32 | AAA33 | AAA35 | AAA36 |
| CCC1 | + | + | + | + | + | + | + | + | + | + | + | + |
| CCC4 | + | + | + | + | + | + | + | + | + | + | + | + |
| CCC6 | + | + | + | + | + | + | + | + | + | + | + | + |
| CCC7 | + | + | + | + | + | + | + | + | + | + | + | + |
| CCC8 | + | + | + | + | + | + | + | + | + | + | + | + |
| CCC9 | + | + | + | + | + | + | + | + | + | + | + | + |
| CCC11 | + | + | + | + | + | + | + | + | + | + | + | + |
| CCC14 | + | + | + | + | + | + | + | + | + | + | + | + |
| CCC15 | + | + | + | + | + | + | + | + | + | + | + | + |
| CCC16 | + | + | + | + | + | + | + | + | + | + | + | + |
| CCC17 | + | + | + | + | + | + | + | + | + | + | + | + |
| CCC18 | + | + | + | + | + | + | + | + | + | + | + | + |
| CCC20 | + | + | + | + | + | + | + | + | + | + | + | + |
| CCC21 | + | + | + | + | + | + | + | + | + | + | + | + |
| CCC22 | + | + | + | + | + | + | + | + | + | + | + | + |
| CCC23 | + | + | + | + | + | + | + | + | + | + | + | + |
| CCC25 | + | + | + | + | + | + | + | + | + | + | + | + |
| CCC26 | + | + | + | + | + | + | + | + | + | + | + | + |
| CCC27 | + | + | + | + | + | + | + | + | + | + | + | + |
| CCC30 | + | + | + | + | + | + | + | + | + | + | + | + |
| CCC31 | + | + | + | + | + | + | + | + | + | + | + | + |
| CCC33 | + | + | + | + | + | + | + | + | + | + | + | + |
| CCC34 | + | + | + | + | + | + | + | + | + | + | + | + |
| CCC35 | + | + | + | + | + | + | + | + | + | + | + | + |

“+” symbol indicates Ct value is 38 or higher

As indicated above in TABLE 27, the final 24 primer by 24 primer matrix used for the qPCR amplification of the ligation-dependent genotyping assay carries no primer pairs that produced a Ct value of less than 38, and therefore all the primer pairs contained in this primer matrix are minimally interacting primer pairs suitable for use in the genotyping assays described herein.

The 24 by 24 primer grid provides 576 unique primer pairs (i.e., features) that can be used to perform consensus versus variant genotyping assays on 288 putative SNV positions (288 consensus plus 288 variant assays=576 PCR reactions). Therefore, the matrix can be used with sets of 288 assays, as demonstrated below in EXAMPLE 7.

### Example 7

This Example demonstrates the use of the 24 by 24 primer matrix described in Example 6 for use in the ligation-dependent genotyping assay for genotyping 799 putative SNV locations identified during DNA sequencing of 14 Pichia pastoris yeast strains.

Rationale: High throughput sequencing of 14 Pichia pastoris yeast strains indicated that as many as 799 SNVs that differed from the Pichia pastoris reference sequence may be present in one or more strains that were examined. In order to further examine these putative SNV locations, we generated 799 consensus and variant genotyping assays with synthetic consensus and variant DNA templates.

Methods:

1. Preparation of Assay Oligos:

A set of 799 genotyping reagents was generated for the 799 SNV positions of interest, including 5′ ligation oligos (consensus and variant), 3′ common ligation oligos and synthetic consensus and variant templates for each SNV position of interest, using the same design criteria as described above in Example 5 (oligo sequences not shown).

2. Pooling of Oligos:

To perform the genotyping assays, the 799 genotyping oligos were divided into two sets of 288 assays and one set of the remaining 223 assays.

For each set of assays (i.e., the first set of 288 assays, the second set of 288 assays and the third set of 223 assays), consensus and variant 5′ ligation oligos were pooled and diluted to 500 nM (860 pM in each unique oligo).

Similarly, the common 3′ ligation oligos for each set of assays was pooled, treated with kinase to add a 5′ terminal phosphate as described in Example 5, and diluted to a final working concentration of 250 nM (860 pM in individual oligos).

Finally, for each set of assays, pools of 288 or 233 consensus template oligos, and pools of 288 or 233 variant template oligos were pooled and diluted to 100 pM.

3. Annealing

The ligation-dependent genotyping assays were performed by the decoupled annealing and ligation method, as follows.

Annealing Reaction

For each assay reaction, the following reagents were combined:

10 μl of 100 pM pooled templates (variant or consensus)

10 μl of 500 nM pooled consensus and variant 5′ ligation oligos

10 μl of 250 nM kinased 3′ common ligation oligos

2 μl of 5 M NaCl

32 μl total volume

Annealing:

The rapid annealing protocol was carried out as follows:

95° C. for 5 minutes;

75° C. for 15 minutes;

70° C. for 15 minutes;

65° C. for 30 minutes;

25° C. rest.

4. Ligation

For each assay, 68 μl of a ligation cocktail was added to the 32 μl annealed mixture, the ligation cocktail containing:

10 μl of 10× Taq DNA ligase buffer (NEB)

57 μl of H₂O

1 μl of 40 U/μl Taq DNA Ligase (NEB)

100 μl total volume

Note: 3 different reaction mixtures were prepared, one for each assay: the first set of 288 assays, the second set of 288 assays and the third set of 223 assays.

The ligation mixtures were incubated at 45° C. for 5 minutes and diluted to 1 ml with 900 μl of TEzero (10 mM Tris pH 7.6, 0.1 mM EDTA). Six such identical reactions were run for each set of 288 consensus or 288 variant assays in order to provide enough material to assay on PCR plates.

5. qPCR Assay

For the qPCR assay readout, 2 μl of the ligation mixture was assayed in a 10 μl reaction volume containing 5 μl of 2× power SYBR master mix (Applied Biosystems, Foster City, Calif.), 1.4 μl H₂O, 0.8 μl column matrix primer and 0.8 μl row matrix primer.

Each mixture was aliquoted in quadruplicate across a matrix of 24 by 24 separate PCR reactions, as described in Example 6, which translated into 6 independent 384 well optical PCR plates per set of 288 genotyping assays.

The PCR plates were run on an Applied Biosystems 7900 qPCR instrument according to the manufacturer's instructions.

Results:

The average Cts across quadruplicate wells were calculated for each consensus and variant pair of the 799 SNV assay set. It was determined that all of the assays involving the column primer AAA29 (SEQ ID NO:461: 5′ATCTATCTTGAACCCGGGCGATAAA 3′) yielded Ct values greater than 35, whereas the standard genotyping Ct readout was below 30 for all the other primer pairs. This indicated that the AAA29 primer (SEQ ID NO:461) does not support robust PCR amplification, and therefore all the assays (32 total assays) using this primer SEQ ID NO:461 were not evaluated further. To avoid inclusion of poor performing primers such as the AAA29 primer, in the future, matrix primer screening, as described in Example 6, will also include a positive test against synthetic templates for functional PCR amplification performance. In the present example, a 36×36 matrix of primers was screened, using the methods described in Example 6, and it was determined that only about 5 to 6 row primers and 5 to 6 column primers were poor performers (i.e., high background, low Cts). In the process of choosing primers from this screen for use in the 24×24 matrix, many primers were excluded that would fit the criteria of good performers. One of these good but previously excluded primers was substituted for primer SEQ ID NO:461 in the matrix and the assay worked well with the substituted primer (data not shown).

For the remaining 767 assays, the Ct(variant)−Ct(consensus)=Δ consensus for consensus templates and the Ct(consensus)−Ct(variant)=Δ variant for variant templates was calculated. The performance of the ligation-dependent genotyping assay was evaluated based on the sum of Δ consensus+Δ variant. It was empirically determined that if the sum of Δ consensus+Δ variant is greater than 3, then genotyping calls can be made with confidence in diploid organisms. This was established in separate experiments by genotyping of two inbred mouse strains and their F1 progeny at known SNPs. In this system, the parental strains were uniformly homozygous and the progeny were uniformly heterozygous at every SNP location. A survey of 576 independent SNP assays in this system revealed the greatest accuracy when only the genotyping assays were considered that had a Δ consensus+Δ variant value of greater than 3 (data not shown).

For haploid organisms such as P. pastoris, the genotyping results are expected to be even more accurate, because only two genotypes are possible (consensus or variant), in contrast to the case in diploid species where three genotypes are possible (consensus, variant, or heterozygote). Hence, in haploid organisms, the expected genotype will only be consensus or variant, and not potentially a heterozygous blend of the two as is found in a diploid organism such as a human. Therefore, for haploid organisms, the value of Ct(variant)−Ct(consensus) is predicted to resemble either Δ consensus or −Δ variant.

The Δ consensus+Δ variant values for all 767 functional assays were calculated. Of these, 730 (95%) had values greater than or equal to 3, indicating that the genotyping calls can be made with confidence. Of the 37 failed assays (values below 3), it is interesting to note that 19 of them shared overlapping DNA sequences in two groups of 7 assays and 12 assays, respectively. Subsequent in-house comparisons of two independently generated draft genome sequences of Pichia pastoris revealed almost perfect identity except in these regions, where the assembled sequences disagreed. While not wishing to be bound by any particular theory, this suggests that these regions are generally difficult to sequence and that the sequences that were genotyped may not exist in P. pastoris. If the DNA sequences of the genotyping primers do not match those of the target region, then the genotyping assays would be expected to fail. The remaining 18 failed assays occurred across unrelated sequence groups.

In summary, this Example demonstrates that of the 767 ligation-dependent genotyping assays carried out that were designed to query random SNVs, 95% of the assays returned useful data. This percent of discovered SNVs that can be assayed in a particular technology platform with high confidence, otherwise referred to as “conversion rate” in the genotyping field, is very high and comparable to other commercially available platforms such as the Affymetrix SNP array or the Illumina Bead array.

Unlike commercially available genotyping solutions, which are fixed and can only monitor known SNPS, the ligation-dependent genotyping assays described herein combine the advantages of a highly successful conversion rate and the flexibility to monitor novel single-nucleotide variants. The ligation-dependent genotyping assays as described herein are therefore a unique, low cost solution to the validation of putative sequence variants that are suggested by high-throughput resequencing technologies.

While the preferred embodiment of the invention has been illustrated and described, it will be appreciated that various changes can be made therein without departing from the spirit and scope of the invention.

What is claimed:

1. A method of determining the genotype of a test sample at one or more polymorphic loci of interest, the method comprising:
a) contacting, in a reaction mixture, one or more set(s) of query oligonucleotides with the test sample having one or more polymorphic loci of interest within one or more target nucleic acid region(s) of interest, wherein each set of query oligonucleotides comprises:
(i) at least one 5′ ligation oligonucleotide having, from the 5′ to 3′ end, a first PCR primer binding region, a target-specific binding region selected to hybridize 5′ of a polymorphic locus of interest, and a 3′ region chosen to hybridize to either a consensus or variant nucleotide sequence at the polymorphic loci of interest; and
(ii) a phosphorylated 3′ ligation oligonucleotide having, from the 5′ to 3′ end, a target-specific binding region selected to hybridize 3′ of the polymorphic loci of interest and a second PCR primer binding region;
under conditions that allow hybridization between the one or more set(s) of query oligonucleotides and the target nucleic acid region(s) of interest such that the 5′ ligation oligonucleotides and the phosphorylated 3′ ligation oligonucleotides hybridize adjacent to each other on the target nucleic acid region(s) of interest;
b) contacting the reaction mixture of step (a) with a DNA ligase under conditions suitable to ligate the 5′ ligation oligonucleotides and the adjacent 3′ phosphorylated ligation oligonucleotides, thereby generating a plurality of ligation products indicative of the genotype of the test sample at the one or more polymorphic loci of interest; and
c) measuring the amount of the plurality of ligation products in the reaction mixture of step (b).

2. The method of claim 1, further comprising the step of comparing the amount of plurality of ligation products measured according to step (c) with at least one reference standard that is indicative of the presence or absence of the consensus or variant nucleotide at each polymorphic loci of interest.

3. The method of claim 1, wherein in the reaction mixture of step a) the test sample is contacted with the one or more set(s) of query oligonucleotides and the DNA ligase under conditions that allow hybridization between the one or more set(s) of query oligonucleotides and the target nucleic acid region(s) of interest and to allow the query oligonucleotides to hybridize adjacent to each other on the target nucleic acid region of interest, and to allow ligation of the 5′ ligation oligonucleotide and the adjacent 3′ phosphorylated ligation oligonucleotides, thereby generating a plurality of ligation products, so as to couple hybridization and ligation in the reaction mixture.

4. The method of claim 1, wherein the test sample comprises a haploid or diploid genome.

5. The method of claim 1, wherein the test sample comprises non-amplified target nucleic acid region(s) of interest.

6. The method of claim 1, wherein each set of query oligonucleotides according to step (a) comprises a pair of allele-specific 5′ ligation oligonucleotides for each polymorphic loci of interest, the pair comprising (i) a first 5′ ligation oligonucleotide comprising a 3′ region chosen to hybridize to a consensus nucleotide sequence at the polymorphic loci of interest and (ii) a second 5′ ligation oligonucleotide comprising a 3′ region chosen to hybridize to a variant nucleotide sequence at the polymorphic loci of interest.

7. The method of claim 1, wherein the 5′ ligation oligonucleotides comprise the first PCR primer binding region having different nucleotide sequences.

8. The method of claim 1, wherein step (a) comprises contacting, in the single reaction mixture, the test sample with at least 10 sets of query oligonucleotides for genotyping at least 10 different polymorphic loci positions of interest.

9. The method of claim 1, wherein the DNA ligase is thermostable.

10. The method of claim 1, wherein the measuring in step (c) comprises amplifying the plurality of ligation products with one or more pair(s) of detection primers, each detection primer pair having (i) a forward PCR primer that binds to the first PCR primer binding region in the 5′ ligation oligonucleotide and (ii) a reverse PCR primer that binds to the second PCR primer binding region in the 3′ ligation oligonucleotide.

11. The method of claim 1, wherein the measuring in step (c) comprises amplifying the plurality ligation products with: (i) a first pair of detection primers having a forward PCR primer that binds to the PCR binding region of the 5′ ligation oligonucleotide comprising the consensus binding region; and with (ii) a second pair of detection primers comprising a forward PCR primer that binds to the PCR binding region of the 5′ ligation oligonucleotide comprising the variant binding region.

12. The method of claim 10, wherein the penultimate 2 or 3 nucleotides at the 3′ end of the first pair or second pair of detection primers are selected to reduce primer-dimer formation by selecting 2 or 3 nucleotide that reduce annealing between the first and second pair of detection primers or that reduce self-annealing of the first and second pair of detection primers.

13. The method of claim 1, wherein the measuring in step (c) comprises measuring fluorescence.

14. The method of claim 1, wherein the measuring in step (c) includes contacting the ligation product with a dye that intercalates double-stranded DNA.

15. The method of claim 10, wherein the one or more pair(s) of detection primers comprise a fluorescent label.

16. The method of claim 11, wherein the first or second pair of detection primers comprise a fluorescent label.

17. The method of claim 1, wherein the test sample is enriched for the one or more target region(s) of interest prior to the contacting of step (a).

18. The method of claim 1, wherein the query oligonucleotides each have a length of about 40 nucleotides to about 200 nucleotides.

19. The method of claim 1, wherein the target-specific binding region of the query oligonucleotides have a length of about 10 nucleotides to about 150 nucleotides in length.

20. A method of genotyping a test sample at one or more single nucleotide variant(s) (SNVs) position(s) of interest, the method comprising: for each SNV position of interest,
a) contacting in three separate reaction mixtures:
(i) a synthetic template comprising the target region of interest having a consensus nucleotide at the SNV position of interest;
(ii) a synthetic template comprising the target region of interest having a variant nucleotide at the SNV position of interest; and
(iii) a test sample comprising the target region of interest comprising the SNV position of interest to be genotyped;
with one or more set(s) of SNV query oligonucleotides, each set comprising (i) a pair of allele-specific 5′ ligation oligonucleotides, the pair comprising a first 5′ ligation oligonucleotide comprising, from the 5′ to 3′ end, a first PCR primer binding region, a target-specific binding region selected to hybridize 5′ of the SNV nucleotide position of interest, and a 3′ region chosen to hybridize to the consensus nucleotide sequence at the SNV position of interest and a second 5′ ligation oligonucleotide comprising, from the 5′ to 3′ end, a first PCR primer binding region, a target-specific binding region selected to hybridize 5′ of the SNV nucleotide position of interest, and a 3′ region chosen to hybridize to the variant nucleotide sequence at the SNV position of interest and (ii) a phosphorylated 3′ ligation oligonucleotide comprising from the 5′ to 3′ end, a target-specific binding region selected to hybridize 3′ of the SNV position of interest and a second PCR primer binding region, under conditions that allow hybridization between the one or more sets of SNV query oligonucleotides and the target regions of interest having the consensus nucleotide, the variant nucleotide, and the SNV position of interest, such that the 5′ ligation oligonucleotides and the phosphorylated 3′ ligation oligonucleotides hybridize adjacent to each other on the target region of interest;
b) contacting the three separate reaction mixtures of step (a) with a DNA ligase under conditions suitable to ligate the 5′ ligation oligonucleotides and the adjacent 3′ phosphorylated ligation oligonucleotides, thereby generating three separate mixtures each having a plurality of ligation products; and
c) measuring the amount of the plurality of ligation products in each of the three separate mixtures of step (b).

21. The method of claim 20, wherein in at least one of the three separate reaction mixtures the of step a) the test sample is contacted with the one or more set(s) of query oligonucleotides and the DNA ligase under conditions that allow hybridization between the one or more set(s) of query oligonucleotides and the target nucleic acid region(s) of interest to allow the query oligonucleotides to hybridize adjacent to each other on the target nucleic acid region of interest, and to allow ligation of the 5′ ligation oligonucleotide and the adjacent 3′ phosphorylated ligation oligonucleotides, thereby generating a plurality of ligation products.

22. The method of claim 20, wherein the test sample comprises a haploid or diploid genome.

23. The method of claim 20, wherein the test sample comprises a non-amplified target region of interest.

24. The method of claim 20, wherein the first 5′ ligation oligonucleotides comprise the first PCR primer binding regions having different nucleotide sequences.

25. The method of claim 20, wherein step (a) comprises contacting the test sample with at least 10 sets of SNV query oligonucleotides for genotyping at least 10 different SNV positions of interest.

26. The method of claim 20, wherein the DNA ligase is thermostable.

27. The method of claim 20, wherein the measuring in step (c) comprises amplifying the plurality ligation products with (i) a set of detection primers comprising forward PCR primers that bind to the first PCR binding region of the first 5′ ligation oligonucleotide comprising the consensus binding region, (ii) a set of detection primers comprising forward PCR primers that bind to the first PCR binding region of the second 5′ ligation oligonucleotide comprising the variant binding region, and (iii) a set of detection primers comprising reverse PCR primers that bind to the second PCR primer binding region in the 3′ ligation oligonucleotide.

28. The method of claim 27, wherein the penultimate 2 or 3 nucleotides at the 3′ end of the forward or reverse PCR primers are selected to reduce primer-dimer formation by selecting 2 or 3 nucleotide that reduce annealing between the first and second pair of detection primers or that reduce self-annealing of the first and second pair of detection primers.

29. The method of claim 20, wherein the measuring in step (c) comprises measuring fluorescence.

30. The method of claim 20, wherein the measuring in step (c) comprises contacting the plurality of ligation products with a dye that intercalates double-stranded DNA.

31. The method of claim 27, wherein the forward PCR primer or the reverse PCR primer comprises a fluorescent label.

32. The method of claim 20, wherein the test sample is enriched for the one or more target region(s) of interest prior to the contacting in step (a).

33. The method of claim 20, wherein the SNV query oligonucleotides have a length of about 40 nucleotides to about 200 nucleotides.

34. The method of claim 20, wherein the target-specific binding region of the SNV query oligonucleotides have a length of about 10 nucleotides to about 150 nucleotides in length.

35. A two-dimensional nucleic acid matrix comprising forward and reverse primer pairs and ligation products distributed into positionally addressable wells, wherein the wells include:
a) the forward PCR primers each having
(i) a 5′ region that hybridizes to a 5′ primer binding region of a target nucleic acid molecule of interest and
(ii) a 3′ region selected to avoid primer-dimer formation with the reverse primer
b) the reverse PCR primers each having
(i) a 5′ region that hybridizes to a 3′ primer binding region of the target nucleic acid molecule of interest and
(ii) a 3′ region selected to avoid primer-dimer formation with the forward primer; and
c) ligation products generated by annealing the target nucleic acid molecule of interest with
(i) a 5′ ligation oligonucleotide having from the 5′ to 3′ end, the reverse PCR primer binding region, a target-specific binding region selected to hybridize 5′ of a polymorphic locus of interest, and a 3′ region chosen to hybridize to either a consensus or variant nucleotide sequence at the polymorphic locus of interest and
(ii) an adjacent phosphorylated 3′ ligation oligonucleotide having from the 5′ to 3′ end, a target-specific binding region selected to hybridize 3′ of the polymorphic locus of interest and a forward PCR primer binding region and
(iii) ligating the 5′ ligation oligonucleotides and the adjacent 3′ phosphorylated ligation oligonucleotides so as to generate the ligation products.

36. The matrix of claim 35, wherein the 5′ ligation oligonucleotides comprise the reverse PCR primer binding region having different sequences.

37. The matrix of claim 35, wherein the penultimate 2 or 3 nucleotides of the 3′ region in the forward and reverse PCR primers are selected to reduce primer-dimer formation by selecting 2 or 3 nucleotide that reduce annealing between the first and second pair of detection primers or that reduce self-annealing of the first and second pair of detection primers.

38. The matrix of claim 37, wherein the 3′ region selected to avoid primer-dimer formation in the forward PCR primers comprises the nucleotide sequence “CT” and the 3′ region selected to avoid primer-dimer formation in the reverse primers comprises the nucleotide sequence “GA.”

39. The matrix of claim 37, wherein the 3′ region selected to avoid primer-dimer formation in the forward PCR primers comprises the nucleotide sequence “ACA” and the 3′ region selected to avoid primer-dimer formation in the reverse primers comprises of the nucleotide sequence “CAC.”

40. The matrix of claim 37, wherein the 3′ region selected to avoid primer-dimer formation excludes “TTT” and “GGG” sequences.

41. The matrix of claim 37, wherein the 3′ region selected to avoid primer-dimer formation in the forward PCR primer comprises a terminal sequence of “CCC” and the 3′ region selected to avoid primer-dimer formation in the reverse PCR primer comprises a terminal sequence of terminal sequence of “AAA”.

42. The matrix of claim 37, wherein the 3′ region selected to avoid primer-dimer formation in the forward PCR primer comprises a terminal sequence of “AAA” and the 3′ region selected to avoid primer-dimer formation in the reverse PCR primer comprises a terminal sequence of terminal sequence of “CCC”.

43. The matrix of claim 37, wherein the last nine nucleotides of the forward and reverse PCR primer sequences are selected to exclude the sequence “ACA” or “TGT.”

44. The matrix of claim 35, wherein the total length of the forward and reverse PCR primers comprises about 15 to 35 nucleotides.

45. The matrix of claim 35, wherein the 3′ region selected to avoid primer-dimer formation in the forward and reverse PCR primers comprises 6 nucleotides.

46. The matrix of claim 35, further comprising an enzyme reaction mixture for PCR amplification.

47. A kit for genotyping a test sample at one or more polymorphic loci of interest, the kit comprising:
a) at least one set of query oligonucleotides for genotyping a polymorphic loci of interest, the set including: (i) at least one 5′ ligation oligonucleotide having, from the 5′ to 3′ end, a first PCR primer binding region, a target-specific binding region selected to hybridize 5′ of the polymorphic loci of interest, and a 3′ region chosen to hybridize to either a consensus or variant nucleotide sequence at the polymorphic loci of interest, and (ii) a phosphorylated 3′ ligation oligonucleotide having, from the 5′ to 3′ end, a target-specific binding region selected to hybridize 3′ of the polymorphic loci of interest and a second PCR primer binding region; and
b) one or more pair(s) of detection primers, each detection primer pair having (i) a forward PCR primer that binds to the first PCR primer binding region in the 5′ ligation oligonucleotide and (ii) a reverse PCR primer that binds to the second PCR primer binding region in the 3′ ligation oligonucleotide.

48. The kit of claim 47, wherein the 5′ ligation oligonucleotide comprises the 5′ PCR primer binding region having different nucleotide sequences.

49. The kit of claim 47, wherein the forward and reverse PCR primers comprise penultimate 2 or 3 nucleotides at the 3′ end that are selected to reduce primer-dimer formation by selecting 2 or 3 nucleotide that reduce annealing between the first and second pair of detection primers or that reduce self-annealing of the first and second pair of detection primers.

50. The kit of claim 47, further comprising a DNA ligase.

51. The kit of claim 50, wherein the ligase is thermostable.

52. The kit of claim 47, further comprising at least one nucleic acid sample having a consensus nucleotide sequence or a variant nucleotide sequence at the polymorphic locus of interest.

53. The kit of claim 47, wherein the one or more pair(s) of detection primers are disposed in a multi-well container.
